SEQUENCE LISTING (40 sequences)

SEQ ID NO: 1 - DNA, 39 bp, synthetic construct
  source 1..39: mol_type = other DNA
gcctaatgtggtgcgtagactacttatccggagatctgc

SEQ ID NO: 2 - DNA, 40 bp, synthetic construct
  source 1..40: mol_type = other DNA
gtctacgcaccacattaggctgaacctgtagagatttttc

SEQ ID NO: 3 - DNA, 39 bp, synthetic construct
  source 1..39: mol_type = other DNA
gcctaataccgtgcgtagactacttatccggagatctgc

SEQ ID NO: 4 - DNA, 40 bp, synthetic construct
  source 1..40: mol_type = other DNA
gtctacgcacggtattaggctgaacctgtagagatttttc

SEQ ID NO: 5 - DNA, 27 bp, synthetic construct
  source 1..27: mol_type = other DNA
gcgaagctggggndttccctgataccc

SEQ ID NO: 6 - DNA, 27 bp, synthetic construct
  source 1..27: mol_type = other DNA
gcgaagctggggvhgtccctgataccc

SEQ ID NO: 7 - DNA, 27 bp, synthetic construct
  source 1..27: mol_type = other DNA
gcgaagctggggtggtccctgataccc

SEQ ID NO: 8 - DNA, 25 bp, synthetic construct
  source 1..25: mol_type = other DNA
ccccagcttcgccagtatgcccttg

SEQ ID NO: 9 - DNA, 29 bp, synthetic construct
  source 1..29: mol_type = other DNA
gcatactggcgaagndtgggatgtccctg

SEQ ID NO: 10 - DNA, 29 bp, synthetic construct
  source 1..29: mol_type = other DNA
gcatactggcgaagvhggggatgtccctg

SEQ ID NO: 11 - DNA, 29 bp, synthetic construct
  source 1..29: mol_type = other DNA
gcatactggcgaagtgggggatgtccctg

SEQ ID NO: 12 - DNA, 28 bp, synthetic construct
  source 1..28: mol_type = other DNA
cttcgccagtatgcccttgccttctggg

SEQ ID NO: 13 - DNA, 28 bp, synthetic construct
  source 1..28: mol_type = other DNA
catactggcgaagndtgggcgttccctg

SEQ ID NO: 14 - DNA, 28 bp, synthetic construct
  source 1..28: mol_type = other DNA
catactggcgaagvhggggcgttccctg

SEQ ID NO: 15 - DNA, 28 bp, synthetic construct
  source 1..28: mol_type = other DNA
catactggcgaaatgggggcgttccctg

SEQ ID NO: 16 - DNA, 36 bp, synthetic construct
  source 1..36: mol_type = other DNA
gattgatcgtgcgtcatattcatctcatggggaaag

SEQ ID NO: 17 - DNA, 30 bp, synthetic construct
  source 1..30: mol_type = other DNA
gaatatgacgcacgatcaatccttcgcccg

SEQ ID NO: 18 - protein, 398 aa, synthetic construct
MASTSSTSTDGHLNIRPMVHGSDKKLNFGAYITGLDLNNASDAEVDQLREAILRHKIVVIKGQQAEKPDKNWEMIKKLDPMHHMITQEEFGQLFHPTGEGLIAMLKLATVPTTEHGHIHLMGKGYQGDDHYGLKKLNLGEAFAGNYYSKPLAEEDFRAGVTRFQSWHMDGPLYKVHPPYISSLRFIQLPDGEQTVEWADGSGLSLKTKPGRTAFFSTSQLYDMLTDEERAMVDNSAVEYMYYPYEWIRGCRGNPNGLNVADEGREKPLDAMEEIARDERWTKTYPMVWFNELTKEKSLQVQPNCVRRLLIRRSADQKEPEIIEGPERVREFMNKLQQRIVRPEYVYVGPEEEGDHVFWYNWGMMHSKIDYPIAYGPRIVHQGWIPSHRVPRGPTAVAH

SEQ ID NO: 19 - DNA, 1197 bp, synthetic construct
  source 1..1197: mol_type = other DNA
atggcttcaactagttcgacctcgaccgatggacatcttaatatacgccctatggtccacggctccgataaaaaactgaattttggtgcgtacatcactggacttgacttaaataacgcatcggatgcagaagtcgatcagctccgcgaggcgattctgcgccataaaattgttgtaattaaaggccagcaagcggaaaaaccagacaaaaattgggaaatgataaagaaattagatcctatgcatcacatgataacgcaagaggaatttgggcagctttttcacccaacgggcgaaggattgatcgcaatgctgaaactggccactgtaccgacaaccgagcatgggcatattcatctcatggggaaaggctatcagggcgacgatcattacgggctgaagaaactgaatctaggcgaggcattcgccgggaattattacagtaaacccttagcggaggaggacttccgcgccggagtaacgcgttttcaatcttggcacatggatggtccattatataaagttcacccgccgtacataagcagcctacggttcatccaacttccggatggcgagcaaacagtcgagtgggcagatggctctggcctgagtcttaaaacgaagcccggacgcaccgccttttttagcacctctcagctttatgatatgttaacagatgaagaacgagcaatggtggataactctgcagtagagtatatgtattatccttatgagtggattcgcggttgccgcgggaacccgaacgggttaaacgtcgccgacgagggtagagaaaaacccctggatgcaatggaagaaatcgcacgagacgaacgctggactaaaacctatcctatggtatggttcaatgaactgactaaagaaaaatctctacaggttcagcctaattgtgtgcgtagactacttatccggagatctgcagatcagaaagagccagaaattattgagggcccagaaagagtacgtgaattcatgaacaagttacaacagagaattgtgcggcccgaatacgtctatgtcggtccggaggaagaaggcgatcatgttttctggtacaactggggaatgatgcatagtaagattgattatcccattgcctatggtccgcgcattgttcatcaaggttggattccaagtcatcgtgtgccgcgcggacctacggccgtagcacattaa

SEQ ID NO: 20 - protein, 351 aa, synthetic construct
FGAYITGLDLNSASDAEIDQLREAILRHKVVIVKGQQAETPDRNWEVIKKLDPMHHMITQEEFGQLFHPTGEGLIVRHIHLMGKGYQGDDHYGLKKLDLGEAFAGNYYSKPLAEEDFQAGITRFQSWHMDGPLYKVHPPYISSLRFIKLPQGEQTIEWADGSGMSLKAKPGRTGFFSTSQLYEMLTDEEKVMVDNSSVEYMHYPYEWIRSCRGNPNGLGVADEGREKSPETMEEIAREERWTKTLTGEKSLQVQPNCVRRLLIRRSVDQKEPEVIEGAEHARAFMHQLQQRILRPEYVYVGPEEEGDHVFWYNWGMMHSKIDYPVSFGPRIVHQGWIPSHRVPQPPAVAAH

SEQ ID NO: 21 - DNA, 1059 bp, synthetic construct
  source 1..1059: mol_type = other DNA
atgttcggggcttatattacggggctggatttaaattcagcgtcggatgctgaaatcgaccaactgcgggaagcaattctacgtcataaggtagtgattgtaaaaggccaacaggcagagactccggatagaaactgggaggtgattaagaaactagatccaatgcaccacatgatcacgcaggaagaatttgggcagcttttccatccaacaggtgaaggattgattgttcgtcacatacacttgatggggaaaggttaccagggggatgatcactatggtcttaaaaaactggatctcggggaagcctttgccgggaattattattcaaaaccgcttgctgaagaggattttcaggcgggtattactcgcttccagtcatggcacatggatgggcccctgtataaagttcatccaccttacatcagtagcctgcggtttattaagctgccccaaggcgagcagactatcgaatgggccgatggtagtggtatgtccctgaaggcgaaaccgggtcgaacgggtttttttagcacttcccaactgtatgaaatgctgaccgatgaggaaaaggttatggttgataatagttctgttgaatacatgcattatccatacgaatggattcggtcatgccggggcaaccctaatggcctgggtgtcgcggatgaaggccgggaaaaaagtcctgagacaatggaagagattgccagagaagaacgctggacgaaaactcttaccggagaaaagagcctacaggtacagcccaactgcgttcgccggctgcttatccgccggagcgtggatcagaaagagccagaagttattgagggagcggaacatgcacgcgcctttatgcatcagctgcaacagagaattctacgtcccgagtatgtgtatgttgggcctgaagaagagggtgatcatgtattttggtacaattggggtatgatgcacagcaaaatcgattatcctgttagtttcggtccacgtattgtccatcaaggttggatacctagtcaccgcgtaccgcaaccgccagccgttgcagcgcactaa

SEQ ID NO: 22 - protein, 397 aa, synthetic construct
SPSRIDNVSSKFRISPLVHSPDKKCNFGATVEGIDLNNLTDEEILVLREAIWTHKFLVIKDQTALVPKKNWELVTRLDPEAPILNQMDFAKCFHPTGEGILKKITLTLLPGVEDIHLMGKGYQGEDHYGVKNVTLNEAFSSAFHSTPLSEEDFENGNTRFQSWHMDGPGYRIDPPWFSSFRTIKLPNGPDQTVNWDDGSGLSMKAKPGRTAFFSSAQLYDLLTDEEKTMADHSWVEHMHHPYEWVRDCHGNPNGLSVACEGRETSMEEMDTFERDPSWTKKYPMVWVNPVTKEKSFQVQPNIVRKIFIRNNATDTPKVIDDLTEVRAFLNDIQLRILKPEYITAQPQNEGDMLLWDNYGMMHSRIDYPVKYGTRTAHQCWLGASRGPVGPVALPVDA

SEQ ID NO: 23 - DNA, 1194 bp, synthetic construct
  source 1..1194: mol_type = other DNA
tctccttcgcgtatcgataacgtcagctctaagtttcggataagcccgctcgtacacagcccggacaagaagtgcaattttggcgctactgttgagggtatagatctgaacaacttaacagacgaggagattctggtgttgcgtgaggcaatttggactcacaagttcttggttatcaaggaccaaacggcgctggtgcctaagaagaattgggagctggttacgcgcctggaccctgaggctccgatactcaaccaaatggacttcgccaaatgttttcatcctacaggggaaggtatccttaagaaaattacccttacgctgttaccaggcgttgaggacatccatctgatgggcaaaggatatcaaggcgaggaccactatggcgtgaagaatgtcacgcttaacgaggctttctcctctgcttttcatagcacaccactgtcggaagaggatttcgagaatgggaacactcgcttccaatcatggcacatggatggccccggctaccgcatagacccaccttggttttcttccttccgcacgatcaagcttcctaatggccccgatcaaaccgtcaattgggatgacggatcgggactttccatgaaggcgaaaccaggacgtacagcattcttctcatctgcacaactgtacgaccttttaactgatgaggaaaagactatggccgatcattcatgggtggaacacatgcaccatccgtatgagtgggttcgtgactgtcacggtaacccgaacggtttatctgtcgcctgtgagggccgggaaacaagtatggaagagatggacacatttgagcgggacccgtcttggacaaagaagtatccaatggtttgggtcaacccagtgacaaaagaaaagtcgtttcaggttcagcctaacatagttcggaagatcttcatacgtaacaatgccacagacaccccaaaggtaatcgatgaccttactgaggttcgcgcattcttaaatgacatccagttgcgtatcttaaagcccgaatatatcacggcccaacctcaaaacgaaggtgacatgcttctgtgggacaactatgggatgatgcatagtcgtattgattatcctgttaagtatggcacccgtacagcacaccaatgttggctgggagcctctcgcggccctgtgggtccggtggcgctccctgtcgacgcttaa

SEQ ID NO: 24 - protein, 398 aa, synthetic construct
MAFTSSNSTDGHLNIRPMVHGSDKKLNFGAYITGLDLNNASDDEVDQLREAILRHKVVVVKGQQAETPDKNWEIITKLDPMHHMITQEEFGRLFHPTGEGLIAMLKLATVPTAQDGHIHLMGKGYQGDDHYGLKKLTLGEAFAGNYYSKPLANEDFQAGIARFQSWHIDGPLYKVHPPYISSLRFIQLPVGEQKVEWADGSGLSLKTKPGRTGFFSTSQLYEMLTDEERVMVDNSTVEYMYYPYEWIRGCRGNPNGLSVADEGREKPLELMEEMARDEQWTKTYPMVWFNELTKEKSLQVQPNCARRLLIRRSADQKDPEIIEGPERVREFMDKLQQRIVRPEYIYVGPEEEGDHVFWYNWGLMHSRIDYPVAYGPRIVHQGWVPTHRIPRGSPAVAH

SEQ ID NO: 25 - DNA, 1197 bp, synthetic construct
  source 1..1197: mol_type = other DNA
atggctttcacttcgagcaattctacagatgggcatttaaacatacgtccaatggttcacggcagtgacaagaagttaaatttcggtgcgtatatcaccggcctggacctgaacaacgctagcgacgacgaggtggaccagttgagagaagccatactccgacacaaggtagtagtcgttaaggggcaacaggccgaaacacctgataagaactgggagattatcaccaagcttgatccaatgcaccacatgatcactcaagaggaattcggtcgtcttttccatccaaccggagaggggttgatagcaatgctcaagctcgctactgttcctacagcacaagatggccacattcaccttatgggtaaggggtaccagggtgacgatcattacggtttgaagaagttaacactgggtgaggctttcgcaggaaattattattcgaagccgctggcaaacgaggacttccaagctggaatcgcccggtttcaatcttggcacattgacggccccctctacaaggtacacccaccttatatctcgtccttacgtttcatccaacttcccgtcggggaacaaaaggtggagtgggccgacggttctggcctgtcccttaaaaccaaaccagggcgtacaggcttcttctcaacgagtcaattatacgagatgctgacggacgaggaacgggtcatggtggacaactcaacagtagagtacatgtattacccatacgagtggatacgcgggtgccgaggcaatccgaatggcttatcggtcgctgacgaggggcgagagaagccgctggaattaatggaagaaatggctagagatgaacagtggacaaagacctatccgatggtgtggtttaacgaattaacaaaggagaaatcgctgcaagttcaaccaaactgcgcacgtcgcctgcttatacgccgaagcgcggaccaaaaggaccctgagatcatagagggtcctgagcgtgtgagagaattcatggataagttacaacagcgtattgtacggcccgaatatatttatgtcggtccggaagaagagggagaccatgttttctggtacaattggggattgatgcacagtcgtatagactacccggttgcgtatgggcctcgtatagtacatcaaggatgggtcccaactcaccgtattccgcgagggtcgcctgcagtagcccattaa

SEQ ID NO: 26 - protein, 388 aa, synthetic construct
MGSTEETFRVKPFGGDLTFGAEVYGLDLNEISDAGIDQLRDLLQQHLVLVIKGQQNELPRKNWELLQKLDPGAPEFTDEQWAKFYNPEGKGILAKLGYNTIPDGGRLYLMGKGYQGEDHYGLKNVNMSESFADLYYSKPLPKEDFQKGITRFQSWHMDGPQYAINPPLFTSFRCIKLPEGEQTVDWADGSGLTKKIKPGRTGFFSTAQLYDMLSDEEKQMVDHSWCEYMYYPYEWILGCRGNPNGINVACEGREVPEEVMEAMPRNPNDQHILPLVWMNSVTGKKHLQVQPNVVRRLYIRSSPQDKPKVIEDVKEVRDFLTKLQIRILRPENIYVGPEEEGDHVLWYNWGVMHTKIDYPVEFGPRSAHQGWLPATRKPSGPVPIPVSQ

SEQ ID NO: 27 - DNA, 1167 bp, synthetic construct
  source 1..1167: mol_type = other DNA
atgggatctactgaggaaactttccgtgtgaaaccttttggtggcgaccttaccttcggtgcagaggtgtacgggttagatctcaacgagatcagcgatgccggtatcgatcaattgcgagaccttctgcaacagcacctcgttcttgtaattaaggggcagcaaaacgaactgccgcgaaagaactgggagctgctgcagaaattagacccaggggctccagagtttacagatgaacaatgggctaaattctacaacccggaaggtaaaggaattctggctaaattgggctacaacacaatcccagacggtggccgcttgtacctcatggggaaaggctaccaaggagaagaccactacggattgaagaacgttaacatgtcggagtctttcgcggacttatactactctaaaccactgcccaaagaggatttccaaaagggaatcactcgtttccaaagctggcacatggatggtccccaatatgctatcaacccgccgttgttcacgagcttccgatgcatcaagcttcccgagggtgagcaaaccgtggactgggcagatggttccggcttaaccaagaagatcaagcctgggcgaaccgggttcttttccaccgcgcaactgtatgatatgctgagcgacgaagagaagcaaatggtagaccattcatggtgtgagtatatgtactatccatacgagtggattctggggtgcagaggtaacccgaacgggatcaacgtagcctgcgagggccgtgaagtccccgaagaagttatggaagctatgcctcgaaaccccaacgaccaacatatattgcccctggtatggatgaattcagtcaccggtaagaagcacttgcaagtgcaaccgaacgtcgtacgtcgcttgtacatacggtcttcaccccaagacaagcctaaggttattgaggatgttaaagaggtacgtgactttctgacaaagctccagatacgcatcctgcgcccggagaatatctatgtagggccagaggaagaaggggaccacgtcttatggtacaactggggcgtcatgcataccaagatagactacccggtagaattcgggcctcgttcggcccaccaaggttggttacccgcgacccgtaaaccatcgggtcctgtgccaatcccagtaagccagtga

SEQ ID NO: 28 - protein, 399 aa, synthetic construct
MGSMPGFTVEPFDNGLKFGAEIHGLDINKITDSPEYQVLMTRPDSEVDKFRETIQKYLVVVVKGQQDELPSKNWELLQKLDPGAPEFTDEEWARFYNPEGKGILAKLGYNAIPDAGRLYLMGKGYQGKDHYGLKDVEINEPFADAYYSKPLPVEDFKKGVARFQSWHMDGPQYVINPPKFSSFRSIRVPKGEQTVEWADGSGLTKKVKPGRTAFISTAQMYDALSEEEKRMADHSWCEYMYYPYEWILGCRGNPNGLNVACEGREVPEEVMEAMPRDPKHQQLLPLVWLNDFTGGKHLQVQPNIVRRIFVRSGPNSNPKIIEDVKEVRNFLTNLQGRILRPENVYVGPEDEGDHLFWYNWGVMHSKIDYPVEFGVRTAHQGWLPGSKAPTGPVPIPAQR

SEQ ID NO: 29 - DNA, 1200 bp, synthetic construct
  source 1..1200: mol_type = other DNA
atgggatcaatgcctggcttcacagtcgaaccttttgataacggcttaaagttcggtgccgagatacacggactggacatcaataagatcaccgacagtccggagtaccaagtactgatgacccgcccggattccgaggtcgacaagtttcgcgaaaccatccagaaatacttagtggtggtggtcaaggggcaacaagatgagttgccttcaaagaactgggagctgcttcagaagctggaccccggcgctccggagtttactgacgaggagtgggcacgattctacaaccccgaagggaagggaatactcgcgaaattaggatacaacgcaatcccggatgctgggcggttgtacctgatgggcaaggggtaccaaggtaaggatcactatggactgaaggatgtggagattaatgagccatttgcagacgcctactattcgaaacctctgccagttgaagacttcaagaaaggcgtagcacgtttccagtcatggcacatggatgggccacagtacgtaattaacccgccgaagttttcctcttttcgctccatacgggttccaaagggtgagcaaacagttgagtgggctgacggatcaggcttgacaaagaaggtgaagccagggagaactgctttcatatccacagcacagatgtacgatgcattatctgaagaggaaaagcgtatggcggaccactcatggtgtgagtacatgtactatccttacgagtggatacttggctgccggggtaatcctaacggtttaaacgtagcttgtgagggtagagaggttcctgaagaggtgatggaagctatgccccgtgacccaaagcaccaacaactgttgccgctcgtatggctcaatgacttcaccggcgggaagcatctgcaagttcaacctaacatagtccgtcgcatattcgtacgtagcggtcctaactcgaacccgaaaataatagaagacgttaaagaggtgcgtaatttccttaccaatctgcaaggaagaattctgcgccctgagaacgtttatgtgggaccggaagacgagggcgaccatttgttttggtacaactggggagtaatgcacagcaaaattgattaccccgtagaattcggcgtgcggaccgcgcaccagggatggctgccgggttctaaggcccccacgggaccagtccctatccctgctcaacgttaa

SEQ ID NO: 30 - protein, 402 aa, synthetic construct
MTTSATMTTSATMNTTTKANSFDVKPFGQGLNFGAEIRGLDLNNMTDDDVNKFREVIQQYLVVVVKGQGNELPSKNWELLRRLDPDSPELTDDEWAKMYNPEGKGILAKLGYSLIPGAGRLYLMGKGYQGEDHFGLKDVTIEEAFADLYYSKPLPKEDFEKGITRFQSWHMDGPQYVVHGPIYTSFRCIKLPEGEQTVDWADGSGLTKKVKPGRTAFFSTAQLYDMFTPEEQQMADHSWCEPMFYPYEWILGCRGNPNGLNVACQGREVPFDVMEAMARKPKNQLVLPLVWMNSVTGKKHFQVQPNTVRRLFIRNNAEETPRVIEDVKEIRDFLTKLQIRVLRPENIYVGPEEEGDHVFWYNWGVMHTKIDYPVKFGVRTAHQGWIPASRAPTGPVPIPGQE

SEQ ID NO: 31 - DNA, 1209 bp, synthetic construct
  source 1..1209: mol_type = other DNA
atgacaacttcagcaactatgactaccagcgccactatgaacacaaccaccaaggctaactccttcgacgtaaagccgttcggacaaggattaaatttcggtgcggagatacggggactggaccttaacaacatgacggacgacgacgttaacaagtttagagaggtcatccaacaatacctcgttgtcgttgtgaagggccaaggtaacgagctgccaagcaagaattgggagttgcttcggcgcctggacccggatagccccgagttaaccgacgacgaatgggcgaagatgtacaacccagaaggcaagggcatactggcgaagctggggtactccctgatacccggagccggacgactgtatcttatgggtaagggatatcaaggggaagaccacttcggattaaaggacgtgaccattgaagaggcgtttgcggacctgtattatagtaagccactcccaaaagaggacttcgagaagggaataactagattccagtcatggcacatggacggaccacaatacgtggtgcacggcccgatctacaccagcttccgctgcatcaagttaccagaaggggagcaaactgttgattgggccgatggcagtggtcttacaaagaaagttaaacccgggcgtaccgctttcttctcaactgcccaattatacgatatgttcacgcctgaggagcaacaaatggcggaccacagttggtgcgagccgatgttctacccgtacgagtggatcttaggctgccggggcaacccgaacggtttaaatgtagcttgccagggtcgtgaagttccgttcgacgtgatggaagcgatggcgcgtaaaccaaagaatcaactcgttttgccgttagtgtggatgaattccgtcacaggaaagaaacatttccaggtacaacctaacacagtgcgtagactgttcatccgcaataatgcagaggagaccccacgggtcattgaggacgtaaaggagatcagagacttcttaaccaagctgcaaatcagagtattacgaccggaaaacatctatgtcggtcctgaagaggaaggtgaccacgtattttggtataactggggagttatgcacacaaagattgattatccggtaaagttcggggtgcgtacagcccatcagggctggataccggcttctcgcgccccgactggcccagtacctattcctgggcaggagtaa

SEQ ID NO: 32 - protein, 1049 aa, synthetic construct
MTIKEMPQPKTFGELKNLPLLNTDKPVQALMKIADELGEIFKFEAPGRVTRYLSSQRLIKEACDESRFDKNLSQAAKFARDFGGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRALDEVMNKLQRANPDDPAYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 33 - protein, 398 aa, synthetic construct
MASTSSTSTDGHLNIRPMVHGSDKKLNFGAYITGLDLNNASDAEVDQLREAILRHKIVVIKGQQAEKPDKNWEMIKKLDPMHHMITQEEFGQLFHPTGEGLIAMLKLATVPTTEHGHIHLMGKGYQGDDHYGLKKLNLGEAFAGNYYSKPLAEEDFRAGVTRFQSWHMDGPLYKVHPPYISSLRFIQLPDGEQTVEWADGSGLSLKTKPGRTAFFSTSQLYDMLTDEERAMVDNSAVEYMYYPYEWIRGCRGNPNGLNVADEGREKPLDAMEEIARDERWTKTYPMVWFNELTKEKSLQVQPNVVRRLLIRRSADQKEPEIIEGPERVREFMNKLQQRIVRPEYVYVGPEEEGDHVFWYNWGMMHSKIDYPIAYGPRIVHQGWIPSHRVPRGPTAVAH

SEQ ID NO: 34 - DNA, 1197 bp, synthetic construct
  source 1..1197: mol_type = other DNA
atggcttcaactagttcgacctcgaccgatggacatcttaatatacgccctatggtccacggctccgataaaaaactgaattttggtgcgtacatcactggacttgacttaaataacgcatcggatgcagaagtcgatcagctccgcgaggcgattctgcgccataaaattgttgtaattaaaggccagcaagcggaaaaaccagacaaaaattgggaaatgataaagaaattagatcctatgcatcacatgataacgcaagaggaatttgggcagctttttcacccaacgggcgaaggattgatcgcaatgctgaaactggccactgtaccgacaaccgagcatgggcatattcatctcatggggaaaggctatcagggcgacgatcattacgggctgaagaaactgaatctaggcgaggcattcgccgggaattattacagtaaacccttagcggaggaggacttccgcgccggagtaacgcgttttcaatcttggcacatggatggtccattatataaagttcacccgccgtacataagcagcctacggttcatccaacttccggatggcgagcaaacagtcgagtgggcagatggctctggcctgagtcttaaaacgaagcccggacgcaccgccttttttagcacctctcagctttatgatatgttaacagatgaagaacgagcaatggtggataactctgcagtagagtatatgtattatccttatgagtggattcgcggttgccgcgggaacccgaacgggttaaacgtcgccgacgagggtagagaaaaacccctggatgcaatggaagaaatcgcacgagacgaacgctggactaaaacctatcctatggtatggttcaatgaactgactaaagaaaaatctctacaggttcagcctaatgtggtgcgtagactacttatccggagatctgcagatcagaaagagccagaaattattgagggcccagaaagagtacgtgaattcatgaacaagttacaacagagaattgtgcggcccgaatacgtctatgtcggtccggaggaagaaggcgatcatgttttctggtacaactggggaatgatgcatagtaagattgattatcccattgcctatggtccgcgcattgttcatcaaggttggattccaagtcatcgtgtgccgcgcggacctacggccgtagcacattaa

SEQ ID NO: 35 - protein, 398 aa, synthetic construct
MASTSSTSTDGHLNIRPMVHGSDKKLNFGAYITGLDLNNASDAEVDQLREAILRHKIVVIKGQQAEKPDKNWEMIKKLDPMHHMITQEEFGQLFHPTGEGLIAMLKLATVPTTEHGHIHLMGKGYQGDDHYGLKKLNLGEAFAGNYYSKPLAEEDFRAGVTRFQSWHMDGPLYKVHPPYISSLRFIQLPDGEQTVEWADGSGLSLKTKPGRTAFFSTSQLYDMLTDEERAMVDNSAVEYMYYPYEWIRGCRGNPNGLNVADEGREKPLDAMEEIARDERWTKTYPMVWFNELTKEKSLQVQPNTVRRLLIRRSADQKEPEIIEGPERVREFMNKLQQRIVRPEYVYVGPEEEGDHVFWYNWGMMHSKIDYPIAYGPRIVHQGWIPSHRVPRGPTAVAH

SEQ ID NO: 36 - DNA, 1197 bp, synthetic construct
  source 1..1197: mol_type = other DNA
atggcttcaactagttcgacctcgaccgatggacatcttaatatacgccctatggtccacggctccgataaaaaactgaattttggtgcgtacatcactggacttgacttaaataacgcatcggatgcagaagtcgatcagctccgcgaggcgattctgcgccataaaattgttgtaattaaaggccagcaagcggaaaaaccagacaaaaattgggaaatgataaagaaattagatcctatgcatcacatgataacgcaagaggaatttgggcagctttttcacccaacgggcgaaggattgatcgcaatgctgaaactggccactgtaccgacaaccgagcatgggcatattcatctcatggggaaaggctatcagggcgacgatcattacgggctgaagaaactgaatctaggcgaggcattcgccgggaattattacagtaaacccttagcggaggaggacttccgcgccggagtaacgcgttttcaatcttggcacatggatggtccattatataaagttcacccgccgtacataagcagcctacggttcatccaacttccggatggcgagcaaacagtcgagtgggcagatggctctggcctgagtcttaaaacgaagcccggacgcaccgccttttttagcacctctcagctttatgatatgttaacagatgaagaacgagcaatggtggataactctgcagtagagtatatgtattatccttatgagtggattcgcggttgccgcgggaacccgaacgggttaaacgtcgccgacgagggtagagaaaaacccctggatgcaatggaagaaatcgcacgagacgaacgctggactaaaacctatcctatggtatggttcaatgaactgactaaagaaaaatctctacaggttcagcctaataccgtgcgtagactacttatccggagatctgcagatcagaaagagccagaaattattgagggcccagaaagagtacgtgaattcatgaacaagttacaacagagaattgtgcggcccgaatacgtctatgtcggtccggaggaagaaggcgatcatgttttctggtacaactggggaatgatgcatagtaagattgattatcccattgcctatggtccgcgcattgttcatcaaggttggattccaagtcatcgtgtgccgcgcggacctacggccgtagcacattaa

SEQ ID NO: 37 - protein, 406 aa, synthetic construct
MGSSHHHHHHSSGLVPRGSHMASTSSTSTDGHLNIRPMVHGSDKKLNFGAYITGLDLNNASDAEVDQLREAILRHKIVVIKGQQAEKPDKNWEMIKKLDPMHHMITQEEFGQLFHPTGEGLIVRHIHLMGKGYQGDDHYGLKKLNLGEAFAGNYYSKPLAEEDFRAGVTRFQSWHMDGPLYKVHPPYISSLRFIQLPDGEQTVEWADGSGLSLKTKPGRTAFFSTSQLYDMLTDEERAMVDNSAVEYMYYPYEWIRGCRGNPNGLNVADEGREKPLDAMEEIARDERWTKTYPMVWFNELTKEKSLQVQPNCVRRLLIRRSADQKEPEIIEGPERVREFMNKLQQRIVRPEYVYVGPEEEGDHVFWYNWGMMHSKIDYPIAYGPRIVHQGWIPSHRVPRGPTAVAH

SEQ ID NO: 38 - DNA, 3150 bp, synthetic construct
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcggccaaattcgcgcgtgattttggcggagacgggttagttacaagctggacgcatgaaaaaaattggaaaaaagcgcataatatcttacttccaagctttagtcagcaggcaatgaaaggctatcatgcgatgatggtcgatatcgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcactggatgaagtaatgaacaagctgcagcgagcaaatccagacgacccagcttatgatgaaaacaagcgccagtgtcaagaagatatcaaggtgatgaacgacctagtagataaaattattgcagatcgcaaagcaaggggtgaacaaagcgatgatttattaacgcagatgctaaacggaaaagatccagaaacgggtgagccgcttgatgacgggaacattagctatcaaattattacattcttaattgcgggacacgaaacaacaagtggtcttttatcatttgcgctgtatttcttagtgaaaaatccacatgtattacaaaaagtagcagaagaagcagcacgagttctagtagatcctgttccaagctacaaacaagtcaaacagcttaaatatgtcggcatggtcttaaacgaagcgctgcgcttatggccaactgcgcctgcgttttccctatatgcaaaagaagatacggtgcttggaggagaatatcctttagaaaaaggcgacgaagtaatggttctgattcctcagcttcaccgtgataaaacaatttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctcgatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

SEQ ID NO: 39 - protein, 1049 aa, synthetic construct
MTIKEMPQPKTFGELKNLPLLNTDKPVQALMKIADELGEIFKFEAPGRVTRYLSSQRLIKEACDESRFDKNLSQALKFARDFGGDGLVTSWTHEKNWKKAHNILLPSFSQQAMKGYHAMMVDIAVQLVQKWERLNADEHIEVSEDMTRLTLDTIGLCGFNYRFNSFYRDQPHPFIISMVRAFDETMNKLQRANPDDPAYDENKRQCQEDIKVMNDLVDKIIADRKARGEQSDDLLTQMLNGKDPETGEPLDDGNISYQIITFLIAGHETTSGLLSFALYFLVKNPHVLQKVAEEAARVLVDPVPSYKQVKQLKYVGMVLNEALRLWPTAPAFSLYAKEDTVLGGEYPLEKGDEVMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVRKKAENAHNTPLLVLYGSNMGTAEGTARDLADIAMSKGFAPQVATLDSHAGNLPREGAVLIVTASYNGHPPDNAKQFVDWLDQASADEVKGVRYSVFGCGDKNWATTYQKVPAFIDETLAAKGAENIADRGEADASDDFEGTYEEWREHMWSDVAAYFNLDIENSEDNKSTLSLQFVDSAADMPLAKMHGAFSTNVVASKELQQPGSARSTRHLEIELPKEASYQEGDHLGVIPRNYEGIVNRVTARFGLDASQQIRLEAEEEKLAHLPLAKTVSVEELLQYVELQDPVTRTQLRAMAAKTVCPPHKVELEALLEKQAYKEQVLAKRLTMLELLEKYPACEMKFSEFIALLPSIRPRYYSISSSPRVDEKQASITVSVVSGEAWSGYGEYKGIASNYLAELQEGDTITCFISTPQSEFTLPKDPETPLIMVGPGTGVAPFRGFVQARKQLKEQGQSLGEAHLYFGCRSPHEDYLYQEELENAQSEGIITLHTAFSRMPNQPKTYVQHVMEQDGKKLIELLDQGAHFYICGDGSQMAPAVEATLMKSYADVHQVSEADARLWLQQLEEKGRYAKDVWAG

SEQ ID NO: 40 - DNA, 3150 bp, synthetic construct
  source 1..3150: mol_type = other DNA
atgacaattaaagaaatgcctcagccaaaaacgtttggagagcttaaaaatttaccgttattaaacacagataaaccggttcaagctttgatgaaaattgcggatgaattaggagaaatctttaaattcgaggcgcctggtcgtgtaacgcgctacttatcaagtcagcgtctaattaaagaagcatgcgatgaatcacgctttgataaaaacttaagtcaagcgcttaaattcgcgcgtgattttggcggagacgggttagttacaagctggacgcatgaaaaaaattggaaaaaagcgcataatatcttacttccaagctttagtcagcaggcaatgaaaggctatcatgcgatgatggtcgatatcgccgtgcagcttgttcaaaagtgggagcgtctaaatgcagatgagcatattgaagtatcggaagacatgacacgtttaacgcttgatacaattggtctttgcggctttaactatcgctttaacagcttttaccgagatcagcctcatccatttattataagtatggtccgtgcattcgatgaaaccatgaacaagctgcagcgagcaaatccagacgacccagcttatgatgaaaacaagcgccagtgtcaagaagatatcaaggtgatgaacgacctagtagataaaattattgcagatcgcaaagcaaggggtgaacaaagcgatgatttattaacgcagatgctaaacggaaaagatccagaaacgggtgagccgcttgatgacgggaacattagctatcaaattattacattcttaattgcgggacacgaaacaacaagtggtcttttatcatttgcgctgtatttcttagtgaaaaatccacatgtattacaaaaagtagcagaagaagcagcacgagttctagtagatcctgttccaagctacaaacaagtcaaacagcttaaatatgtcggcatggtcttaaacgaagcgctgcgcttatggccaactgcgcctgcgttttccctatatgcaaaagaagatacggtgcttggaggagaatatcctttagaaaaaggcgacgaagtaatggttctgattcctcagcttcaccgtgataaaacaatttggggagacgatgtggaggagttccgtccagagcgttttgaaaatccaagtgcgattccgcagcatgcgtttaaaccgtttggaaacggtcagcgtgcgtgtatcggtcagcagttcgctcttcatgaagcaacgctggtacttggtatgatgctaaaacactttgactttgaagatcatacaaactacgagctcgatattaaagaaactttaacgttaaaacctgaaggctttgtggtaaaagcaaaatcgaaaaaaattccgcttggcggtattccttcacctagcactgaacagtctgctaaaaaagtacgcaaaaaggcagaaaacgctcataatacgccgctgcttgtgctatacggttcaaatatgggaacagctgaaggaacggcgcgtgatttagcagatattgcaatgagcaaaggatttgcaccgcaggtcgcaacgcttgattcacacgccggaaatcttccgcgcgaaggagctgtattaattgtaacggcgtcttataacggtcatccgcctgataacgcaaagcaatttgtcgactggttagaccaagcgtctgctgatgaagtaaaaggcgttcgctactccgtatttggatgcggcgataaaaactgggctactacgtatcaaaaagtgcctgcttttatcgatgaaacgcttgccgctaaaggggcagaaaacatcgctgaccgcggtgaagcagatgcaagcgacgactttgaaggcacatatgaagaatggcgtgaacatatgtggagtgacgtagcagcctactttaacctcgacattgaaaacagtgaagataataaatctactctttcacttcaatttgtcgacagcgccgcggatatgccgcttgcgaaaatgcacggtgcgttttcaacgaacgtcgtagcaagcaaagaacttcaacagccaggcagtgcacgaagcacgcgacatcttgaaattgaacttccaaaagaagcttcttatcaagaaggagatcatttaggtgttattcctcgcaactatgaaggaatagtaaaccgtgtaacagcaaggttcggcctagatgcatcacagcaaatccgtctggaagcagaagaagaaaaattagctcatttgccactcgctaaaacagtatccgtagaagagcttctgcaatacgtggagcttcaagatcctgttacgcgcacgcagcttcgcgcaatggctgctaaaacggtctgcccgccgcataaagtagagcttgaagccttgcttgaaaagcaagcctacaaagaacaagtgctggcaaaacgtttaacaatgcttgaactgcttgaaaaatacccggcgtgtgaaatgaaattcagcgaatttatcgcccttctgccaagcatacgcccgcgctattactcgatttcttcatcacctcgtgtcgatgaaaaacaagcaagcatcacggtcagcgttgtctcaggagaagcgtggagcggatatggagaatataaaggaattgcgtcgaactatcttgccgagctgcaagaaggagatacgattacgtgctttatttccacaccgcagtcagaatttacgctgccaaaagaccctgaaacgccgcttatcatggtcggaccgggaacaggcgtcgcgccgtttagaggctttgtgcaggcgcgcaaacagctaaaagaacaaggacagtcacttggagaagcacatttatacttcggctgccgttcacctcatgaagactatctgtatcaagaagagcttgaaaacgcccaaagcgaaggcatcattacgcttcataccgctttttctcgcatgccaaatcagccgaaaacatacgttcagcacgtaatggaacaagacggcaagaaattgattgaacttcttgatcaaggagcgcacttctatatttgcggagacggaagccaaatggcacctgccgttgaagcaacgcttatgaaaagctatgctgacgttcaccaagtgagtgaagcagacgctcgcttatggctgcagcagctagaagaaaaaggccgatacgcaaaagacgtgtgggctgggtaa

(19) United States
(12) Patent Application Publication
(10) Pub. No.: US 2026/0366053 A1
(43) Pub. Date: Nov. 19, 2026

(54) SYNTHESIS OF COTYLENIN AND ANALOGS THEREOF FOR THE DEVELOPMENT OF SELECTIVE MODULATORS OF 14-3-3 PROTEIN-PROTEIN INTERACTION

(71) Applicant: UNIVERSITY OF FLORIDA RESEARCH FOUNDATION, INCORPORATED, Gainesville, FL (US)

(72) Inventors: Hans Renata, Gainesville, FL (US); Yanlong Jiang, Gainesville, FL (US)

(73) Assignee: UNIVERSITY OF FLORIDA RESEARCH FOUNDATION, INCORPORATED, Gainesville, FL (US)

(21) Appl. No.: 19/107,314

(22) PCT Filed: Sep. 1, 2023
(86) PCT No.: PCT/US2023/073376

§ 371 Date: Feb. 27, 2025

Related U.S. Application Data
(60) Provisional application No. 63/374,522, filed on Sep. 2, 2022.

(51) Int. Cl. C12P 19/56; C12N 1/06; C12N 1/145; C12N 1/205; C12N 9/02; C12P 15/00

(52) U.S. Cl. CPC C12P 19/56; C12N 1/06; C12N 1/145; C12N 1/205; C12N 9/0071; C12P 15/00; C12Y 114/11; C12Y 114/14001

(57) ABSTRACT

The present disclosure provides methods of preparing cotylenol, and analogs thereof, comprising a selective hydroxylation catalyzed by a non-heme dioxygenase or cytochrome P450 hydroxylase.

### RELATED APPLICATIONS

This patent application claims priority to U.S. Provisional Patent Application No. 63/374,522, filed Sep. 2, 2022, which is incorporated herein by reference in its entirety.

### GOVERNMENT SUPPORT

This invention was made with government support under R35 GM128895 awarded by the National Institutes of Health. The government has certain rights in the invention.

### REFERENCE TO AN ELECTRONIC SEQUENCE LISTING

The contents of the electronic sequence listing (U119770210W000-SEQ-WWZ.xml; Size: 59,010 bytes; and Date of Creation: Aug. 31, 2023) is herein incorporated by reference in its entirety.

### BACKGROUND OF THE DISCLOSURE

Cotylenin A and fusicoccin A are two flagship members of the fusicoccane diterpenoid family that are capable of acting as molecular glues to stabilize the interaction between the 14-3-3 proteins and their clients. Modulation of such interactions has significance in cancer and neurodegeneration. However, the structural complexity and diverse oxygenation patterns of fusicoccane diterpenoids present enormous synthetic challenges. There is a need for improved synthesis of cotylenin A and fusicoccin A, and their analogs.

### SUMMARY OF THE DISCLOSURE

In one aspect, the present disclosure provides a method of preparing cotylenol, and analogs thereof. Cotylenol is the aglycone of cotylenin A. The method may comprise the union of two cyclopentene fragments in an allylative coupling, a one-pot Prins cyclization-transannular hydride shift sequence, and/or an enzymatic oxidation to install the key tertiary alcohol at C3. In certain embodiments, the present disclosure provides A method of preparing a compound of Formula I-1 or I-2:

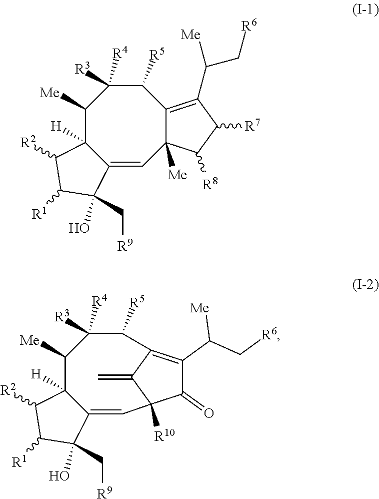

or a salt, solvate, hydrate, polymorph, co-crystal, tautomer, stereoisomer, isotopically labeled compound, or prodrug thereof, the method comprising incubating a first reaction mixture to produce the compound of Formula I-1 or I-2, respectively, or a salt, solvate, hydrate, polymorph, co-crystal, tautomer, stereoisomer, isotopically labeled compound, or prodrug thereof, wherein the first reaction mixture comprises:
(a) a compound of Formula A1 or A2, respectively:

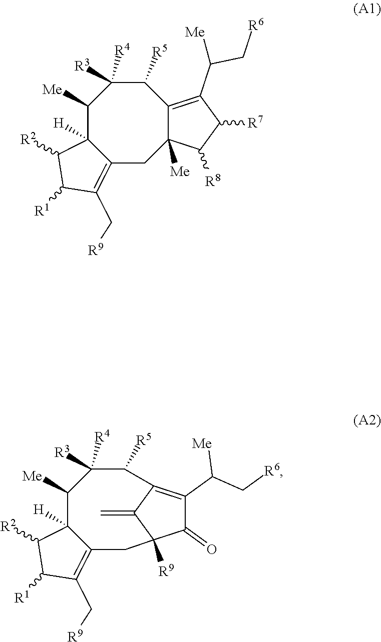

or a salt, solvate, hydrate, polymorph, co-crystal, tautomer, stereoisomer, isotopically labeled compound, or prodrug thereof; and
(b) a non-heme dioxygenase.

In another aspect, the present disclosure provides a method of preparing a compound of Formula II:

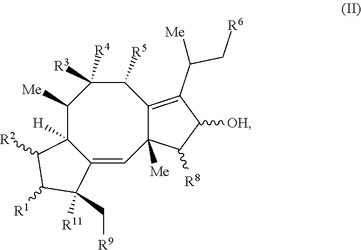

or a salt, solvate, hydrate, polymorph, co-crystal, tautomer, stereoisomer, isotopically labeled compound, or prodrug thereof, the method comprising incubating a first reaction mixture to produce the compound of Formula II, or a salt, solvate, hydrate, polymorph, co-crystal, tautomer, stereoisomer, isotopically labeled compound, or prodrug thereof, wherein the first reaction mixture comprises:
(a) a compound of Formula B:

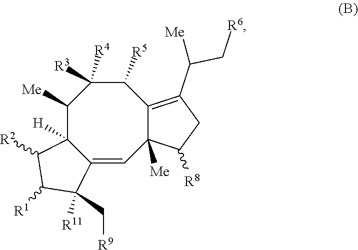

or a salt, solvate, hydrate, polymorph, co-crystal, tautomer, stereoisomer, isotopically labeled compound, or prodrug thereof; and
(b) a cytochrome P450 hydroxylase.

In another aspect, the present disclosure provides a kit comprising:
a non-heme dioxygenase comprising an amino acid sequence that is at least 80% identical to the amino acid sequence of SEQ ID NO: 30, 26, 28, 24, 22, 35, 33, or 18; and
instructions for using the non-heme dioxygenase in the method.

In another aspect, the present disclosure provides a kit comprising:
a cytochrome P450 hydroxylase comprising an amino acid sequence that is at least 80% identical to the amino acid sequence of SEQ ID NO: 32; and instructions for using the cytochrome P450 hydroxylase in the method.

The compounds prepared by the methods of the present disclosure may serve as ligands to stabilize 14-3-3-based protein-protein interactions and/or may increase the ligandability of intrinsically disordered protein. The compounds prepared by the methods of the present disclosure may have pharmaceutical uses (e.g., for treating diseases (e.g., cancer and neurodegeneration)).

The methods of the present disclosure may be advantageous over known methods in that the former:
1. may allow larger scale preparations (e.g., to support material demand for subsequent pharmacological investigation);
2. may permit access to analogs that are not readily available from the latter; and/or
3. may permit access to novel chemical entities (e.g., novel chemical entities with pharmaceutical uses for treating diseases (e.g., cancer and neurodegeneration)).

### DEFINITIONS

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC), supercritical fluid chromatography (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The present disclosure additionally encompasses compounds described herein as individual isomers substantially free of other isomers, and alternatively, as mixtures of various isomers.

In a formula, the bond [symbol] is a single bond, the dashed line [symbol] is a single bond or absent, and the bond [symbol] or [symbol] is a single or double bond.

Unless otherwise provided, a formula depicted herein includes compounds that do not include isotopically enriched atoms and also compounds that include isotopically enriched atoms. Compounds that include isotopically enriched atoms may be useful as, for example, analytical tools, and/or probes in biological assays.

The term “aliphatic” includes both saturated and unsaturated, nonaromatic, straight chain (i.e., unbranched), branched, acyclic, and cyclic (i.e., carbocyclic) hydrocarbons. In some embodiments, an aliphatic group is optionally substituted with one or more functional groups (e.g., halo, such as fluorine). As will be appreciated by one of ordinary skill in the art, “aliphatic” is intended herein to include alkyl, alkenyl, alkynyl, cycloalkyl, cycloalkenyl, and cycloalkynyl moieties.

When a range of values (“range”) is listed, it is intended to encompass each value and sub-range within the range. A range is inclusive of the values at the two ends of the range unless otherwise provided. For example, “an integer between 1 and 4” refers to 1, 2, 3, and 4. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₅) and the like. Unless otherwise specified, each instance of an alkyl group is independently optionally substituted, e.g., unsubstituted (an “unsubstituted alkyl”) or substituted (a “substituted alkyl”) with one or more substituents. In certain embodiments, the alkyl group is unsubstituted C₁₋₁₂ alkyl (e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)). The attachment point of alkyl may be a single bond (e.g., as in —CH₃), double bond (e.g., as in ═CH₂), or triple bond (e.g., as in ═CH). The moieties ═CH₂ and ═CH are also alkyl.

In some embodiments, an alkyl group is substituted with one or more halogens. “Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

“Alkenyl” refers to a radical of a straight-chain or branched hydrocarbon group having from 2 to 20 carbon atoms, one or more (e.g., two, three, or four, as valency permits) carbon-16 carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C), and the like. Additional examples of alkenyl include heptenyl (C₇), octenyl (C₅), octatrienyl (C₅), and the like. Unless otherwise specified, each instance of an alkenyl group is independently optionally substituted, e.g., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl. In an alkenyl group, a C═C double bond for which the stereochemistry is not specified (e.g., —CH═CHCH₃ or

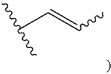

may be in the (E)- or (Z)-configuration.

“Alkynyl” refers to a radical of a straight-chain or branched hydrocarbon group having from 2 to 20 carbon atoms, one or more (e.g., two, three, or four, as valency permits) carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C), and the like. Additional examples of alkynyl include heptynyl (C₇), octynyl (C₅), and the like. Unless otherwise specified, each instance of an alkynyl group is independently optionally substituted, e.g., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 13 ring carbon atoms (“C₃₋₁₃ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃-8 carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged, or spiro ring system such as a bicyclic system (“bicyclic carbocyclyl”). Carbocyclyl can be saturated, and saturated carbocyclyl is referred to as “cycloalkyl.” In some embodiments, carbocyclyl is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. Carbocyclyl can be partially unsaturated. Carbocyclyl may include zero, one, or more (e.g., two, three, or four, as valency permits) C═C double bonds in all the rings of the carbocyclic ring system that are not aromatic or heteroaromatic. Carbocyclyl including one or more (e.g., two or three, as valency permits) C═C double bonds in the carbocyclic ring is referred to as “cycloalkenyl.” Carbocyclyl including one or more (e.g., two or three, as valency permits) C═C triple bonds in the carbocyclic ring is referred to as “cycloalkynyl.” “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, e.g., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl. In certain embodiments, the carbocyclyl is substituted or unsubstituted, 3- to 7-membered, and monocyclic. In certain embodiments, the carbocyclyl is substituted or unsubstituted, 5- to 13-membered, and bicyclic.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl. In certain embodiments, the carbocyclyl includes oxo substituted thereon.

“Heterocyclyl” or “heterocyclic” refers to a radical of a 3- to 13-membered non-aromatic ring system having ring carbon atoms and 1 to 4 ring heteroatoms, wherein each heteroatom is independently selected from nitrogen, oxygen, and sulfur (“3-13 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system such as a bicyclic system (“bicyclic heterocyclyl”). A heterocyclyl group can be saturated or can be partially unsaturated. Heterocyclyl may include zero, one, or more (e.g., two, three, or four, as valency permits) double bonds in all the rings of the heterocyclic ring system that are not aromatic or heteroaromatic. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, e.g., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. In certain embodiments, the heterocyclyl is substituted or unsubstituted, 3- to 7-membered, and monocyclic. In certain embodiments, the heterocyclyl is substituted or unsubstituted, 5- to 13-membered, and bicyclic. In certain embodiments, the heterocyclyl includes oxo substituted thereon.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include azirdinyl, oxiranyl, or thiiranyl. Exemplary 4-membered heterocyclyl groups containing one heteroatom include azetidinyl, oxetanyl and thietanyl. Exemplary 5-membered heterocyclyl groups containing one heteroatom include tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include dioxolanyl, oxasulfuranyl, disulfuranyl, and oxazolidin-2-one. Exemplary 5-membered heterocyclyl groups containing three heteroatoms include triazolinyl, oxadiazolinyl, and thiadiazolinyl. Exemplary 6-membered heterocyclyl groups containing one heteroatom include piperidinyl, tetrahydropyranyl, dihydropyridinyl, and thianyl. Exemplary 6-membered heterocyclyl groups containing two heteroatoms include piperazinyl, morpholinyl, dithianyl, and dioxanyl. Exemplary 6-membered heterocyclyl groups containing two heteroatoms include triazinanyl. Exemplary 7-membered heterocyclyl groups containing one heteroatom include azepanyl, oxepanyl and thiepanyl. Exemplary 8-membered heterocyclyl groups containing one heteroatom include azocanyl, oxecanyl, and thiocanyl. Exemplary 5-membered heterocyclyl groups fused to a C₆ aryl ring (also referred to herein as a 5,6-bicyclic heterocyclic ring) include indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, e.g., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, e.g., unsubstituted (“unsubstituted heteroaryl”) or substituted (“substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In certain embodiments, the heteroaryl group is 5-6 membered, monocyclic. In certain embodiments, the heteroaryl group is 8-14 membered, bicyclic.

Exemplary 5-membered heteroaryl groups containing one heteroatom include pyrrolyl, furanyl and thiophenyl. Exemplary 5-membered heteroaryl groups containing two heteroatoms include imidazolyl, pyrazolyl, oxazolyl, isoxazolyl, thiazolyl, and isothiazolyl. Exemplary 5-membered heteroaryl groups containing three heteroatoms include triazolyl, oxadiazolyl, and thiadiazolyl. Exemplary 5-membered heteroaryl groups containing four heteroatoms include tetrazolyl. Exemplary 6-membered heteroaryl groups containing one heteroatom include pyridinyl. Exemplary 6-membered heteroaryl groups containing two heteroatoms include pyridazinyl, pyrimidinyl, and pyrazinyl. Exemplary 6-membered heteroaryl groups containing three or four heteroatoms include triazinyl and tetrazinyl, respectively. Exemplary 7-membered heteroaryl groups containing one heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl.

“Partially unsaturated” refers to a group that includes at least one double or triple bond. The term “partially unsaturated” is intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., contains all single bonds.

In some embodiments, aliphatic, alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{f} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

In certain embodiments, the carbon atom substituents are independently halogen, substituted (e.g., substituted with one or more halogen) or unsubstituted C₁₋₆ alkyl, —OR^{aa}, SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group. In certain embodiments, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, the carbon atom substituents are independently halogen, substituted (e.g., substituted with one or more halogen moieties) or unsubstituted C₁₋₆ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₆ alkyl, an oxygen protecting group when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₆ alkyl, or a nitrogen protecting group.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{aa}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, the nitrogen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the nitrogen atom substituents are independently substituted (e.g., substituted with one or more halogen) or unsubstituted C₁₋₆ alkyl or a nitrogen protecting group.

In certain embodiments, the substituent present on a nitrogen atom is a nitrogen protecting group (also referred to as an amino protecting group). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}, and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-34 chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R^{aa}) include p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, a nitrogen protecting group is Bn, Boc, Cbz, Fmoc, trifluoroacetyl, triphenylmethyl, acetyl, or Ts.

In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group. In certain embodiments, the oxygen atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the oxygen atom substituents are independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, an oxygen protecting group is silyl, TBDPS, TBDMS, TIPS, TES, TMS, MOM, THP, t-Bu, Bn, allyl, acetyl, pivaloyl, or benzoyl.

In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group. In certain embodiments, the sulfur atom substituents are independently substituted (e.g., substituted with one or more halogen) or unsubstituted C₁₋₆ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₆ alkyl, or an oxygen protecting group when attached to an oxygen atom; and each R^{bb} is independently hydrogen, substituted (e.g., substituted with one or more halogen) or unsubstituted C₁₋₆ alkyl, or a nitrogen protecting group. In certain embodiments, the sulfur atom substituents are independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). Sulfur protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{a}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference. In certain embodiments, a sulfur protecting group is acetamidomethyl, t-Bu, 3-nitro-2-pyridine sulfenyl, 2-pyridine-sulfenyl, or triphenylmethyl.

The “molecular weight” of —R, wherein —R is any monovalent moiety, is calculated by subtracting the atomic weight of a hydrogen atom from the molecular weight of the molecule R—H. The “molecular weight” of -L-, wherein -L- is any divalent moiety, is calculated by subtracting the combined atomic weight of two hydrogen atoms from the molecular weight of the molecule H-L-H.

In certain embodiments, the molecular weight of a substituent is lower than 200, lower than 150, lower than 100, lower than 50, or lower than 25 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, and/or iodine atoms. In certain embodiments, a substituent consists of carbon, hydrogen, and/or fluorine atoms. In certain embodiments, a substituent does not comprise one or more, two or more, or three or more hydrogen bond donors. In certain embodiments, a substituent does not comprise one or more, two or more, or three or more hydrogen bond acceptors.

The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

“Pharmaceutically acceptable salt” refers to those salts which are, within the scope of sound medical judgment, suitable for use in contact with the tissues of humans and other animals without undue toxicity, irritation, allergic response, and the like, and are commensurate with a reasonable benefit/risk ratio. Pharmaceutically acceptable salts are well known in the art. For example, Berge et al., describe pharmaceutically acceptable salts in detail in J. Pharmaceutical Sciences (1977) 66:1-19. Pharmaceutically acceptable salts of the compounds describe herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The provided compounds may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane, that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound is complexed with the acid in a way that proton transfer (e.g., a complete proton transfer) from the acid to a compound easily occurs at room temperature. In the co-crystal, however, a compound is complexed with the acid in a way that proton transfer from the acid to a herein does not easily occur at room temperature. In certain embodiments, in the co-crystal, there is substantially no proton transfer from the acid to a compound. In certain embodiments, in the co-crystal, there is partial proton transfer from the acid to a compound. Co-crystals may be useful to improve the properties (e.g., solubility, stability, and ease of formulation) of a compound.

The term “prodrugs” refer to compounds, including derivatives of the provided compounds, which have cleavable groups and become by solvolysis or under physiological conditions the provided compounds which are pharmaceutically active in vivo. Such examples include, but are not limited to, ester derivatives and the like. Other derivatives of the compounds have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds are particular prodrugs. In some cases it is desirable to prepare double ester type prodrugs such as (acyloxy)alkyl esters or ((alkoxycarbonyl)oxy)alkylesters. C₁ to C₈ alkyl, C₂-C₈ alkenyl, C₂-C₈ alkynyl, aryl, C₇-C₁₂ substituted aryl, and C₇-C₁₂ arylalkyl esters of the provided compounds may be preferred.

These and other exemplary substituents are described in more detail in the Detailed Description, Examples, Figures, and Claims. The present disclosure is not intended to be limited in any manner by the above exemplary listing of substituents.

### BRIEF DESCRIPTION OF THE DRAWINGS

FIG. 1A shows representative members of the fusicoccane diterpenoid family and their activity as modulators of protein-protein interactions with the 14-3-3 adaptor proteins.

FIG. 1B shows a retrosynthetic strategy for accessing a diverse range of fusicoccanes by relying on late-stage hybrid oxidations and efficient skeletal construction.

FIG. 2A shows a synthesis of cotylenol from (+)-limonene oxide and (+)-limonene.

FIG. 2B shows a fragment coupling approach to synthesize key intermediate 8 and its conversion to cotylenol.

FIG. 3 shows an optimization of enzymatic oxidation via homolog screening and enzyme engineering to achieve site- and chemoselective oxidation at C3.

FIG. 4 shows a homology model for MoBsc9.

FIG. 5 shows an X-ray structure of compound S4.

FIGS. 6A to 6C show completion of the synthesis of additional fusicoccanes. FIG. 6A 8 shows C13 oxidation of 4 by P450BM3 variant MERO1 L75A and its application in the synthesis of brassicicenes A, L and R. FIG. 6B shows the synthesis of brassicicenes C, F, H, J and K through biomimetic skeletal rearrangement from 28. FIG. 6C shows additional C—H oxidation results to synthesize unnatural fusicoccanes.

### DETAILED DESCRIPTION OF CERTAIN EMBODIMENTS OF THE DISCLOSURE

Many protein targets are regarded as “undruggable” due to their intrinsic disorder and lack of ligandable conformation. One emerging modality to address this issue is by modulating protein-protein interaction (PPI) to provide a more ordered conformation for further targeting by small molecules. There exists a subset of small molecules (so-called molecular glues) that are capable of stabilizing the PPI between two proteins by inducing ternary complex formation. Though this property is highly valued, it is only exhibited by a few small molecules, as it remains difficult to predict the interactome of disordered proteins.

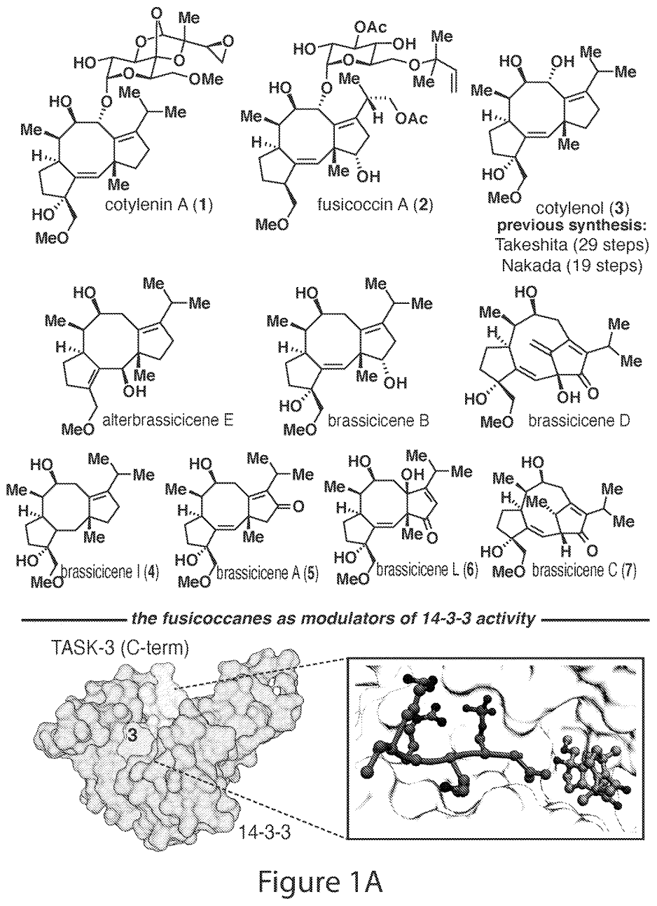

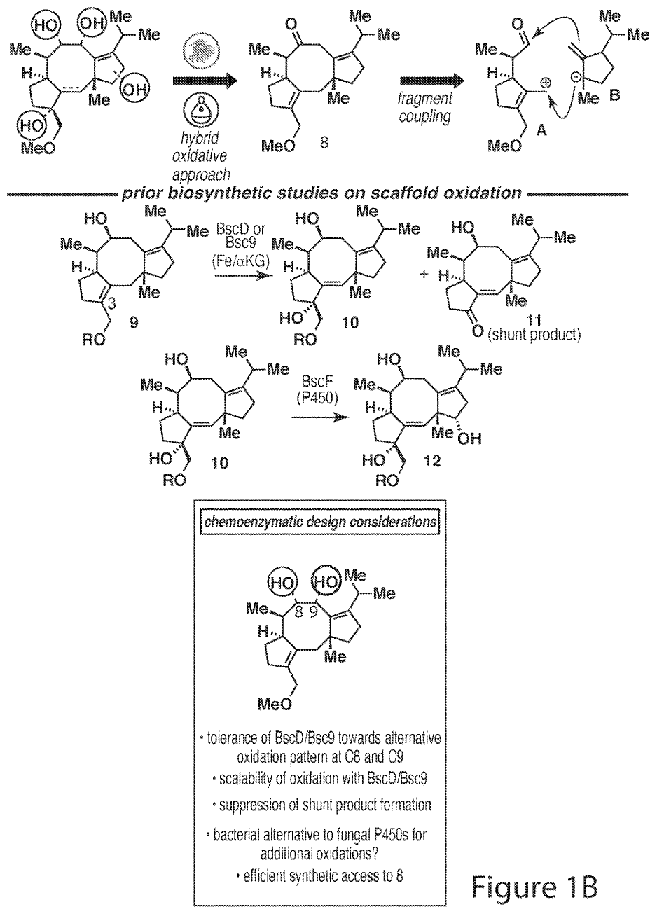

The 14-3-3 proteins serve as adaptor proteins that regulate many cellular processes by binding to a variety of disease-relevant signaling proteins (FIG. 1). For example, Raf kinases, YAP transcription factor, p53 and synucleins are known clients for these proteins. The 14-3-3 family itself consists of 7 different isoforms and in addition to the above validated clients, it has been estimated that there are ca. 3000 additional 14-3-3 clients across the interactome. Specifically, stabilization of 14-3-3 interaction is thought to elicit a variety of pharmacological effects, such as promoting degradation or functional inhibition of disease-driving clients. As such, small molecules that can serve as molecular glues to modulate 14-3-3 interaction are highly prized. One such molecule can be found in cotylenin A, a diterpene glycoside natural product that was initially identified as cytotoxic and capable of stabilizing the interaction between 14-3-3 and C-RAF. Modulation of PPI between 14-3-3 proteins and different binding partners may be possible through the use of natural and unnatural analogs (see Ottmann's work). For example, cotylenol, the aglycone of cotylenin A, is cytotoxic toward human myeloid leukemia cells and has been shown structurally to form a ternary complex with 14-3-3sigma and the C-terminal of TASK-3. Thus far, analogs of cotylenin have been developed through semisynthetic elaboration of fusicoccin A (a structurally-related natural product) and the native fungal producer of cotylenin A, Cladosporium sp. 501-7W, was reported to lose its ability to proliferate during preservation. In contrast to semisynthesis, de novo chemical synthesis allows for a more versatile and deep-seated modification to the scaffold to enable the discovery of novel molecular glues for further pharmacological investigation.

The fusicoccanes are fungal diterpenoids that are defined by their 5/8/5 ring system. Several members of the family, such as cotylenin A (1) and derivatives of fusicoccin A (2), are known to induce apoptosis and act as “molecular glues” to stabilize the protein-protein interaction (PPI) between the 14-3-3 hub proteins and several of their partners. In particular, cotylenin A and fusicoccin A are phytotoxins that target the 14-3-3 PPI. Conserved across eukaryotes, the 14-3-3 proteins may serve as adaptor proteins that regulate many cellular processes by binding to a variety of disease-relevant signaling proteins and phosphoprotein clients, such as Raf kinases and the YAP transcriptional modulator. Given this role, there has been interest in the development of small molecules that are able to modulate these PPIs. Studies by Ottmann and others on cotylenin A and related natural and semisynthetic fusicoccanes have begun to suggest that modulation of PPI between 14-3-3 proteins and different binding partners is possible by adjusting the oxidation patterns of the scaffold. In line with its 14-3-3 modulatory activity, 1 is known to induce apoptosis and has been used in combination with anti-epidermal growth factor receptor antibody and kinase inhibitors to suppress tumor growth. Studies by Ohkanda and co-workers have suggested that the absence of a secondary alcohol at C₁₂ may be a key molecular determinant for cellular cytotoxicity. Though the sugar moieties contribute to a tighter ternary complex formation, they may not be critical for activity. X-ray diffraction studies lend further support to this observation as the sugar moieties are found to be partially solvent exposed in the crystal structures. In agreement, cotylenol was observed to exhibit moderate cytotoxicity on human myeloid leukemia cells and a crystal structure of cotylenol in complex with 14-3-3s and a synthetic C-terminal hexapeptide of TASK-3 has also been solved.

Despite their promising biological activities, access to fusicoccanes and their derivatives, including 1 and 3, has been challenging as many family members can only be isolated in small quantities as a mixture of congeners. The native fungal producer of 1 and 3, Cladosporium sp. 501-7W, was reported to lose its ability to proliferate during preservation. Synthetic access to these compounds has also proven nontrivial. A number of total syntheses of cotylenol have been reported so far. However, they range between 15 and 29 steps (Kato and Takeshita, 1996 and Nakada, 2020) in length, highlighting the synthetic challenges posed by these molecules.

In one aspect, the present disclosure provides a method of preparing a compound of Formula I-1 or I-2:

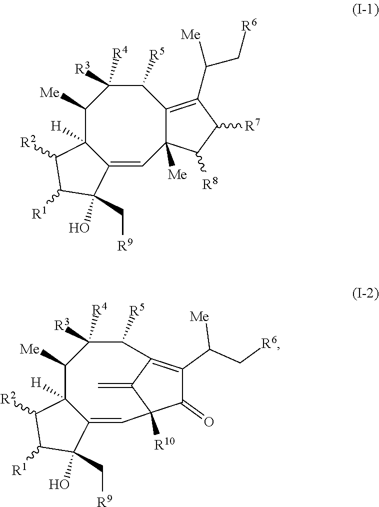

or a salt, solvate, hydrate, polymorph, co-crystal, tautomer, stereoisomer, isotopically labeled compound, or prodrug thereof, the method comprising incubating a first reaction mixture to produce the compound of Formula I-1 or I-2, respectively, or a salt, solvate, hydrate, polymorph, co-crystal, tautomer, stereoisomer, isotopically labeled compound, or prodrug thereof, wherein the first reaction mixture comprises:
(a) a compound of Formula A1 or A2, respectively:

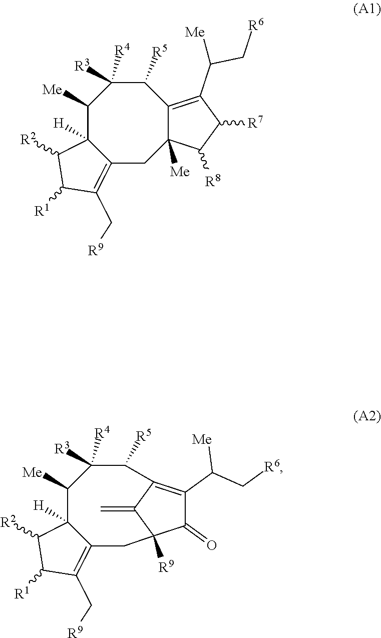

or a salt, solvate, hydrate, polymorph, co-crystal, tautomer, stereoisomer, isotopically labeled compound, or prodrug thereof; and
(b) a non-heme dioxygenase;
   wherein:
each one of R¹, R², R⁶, R⁷, and R⁸ is independently hydrogen, —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, or —OS(═O)₂N(R^{a})₂;
R³ and R⁴ taken together form ═O;
or: R³ is —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, or —OS(═O)₂N(R^{a})₂, and R⁴ is hydrogen;
each one of R⁵, R⁹, and R¹⁰ is independently —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, or hydrogen; and
each R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl.

In certain embodiments, the method is an eight- to thirteen-step synthesis of cotylenol, or brassicicene A, brassicicene C, brassicicene F, brassicicene H, brassicicene I, brassicicene J, brassicicene K, brassicicene L, or brassicicene R. In certain embodiments, the method is an eleven-step synthesis of 3. The methods may be a significant improvement from previous syntheses. The methods may provide the first foray towards establishing the biocatalytic utility and substrate promiscuity of the key dioxygenase from the native biosynthetic pathway of cotylenol. The methods may also be useful for preparing brassicicene congeners, including those with alternative skeletal connectivities, to enable further pharmacological exploration. The methods may also be useful for preparing C3-oxidized fusicoccane diterpenoids.

The methods may comprise a hybrid oxidative approach by combining enzymatic and chemical C—H oxidations. The methods may comprise oxidative reactions to install the hydroxyl groups at C3, C8, and/or C9. The C8,C9-trans-diol of 3 may be accessed from the C8-keto counterpart (8) through judicious redox manipulations (see Nakada's work). Ketone 8 would arise from synthons A and B through two C—C bond forming events and the appropriate synthetic equivalents of A and B could be found in cyclopentenes 16 and 17. Nozaki-Hiyama-Kishi-based allylation may be able to construct the key quaternary center at C11 with high stereoselectivity (see Takeshita's work). The non-heme dioxygenases (NHDs) BscD and Bsc9 may be useful in the generation of the C3 alcohol (see Dairi's and Oikawa's prior biosynthetic studies on 3 and the brassicicenes). In the native biosynthetic pathway, alcohol 9 may undergo an initial hydrogen atom abstraction at C1, which may lead to subsequent olefin isomerization and a radical rebound at C3 to produce 10 along with shunt product 11. The synthetic application of BscD and Bsc9 has not been demonstrated. There have been challenges involved in installing the C3 alcohol in prior 8 syntheses. The methods of the present disclosure would also apply the enzymatic oxidation on alternative oxidation patterns at C8 and C9 relative to 9. The promiscuity of the two enzymes has not been demonstrated. In the native biosynthetic pathway, several membrane-bound P450s in addition to BscD and Bsc9 are involved in the oxidative tailoring of the scaffold (see Tazawa's work). The method may comprise bacterial P450s to oxidize other remote positions on the 5/8/5 tricycle.

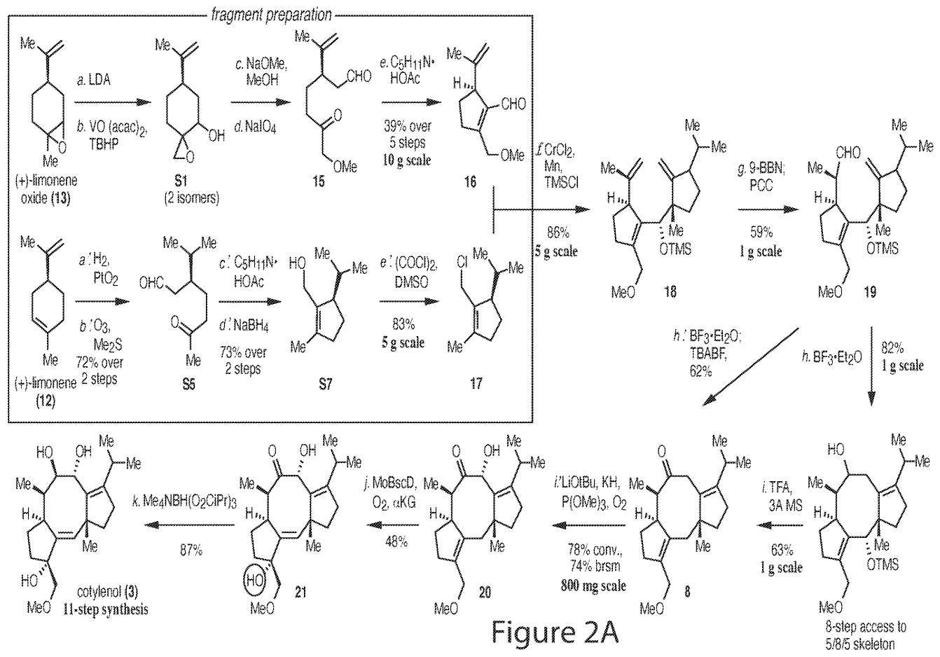

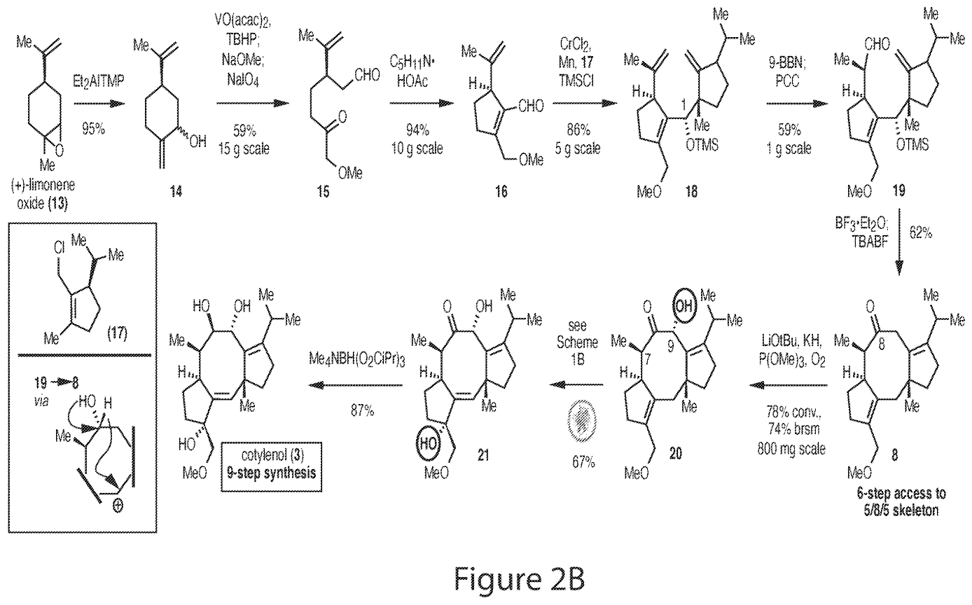

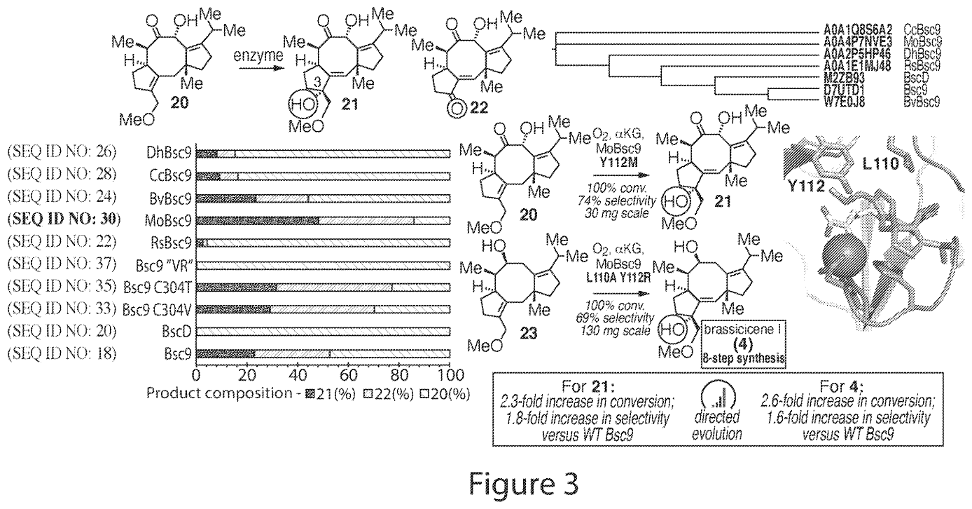

The key cyclopentane fragments 16 and 17 were prepared in five steps each from (+)-limonene oxide (13) and (+)-limonene (S3) (FIG. 2A). Though simplified variants of 16 have previously been prepared from limonene, an efficient strategy to introduce the primary allylic alcohol has yet to be described. Towards this goal, 13 was subjected to a regioselective epoxide opening, followed by VO(acac)₂-catalyzed epoxidation to provide S1 as an inconsequential mixture of two diastereomers. Treatment of this intermediate with NaOMe effected an epoxide ring opening from the less-hindered position and the resulting diol was oxidatively cleaved in the presence of NaIO₄. An intramolecular aldol condensation completed the synthesis of cyclopentenal 16. This five-step sequence could be routinely conducted on decagram scale with satisfactory overall yield (39%).

Alternatively, cyclopentenal 16 was prepared in three steps from (+)-limonene (FIG. 2B). Though simplified variants of 16 have previously been prepared from limonene, an efficient strategy to introduce the allylic methyl ether has yet to be described. Towards this goal, 13 was first subjected to a regioselective epoxide opening using a known literature procedure. A telescoped protocol involving VO(acac)₂-catalyzed epoxidation, epoxide ring opening with NaOMe and oxidative cleavage with NaIO₄ furnished aldehyde 15. Finally, an intramolecular aldol condensation completed the synthesis of 16. This three-step sequence could be routinely conducted on decagram scale with satisfactory overall yield (55%).

Toward 17, catalytic hydrogenation of the isopropenyl unit of S3 in the presence of PtO₂ and ozonolysis of its trisubstituted olefin furnished S5 in 72% yield. Construction of cyclopentenyl alcohol S7 from this intermediate was achieved through an intramolecular aldol condensation and reduction of the aldehyde with NaBH₄. Chlorination in the presence of (COCl)₂ and DMSO completed the synthesis of allylic chloride 7. Similar to the preparation of 16, this five-step route proved to be robust, proceeding routinely on five-gram scale with 44% overall yield (FIG. 2A).

Union of 16 and 17 was realized by employing Fürstner's modification of the Nozaki-Hiyama-Kishi (NHK) reaction. This method (1) features the catalytic use of low-valent chromium salt, to minimize potential physiological hazards, and (2) uses TMSCl as the reaction mediator, which allows for simultaneous capping of the C1 alcohol as the TMS ether. Selective hydroboration of the isopropenyl unit and oxidation of the corresponding primary alcohol to the aldehyde were combined in a telescoped protocol to afford 19, whose stereochemical configuration was verified through single-crystal X-ray diffraction analysis of a related derivative (S9).

Submission of this intermediate 19 to a Prins reaction in the presence of BF₃·Et₂O generated a 5/8/5 tricycle (S10) that contains all the necessary chemical handles for accessing 8. At this stage, the extraneous hydroxyl group at C1 needed to be excised. During attempts to desilylate, it was found that the use of TFA on S10 could lead to a complete deoxygenation at C1 16 along with the formation of a ketone moiety at C8 (i.e., compound 8). Furthermore, the C8 stereochemical configuration of S10 permitted this process, as the corresponding C8-epimer did not undergo the same deoxygenation. This transformation may proceed through selective ionization of the C1 hydroxyl group, which may initiate a transannular hydride transfer from the C8 carbon.

A suitable protocol was sought to convert 19 to 8 in one step. An extensive test of Lewis and Brönsted acids for this conversion eventually led to the development of a one-pot protocol featuring an initial treatment with BF₃·Et₂O, followed by the addition of tetra-n-butylammonium bifluoride (TBABF), which likely reacts with BF₃ to generate HBF₄ in situ. In comparison, stronger conditions tested led to simple elimination of the Cl—OH. Prior syntheses of 3 had established the viability of installing the C9 hydroxyl group via enolate α-oxidation with MoOPH. Due to safety and toxicity issues associated with preparing MoOPH on large scale, an alternative set of conditions to effect this transformation was sought. After extensive screening, it was found that a combination of LiOtBu and KH and molecular oxygen in the presence of P(OMe)₃ could be employed to afford 20 in high conversion, selectivity and isolated yield, setting the stage for investigating the key enzymatic oxidation with BscD and Bsc9, though a minor byproduct arising from α-oxidation at C7 was observed. As a testament to the scalability of the route, the entire sequence toward 20 could be routinely conducted on gram-scale or near gram-scale to provide ample material supply for biocatalytic exploration of BscD and Bsc9.

The biocatalytic use of BscD and Bsc9 was investigated for the oxidation of 20. While Bsc9 could be heterologously expressed as a N-His₆-tagged protein in E. coli, BscD was found to be insoluble. BscD and Bsc9 share ca. 70% sequence identity, and the regions of divergence are primarily found in their N-terminal domains, in which Bsc9 contains an additional 26-residue sequence, and two insertion segments. Grafting of the N-terminal domain of Bsc9 onto BscD was attempted, but this effort did not lead to any improvement in soluble protein yield. Reaction of 20 with Bsc9 in cell lysates provided a mixture of the desired tertiary alcohol product (21) and shunt product 22 with moderate conversion. In accordance, no reaction was observed with E. coli lysates containing BscD. The shunt product may arise from trapping of the tertiary radical at C3 with molecular oxygen through the intermediacy of a dioxetane species (see Dairi's work). From the perspective of reaction optimization, we speculated that if 21 is actually derived from 22, the ratio of 21:22 could be readily modulated by adjusting the reaction conditions. Subjecting 22 to the enzymatic transformation provided no further conversion, undermining this hypothesis. Importantly, this observation suggested that optimization of the product composition in the reaction may need to be achieved through alternative means.

During initial tests of Bsc9 in cell lysates, batch-to-batch variability of the reaction outcome was observed. Eventually, this issue may be rectified by developing a consistent and rigorous lysis protocol and ensuring that the lysates were used immediately for reaction. Addition of TCEP to the lysate was also found to improve conversion. In line with this observation, purified Bsc9 readily formed precipitates, suggesting rapid denaturation. Bsc9 contains two cysteine residues, one of which (C304) is predicted by homology model to be surface exposed. We speculated that this residue could be targeted for mutagenesis to enhance the reaction outcome. While mutations C304V and C304T in Bsc9 resulted in small improvements in conversion, the ratio of 21:22 did not improve. Our homology model also predicted that one of the insertion segments in Bsc9 (Ala103-Gly116, hereby referred to as “active-site insertion”) lies opposite of the His-His-Asp iron-binding triad in the active site. As this region might be implicated in substrate binding, we generated a Bsc9-BscD chimera by swapping the Ala103-Gly116 sequence of Bsc9 with the corresponding Val-Arg diad from BscD. This chimera (Bsc9 “VR”) provided no reaction on 20, suggesting the importance of the active-site insertion segment for hydroxylation activity on 20.

The above observation prompted us to test additional homologs of Bsc9 that diverge at the N-terminal and the active-site insertion regions. Ten homologs were identified by picking the top ten hits from Genome Neighborhood Diagram (GND) analysis and five of the ten were arbitrarily chosen for further characterization. A homolog from Magnaporthe oryzae (MoBsc9, 54% sequence identity to Bsc9) displayed improved conversion and ratio of 21:22, which translates to ca. two-fold improvement in assay yield of 21. Interestingly, no terpene synthase encoding genes could be identified within the vicinity of MoBsc9, suggesting that this dioxygenase might be involved in the biosynthesis of an entirely different natural product scaffold.

A brief directed evolution campaign was conducted to further improve the ratio of 21:22 in the enzymatic reaction. Based on our homology model of MoBsc9, two hydrophobic residues in the putative active site, L110 and Y112, were targeted for site-saturation mutagenesis. A “22-c trick” approach (see Reetz's work) to reduce codon redundancy was employed in combination with a thin layer chromatography-based (TLC-based) screening (see Li's work). In this round of screening, we identified mutation Y112M that afforded 8% improvement in selectivity while maintaining the high reaction conversion. Concurrently, screening of the Y112X library for reaction with 23, prepared via one-step reduction of 8, yielded variant Y112R that produced brassicicene I (4) with 67% conversion and 51% selectivity (defined as percent ratio of the desired product:total products). As a comparison, WT Bsc9 provided 37% conversion and 43% selectivity in this reaction. Using these two single mutants as parents, another round of evolution was performed by randomly mutagenizing L110. While only marginal improvement (less than 5%) was obtained in testing the Y112M L110X library on 20, the Y112R L110X library yielded variant L110A that further improved the selectivity for the production of 4 by 18%. Overall, the directed evolution campaign allowed for 2.3-fold improvement in conversion and 1.8-fold improvement in selectivity for the production of 21 and 2.6-fold improvement in conversion and 1.6-fold improvement in selectivity for the production of 4 relative to WT Bsc9. Preparative scale biocatalytic reaction on 30-50 mg scale with MoBscD Y112M could be conducted to afford 21 in 67% isolated yield. A corresponding reaction on 23 with MoBsc9 L110A Y112R on 100 mg scale provided brassicicene I (4) in 64% isolated yield. The final reduction to provide cotylenol proceeded uneventfully under Nakada's conditions, completing the synthesis in 9 (FIG. 2B) or 11 steps (FIG. 2A) (longest linear sequence) from commercial materials. Additionally, we achieved the first synthesis of brassicicene I in 8 steps.

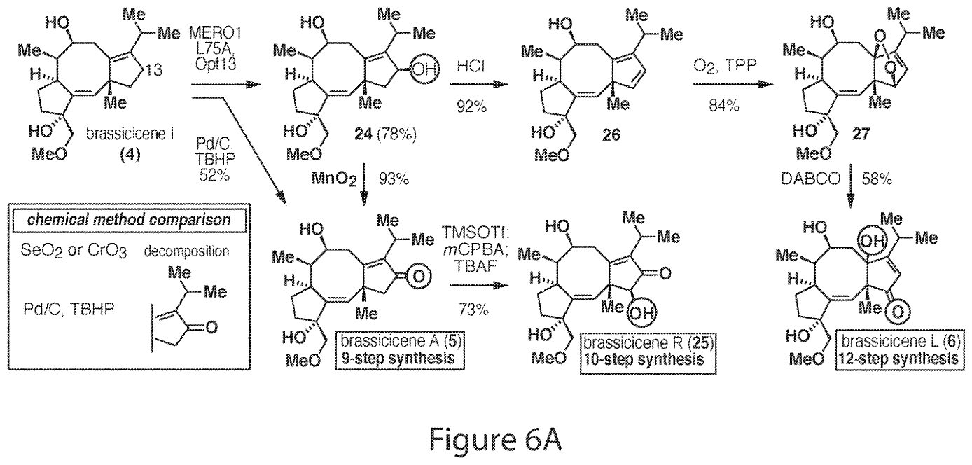

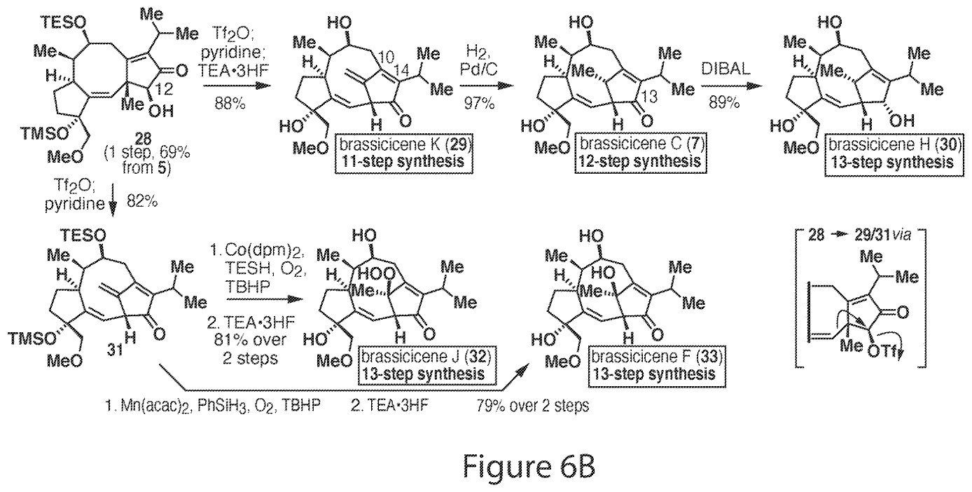

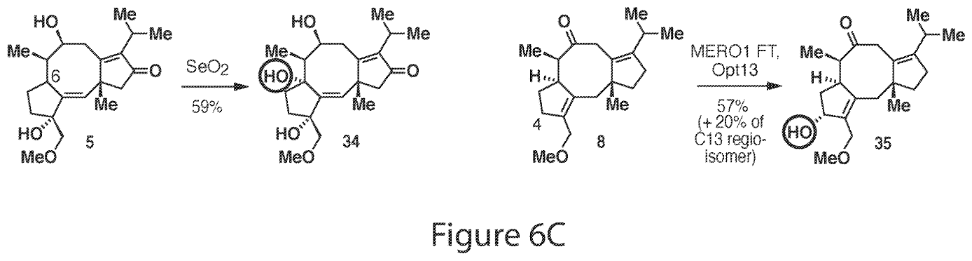

Towards the goal of effecting selective C—H hydroxylation at other positions on the 5/8/5 tricycle, a concurrent screening of additional enzymatic and chemical oxidations was performed. Eight different variants of P450BM3, previously developed for the site-selective C—H oxidation of decalin-containing terpenoids, were tested with brassicicene I, revealing the ability of variant MERO1 L75A to effect selective hydroxylation at C13 to the corresponding allylic alcohol in 78% yield without any observable regioisomers (FIG. 6A). Among the chemical methods surveyed, SeO₂ and CrO₃ yielded a complex mixture of products, but the use of palladium-catalyzed allylic oxidation led to the direct formation of brassicicene A (5) in moderate yield, completing a nine-step synthesis of this natural product. Alternatively, this compound could also be obtained via MnO₂-mediated oxidation of 24. Rubottom oxidation of 5, achieved via enol ether formation, reaction with mCPBA and TBAF desilylation, afforded brassicicene R (25) with a total step count of 10. The ability of MERO1 L75A to effect allylic oxidation at C13 without any over-oxidation paved the way for accessing alternative oxidation patterns through the intermediacy of the corresponding diene. Treatment of 24 with HCl effected a clean elimination to cyclopentadiene 26, which was then subjected to 1O2 [4+2]cycloaddition and Kornblum-DeLaMare rearrangement to complete the first synthesis of brassicicene L (6) in 12 steps.

Ready access to 5 inspired us to design a biomimetic approach toward rearranged family members that share a common C10-C14 bridgehead double bond motif, such as brassicicenes C, H and K (FIG. 6B). To date, compounds within this series have not been synthesized. Initial attempts to realize the rearrangement involved the direct functionalization of C12 with hypervalent iodine reagents, in the hope that the resulting iodinated species would spontaneously undergo a 1,2-rearrangement. However, this approach yielded no desired rearranged product. After a similar failure with α-bromination at C12, 5 or its protected derivatives were examined as a suitable starting point for the proposed rearrangement. Treatment of partially protected triol 28, obtained from 5 in 1 step, with triflic anhydride followed by pyridine led to the spontaneous formation of a product with the desired rearranged skeleton, which was deprotected in the same pot to complete our synthesis of brassicicene K (29) in 10 steps. While it was previously proposed that the rearranged brassicicenes could arise from either radical or carbocationic pathway (see Oikawa's work), this result lent further support to the latter. The exo-methylene of 29 was chemo- and diastereoselectively reduced under hydrogenation conditions to furnish brassicicene C (7). Finally, reduction of the C13 ketone of 7 afforded brassicicene H (30), thereby completing the first synthesis of these three rearranged brassicicenes. Though the autoxidation of 7 to brassicicene J (32) was previously reported in the literature, the reported reaction proceeded with low conversion. The use of ¹O₂ to drive the conversion was also not effective. Instead, protected brassicicene K (31, accessible in one step from 28) was subjected to Mukaiyama hydroperoxidation conditions, followed by deprotection with Et3N·3HF to yield 32. Attempts to effect this transformation in the absence of protecting group led to no reaction at all. Adjusting the metal hydride hydrogen atom transfer conditions for alkene hydration led to tertiary alcohol formation and provided brassicicene F (33) after silyl group removal.

During initial attempts to convert 5 to 25, it was found that the C6 position of 5 could be selectively oxidized with SeO₂ to provide an unnatural fusicoccane diterpenoid (FIG. 6C). Additionally, screening of our P450BM3 library on 8 revealed that variant MERO1 FT is capable of oxidize primarily at C4 to afford yet another unnatural fusicoccane diterpenoid.

In certain embodiments, the non-heme dioxygenase is a non-heme dioxygenase existing in a strain of the fungi Magnaporthe oryzae.

In certain embodiments, the non-heme dioxygenase comprises an amino acid sequence that is at least 70% (e.g., at least 75%, at least 80%, at least 85%, at least 90%, at least 95%, at least 96%, at least 97%, at least 98%, or at least 99%) identical to the amino acid sequence of SEQ ID NO: 30, 26, 28, 24, 22, 35, 33, or 18. In certain embodiments, the non-heme dioxygenase comprises an amino acid sequence that is at least 80% identical to the amino acid sequence of SEQ ID NO: 30, 26, 28, 24, 22, 35, 33, or 18. In certain embodiments, the non-heme dioxygenase comprises an amino acid sequence that is at least 70% (e.g., at least 75%, at least 80%, at least 85%, at least 90%, at least 95%, at least 96%, at least 97%, at least 98%, or at least 99%) identical to the amino acid sequence of SEQ ID NO: 30. In certain embodiments, the non-heme dioxygenase comprises an amino acid sequence that is at least 80% identical to the amino acid sequence of SEQ ID NO: 30.

In certain embodiments, the non-heme dioxygenase comprises a mutation. In certain embodiments, the mutation is a mutation of L110 or Y112, or a combination thereof. In certain embodiments, the mutation is L110A, Y112M, or Y112R, or a combination thereof.

In certain embodiments, the non-heme dioxygenase comprises an amino acid sequence of SEQ ID NO: 30. In certain embodiments, the non-heme dioxygenase comprises an amino acid sequence of SEQ ID NO: 26, 28, 24, 22, 35, 33, or 18.

In certain embodiments, the method further comprises lysing a cell to provide a cell lysate, wherein the step of incubating a first reaction mixture is performed after the step of lysing a cell. In certain embodiments, the step of incubating a first reaction mixture is performed immediately (e.g., no longer than 1, 3, 10, 30, or 60 minutes) after the step of lysing a cell. In certain embodiments, the cell lysate comprises the non-heme dioxygenase. In certain embodiments, the non-heme dioxygenase is isolated from a cell lysate. In certain embodiments, the cell is a cell of a fungus, yeast, plant, alga, or bacterium. In certain embodiments, the cell is a cell of a species of the bacteria Escherichia. In certain embodiments, the cell is a cell of a strain of the bacteria Escherichia coli. In certain embodiments, the cell is a cell of a species of the fungi Magnaporthe. In certain embodiments, the cell is a cell of a strain of the fungi Magnaporthe oryzae. In certain embodiments, the cell is modified to express the non-heme dioxygenase. In certain embodiments, the non-heme dioxygenase is a recombinant non-heme dioxygenase.

In another aspect, the present disclosure provides a method of selective oxidizing at C13 on the cotylenol skeleton. In certain embodiments, the present disclosure provides method of preparing a compound of Formula II:

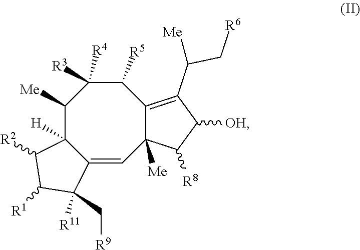

or a salt, solvate, hydrate, polymorph, co-crystal, tautomer, stereoisomer, isotopically labeled compound, or prodrug thereof, the method comprising incubating a first reaction mixture to produce the compound of Formula II, or a salt, hydrate, polymorph, co-crystal, tautomer, stereoisomer, isotopically labeled compound, or prodrug thereof, wherein the first reaction mixture comprises:
(a) a compound of Formula B:

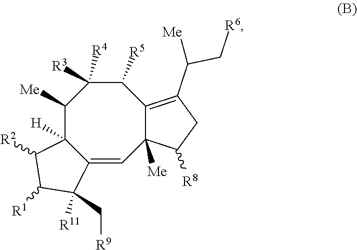

or a salt, solvate, hydrate, polymorph, co-crystal, tautomer, stereoisomer, isotopically labeled compound, or prodrug thereof; and
(b) a cytochrome P450 hydroxylase;
   wherein:
each one of R¹, R², R⁵, R⁶, and R⁸ is independently hydrogen, —OR^{a}, —N(R^{a})₂, —SR^{a}, —SCN, —NO₂, —N₃, —NR^{a}C(═NR^{a})R^{a}, —NR^{a}C(═O)OR^{a}, —NR^{a}C(═)N(R^{a})₂, —NR^{a}C(═NR^{a})R^{a}, —NR^{a}C(═NR^{a})OR^{a}, —NR^{a}C(═NR^{a})N(R^{a})₂, -optionally substituted (=O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR aS(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, or —OS(═O)₂N(R^{a})₂;
R³ is —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, or —OS(═O)₂N(R^{a})₂, and R⁴ is hydrogen;
or: R³ and R⁴ taken together form ═O;
each one of R⁹ and R¹¹ is independently —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, or hydrogen; and
each R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl.

In certain embodiments,

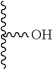

is

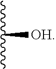

In certain embodiments,

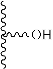

is

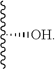

In certain embodiments, the cytochrome P450 hydroxylase is a cytochrome P450 BM3 hydroxylase. In certain embodiments, the cytochrome P450 hydroxylase comprises an amino acid sequence that is at least 70% (e.g., at least 75%, at least 80%, at least 85%, at least 90%, at least 95%, at least 96%, at least 97%, at least 98%, or at least 99%) identical to the amino acid sequence of SEQ ID NO: 32. In certain embodiments, the cytochrome P450 hydroxylase comprises an amino acid sequence that is at least 80% identical to the amino acid sequence of SEQ ID NO: 32. In certain embodiments, the cytochrome P450 hydroxylase comprises a mutation. In certain embodiments, the mutation is a mutation of L75. In certain embodiments, the mutation is L75A. In certain embodiments, the cytochrome P450 hydroxylase comprises an amino acid sequence of SEQ ID NO: 32.

In certain embodiments, the method further comprises lysing a cell to provide a cell lysate, wherein the step of incubating a first reaction mixture is performed after the step of lysing a cell. In certain embodiments, the step of incubating a first reaction mixture is performed immediately after the step of lysing a cell. In certain embodiments, “immediately after” is between 1 and 10 seconds, between 10 and 60 seconds, between 1 and 3 minutes, or between 3 and 10 minutes, inclusive, after. In certain embodiments, the cell lysate comprises the cytochrome P450 hydroxylase. In certain embodiments, the cytochrome P450 hydroxylase is isolated from a cell lysate. In certain embodiments, the cell is a cell of a fungus, yeast, plant, alga, or bacterium. In certain embodiments, the cell is a cell of a species of the bacteria Escherichia. In certain embodiments, the cell is a cell of a strain of the bacteria Escherichia coli. In certain embodiments, the cell is modified to express the cytochrome P450 hydroxylase. In certain embodiments, the cytochrome P450 hydroxylase is a recombinant cytochrome P450 hydroxylase.

In certain embodiments, R¹ is hydrogen. In certain embodiments, R¹ is —OR^{a}. In certain embodiments, R¹ is —OH. In certain embodiments, R¹ is —O(substituted or unsubstituted C₁₋₆ alkyl) (e.g., —OCH₃ or -OBn). In certain embodiments, R¹ is —O(oxygen protecting group). In certain embodiments,

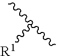

is

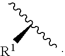

In certain embodiments,

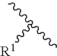

is

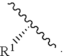

In certain embodiments, R² is hydrogen. In certain embodiments, R² is —OR^{a}. In certain embodiments, R² is —OH. In certain embodiments, R² is —O(substituted or unsubstituted C₁₋₆ alkyl) (e.g., —OCH₃ or -OBn). In certain embodiments, R² is —O(oxygen protecting group). In certain embodiments,

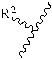

is

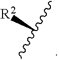

In certain embodiments,

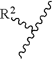

is

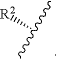

In certain embodiments, R³ and R⁴ taken together form ═O. In certain embodiments, R³ is —OR^{a}; and R⁴ is hydrogen. In certain embodiments, R³ is —O(substituted or unsubstituted C₁₋₆ alkyl) (e.g., —OCH₃ or -OBn); and R⁴ is hydrogen. In certain embodiments, R³ is —O(oxygen protecting group); and R⁴ is hydrogen. In certain embodiments, R³ is —OH; and R⁴ is hydrogen.

In certain embodiments, R⁵ is —OR^{a}. In certain embodiments, R⁵ is —O(substituted or unsubstituted heterocyclyl). In certain embodiments, R⁵ is —O(substituted or unsubstituted heterocyclyl comprising oxygen as the only heteroatoms in the heterocyclyl ring system). In certain embodiments, R⁵ is —O(substituted or unsubstituted C₁₋₆ alkyl) (e.g., —OCH₃ or -OBn). In certain embodiments, R⁵ is —O(oxygen protecting group). In certain embodiments, R⁵ is —OH. In certain embodiments, R⁵ is hydrogen.

In certain embodiments, R⁶ is hydrogen. In certain embodiments, R⁶ is —OR^{a}. In certain embodiments, R⁶ is —O(substituted or unsubstituted acyl). In certain embodiments, R⁶ is —OC(═O)(substituted or unsubstituted C₁₋₆ alkyl). In certain embodiments, R⁶ is —OC(═O)CH₃. In certain embodiments, R⁶ is —O(substituted or unsubstituted C₁₋₆ alkyl) (e.g., —OCH₃ or -OBn). In certain embodiments, R⁶ is —O(oxygen protecting group). In certain embodiments, R⁶ is —OH.

In certain embodiments, R⁷ is hydrogen. In certain embodiments, R⁷ is —OR^{a}. In certain embodiments, R⁷ is —O(substituted or unsubstituted C₁₋₆ alkyl) (e.g., —OCH₃ or -OBn). In certain embodiments, R⁷ is —O(oxygen protecting group). In certain embodiments, R⁷ is —OH. In certain embodiments,

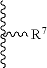

is

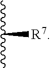

In certain embodiments,

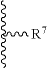

is

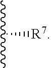

When R⁷ is hydrogen or —OH, the method may further comprise incubating a second reaction mixture to produce the compound of Formula III-1:

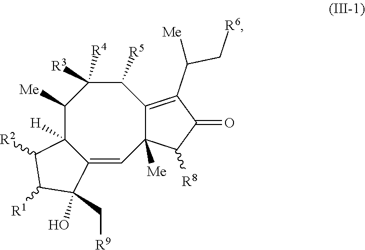

or a salt, solvate, hydrate, polymorph, co-crystal, tautomer, stereoisomer, isotopically labeled compound, or prodrug thereof, wherein the second reaction mixture comprises:
(a) a compound of Formula I-1, or a salt, solvate, hydrate, polymorph, co-crystal, tautomer, stereoisomer, isotopically labeled compound, or prodrug thereof; and
(b) an oxidizing agent.

In certain embodiments, the oxidizing agent is a transition metal oxidizing agent. In certain embodiments, the transition metal is Mn(IV), Mn(VII), Cr(VI), Fe(III), Ru(VIII), Sb(V), Ce(IV), Os(VIII), Pt(VI), Tl(III), Pb(IV), or Bi(V). In certain embodiments, the transition metal oxidizing agent is MnO₂.

In certain embodiments, the oxidizing agent is a peroxide. In certain embodiments, the peroxide is an organic peroxide, hydrogen peroxide, peroxy acid, main group peroxide, or metal peroxide. In certain embodiments, the peroxide is an organic hydroperoxide. In certain embodiments, the peroxide is (unsubstituted C₁₋₆ alkyl)OOH. In certain embodiments, the peroxide is tert-BuOOH.

In certain embodiments, Formula III is:

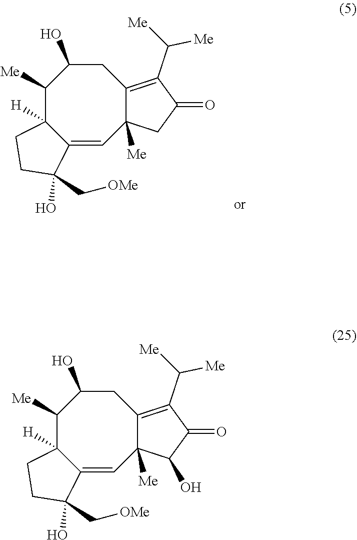 [SMILES: COC[C@@]1(O)CC[C@@H]2/C1=C\[C@@]1(C)CC(=O)C(C(C)C)=C1C[C@H](O)[C@@H]2C ; COC[C@@]1(O)CC[C@@H]2/C1=C\[C@]1(C)C(=C(C(C)C)C(=O)[C@H]1O)C[C@H](O)[C@@H]2C]

In certain embodiments, the second reaction mixture further comprises palladium(0) on carbon.

In certain embodiments, the second reaction mixture further comprises a base. In certain embodiments, the base is an inorganic base. In certain embodiments, the base is an alkali metal hydroxide or an alkaline earth metal hydroxide. In certain embodiments, the base is LiOH, NaOH, or KOH. In certain embodiments, the base is KOH. In certain embodiments, the base is Li₂CO₃, Na₂CO₃, or K₂CO₃. In certain embodiments, the base is LiHCO₃, NaHCO₃, or KHCO₃. In certain embodiments, the base is ammonia, ammonium carbonate, or ammonium hydroxide. In certain embodiments, the base is an organic base (e.g., mono-, di-, or tri-(unsubstituted C₁₋₆ alkyl) amine, cyclic non-aromatic amine, or aromatic amine (e.g., pyridine)).

In certain embodiments, the amount of the oxidizing agent is in excess to the amount of (a) of the second reaction mixture. In certain embodiments, the molar ratio of the oxidizing agent to (a) of the second reaction mixture is between 1:1 and 10:1, between 10:1 and 100:1, between 100:1 and 1,000:1, or between 1,000:1 and 10,000:1, inclusive.

In certain embodiments, the second reaction mixture is in vitro.

In certain embodiments, the temperature of the second reaction mixture is between 0 and 10, between 10 and 20, between 20 and 30, or between 30 and 40 °C, inclusive.

In certain embodiments, the pressure of the second reaction mixtures is about 0.5 and 1.1 atm, inclusive.

In certain embodiments, the second time duration is between 1 and 3 hours, between 3 and 6 hours, between 6 and 12 hours, between 12 and 24 hours, between 1 and 3 days, or between 3 and 7 days, inclusive.

In certain embodiments, the method further comprises incubating a third reaction mixture to produce the compound of Formula IV-2:

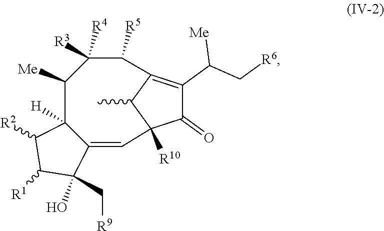

or a salt, solvate, hydrate, polymorph, co-crystal, tautomer, stereoisomer, isotopically labeled compound, or prodrug thereof, wherein the third reaction mixture comprises:
(a) a compound of Formula I-2, or a salt, solvate, hydrate, polymorph, co-crystal, tautomer, stereoisomer, isotopically labeled compound, or prodrug thereof; and
(b) a reducing agent.

In certain embodiments, Formula IV-2 is:

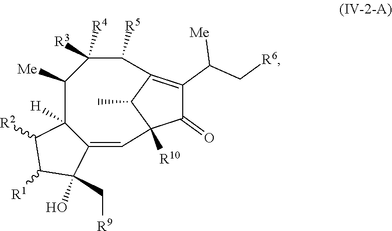

In certain embodiments, Formula IV-2 is:

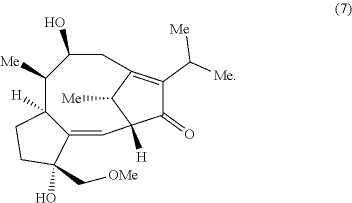 [SMILES: COC[C@@]1(O)CC[C@@H]2/C1=C\[C@H]1C(=O)C(C(C)C)=C(C[C@H](O)[C@@H]2C)[C@H]1C]

In certain embodiments, the amount of the reducing agent is in excess to the amount of (a) of the third reaction mixture. In certain embodiments, the molar ratio of the reducing agent to (a) of the third reaction mixture is between 1:1 and 10:1, between 10:1 and 100:1, between 100:1 and 1,000:1, or between 1,000:1 and 10,000:1, inclusive.

In certain embodiments, the third reaction mixture is in vitro.

In certain embodiments, the temperature of the third reaction mixture is between 0 and 10, between 10 and 20, between 20 and 30, or between 30 and 40 °C, inclusive.

In certain embodiments, the pressure of the third reaction mixtures is about 0.5 and 1.1 atm, inclusive.

In certain embodiments, the third time duration is between 10 and 60 minutes, between 1 and 3 hours, between 3 and 6 hours, between 6 and 12 hours, between 12 and 24 hours, between 1 and 3 days, or between 3 and 7 days, inclusive.

In certain embodiments, the method further comprises incubating a fourth reaction mixture to produce the compound of Formula V-2:

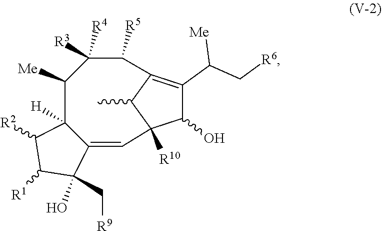

or a salt, solvate, hydrate, polymorph, co-crystal, tautomer, stereoisomer, isotopically labeled compound, or prodrug thereof, wherein the fourth reaction mixture comprises:
(a) a compound of Formula IV-2, or a salt, solvate, hydrate, polymorph, co-crystal, tautomer, stereoisomer, isotopically labeled compound, or prodrug thereof; and
(b) a reducing agent.

In certain embodiments, Formula V-2 is:

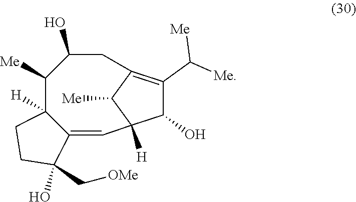 [SMILES: COC[C@@]1(O)CC[C@@H]2/C1=C\[C@@H]1[C@H](C)C(=C(C(C)C)[C@@H]1O)C[C@H](O)[C@@H]2C]

In certain embodiments, the amount of the reducing agent is in excess to the amount of (a) of the fourth reaction mixture. In certain embodiments, the molar ratio of the reducing agent to (a) of the fourth reaction mixture is between 1:1 and 10:1, between 10:1 and 100:1, between 100:1 and 1,000:1, or between 1,000:1 and 10,000:1, inclusive.

In certain embodiments, the fourth reaction mixture is in vitro.

In certain embodiments, the temperature of the fourth reaction mixture is between −100 and −70, between −70 and −50, between −50 and −30, between −30 and −10, between −10 and 15, or between 15 and 25 °C, inclusive.

In certain embodiments, the pressure of the fourth reaction mixtures is about 0.5 and 1.1 atm, inclusive.

In certain embodiments, the fourth time duration is between 5 and 20 minutes, between 20 and 60 minutes, between 1 and 3 hours, between 3 and 6 hours, between 6 and 12 hours, between 12 and 24 hours, or between 1 and 3 days, inclusive.

In certain embodiments, R⁸ is hydrogen. In certain embodiments, R⁸ is —OR^{a}. In certain embodiments, R⁸ is —O(substituted or unsubstituted C₁₋₆ alkyl) (e.g., —OCH₃ or -OBn). In certain embodiments, R⁸ is —O(oxygen protecting group). In certain embodiments, R⁸ is —OH. In certain embodiments,

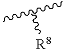

is

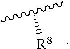

In certain embodiments,

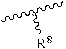

is

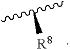

In certain embodiments, R⁹ is —OR^{a}. In certain embodiments, R⁹ is —O(substituted or unsubstituted C₁₋₆ alkyl). In certain embodiments, R⁹ is —OCH₃. In certain embodiments, R⁹ is -OBn. In certain embodiments, R⁹ is —OH. In certain embodiments, R⁹ is hydrogen. In certain embodiments, R⁹ is not hydrogen.

In certain embodiments, R¹⁰ is —OR^{a}. In certain embodiments, R¹⁰ is —O(substituted or unsubstituted C₁₋₆ alkyl) (e.g., —OCH₃ or -OBn). In certain embodiments, R¹⁰ is —O(oxygen protecting group). In certain embodiments, R¹⁰ is —OH. In certain embodiments, R¹⁰ is hydrogen.

In certain embodiments, R¹¹ is —OR^{a}. In certain embodiments, R¹¹ is —O(substituted or unsubstituted C₁₋₆ alkyl) (e.g., —OCH₃ or -OBn). In certain embodiments, R¹¹ is —O(oxygen protecting group). In certain embodiments, R¹¹ is —OH. In certain embodiments, R¹¹ is hydrogen.

In certain embodiments, at least one instance of R^{a} is hydrogen. In certain embodiments, each instance of R^{a} is hydrogen. In certain embodiments, at least one instance of R^{a} is not hydrogen. In certain embodiments, no instance of R^{a} is hydrogen. In certain embodiments, at least one instance of R^{a} is unsubstituted acyl (e.g., —C(═O)H). In certain embodiments, at least one instance of R^{a} is substituted acyl. In certain embodiments, at least one instance of R^{a} is substituted alkyl (e.g., alkyl substituted with one or more instances of halogen (e.g., F)). In certain embodiments, at least one instance of R^{a} is unsubstituted alkyl. In certain embodiments, at least one instance of R^{a} is unsubstituted C₁₋₆ alkyl. In certain embodiments, at least one instance of R^{a} is Me. In certain embodiments, at least one instance of R^{a} is Et, Pr, or Bu. In certain embodiments, at least one instance of R^{a} is substituted C₁₋₆ alkyl. In certain embodiments, at least one instance of R^{a} is substituted methyl (e.g., fluorinated methyl or Bn). In certain embodiments, at least one instance of R^{a} is substituted ethyl, substituted propyl, or substituted butyl. In certain embodiments, at least one instance of R^{a} is substituted or unsubstituted alkenyl. In certain embodiments, at least one instance of R^{a} is substituted or unsubstituted, C₂₋₆ alkenyl (e.g., substituted or unsubstituted vinyl or substituted or unsubstituted allyl). In certain embodiments, at least one instance of R^{a} is substituted or unsubstituted alkynyl. In certain embodiments, at least one instance of R^{a} is substituted or unsubstituted, C₂₋₆ alkynyl (e.g., substituted or unsubstituted ethynyl). In certain embodiments, at least one instance of R^{a} is substituted or unsubstituted carbocyclyl (e.g., substituted or unsubstituted, monocyclic, 3- to 7-membered carbocyclyl comprising 0, 1, or 2 double bonds in the carbocyclic ring system, as valency permits). In certain embodiments, at least one instance of R^{a} is substituted or unsubstituted cyclopropyl, substituted or unsubstituted cyclobutyl, substituted or unsubstituted cyclopentyl, substituted or unsubstituted cyclohexyl, or substituted or unsubstituted cycloheptyl. In certain embodiments, at least one instance of R^{a} is substituted or unsubstituted heterocyclyl (e.g., substituted or unsubstituted, 3- to 7-membered, monocyclic heterocyclyl). In certain embodiments, at least one instance of R^{a} is substituted or unsubstituted oxetanyl, substituted or unsubstituted tetrahydrofuranyl, substituted or unsubstituted tetrahydropyranyl, substituted or unsubstituted azetidinyl, substituted or unsubstituted pyrrolidinyl, substituted or unsubstituted piperidinyl, substituted or unsubstituted morpholinyl, or substituted or unsubstituted piperazinyl. In certain embodiments, at least one instance of R^{a} is substituted or unsubstituted aryl. In certain embodiments, at least one instance of R^{a} is substituted or unsubstituted phenyl. In certain embodiments, at least one instance of R^{a} is substituted or unsubstituted naphthyl. In certain embodiments, at least one instance of R^{a} is substituted or unsubstituted heteroaryl. In certain embodiments, at least one instance of R^{a} is substituted or unsubstituted, 5- to 6-membered, monocyclic heteroaryl. In certain embodiments, at least one instance of R^{a} is substituted or unsubstituted furanyl, substituted or unsubstituted thienyl, substituted or unsubstituted pyrrolyl, substituted or unsubstituted imidazolyl, substituted or unsubstituted oxazolyl, substituted or unsubstituted isoxazolyl, substituted or unsubstituted thiazolyl, or substituted or unsubstituted isothiazolyl. In certain embodiments, at least one instance of R^{a} is substituted or unsubstituted pyridinyl, substituted or unsubstituted pyrazinyl, substituted or unsubstituted pyrimidinyl, or substituted or unsubstituted pyridazinyl. In certain embodiments, at least one instance of R^{a} is substituted or unsubstituted, 9- to 10-membered, bicyclic heteroaryl. In certain embodiments, at least one instance of R^{a} is a nitrogen protecting group (e.g., Bn, Boc, Cbz, Fmoc, trifluoroacetyl, triphenylmethyl, acetyl, or Ts) when attached to a nitrogen atom. In certain embodiments, at least one instance of R^{a} is an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom. In certain embodiments, two instances of R^{a} are joined to form substituted or unsubstituted heterocyclyl (e.g., substituted or unsubstituted, 3- to 7-membered, monocyclic heterocyclyl). In certain embodiments, two instances of R^{a} are joined to form substituted or unsubstituted heteroaryl (e.g., substituted or unsubstituted, 5- to 6-membered, monocyclic heteroaryl).

In certain embodiments, Formula I-1 is:

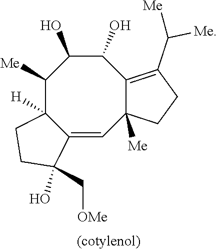 [SMILES: COC[C@@]1(O)CC[C@@H]2/C1=C\[C@@]1(C)CCC(C(C)C)=C1[C@@H](O)[C@H](O)[C@@H]2C]

In certain embodiments, Formula I-1 is:

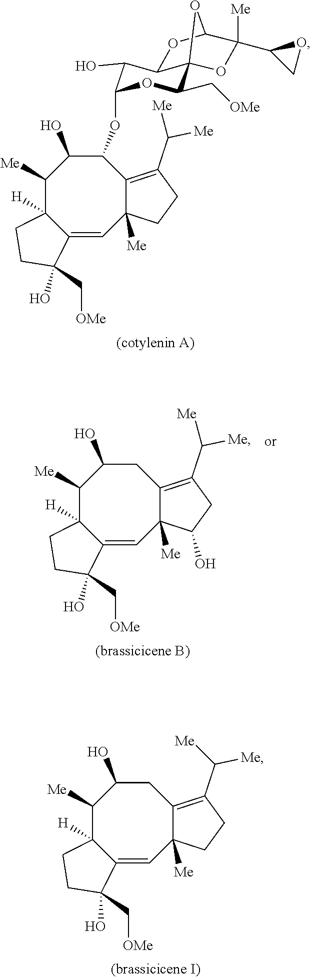

### or Formula I-2 is:

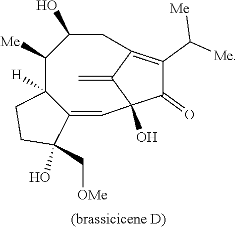 [SMILES: C=C1C2=C(C(C)C)C(=O)[C@]1(O)/C=C1\[C@@H](CC[C@]1(O)COC)[C@@H](C)[C@@H](O)C2]

In certain embodiments, Formula I-1 is:

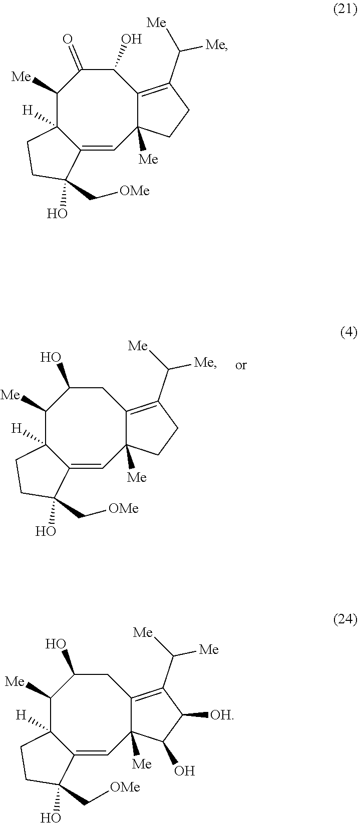 [SMILES: COC[C@@]1(O)CC[C@@H]2/C1=C\[C@@]1(C)CCC(C(C)C)=C1[C@@H](O)C(=O)[C@@H]2C ; COC[C@@]1(O)CC[C@@H]2/C1=C\[C@@]1(C)CCC(C(C)C)=C1C[C@H](O)[C@@H]2C ; COC[C@@]1(O)CC[C@@H]2/C1=C\[C@]1(C)C(=C(C(C)C)[C@@H](O)[C@H]1O)C[C@H](O)[C@@H]2C]

In certain embodiments, Formula I-2 is:

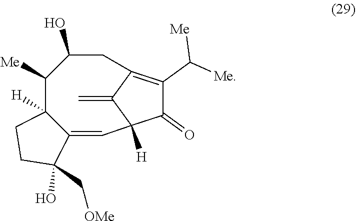 [SMILES: C=C1C2=C(C(C)C)C(=O)[C@@H]1/C=C1\[C@@H](CC[C@]1(O)COC)[C@@H](C)[C@@H](O)C2]

In certain embodiments, Formula II is:

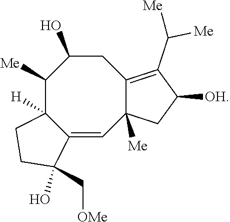 [SMILES: COC[C@@]1(O)CC[C@@H]2/C1=C\[C@@]1(C)C[C@H](O)C(C(C)C)=C1C[C@H](O)[C@@H]2C]

In certain embodiments, a salt of a compound described herein is a pharmaceutically acceptable salt of the compound described herein. In certain embodiments, a compound of Formula I-1 or I-2, or a salt, solvate, hydrate, polymorph, co-crystal, tautomer, stereoisomer, isotopically labeled compound, or prodrug thereof, is the compound of Formula I-1 or I-2, respectively, or the salt thereof. In certain embodiments, a compound of Formula A1 or A2, or a salt, solvate, hydrate, polymorph, co-crystal, tautomer, stereoisomer, isotopically labeled compound, or prodrug thereof, is the compound of Formula A1 or A2, respectively, or the salt thereof. In certain embodiments, a compound of Formula II, or a salt, solvate, hydrate, polymorph, co-crystal, tautomer, stereoisomer, isotopically labeled compound, or prodrug thereof, is the compound of Formula II, or the salt thereof. In certain embodiments, a compound of Formula B, or a salt, solvate, hydrate, polymorph, co-crystal, tautomer, stereoisomer, isotopically labeled compound, or prodrug thereof, is the compound of Formula B, or the salt thereof. In certain embodiments, a compound of Formula U-1 or U-2, or a salt, solvate, hydrate, polymorph, co-crystal, tautomer, stereoisomer, isotopically labeled compound, or prodrug thereof, is the compound of Formula U-1 or U-2, respectively, or the salt thereof.

In certain embodiments, the ratio of the non-heme dioxygenase to (a) of the first reaction mixture is between 10 mg:0.03 mmol and 10 mg:0.1 mmol, between 10 mg:0.1 mmol and 10 mg:0.3 mmol, between 10 mg:0.3 mmol and 10 mg:1 mmol, between 10 mg:1 mmol and 10 mg:3 mmol, between 10 mg:3 mmol and 10 mg:10 mmol, between 10 mg:10 mmol and 10 mg:30 mmol, between 10 mg:30 mmol and 10 mg:100 mmol, or between 10 mg:100 mmol and 10 mg:300 mmol, inclusive.

In certain embodiments, the first reaction mixture further comprises a reducing agent.

In certain embodiments, the reducing agent is tris (2-carboxyethyl) phosphine, ascorbic acid, a borohydride, carbon, carbon monoxide, diborane, a dithionate, dithiothreitol, a ferrous salt, a ferrous compound, formic acid, hydrazine, a hydride, hydrogen, a hypophosphite, an iodide, oxalic acid, palladium(0) on carbon, a phosphite, phosphorous acid, red-A1, a reducing sugar, sodium amalgam, sodium-lead alloy, a stannous salt, a stannous compound, sulfur dioxide, a thiosulfate, or zinc amalgam, or a salt or solvate thereof. In certain embodiments, the reducing agent is tris (2-carboxyethyl) phosphine, or a salt or solvate thereof. In certain embodiments, the reducing agent is palladium(0) on carbon. In certain embodiments, the reducing agent is a metal hydride. In certain embodiments, the reducing agent is diisobutylaluminium hydride. In certain embodiments, the reducing agent is lithium aluminum hydride. In certain embodiments, the reducing agent is sodium borohydride.

In certain embodiments, the amount of the reducing agent is in excess to the amount of (a) of the first reaction mixture. In certain embodiments, the molar ratio of the reducing agent to (a) of the first reaction mixture is between 1:1 and 10:1, between 10:1 and 100:1, between 100:1 and 1,000:1, or between 1,000:1 and 10,000:1, inclusive.

In certain embodiments, the first reaction mixture further comprises an aqueous buffer solution, wherein the pH of the aqueous buffer solution at about 25 °C is between 5 and 6, between 6 and 7, between 7 and 8, or between 8 and 9, inclusive. In certain embodiments, the pH of the aqueous buffer solution at about 25 °C is between 7 and 9, inclusive. In certain embodiments, the aqueous buffer solution is a tris buffer solution.

In certain embodiments, the first reaction mixture is in vitro.

In certain embodiments, the temperature of the first reaction mixture is between 0 and 10, between 10 and 20, between 20 and 30, or between 30 and 40 °C, inclusive.

In certain embodiments, the pressure of the first reaction mixtures is about 0.5 and 1.1 atm, inclusive.

In certain embodiments, the first time duration is between 1 and 3 hours, between 3 and 6 hours, between 6 and 12 hours, between 12 and 24 hours, between 1 and 3 days, or between 3 and 7 days, inclusive.

In certain embodiments, the step of incubating a first reaction mixture further provides a compound of Formula U-1 or U-2:

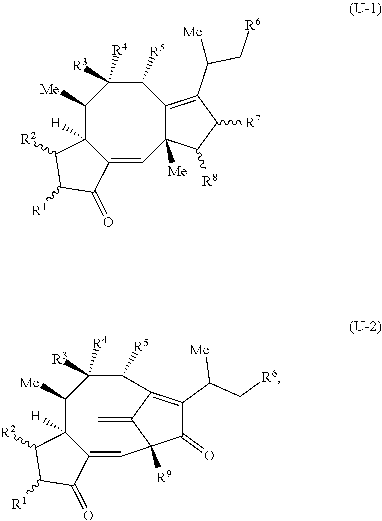

respectively, or a salt, solvate, hydrate, polymorph, co-crystal, tautomer, stereoisomer, isotopically labeled compound, or prodrug thereof.

In certain embodiments, Formula U-1 is:

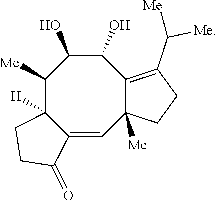 [SMILES: CC(C)C1=C2[C@@H](O)[C@H](O)[C@H](C)[C@@H]3CCC(=O)/C3=C/[C@@]2(C)CC1]

In certain embodiments, the compound of Formula U-1 or U-2, or a salt, solvate, hydrate, polymorph, co-crystal, tautomer, stereoisomer, isotopically labeled compound, or prodrug thereof, is a byproduct of the methods of the present disclosure. The methods of the present disclosure may be advantageous over known methods at least because the former may provide higher product (e.g., the compound of Formula I-1 or I-2, or a salt, solvate, hydrate, polymorph, co-crystal, tautomer, stereoisomer, isotopically labeled compound, or prodrug thereof)-to-byproduct (e.g., the compound of Formula U-1 or U-2, or a salt, solvate, hydrate, polymorph, co-crystal, tautomer, stereoisomer, isotopically labeled compound, or prodrug thereof) ratio. In certain embodiments, the molar ratio of the compound of Formula I-1 or I-2, or a salt, solvate, hydrate, polymorph, co-crystal, tautomer, stereoisomer, isotopically labeled compound, or prodrug thereof, to the compound of Formula U-1 or U-2, respectively, or a salt, solvate, hydrate, polymorph, co-crystal, tautomer, stereoisomer, isotopically labeled compound, or prodrug thereof, is between 60:40 and 70:30, between 70:30 and 80:20, between 80:20 and 90:10, between 90:10 and 95:5, between 95:5 and 99:1, or between 99:1 and 99.9:0.1, inclusive.

In certain embodiments, the rate of conversion of (a) of the first reaction mixture to the compound of Formula I-1 or I-2, or a salt, solvate, hydrate, polymorph, co-crystal, tautomer, stereoisomer, isotopically labeled compound, or prodrug thereof, is between 20% and 40%, between 40% and 50%, between 50% and 60%, between 60% and 80%, or between 80% and 99%, inclusive.

In certain embodiments, the method further comprises purifying the compound of Formula I-1 or I-2, or a salt, solvate, hydrate, polymorph, co-crystal, tautomer, stereoisomer, isotopically labeled compound, or prodrug thereof.

In certain embodiments, the step of purifying comprises liquid-liquid phase separation, drying, filtration, concentration, chromatography, decolorization, or recrystallization, or a combination thereof.

In certain embodiments, the method further comprising one or more steps prior to the step of incubating a first reaction mixture. In certain embodiments, the one or more steps prior to the step of incubating a first reaction mixture provides the compound of Formula I-1 or I-2, or a salt, solvate, hydrate, polymorph, co-crystal, tautomer, stereoisomer, isotopically labeled compound, or prodrug thereof,

In certain embodiments, the method further comprising one or more steps subsequent to the step of incubating a first reaction mixture. In certain embodiments, the one or more steps subsequent to the step of incubating a first reaction mixture provides cotylenin, or an analog thereof (e.g., cotylenin A, brassicicene B, brassicicene I, brassicicene D).

In another aspect, the present disclosure provides a kit comprising:
a non-heme dioxygenase comprising an amino acid sequence that is at least 80% identical to the amino acid sequence of SEQ ID NO: 30, 26, 28, 24, 22, 35, 33, or 18; and
instructions for using the non-heme dioxygenase in the method.

The non-heme dioxygenase comprised in the kit is as described herein.

In another aspect, the present disclosure provides kit comprising:
a cytochrome P450 hydroxylase comprising an amino acid sequence that is at least 80% identical to the amino acid sequence of SEQ ID NO: 32; and
instructions for using the cytochrome P450 hydroxylase in the method.

The cytochrome P450 hydroxylase comprised in the kit is as described herein.

In another aspect, the present disclosure provides a compound described herein, or a salt (e.g., pharmaceutically acceptable salt), solvate, hydrate, polymorph, co-crystal, tautomer, stereoisomer, isotopically labeled compound, or prodrug thereof. In certain embodiments, the compound is a compound described in the EXAMPLES section. In certain embodiments, the compound is not naturally occurring.

In another aspect, the present disclosure provides a pharmaceutical composition comprising a provided compound and optionally a pharmaceutically acceptable excipient.

In another aspect, the present disclosure provides a kit comprising a provided compound or pharmaceutical composition, and instructions for using the provided compound or pharmaceutical composition.

### EXAMPLES

Abbreviations: VO(acac)₂, vanadyl acetylacetonate; DMSO, dimethylsulfoxide; TMS, trimethylsilyl; TBABF; tert-butylammonium bifluoride; MoOPH, oxodiperoxymolybdenum(pyridine)-(hexamethylphosphoric triamide).

### General Information

Unless otherwise noted, all reagents were purchased from commercial suppliers without further purification. Reactions were monitored by thin layer chromatography on plates (60F-254) supplied by Merck Co., visualized by UV (254 nm), Hanessian's stain, KMnO₄, or iodine vapor. Flash column chromatography was performed using E. Merck silica gel (60, particle size 0.040-0.063 mm). Yields refer to chromatographically and spectroscopically (¹H NMR) pure materials. NMR spectra were recorded on Bruker AV400 (400 MHz for ¹H NMR) or AV600 (600 MHz for ¹H NMR) instruments and calibrated by using residual CHCl₃ (δH=7.26 ppm) and CDCl₃ (δC=77.16 ppm), C₆D₅H (δH=7.16 ppm) and C₆D₆(δC=128.06 ppm), CD₃COCD₂H (δH=2.05 ppm) and CD₃COCD₃ (δC=206.26, 29.84 ppm), CD₂HOD (δH=3.31 ppm) and CD₃OD (δC=49.00 ppm) as internal references. The following abbreviations were used to explain the multiplicities: s=singlet, d=doublet, t=triplet, q=quartet, hept=heptet, br=broad, td=triple doublet, dt=double triplet, dq=double quartet, m=multiplet. Optical rotations were measured on Autopol IV polarimeter (Rudolph Research Analytical). Enzymes for routine cloning were purchased from New England Biolabs (NEB, Ipswich, MA). Sonication was performed using a Qsonica Q500 sonicator. Purified enzymes were accessed via immobilized metal ion affinity chromatography with HisTrap HP column. Biochemicals and media components were purchased from standard commercial sources. Electrocompetent E. coli BL21(DE3) strains were purchased from Lucigen. All E. coli strains generated in the present disclosure are stored as glycerol stocks at −80 °C.

### Molecular Biology Procedures

pET28a(+)-Bsc9 and pET28a(+)-BscD were provided by the U.S. Department of Energy Joint Genome Institute, a DOE Office of Science User Facility, under contract DE-AC02-05CH11231. pET28a(+)-RsBsc9, pET28a(+)-BvBsc9, pET28a(+)-DhBsc9, pET28a(+)-CcBsc9 and pET28a(+)-MoBsc9 were obtained via DNA synthesis from Twist Bioscience. Briefly, the codon optimized DNA sequences below were inserted between the NdeI and XhoI restriction sites within the commercial pET28a(+) vector.

Generation of pET22b-Based Expression Vector for Bsc9 Homologs

Following digestion of the above pET28 plasmids with NdeI and XhoI, the desired gene fragments were inserted into the MCS of pET22b, obtained by NdeI and XhoI digestion of a pET22b plasmid, by using NEB Quick Ligation kit.

### Site-Directed Mutagenesis of Bsc9

Site-directed mutagenesis on pET28a-Bsc9 was performed by using standard 34 QuikChange PCR method with primers containing the desired mutation at the appropriate position(s). The resulting PCR products were digested with DpnI, gel purified, repaired using NEBuilder HiFi DNA assembly (NEB, product number: E2621), and used directly to transform electrocompetent E. coli BL21(DE3), which already contains pGro7 to express the chaperones GroES/GroEL.

### Generation of Bsc9 “VR”

A standard QuikChange PCR was performed on pET28a-Bsc9 with primers containing the desired replacement of the Ala103-Gly116 loop with the Val-Arg diad from BscD. The resulting PCR products were digested with DpnI, gel purified, repaired using NEBuilder HiFi DNA assembly (NEB, product number: E2621), and used directly to transform electrocompetent E. coli BL21(DE3), which already contains pGro7 to express the chaperones GroES/GroEL.

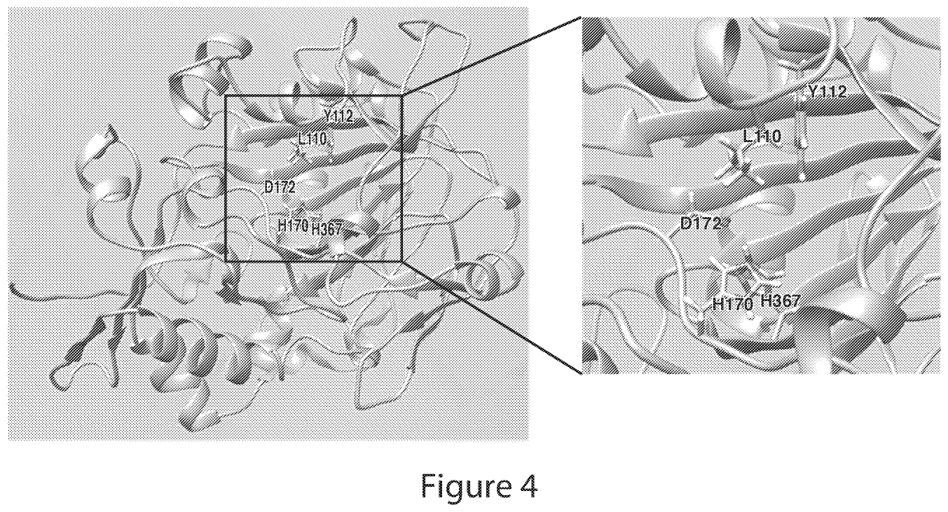

According to structural prediction by RoseTTAfold^{S3}, a homology model for MoBsc9 is shown as in FIG. 4. L110 and Y112 are positioned opposite to the central HXDXₙH triad and hypothesized to be crucial for substrate binding during catalysis. We thus posited that site-saturation mutagenesis on L110 and Y112 will allow us to modulate the catalytic activity of MoBsc9.

### Site-Saturation Mutagenesis of MoBsc9

Site saturation mutagenesis on pET22b-MoBsc9 was performed by using standard QuikChange PCR method with primers containing the desired mutation at the appropriate position(s). The resulting PCR products were digested with DpnI, gel purified, repaired using NEBuilder HiFi DNA assembly (NEB, product number: E2621), and used directly to transform electrocompetent E. coli BL21(DE3), which already contains pGro7 to express the chaperones GroES/GroEL.

### Screening of MoBsc9 Variants in Deep-Well Plates

Single colonies of recombinant E. coli above on agar plates (50 g/mL ampicillin and 25 g/mL chloramphenicol) were picked, transferred into deep-well plates containing LB broth (400 μL, with 50 g/mL ampicillin and 25 g/mL chloramphenicol). For each row, there were two extra wells for control (parent without mutation) and blank (not inoculated). The plates were shaken at 250 rpm at 37 °C for 12 h. Aliquots of these starter cultures (100 μL/well) were transferred to shallow-well plates, mixed with 100 μL 50% glycerol for glycerol stocks and stored at −80 °C. 40 μL aliquots of these starter cultures were transferred to deep-well plates containing terrific broth (TB, 660 μL, with 50 g/mL ampicillin and 25 g/mL chloramphenicol) and shaken at 250 rpm at 37 °C for 2.5 h. The plates were cooled on ice for 20 min, and then induced by the addition of 10 μL solution of isopropyl β-D-1-thiogalactopyranoside (1.75 mM, 70×, final concentration=25 μM) and L-arabinose (70 mg/mL, 70×, final concentration=1 mg/mL). The plates were shaken at 250 rpm at 15 °C for 24 h and then cooled to 0 °C. The cells were harvested by centrifugation (4 °C, 10 min, 2035×g) and resuspended in tris buffer (200 μL, 55 mM, pH 8.5, containing 11 mM tris(2-carboxyethyl)phosphine hydrochloride). The suspension was frozen in liquid nitrogen for 5 min, then put in −78 °C acetone for 5 min, in −20 °C NaCl (sat. aq.) for 10 min and in ice-water for 20 min until totally thawed. 10 μL solution of FeSO₄·7H₂O (20 mM, 20×, final concentration=1 mM) and ascorbic acid (100 mM, 20×, final concentration 5 mM) were added. Another 10 μL disodium α-ketoglutarate dihydrate solution (300 mM, 20×, final concentration=15 mM) was added. Finally, 0.1 mg substrate (20 or 23) in 10 μL DMSO was added with gentle shaking. The plates were shaken at 180 rpm at 15 °C for 16 h. Then, 100 μL Na₂SO₄ (sat. aq.) and 200 μL ethyl acetate were added and mixed well with pipette to quench the reaction. The mixture was separated by centrifugation (r.t., 10 min, 2035×g). 0.5 μL organic phase from each well was spotted onto TLC with pipette, and the TLC plates were developed with hexanes:ethyl acetate=2:1 for substrate 20, or dichloromethane:acetone=40:1 for substrate 23, and visualized by both UV absorption and Hanessian's stain. The conversion and products ratio were evaluated according to TLC readouts, and hits were verified by scaling up.

For scaling up verification, LB (4 mL, with 50 g/mL ampicillin and 25 g/mL chloramphenicol) was inoculated with glycerol storks of prioritized strains above, and shaken at 250 rpm at 37 °C for 12 h. 200 μL of the culture was used to inoculate 50 mL terrific broth media (in 250 mL non-beveled Erlenmeyer flask, with 50 g/mL ampicillin and 25 g/mL chloramphenicol). The culture was shaken at 250 rpm at 37 °C for roughly 3 hours or until an optical density (OD₆₀₀) of 0.9-1.0 was reached. The culture was cooled on ice for 20 min, and then induced by the addition of isopropyl β-D-1-thiogalactopyranoside (50 L, 1000×, final concentration=25 μM) and L-arabinose (50 mg, final concentration=1 mg/mL). The culture was shaken at 250 rpm at 15 °C for 24 h and then cooled to 0 °C. The cells were harvested by centrifugation (4 °C, 10 min, 3234×g) and resuspended in tris buffer (50 mM, pH 8.5) to an OD₆₀₀ of around 60. Tris(2-carboxyethyl)phosphine hydrochloride (neutralized with 3.8 eq. of NaOH to make the pH around 8.5) was added to a final concentration of 10 mM. The suspension was disrupted by sonication cooled by ice-water. The crude lysate was centrifuged (4 °C, 10 min, 3234×g) again to make clarified lysate, which was kept at 0 °C and used within 1 h.

20 mL borosilicate glass scintillation vial was cooled to 0 °C and charged with the above clarified lysate (1 mL). 10 μL solution of FeSO₄·7H₂O (100 mM, 100×, final concentration=1 mM) and ascorbic acid (500 mM, 100×) was added. Another 10 μL disodium α-ketoglutarate dihydrate solution (1.5 M, 100×, final concentration=5 mM) was added. Finally, substrate 20 or (1 mg, ca. 3 mol, dissolved in 50 L DMSO) was added dropwise with gentle shaking. The vial was shaken at 160 rpm at 15 °C for 10 h. The reaction mixture was treated with Na₂SO₄ (sat. aq., 1 mL) and extracted with EtOAc (2 mL×2). The combined organic phase was concentrated and the residue was checked by crude ¹H NMR (in CDCl₃). The conversion and products ratio were determined by integration of characteristic proton signal: 20 (4.29, s, 1H), 21 (5.81, d, 1H), 22 (6.85, d, 1H), 23 (3.28, s, 3H), 4 (5.59, d, 1H), S15 (6.72, d, 1H).

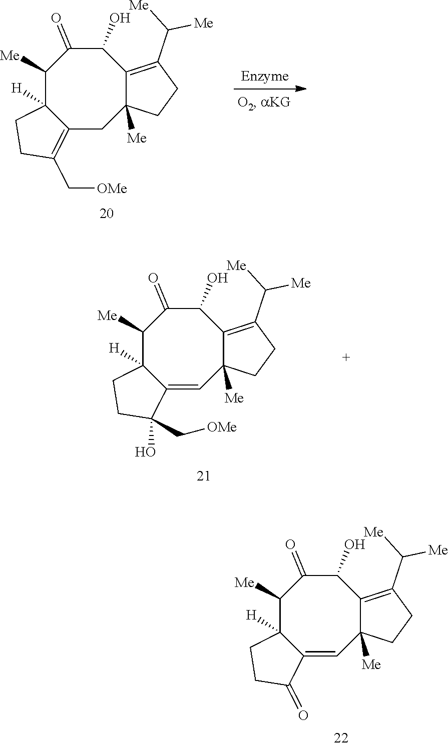 [SMILES: COCC1=C2C[C@@]3(C)CCC(C(C)C)=C3[C@@H](O)C(=O)[C@H](C)[C@@H]2CC1 ; COC[C@@]1(O)CC[C@@H]2/C1=C\[C@@]1(C)CCC(C(C)C)=C1[C@@H](O)C(=O)[C@@H]2C ; CC(C)C1=C2[C@@H](O)C(=O)[C@H](C)[C@@H]3CCC(=O)/C3=C/[C@@]2(C)CC1]

TABLE S1
Enzyme screening for oxidation of 20 at 1 mg scale.

| Entry | Enzyme | Ratio of 21:22 | conversion |
|---|---|---|---|
| 1 | Wild-type Bsc9 | 1:1.46 | 43% |
| 2 | MoBsc9 | 1:0.52 | 100% |
| 3 | MoBsc9 Y112M | 1:0.36 | 100% |

Further scaling up of entry 3 to 30 mg scale yield 21:22 in a ratio of 1:0.40, with 100% conversion.

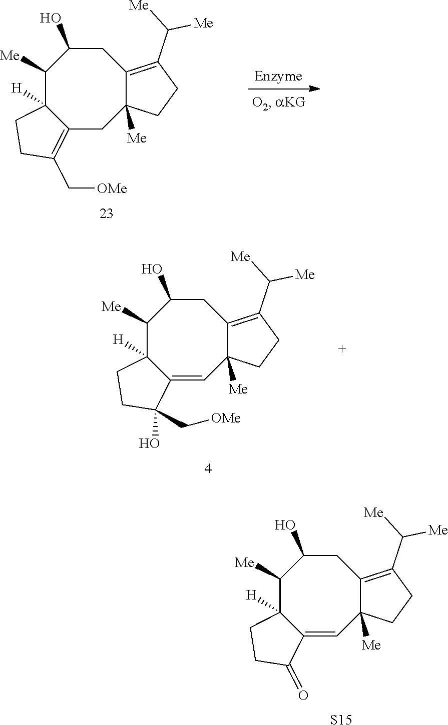 [SMILES: COCC1=C2C[C@@]3(C)CCC(C(C)C)=C3C[C@H](O)[C@H](C)[C@@H]2CC1 ; COC[C@@]1(O)CC[C@@H]2/C1=C\[C@@]1(C)CCC(C(C)C)=C1C[C@H](O)[C@@H]2C ; CC(C)C1=C2C[C@H](O)[C@H](C)[C@@H]3CCC(=O)/C3=C/[C@@]2(C)CC1 ; "S15"]

TABLE S2
Enzyme screening for oxidation of 23 at 1 mg scale.

| Entry | Enzyme | Ratio of 4:S15 | conversion |
|---|---|---|---|
| 1 | Wild-type Bsc9 | 1:1.30 | 37% |
| 2 | MoBsc9 | 1:1.23 | 30% |
| 3 | MoBsc9 Y112R | 1:0.96 | 67% |
| 4 | MoBsc9 Y112R L110A | 1:0.47 | 98% |

Further scaling up of entry 4 to 130 mg scale yield 4: S15 in a ratio of 1:0.47, with 98% conversion.

TABLE S4
Optimization of Prins cyclization-transannular hydride shift cascade.

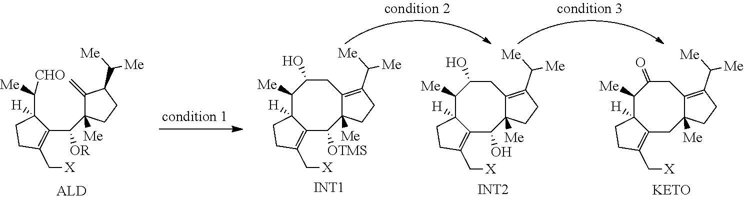

| substrates | | condition 1 | results |
|---|---|---|---|
| R = TMS | X = H | PPTS, MeOH, 50 °C | INT2, 30~35% |
| R = H | X = H | BF₃·Et₂O (3 eq.), DCM, −78 °C | INT2, 83% at 50 mg scale |
| | | | ~70% conv., ~50% yield at 800 mg scale |
| R = TMS | X = H | BF₃·Et₂O (3 eq.), DCM, −78 °C, | INT1, 78% |
| | | quenching in situ by pyridine | |
| R = H | X = OBn | BF₃·Et₂O (3 eq.), DCM, −78 °C | INT2 72%, 200 mg scale |
| | | | INT2, 35% + INT2 C7-epimer ~30%, 1 g scale |
| R = TMS | X = OBn | BF₃·Et₂O (3 eq.), DCM, −78 °C | INT1 35% + INT2 45% |
| R = TMS | X = OBn | BF₃·Et₂O (1.2 eq.), DCM, -42 °C | INT1 ~40% + INT2 ~20% + ALD ~40% |
| R = TMS | X = OBn | BF₃·Et₂O (3 eq.), DCM, −78 ~ 0 °C | elimination S12 |
| R = TMS | X = OMe | BF₃·Et₂O (3 eq.), DCM, −78 °C, | INT2 + trace KETO |
| | | then pinacol (3 eq.), 0 °C | |
| R = TMS | X = OMe | BF₃·Et₂O (3 eq.), DCM, −78 °C, | INT2 + trace KETO |
| | | then pinacol (6 eq.), 0 °C | |
| R = TMS | X = OMe | BF₃·Et₂O (3 eq.), DCM, −78 °C, | INT2 + trace KETO + decomposition |
| | | then KHF₂ (2 eq.), THF, 0 °C | |
| R = TMS | X = OMe | BF₃·Et₂O (3 eq.), DCM, −78 °C, | INT2 |
| | | then Bu₄NHF₂ (3 eq.), 0 °C | |
| R = TMS | X = OMe | BF₃·Et₂O (3 eq.), DCM, −78 °C, | KETO 62% at 200 mg scale, 57% at 1 g scale |
| | | then Bu₄NHF₂ (1.5 eq.), 0 °C | |
| R = TMS | X = OMe | HBF₄ (10 mM), DCM, −78 °C | ALD (X = OMe, R = H), desilylation |
| R = TMS | X = OMe | B(C₆F₅)₃ (1 eq.), DCM, −78 ~0 °C | slow decomposition |

| substrates | condition 2 | results |
|---|---|---|
| X = H | TBAF, THF, 0 °C | decomposition |
| X = H | TASF, DMF, r.t. | decomposition |
| X = H | PPTS, THF/H₂O (10/1), 40 °C | ~80% at 40 mg scale, ~40% at 500 mg scale |
| X = OBn | PPTS, THF/H₂O (10/1), 40 °C | very slow conversion at >400 mg scale, decomposition |
| X = OBn | PPTS, DMF/H₂O (10/1), r.t. | complex mixture |
| X = OBn | PyHBF₄, THF, 40 °C | very slow conversion |
| X = OBn | PyHBF₄, DMF, r.t. | ~70% conversion |
| X = OBn | NH₄HF₂, THF, r.t. | ~30% conversion |
| X = OBn | NH₄HF₂, MeOH, r.t. | complex mixture |
| X = OBn | NH₄HF₂, DMF, r.t. | slow conversion, decomposition |
| X = OBn | TBAF-AcOH (0.2 M), THF | ~80% conversion |
| X = OBn | TBAF-ACOH (1 M), THF | quant. |
| substrates | condition 3 | results |
| X = OBn | BiCl₃ (2 eq.), DCM, r.t. | KETO: 91% at 20 mg scale, ~60% at 400 mg scale |
| X = OBn | InCl₃ (1 eq.), DCM, r.t. | elimination, S12 |
| X = OBn | ZnCl₂ (1 eq.), DCM, r.t. | KETO: 92% at 60 mg scale, 53% at 400 mg scale |
| X = OBn | Zn(OTf)₂ (1 eq.), DCM, reflux | no reaction |
| X = OBn | Sc(OTf)₃ (1 eq.), DCM, r.t. | KETO: ~50%, butadiene: ~50% |
| X = OBn | TFA/DCM (1/10), r.t. | KETO: 65% at 1 g scale |
| X = OBn | TFA/DCM (1/10), 3A M.S., r.t. | KETO: 59% at 200 mg scale |
| X = OBn | MsOH (10 mM), DCM, r.t. | mixture of KETO, S12 and others |

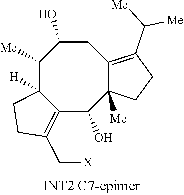

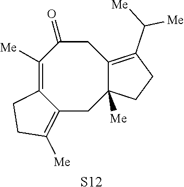 [SMILES: CC1=C2C[C@@]3(C)CCC(C(C)C)=C3CC(=O)/C(C)=C\2CC1 ; "S12"]

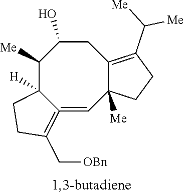

TABLE S5
Optimization of C9 oxidation

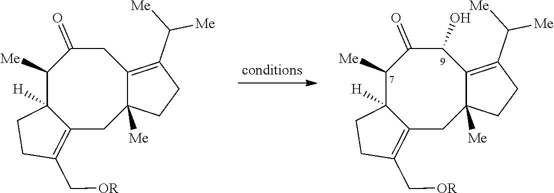

| substrates | conditions | | results |
|---|---|---|---|
| R = Bn | LiHMDS | | no reaction |
| | NaHMDS | Davis′ [O]s | |
| | KHMDS | O₂, P(OR)₃ | |

| | | |
|---|---|---|
| | (1 M), THF, r.t., | |
| | KOtBu | |
| R = Bn | LDA, THF, 40 °C | 1,2/2,3-Wittig rearrangement |
| R = Bn | TESOTf, Py, DCM, then mCPBA | 2,3-epoxide |
| R = Bn | t-BuOK (0.5 M), THF, 40 °C, then O₂, P(OMe)₃, −78 °C ~ r.t. | ~50% conv., 35% C9 oxidation |
| R = H | LiHMDS, THF, 0 °C | decomposition |
| R = Me | t-BuOK (0.5 M), THF, 40 °C, then O₂, P(OMe)₃, −78 °C ~ r.t. | 30~50% C7 oxidation, trace C9 oxidation |
| R = Me | LDA | no reaction |
| | LINEt₂ (1 M), THF, r.t., O₂, P(OMe)₃, −78 °C ~ r.t. | |
| | KHMDS | |
| R = Me | LiHMDS (1 M), THF, r.t., O₂, P(OMe)₃, −78 °C ~ r.t. | ~10% conv. C9 oxidation only |
| R = Me | LiHMDS-Lil (1 M), THF, r.t., O₂, P(OMe)₃, −78 °C ~ r.t. | ~10% conv. C9 oxidation only |
| R = Me | t-BuOLi (1 M), THF, 30 °C, then O₂, P(OMe)₃, −78 °C ~ r.t. | ~60% C7 oxidation, trace C9 oxidation |
| R = Me | t-BuOLi (1 M), KH, THF, 30 °C, then O₂, P(OMe)₃, −78 °C ~ r.t. | 58% C9 oxidation, 4% C7 oxidation, 78% conversion |

1,2/2,3 Wittig rearrangement 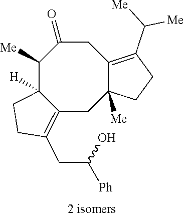 [SMILES: CC(C)C1=C2CC(=O)[C@H](C)[C@@H]3CCC(CC(O)c4ccccc4)=C3C[C@@]2(C)CC1] 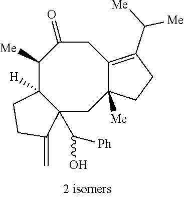 [SMILES: C=C1CC[C@H]2[C@@H](C)C(=O)CC3=C(C(C)C)CC[C@]3(C)CC12C(O)c1ccccc1] 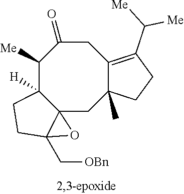 [SMILES: CC(C)C1=C2CC(=O)[C@H](C)[C@@H]3CCC4(COCc5ccccc5)OC34C[C@@]2(C)CC1] 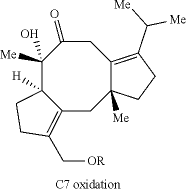
Davis' [O]s:

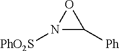

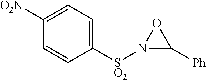 [SMILES: O=[N+]([O-])c1ccc(S(=O)(=O)N2OC2c2ccccc2)cc1]

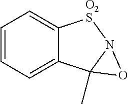

### Syntheses of Compounds S1a and S1b

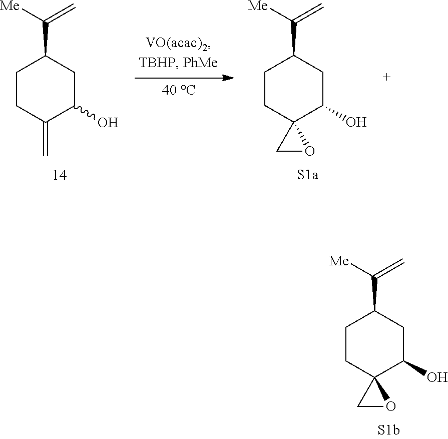

Compound 14 was prepared according to previously reported procedure.

A solution of 14 (28.80 g, 189.2 mmol) in PhMe (900 mL) was treated with t-BuOOH (41 mL, 5.5-6.0 M in decane, ca. 225 mmol, 1.3 eq.) and VO(acac)₂ (2.51 g, 9.46 mmol, 0.05 eq.). The resulting solution was stirred at 40 °C for 36 h under Ar and then cooled to room temperature. The solution was washed with a mixed solution of NaHSO₃ (10 g) and NaOAc (10 g) in H₂O (150 mL), filtered through a short silica gel pad and eluted with hexanes:EtOAc (4:1) to give a mixture of Sla, Sib and additional minor impurities (total crude mass after solvent removal=29.7 g). The crude product could be used in the next step directly without further purification. A pure sample of isomer Sla could not be obtained due to the presence of impurities but isomer Sib was successfully purified by preparative TLC (hexanes:EtOAc=4:1, then CH₂Cl₂:EtOAc=12:1). The stereochemical configuration of S1b was determined by NMR analysis.

Characterization data of S1b: R_{f}=0.3 (silica gel, hexanes:EtOAc=4:1); [α]_{D}^{25.7}=+14.6 (c=0.5 in EtOAc); ¹H NMR (600 MHz, CDCl₃): δ=4.74-4.73 (m, 2H), 3.81 (td, J=11.3, 4.6 Hz, 1H), 3.14 (d, J=4.9 Hz, 1H), 2.60 (d, J=4.8 Hz, 1H), 2.14-2.08 (m, 2H), 1.99-1.94 (m, 1H), 1.97 (td, J=14.4, 4.1 Hz, 1H), 1.74 (t, J=1.2 Hz, 3H), 1.75-1.71 (m, 1H), 1.56 (d, J=11.6 Hz, 1H), 1.53-1.46 (m, 2H), 1.35 (q, J=12.0 Hz, 1H) ppm; ¹³C NMR (151 MHz, CDCl₃): δ=148.6, 109.4, 67.7, 60.2, 50.3, 43.7, 39.3, 32.0, 28.5, 20.9 ppm; HRMS (ESI, m/z): [M+H]⁺ calcd for C₁₀H₁₇O₃, 169.1223; found 169.1223.

### Syntheses of Compounds S2a and S2b

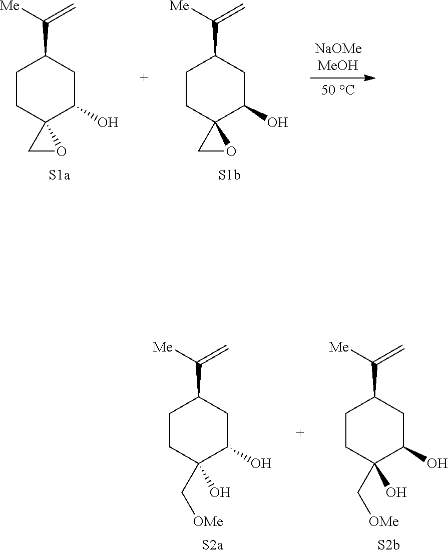

The crude mixture of Sla and Sib (29.7 g) from the previous step was dissolved in MeOH (800 mL) and treated with NaOMe (200 mL, 5 M in MeOH). The solution was stirred at 50 °C for 2 d, cooled to room temperature, quenched by slow addition of NH₄Cl (powder, 80 g) and stirred for another 0.5 h. The suspension was concentrated and resuspended in EtOAc. The suspension was filtered through a short silica gel pad and eluted with EtOAc to give a mixture of S2a, S2b and additional minor impurities (total crude mass after solvent removal=33.5 g). The crude product could be used in the next step directly without further purification. For characterization purposes, S2a and S2b were purified by repeated preparative TLC (hexanes:EtOAc=4:1 and CH₂Cl₂: MeOH=20:1). The stereochemical configuration of S2b was 28 determined by methanolysis of pure S1b.

Characterization data of S2b: R_{f}=0.13 (silica gel, hexanes:EtOAc=2:1); [α]_{D}^{22.0}=+1.3 (c=1.0 in EtOAc); ¹H NMR (600 MHz, CDCl₃): δ=4.71 (dq, J=1.9, 1.0 Hz, 1H), 4.70 (p, J=1.6 Hz, 1H), 3.71 (dd, J=11.3, 4.8 Hz, 1H), 3.47 (d, J=9.2 Hz, 1H), 3.40 (s, 3H), 3.38 (d, J 2=9.2 Hz, 1H), 3.28 (s, 1H), 2.75 (s, 1H), 1.91 (tt, J=12.2, 3.3 Hz, 1H), 1.80-1.74 (m, 2H), 1.72 (t, J=1.2 Hz, 3H), 1.56-1.48 (m, 3H), 1.23 (td, J=13.6, 4.6 Hz, 1H) ppm; ¹³C NMR (151 MHz, CDCl₃): δ=149.1, 109.1, 82.0, 74.2, 71.4, 59.8, 43.1, 34.5, 32.2, 25.3, 20.9 ppm; HRMS (ESI, m/z): [M+H]⁺ calcd for C₁₁H₂₁O₃, 201.1485; found 201.1488.

Characterization data of S2a: R_{f}=0.13 (silica gel, hexanes:EtOAc=2:1); [α]_{D}^{22.2}+15.5 (c=0.2 in EtOAc); ¹H NMR (600 MHz, CDCl₃): δ=4.75 (hex, J=1.4 Hz, 1H), 4.72 (s, 1H), 3.86 (dd, J=5.5, 3.1 Hz, 1H), 3.48 (d, J=9.4 Hz, 1H), 3.40 (s, 3H), 3.36 (d, J=9.4 Hz, 1H), 2.77 (s, 1H), 2.76 (s, 1H), 2.40 (tt, J=9.7, 4.0 Hz, 1H), 1.94 (dddd, J=14.1, 5.8, 4.2, 1.8 Hz, 1H), 1.79-1.73 (m, 2H), 1.73 (s, 3H), 1.58-1.51 (m, 2H), 1.34-1.28 (m, 1H) ppm; ¹³C NMR (151 MHz, CDCl₃): δ=148.7, 109.3, 77.2, 73.1, 69.8, 59.7, 37.4, 33.5, 29.5, 26.6, 21.7 ppm; HRMS (ESI, m/z): [M+H]⁺ calcd for C₁₁H₂₁O₃, 201.1485; found 201.1489.

### Synthesis of Compound 15

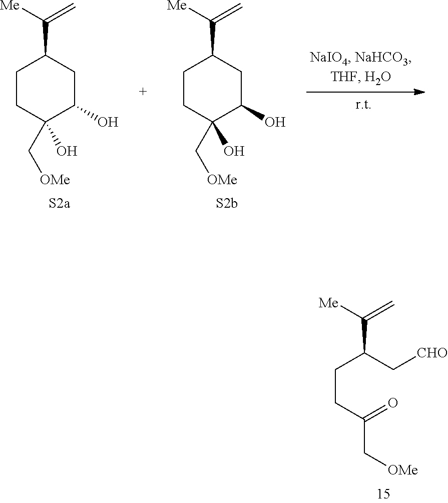

The crude mixture of S2a and S2b (33.3 g from the previous step was dissolved in THF (200 mL) and H₂O (800 mL) and cooled to 0 °C. NaHCO₃ (8.41 g, 100 mmol) and NaIO₄ (powder, 40.64 g, 190 mmol) were added and the mixture was stirred vigorously at room temperature for 12 h under Ar. The biphasic mixture was extracted with hexanes:EtOAc (800 mL, 1:1) twice. The combined organic phase was concentrated and the residue was purified by flash column chromatography (hexanes:EtOAc=10:1 to 6:1) to give product 15 (26.04 g) as a light yellow oil. The three-step sequence of epoxidation/methanolysis/oxidative cleavage provided an overall yield of 69%.

Characterization data of 15: R_{f}=0.25 (silica gel, hexanes:EtOAc=2:1); [α]_{D}^{22.5}=+5.3 (c=1.0 in EtOAc); ¹H NMR (400 MHz, CDCl₃): δ=9.65 (t, J=2.3 Hz, 1H), 4.82 (p, J=1.5 Hz, 1H), 4.76 (dp, J=1.5, 0.8 Hz, 1H), 3.97 (s, 2H), 3.39 (s, 3H), 2.67 (dtd, J=9.8, 7.3, 4.9 Hz, 1H), 2.48-2.36 (m, 4H), 1.73-1.64 (m, 2H), 1.62 (dd, J=1.5, 0.8 Hz, 3H) ppm; ¹³C NMR (100 MHz, CDCl₃): δ=208.2, 201.8, 145.1, 113.5, 77.8, 77.2, 59.4, 47.5, 41.0, 36.3, 26.0, 18.5 ppm; HRMS (ESI, m/z): [M+H]⁺ calcd for C₁₁H₁₉O₃, 199.1329; found 199.1328.

One-Pot Conversion from 14 to 15.

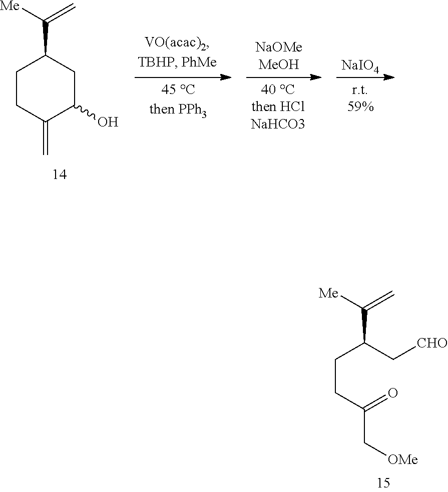

A solution of 14 (15.22 g, 100.0 mmol) in PhMe (20 mL) was treated with t-BuOOH (18.2 mL, 5.5 M in nonane, 100 mmol, 1.0 eq.) and VO(acac)₂ (663 mg, 2.5 mmol, 0.025 eq.). The resulting solution was stirred at 45 °C for 24 h under Ar. At this point, the solution was treated with additional t-BuOOH (1.8 mL, 5.5 M in nonane, 10 mmol, 0.1 eq.), stirred for another 12 h and then cooled to room temperature. PPh₃ (2.62 g, 10 mmol, 0.1 eq.) was added and the resulting solution was stirred for 30 min. MeOH (60 mL) was added, followed by NaOMe (60 mL, 5 M in MeOH, 300 mmol, 3.0 eq.). The solution was stirred at 40 °C for 12 h and then cooled down in ice bath. The solution was diluted with water (600 mL), followed by addition of HCl (12 M in H₂O) slowly with vigorous stirring to adjust pH to around 7 (about 25 mL HCl needed). Appropriate amount of NaHCO₃ (s, ~1 g) was added to stabilize the pH to between 7~8. The biphasic mixture was treated with NaIO₄ (24.50 g, 115 mmol, 1.15 eq.) and stirred vigorously at room temperature for 10 h. The solution was concentrated in vacuo to remove most MeOH and extracted with hexanes:EtOAc (1:1, 400 mL×3). The organic phase was combined, washed with brine (200 mL), concentrated. The residue was purified by flash column chromatography (hexanes:EtOAc=10:1 to 6:1) to give product 15 (11.76 g) as a light yellow oil. This one-pot conversion provided an overall yield of 59%.

### Synthesis of Compound 16

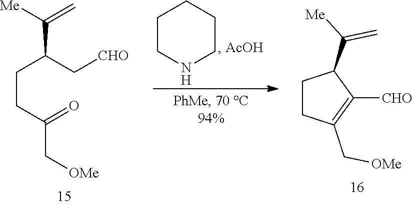

A solution of piperidine (5.59 g, 65.7 mmol, 0.5 eq.) and AcOH (3.94 g, 65.7 mmol, 0.5 eq.) in PhMe (600 mL) was stirred at 70 °C under Ar. A solution of 15 (26.02 g, 131 mmol) in toluene (65 mL) was added dropwise to the mixture via cannula transfer over 0.5 h. The solution was stirred at the same temperature for another 0.5 h and then cooled to room temperature. The solution was washed with NH₄Cl (sat. aq., 100 mL), NaHCO₃ (sat. aq., 100 mL) and then concentrated. The residue was purified by flash column chromatography (hexanes:EtOAc=20:1 to 15:1) to give product 16 (22.27 g, 94%) as a light yellow oil.

Characterization data of 16: R_{f}=0.5 (silica gel, hexanes:EtOAc=4:1); [α]_{D}^{25.8}=+67.2 (c=1.0 in EtOAc); ¹H NMR (400 MHz, CDCl₃): δ=10.02 (s, 1H), 4.70 (penta, J=1.5 Hz, 1H), 4.62 (dt, J=1.8, 0.9 Hz, 1H), 4.42 (penta, J=1.3 Hz, 2H), 3.65 (d, J=9.6 Hz, 1H), 3.37 (s, 3H), 2.66 (ddddt, J=18.8, 8.8, 7.6, 2.4, 1.2 Hz, 1H), 2.56 (dddq, J=18.8, 9.2, 4.0, 1.1 Hz, 1H), 2.10 (dtd, J=13.1, 9.4, 7.7 Hz, 1H), 1.74-1.67 (m, 1H), 1.68 (dd, J=1.5, 0.8 Hz, 3H) ppm; ¹³C NMR (100 MHz, CDCl₃): δ=188.8, 161.1, 146.6, 140.8, 110.2, 69.2, 58.8, 51.4, 35.0, 28.7, 20.6 ppm; HRMS (ESI, m/z): [M+H]⁺ calcd for C₁₁H₁₇O₂, 181.1223; found 181.1223.

### Synthesis of Compound S6

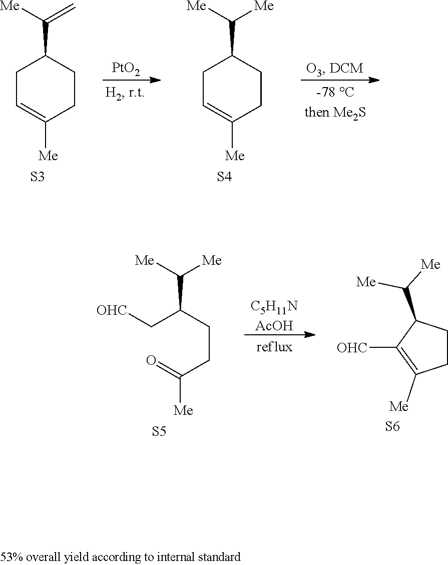

S6 was prepared in one-pot from S3 according to reported procedure with minor modifications^{S4}.

Limonene S3 (5.00 g, 37 mmol) and PtO₂ (50 mg) were added into a high-pressure reactor. The mixture was hydrogenated at 20 atm for approximately 0.5 h at room temperature with vigorous stirring. The dark grey but transparent mixture was transferred to a 1 L round-bottom flask containing S3 (25.20 g, 185 mmol) under H₂, and stirred vigorously at room temperature. The reaction was monitored by crude NMR of aliquots. When the hydrogenation was completed (about 2 d needed), CH₂Cl₂ (500 mL) was added, and the solution was cooled to −78 °C. O₃ was purged through the solution, and the ozonolysis was monitored by TLC analysis (KMnO₄ staining) as the light blue color which usually suggests the ending of ozonolysis is not easy to recognize. Upon completion, Me₂S (20.6 g, 0.33 mol, 1.5 eq.) was added slowly, and the mixture was allowed to warm to room temperature over 1 h. Piperidine (9.43 g, 111 mmol, 0.5 eq.), AcOH (6.65 g, 111 mmol, 0.5 eq.) and Na₂SO₄ (50 g) were added, and the mixture was heated to gentle reflux for 12 h. Water (300 mL) was added with stirring and the biphasic mixture was separated after dissolving all salts. The organic phase was filtered through a short silica gel pad and eluted by pentane:Et₂O (1:1). Further concentration under moderate vacuum gave crude product 33.2 g. It is recommended this crude product be used for the next step without further purification as S6 is volatile. For quantification purposes, 1,3,5-trimethoxybenzene (617.0 mg, 3.668 mmol) was added to the crude solution as internal standard, and the yield was calculated by crude ¹H NMR spectrum (integration of —CHO:OMe=3.54:1, 117 mmol product, yield 53%). The ¹H NMR spectrum of S6 match with previous reports^{S4a}.

### Synthesis of Compound S7

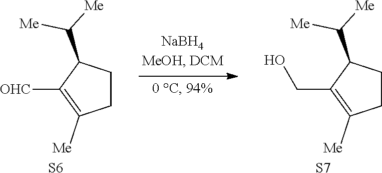 [SMILES: CC1=C(C=O)[C@@H](C(C)C)CC1 ; CC1=C(CO)[C@@H](C(C)C)CC1 ; "S6" ; "S7"]

S7 is a known compound, but the characterization data was not reported yet.

Aldehyde S6 (25.62 g, 168 mmol) was dissolved in MeOH:CH₂Cl₂ (1:1, 500 mL) and cooled to 0 °C. NaBH₄ (6.35 g, 168 mmol, 1.0 eq.) was added in one portion. The solution was stirred at 0 °C for 1 h and then quenched with the addition of NH₄Cl (solid, 20 g). The suspension was stirred vigorously at room temperature for 0.5 h and concentrated in vacuo. MeOH (100 mL) was added and then removed in vacuo, and this operation was repeated for twice. The residue was washed with CH₂Cl₂ thoroughly and loaded onto silica gel column. Elution with hexanes:EtOAc (10:1 to 4:1) gave product S7 24.47 g, yield 94%.

Characterization data of substrate S7: R_{f}=0.3 (silica gel, hexanes:EtOAc=4:1); [α]_{D}^{26.7}=−17.2 (c=1.0 in EtOAc); ¹H NMR (400 MHz, CDCl₃): δ=4.22 (d, J=12.0 Hz, 1H), 3.98 (d, J=11.9 Hz, 1H), 2.81 (s, 1H), 2.26-2.13 (m, 2H), 1.97 (hept d, J=6.9, 3.4 Hz, 1H), 1.77-1.69 (m, 1H), 1.76 (s, 1H), 1.67 (dq, J=2.0, 1.0 Hz, 3H), 1.55 (dddd, J=13.3, 8.4, 5.9, 4.9 Hz, 1H), 0.89 (d, J=6.9 Hz, 3H), 0.64 (d, J=6.9 Hz, 3H) ppm; ¹³C NMR (100 MHz, CDCl₃): δ=137.4, 136.4, 57.5, 51.9, 37.9, 28.7, 21.8, 21.5, 16.0, 13.9 ppm; HRMS (ESI, m/z): [M+H]⁺ calcd for C₁₀H₁₉O, 155.1430; found 155.1430.

### Synthesis of Compound 17

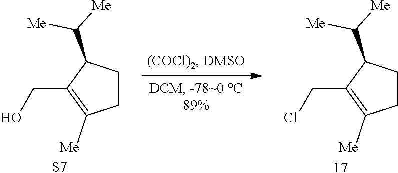

A solution of dimethyl sulfoxide (6.0 mL, 84.5 mmol, 2.4 eq.) in CH₂Cl₂ (120 mL) was cooled to −78 °C and treated with oxalyl chloride (3.6 mL, 41.9 mmol, 1.2 eq.) dropwise over 5 min under Ar. The solution was stirred for another 15 min at the same temperature and treated with substrate S7 (5.347 g, 34.9 mmol) dropwise. The solution was warmed gradually to −20 °C over 1 h and diluted with ice-cold hexanes (130 mL). The solution was poured into NaHCO₃ (sat. aq., 250 mL), stirred for 10 min and separated. The organic phase was washed with Na₂SO₄ (sat. aq., 120 mL), filtered through a column of Na₂SO₄ and eluted by hexanes. The eluent was concentrated to give crude product 17 (5.367 g, 89%) as yellow liquid. The crude product 17 is of sufficient purity for use in the next step without further purification. Note: Product 17 is quite sensitive to acid and cannot be purified by silica gel column. It will decompose upon storage and should be used immediately.

Characterization data of product 17: R_{f}=0.9 (silica gel, hexanes:EtOAc=8:1); ¹H NMR (400 MHz, CDCl₃): δ=4.26 (d, J=11.2 Hz, 1H), 3.99 (d, J=11.2 Hz, 1H), 2.88 (s, 1H), 2.25 (t, J=7.5 Hz, 2H), 1.98 (hept d, J=6.9, 3.4 Hz, 1H), 1.81-1.75 (m, 1H), 1.72 (dq, J=2.1, 1.1 Hz, 3H), 1.57 (dtd, J=13.1, 7.3, 5.6 Hz, 1H), 0.93 (d, J=6.9 Hz, 3H), 0.66 (d, J=6.9 Hz, 3H) ppm; ¹³C NMR (100 MHz, CDCl₃): δ=140.7, 133.3, 51.6, 39.8, 37.8, 28.4, 21.6, 21.4, 15.8, 14.1 ppm; HRMS (ESI, m/z): can not be detected.

### Synthesis of Compound 18

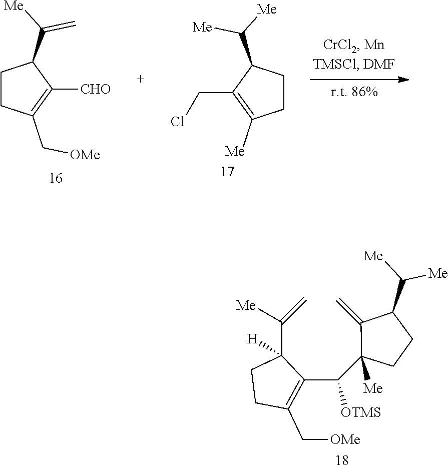

A solution of 17 (5.770 g, 33.4 mmol, 1.15 eq.) and 16 (5.218 g, 28.9 mmol, 1 eq.) in N, N-dimethylformamide (120 mL) was cooled to 0 °C and treated with CrCl₂ (249 mg, 2.0 mmol, 0.07 eq.) and Mn (powder, 50 mesh, 1.836 g, 33.4 mmol, 1.15 eq.). The suspension was degassed with Ar three times. TMSCl (6.290 g, 57.9 mmol, 2.0 eq.) was added via syringe. After stirring at 0 °C for another 15 min, the mixture was warmed to room temperature and stirred for 48 h. Then the reaction was quenched by addition of pyridine (2.4 mL, 1 eq.) and then NH₄Cl (sat. aq., 240 mL). The solution was stirred vigorously for 20 min to decompose excess 17, and then extracted with hexanes:EtOAc (360 mL, 4:1) twice. The combined organic phase was filtered through a silica gel pad and eluted with hexanes:EtOAc (4:1). The eluent was concentrated and purified by flash column chromatography (hexanes:EtOAc=30:1 to 25:1) to give product 18 (9.729 g, 86%) as colorless oil.

Characterization data of 18: R_{f}=0.85 (silica gel, hexanes:EtOAc=8:1); [α]_{D}^{26.3}=+129.7 (c=1.0 in EtOAc); ¹H NMR (400 MHz, CDCl₃): δ=4.90 (d, J=2.8 Hz, 1H), 4.82 (d, J=2.5 Hz, 1H), 4.71-4.69 (m, 1H), 4.67-4.66 (m, 1H), 4.32 (s, 2H), 4.12 (s, 1H), 3.34 (s, 3H), 3.30 (d, J=8.3 Hz, 1H), 2.50-2.36 (m, 2H), 2.24-2.17 (m, 1H), 1.96-1.86 (m, 2H), 1.82-1.74 (m, 1H), 1.67-1.55 (m, 2H), 1.65 (dd, J=1.4, 0.8 Hz, 3H), 1.34-1.24 (m, 2H), 1.02 (s, 3H), 0.97 (d, J=6.8 Hz, 3H), 0.76 (d, J=6.8 Hz, 3H), 0.07 (s, 9H) ppm; ¹³C NMR (100 MHz, CDCl₃): δ=160.3, 148.4, 140.3, 139.6, 111.3, 106.1, 77.8, 70.9, 58.7, 57.8, 52.1, 51.7, 34.1, 33.3, 28.6, 28.5, 8 26.4, 23.8, 22.4, 19.6, 16.8, 0.6 ppm; HRMS (ESI, m/z): [M+H]⁺ calcd for C₂₄H₄₃O₂Si, 391.3027; found 391.3026.

### Synthesis of Compound 19

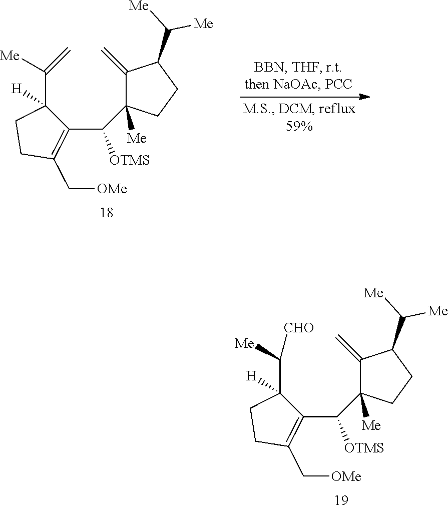

Substrate 18 (958 mg, 2.45 mmol) was dissolved in THF (20 mL) under Ar, and treated with 9-Borabicyclo(3.3.1)nonane (5.9 mL, 0.5 M in THF, 2.95 mmol, 1.2 eq.). The solution was stirred at room temperature for 12 h and the solvent was removed under vacuum. Dichloromethane (24 mL) was added, followed by NaOAc (2.90 g, 35.4 mmol, 14.4 eq.), pyridinium chlorochromate (3.82 g, 17.7 mmol, 7.2 eq.) and dried 3 Å molecular sieves (958 mg). The suspension was stirred at reflux for 4 h. After cooling to room temperature, the suspension was filtered by a short pad of silica gel and eluted by EtOAc. The elution was concentrated in vacuo and the residue was purified by flash column chromatography (hexanes:EtOAc=30:1 to 20:1) to give product 19 (586 mg, 59%) as colorless oil.

Characterization data of 19: R_{f}=0.7 (silica gel, hexanes:EtOAc=4:1); [α]_{D}²⁶.⁴=+18.1 (c=1.0 in EtOAc); ¹H NMR (400 MHz, CDCl₃): δ=9.73 (d, J=1.1 Hz, 1H), 4.91 (d, J=2.9 Hz, 1H), 4.81 (d, J=2.5 Hz, 1H), 4.33 (d, J=11.7 Hz, 2H), 4.32 (d, J=2.4 Hz, 2H), 4.26 (d, J=12.1 Hz, 1H), 3.31 (s, 3H), 2.86 (d, J=9.3 Hz, 1H), 2.66 (qd, J=6.5, 3.1 Hz, 1H), 2.42 (ddd, J=17.0, 9.9, 3.3 Hz, 1H), 2.26 (dt, J=17.1, 8.6 Hz, 1H), 2.19-2.13 (m, 1H), 1.95-1.82 (m, 2H), 1.79-1.72 (m, 1H), 1.60-1.51 (m, 2H), 1.36-1.27 (m, 2H), 1.06 (s, 3H), 1.00 (d, J=6.8 Hz, 3H), 0.97 (d, J=6.8 Hz, 3H), 0.77 (d, J=6.8 Hz, 3H), 0.10 (s, 9H) ppm; ¹³C NMR (100 MHz, CDCl₃): δ=205.8, 161.1, 141.1, 139.0, 105.5, 78.1, 70.7, 58.8, 52.6, 52.3, 52.2, 48.0, 33.7, 32.6, 28.5, 27.2, 25.0, 23.8, 22.3, 16.9, 13.1, 0.6 ppm; HRMS (ESI, m/z): [M+H]⁺ calcd for C₂₄H₄₃O₃Si, 407.2976; found 407.2976.

### Synthesis of Compound S8

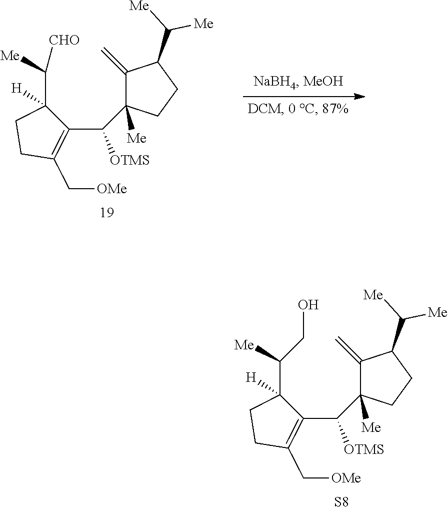

Aldehyde 19 (322 mg, 0.79 mmol) was dissolved in MeOH:CH₂Cl₂ (4 mL:4 mL). The solution was cooled to 0 °C and treated with NaBH₄ (30 mg, 0.79 mmol, 1.0 eq.) in one portion. The mixture was stirred at the same temperature under Ar for 1 h and then quenched with NH₄Cl (100 mg, powder). The suspension was stirred vigorously for 10 min and then concentrated under vacuum. The residue was resuspended with CH₂Cl₂ and loaded onto silica gel column directly. Elution with hexanes:EtOAc (15:1 to 10:1) gave product S8 (280 mg, 87%) as colorless oil. This product can be prepared alternatively from alkene 18 by hydroboration-oxidation in 93% yield.

Characterization data of S8: R_{f}=0.25 (silica gel, hexanes:EtOAc=4:1); [α]_{D}^{26.5}=+49.5 (c=1.0 in EtOAc); ¹H NMR (400 MHz, C₆D₆): δ=5.00 (d, J=2.8 Hz, 1H), 4.84 (d, J=2.5 Hz, 1H), 4.54 (d, J=11.3 Hz, 1H), 4.44 (s, 1H), 4.34 (d, J=11.3 Hz, 1H), 3.62 (dd, J=10.1, 4.1 Hz, 1H), 3.25 (s, 3H), 3.17 (t, J=9.4 Hz, 1H), 2.75 (d, J=8.4 Hz, 1H), 2.58-2.43 (m, 2H), 2.31-2.25 (m, 1H), 2.13-2.04 (m, 1H), 1.96 (ddd, J=12.4, 9.2, 7.0 Hz, 1H), 1.87 (pd, J=6.8, 4.2 Hz, 1H), 1.71 (dtd, J=13.1, 9.6, 8.5 Hz, 1H), 1.61-1.52 (m, 2H), 1.40 (ddd, J=12.4, 7.2, 4.0 Hz, 1H), 1.30 (dtd, J=12.0, 9.4, 7.2 Hz, 1H), 1.19 (s, 3H), 0.98 (d, J=6.8 Hz, 3H), 0.94 (d, J=6.8 Hz, 3H), 0.92 (s, 1H), 0.80 (d, J=6.8 Hz, 3H), 0.24 (s, 9H) ppm; ¹³C NMR (100 MHz, C₆D₆): δ=161.3, 140.8, 140.2, 106.1, 78.5, 71.4, 64.6, 58.9, 54.6, 52.9, 52.6, 38.1, 35.0, 33.7, 29.2, 27.3, 24.4, 24.1, 22.6, 17.8, 17.3, 0.9 ppm; HRMS (ESI, m/z): [M+H]⁺ calcd for C₂₄H₄₅O₃Si, 409.3132; found 409.3133.

### Synthesis of Compound S9

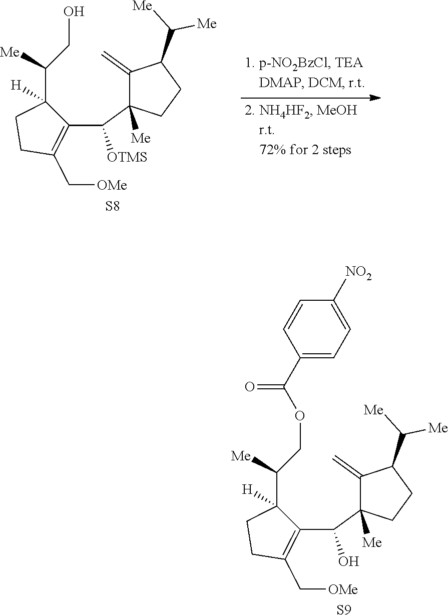

A solution of S8 (68 mg, 0.17 mmol) in CH₂Cl₂ (2 mL) was treated with Et₃N (52 mg, 0.51 mmol, 3 eq.), N,N-dimethyl-4-aminopyridine (4 mg, 0.034 mmol, 0.2 eq.) and 4-nitrobenzoyl chloride (37 mg, 0.2 mmol, 1.2 eq.). The mixture was stirred at room temperature for 4 h and treated with MeOH (1 mL). The mixture was stirred for another 10 min and then concentrated in vacuo. The residue was dissolved in MeOH (2 mL) and treated with NH₄HF₂ (97 mg, 1.7 mmol, 10 eq.). The suspension was stirred at room temperature for 12 h and concentrated in vacuo. The residue was resuspended in CH₂Cl₂ and loaded directly on to silica gel column. Elution with hexanes:EtOAc (15:1 to 10:1) gave product S9 (58 mg, 72% for 2 steps), as colorless oil. The pure product was dissolved in Et₂O and hexanes, and slow evaporation at room temperature yielded single crystals.

Characterization data of S9: R_{f}=0.3 (silica gel, hexanes:EtOAc=4:1); [α]_{D}^{22.8}=+45.4 (c=0.5 in EtOAc); ¹H NMR (400 MHz, CDCl₃): δ=8.30-8.27 (m, 2H), 8.23-8.20 (m, 2H), 5.03 (d, J=2.9 Hz, 1H), 4.86 (d, J=2.4 Hz, 1H), 4.42 (dd, J=10.9, 4.4 Hz, 1H), 4.31 (d, J=11.9 Hz, 1H), 4.25 (d, J=3.8 Hz, 1H), 4.07 (dd, J=10.8, 8.8 Hz, 1H), 4.06 (d, J=12.2 Hz, 1H), 3.33 (s, 3H), 3.32 (d, J=4.3 Hz, 1H), 2.78 (d, J=9.0 Hz, 1H), 2.45-2.28 (m, 4H), 1.95 (pd, J=6.8, 4.0 Hz, 1H), 1.84 (dq, J=13.4, 9.3 Hz, 1H), 1.77-1.69 (m, 2H), 1.64-1.57 (m, 1H), 1.42-1.32 (m, 2H), 1.13 (s, 3H), 1.06 (d, J=6.9 Hz, 3H), 0.98 (d, J=6.8 Hz, 3H), 0.78 (d, J=6.8 Hz, 3H) ppm; ¹³C NMR (100 MHz, CDCl₃): δ=165.1, 161.2, 150.6, 141.3, 138.1, 136.0, 130.9, 123.6, 105.7, 75.8, 70.9, 68.1, 58.6, 54.3, 52.3, 51.3, 35.4, 34.5, 33.9, 28.9, 24.5, 23.6, 23.1, 22.3, 17.3, 16.7 ppm; HRMS (ESI, m/z): [M+H]⁺ calcd for C₂₈H₄₀NO₆, 486.2850; found 486.2850. Crystallographic data for S9 is available free of charge from The Cambridge Crystallographic Data Centre (CCDC 2158016) via www.ccdc.cam.ac.uk/data_request/cif.

### Synthesis of Compound 8

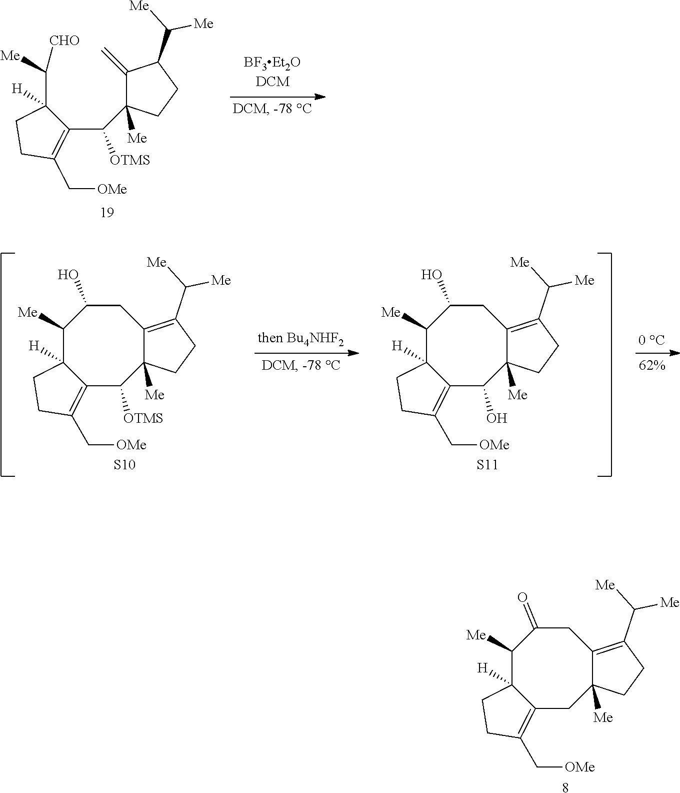

A solution of 19 (192 mg, 0.47 mmol) in CH₂Cl₂ (4.7 mL) was cooled to −78 °C under Ar, and treated with BF₃·Et₂O (201 mg, 1.42 mmol, 3.0 eq.). The solution was stirred at the same temperature for 30 min upon which TLC analysis showed full conversion to intermediates S10 (major) and S11 (minor). n-Bu₄NHF₂ (199 mg, 0.71 mmol, 1.5 eq.) in CH₂Cl₂ (0.5 mL) was added dropwise. After stirring at the same temperature for another 10 min, TLC analysis showed full conversion to intermediate S7. Then the reaction was warmed up to 0 °C and stirred for 6 h. The reaction was quenched by addition of NaHCO₃ (sat. aq., 10 mL) and stirred vigorously for 10 min. The biphasic mixture was diluted by hexanes (5 mL) and then separated. The aqueous phase was extracted with hexanes:EtOAc (4:1, 10 mL) and the combined organic phase was concentrated in vacuo. The residue was purified by flash column chromatography (hexanes:EtOAc=25:1 to 20:1) to give product 8 (94 mg, 62%) as colorless oil. The stereochemistry of intermediates S10 and S11 were tentatively assigned by NOESY spectrum of S11.

Characterization data of intermediate S10: R_{f}=0.6 (silica gel, hexanes:EtOAc=4:1); [α]_{D}^{26.6}=−0.3 (c=0.7 in EtOAc); ¹H NMR (600 MHz, CDCl₃): δ=4.35 (s, 1H), 3.98 (d, J=11.2 Hz, 1H), 3.90 (d, J=11.9 Hz, 1H), 3.52 (s, 1H), 3.30 (s, 3H), 2.94 (hept, J=6.8 Hz, 1H), 2.89 (s, 1H), 2.46-2.38 (m, 2H), 2.34-2.24 (m, 3H), 2.15-2.10 (m, 2H), 1.96-1.91 (m, 1H), 1.46-1.33 (m, 2H), 1.02 (d, J=6.9 Hz, 3H), 0.98 (d, J=7.0 Hz, 3H), 0.88 (d, J=6.8 Hz, 3H), 0.83 (s, 3H), 0.06 (s, 9H) ppm; ¹³C NMR (151 MHz, CDCl₃): δ=69.7, 58.6, 56.5, 33.0, 27.4, 24.7, 21.4, 21.2, −0.1 ppm; HRMS (ESI, m/z): [M+H]⁺ calcd for C₂₄H₄₃O₃Si, 407.2976; found 407.2977.

Characterization data of intermediate S11: R_{f}=0.18 (silica gel, hexanes:EtOAc=4:1); [α]_{D}^{26.4}=+17.0 (c=0.5 in EtOAc); ¹H NMR (600 MHz, CDCl₃): δ=4.44 (s, 1H), 4.01 (d, J=11.6 Hz, 1H), 3.90 (d, J=11.6 Hz, 1H), 3.64 (s, 1H), 3.31 (s, 3H), 3.05 (t, J=9.3 Hz, 1H), 2.95 (hept, J=6.7 Hz, 1H), 2.48 (dd, J=14.7, 5.3 Hz, 1H), 2.38-2.29 (m, 3H), 2.23-2.10 (m, 4H), 1.94 (dtd, J=15.1, 7.4, 3.0 Hz, 1H), 1.63 (s, 1H), 1.54 (ddd, J=12.8, 8.9, 4.4 Hz, 1H), 1.44 (m, 2H), 1.02 (d, J=6.9 Hz, 6H), 0.94 (s, 3H), 0.91 (d, J=6.8 Hz, 3H) ppm; ¹³C NMR (151 MHz, CDCl₃): δ=147.9, 142.3, 136.0, 131.9, 76.3, 73.0, 69.7, 58.4, 56.6, 50.7, 39.2, 33.6, 33.1, 30.3, 27.6, 27.5, 24.8, 21.5, 21.1, 16.6 ppm; HRMS (ESI, m/z): [M+H]⁺ calcd for C₂₁H₃₅O₃, 335.2581; found 335.2582.

Characterization data of product 8: R_{f}=0.65 (silica gel, hexanes:EtOAc=4:1); [α]_{D}^{26.6}=−193.4 (c=1.0 in EtOAc); ¹H NMR (400 MHz, CDCl₃): δ=4.01 (dd, J=11.4, 1.3 Hz, 1H), 3.92 (d, J=11.7 Hz, 1H), 3.33-3.27 (m, 1H), 3.28 (s, 3H), 3.03 (dt, J=17.2, 1.6 Hz, 1H), 2.79 (t, J=9.4 Hz, 1H), 2.61 (d, J=17.1 Hz, 1H), 2.46-2.39 (m, 2H), 2.34-2.18 (m, 4H), 2.18 (d, J=13.3 Hz, 1H), 1.89 (dtd, J=12.9, 7.4, 2.8 Hz, 1H), 1.76-1.72 (m, 2H), 1.10 (dq, J=12.9, 9.9 Hz, 1H), 0.97 (s, 3H), 0.96 (d, J=7.0 Hz, 3H), 0.89 (d, J=6.8 Hz, 3H), 0.86 (d, J=6.8 Hz, 3H) ppm; ¹³C NMR (100 MHz, CDCl₃): δ=213.6, 145.2, 138.7, 137.3, 134.2, 69.7, 58.4, 54.5, 50.6, 42.9, 41.4, 39.4, 38.7, 32.6, 27.4, 27.1, 25.8, 24.1, 20.9, 20.3, 14.1 ppm; HRMS (ESI, m/z): [M+H]⁺ calcd for C₂₁H₃₃O₂, 317.2475; found 317.2476.

### Synthesis of Compound S12

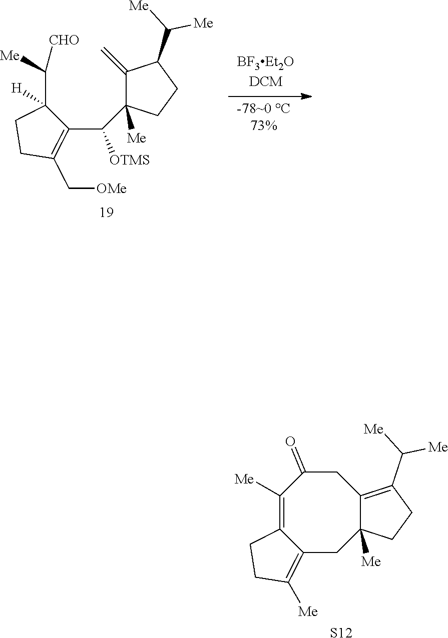

A solution of substrate 19 (32 mg, 79 mol) in CH₂Cl₂ (1 mL) was cooled to −78 °C under Ar, and treated with BF₃·Et₂O (56 mg, 0.39 mmol, 5.0 eq.). The solution was stirred at the same temperature for 30 min and then warmed up slowly 0 °C over 1 h. The mixture turned deep brown and was stirred at 0 °C for another 15 min. The reaction was quenched by the addition of NaHCO₃ (sat. aq., 3 mL) and was stirred vigorously for 10 min. The biphasic mixture was diluted 22 by hexanes (2 mL) and then separated. The aqueous phase was extracted with hexanes:EtOAc (4:1, 3 mL) and the combined organic phase was concentrated in vacuo. The residue was purified by flash column chromatography (hexanes:EtOAc=25:1 to 20:1) to give product S12 (16 mg, 73%) as yellow oil.

Characterization data of product S12: R_{f}=0.68 (silica gel, hexanes:EtOAc=4:1); [α]_{D}^{22.3}=+132.8 (c=0.7 in EtOAc); ¹H NMR (400 MHz, CDCl₃): δ=3.25 (d, J=16.7 Hz, 1H), 3.03 (ddd, J=16.7, 2.7, 1.5 Hz, 1H), 2.52 (hept, J=6.9 Hz, 1H), 2.43-2.30 (m, 5H), 2.20 (dddd, J=15.5, 9.0, 5.6, 2.7 Hz, 1H), 2.05-1.96 (m, 2H), 1.81 (s, 3H), 1.76 (s, 3H), 1.68 (ddd, J=12.8, 9.6, 5.7 Hz, 1H), 1.48 (ddd, J=12.8, 9.4, 5.3 Hz, 1H), 1.09 (s, 3H), 0.93 (d, J=6.8 Hz, 3H), 0.86 (d, J=6.9 Hz, 3H) ppm; ¹³C NMR (100 MHz, CDCl₃): δ=212.0, 151.2, 148.6, 144.8, 2 135.3, 131.8, 120.1, 51.9, 41.7, 36.7, 35.4, 33.7, 29.0, 28.9, 27.6, 27.4, 21.2, 21.1, 18.8, 16.1 ppm; HRMS (ESI, m/z): [M+H]⁺ calcd for C₂₀H₂₉O, 285.2213; found 285.2212.

### Synthesis of Compound 20

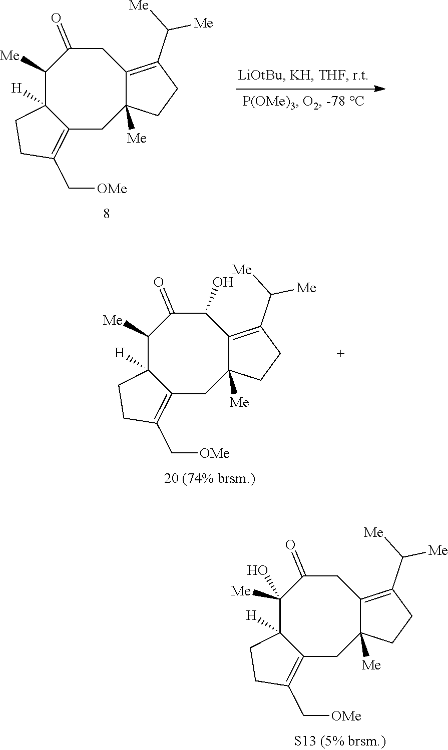 [SMILES: COCC1=C2C[C@@]3(C)CCC(C(C)C)=C3CC(=O)[C@H](C)[C@@H]2CC1 ; COCC1=C2C[C@@]3(C)CCC(C(C)C)=C3[C@@H](O)C(=O)[C@H](C)[C@@H]2CC1 ; COCC1=C2C[C@@]3(C)CCC(C(C)C)=C3CC(=O)[C@@](C)(O)[C@@H]2CC1]

A solution of 8 (813 mg, 2.57 mmol) in THF (1 mL) was cooled to 0 °C under Ar, and treated with LiOt-Bu (25.7 mL, 1.0 M in THF, 25.7 mmol, 10 eq.) and KH (dry, >95%, 1.031 g, 25.7 mmol, 10 eq.). The suspension was degassed with Ar and stirred vigorously at room temperature for 24 h. After cooling to −78 °C, P(OMe)₃ (638 mg, 5.14 mmol, 2.0 eq., to remove any residual water that may present, the reagent can be diluted by THF and pretreated with excess KH) was added, followed by purging with O₂. The viscous solution was stirred vigorously at the same temperature for another 0.5 h and then quenched by the addition of HCOOH (2.36 g, 51.4 mmol, 20 eq.) in THF (10 mL) (CAUTION: HCOOH solution should be added slowly along the wall of the flask with vigorous stirring to minimize fire risk). NH₄Cl (sat. aq., 30 mL) was added and the mixture was warmed to room temperature. The biphasic mixture was separated and the aqueous phase was extracted with hexanes:EtOAc (2:1, 20 mL) twice. The combined organic phase was washed with NaHCO₃ (20 mL) and concentrated in vacuo. The residue was purified by flash column chromatography (hexanes:EtOAc=25:1 to 10:1) to give product 20 (482 mg, 58%) as colorless oil, together with recovered starting material 8 (177 mg, 22%) and byproduct S13 (33 mg, 4%).

Characterization data of product 20: R_{f}=0.35 (silica gel, hexanes:EtOAc=4:1); [α]_{D}^{26.2}==−180.8 (c=1.0 in EtOAc); ¹H NMR (600 MHz, CDCl₃): δ=4.29 (s, 1H), 4.06 (d, J=11.4 Hz, 1H), 3.94 (d, J=11.4 Hz, 1H), 3.81 (s, 1H), 3.34-3.30 (m, 1H), 3.31 (s, 3H), 2.82 (s, 1H), 2.50 (d, J=13.2 Hz, 1H), 2.46 (hept, J=6.8 Hz, 1H), 2.35-2.29 (m, 4H), 2.18 (d, J=13.2 Hz, 1H), 1.99 (ddt, J=12.1, 7.4, 4.5 Hz, 1H), 1.86-1.77 (m, 2H), 1.16 (s, 3H), 1.11 (d, J=6.9 Hz, 3H), 0.95 (d, J=6.7 Hz, 3H), 0.91 (t, J=11.4 Hz, 1H), 0.78 (d, J=6.9 Hz, 3H) ppm; ¹³C NMR (151 MHz, CDCl₃): δ=213.2, 148.0, 138.6, 138.1, 137.9, 73.1, 69.7, 58.5, 54.2, 50.3, 41.9, 39.7, 38.3, 32.5, 27.6, 27.4, 25.5, 25.2, 21.3, 19.9, 14.4 ppm; HRMS (ESI, m/z): [M+H]⁺ calcd for C₂₁H₃₃O₃, 333.2424; found 333.2426.

Characterization data of byproduct S13: R_{f}=0.33 (silica gel, hexanes:EtOAc=4:1); [α]^{22.9}=+82.7 (c=0.3 in EtOAc); ¹H NMR (600 MHz, CDCl₃): δ=3.99 (d, J=11.7 Hz, 1H), 3.96 (d, J=12.0 Hz, 1H), 3.59-3.56 (m, 2H), 3.31 (s, 3H), 3.19 (t, J=7.8 Hz, 1H), 3.15 (ddd, J=13.8, 2.2, 1.0 Hz, 1H), 2.77 (hept, J=6.8 Hz, 1H), 2.42 (d, J=15.3 Hz, 1H), 2.40-2.35 (m, 1H), 2.29-2.19 (m, 2H), 2.14 (dd, J=15.2, 9.0 Hz, 1H), 1.98 (dtd, J=13.3, 8.4, 3.5 Hz, 1H), 1.86 (ddt, J=13.4, 9.4, 8.1 Hz, 1H), 1.50 (dt, J=12.4, 9.3 Hz, 1H), 1.44 (ddd, J=12.2, 7.8, 2.1 Hz, 1H), 1.28 (s, 3H), 0.97 (s, 3H), 0.92 (d, J=6.9 Hz, 3H), 0.91 (d, J=6.9 Hz, 3H) ppm; ¹³C NMR (151 MHz, CDCl₃): δ=211.4, 145.3, 140.6, 137.1, 131.9, 80.5, 69.1, 58.2, 56.1, 50.2, 37.5, 35.8, 34.1, 32.3, 27.5, 27.2, 25.8, 25.7, 20.8, 20.1, 19.8 ppm; HRMS (ESI, m/z): [M+H]⁺ calcd for C₂₁H₃₃O₃, 333.2424; found 333.2425.

### Synthesis of Compound 21

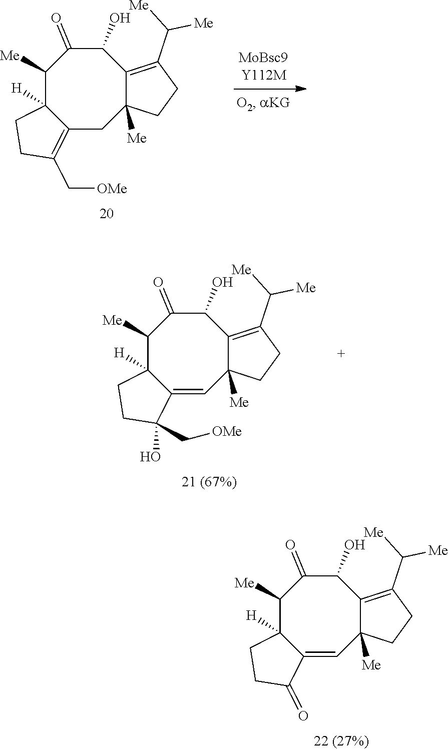 [SMILES: COCC1=C2C[C@@]3(C)CCC(C(C)C)=C3[C@@H](O)C(=O)[C@H](C)[C@@H]2CC1 ; COC[C@@]1(O)CC[C@@H]2/C1=C\[C@@]1(C)CCC(C(C)C)=C1[C@@H](O)C(=O)[C@@H]2C ; CC(C)C1=C2[C@@H](O)C(=O)[C@H](C)[C@@H]3CCC(=O)/C3=C/[C@@]2(C)CC1]

For a similar procedure with detailed pictorial guides, see ref S5.

### Procedure for Enzyme Expression and Lysate Preparation of MoBsc9 Y112M:

Recombinant E. coli BL21(DE3) harboring plasmids pET22b(+)-MoBsc9 Y112M and pGro7 (groES/groEL) was grown in LB (4 mL, with 50 g/mL ampicillin and 25 g/mL chloramphenicol) for 12 h, and 2 mL of the culture was used to inoculate 500 mL terrific broth media (in 2 L non-beveled Erlenmeyer flask, with 50 g/mL ampicillin and 25 μg/mL chloramphenicol). The culture was shaken at 250 rpm at 37 °C for roughly 3 hours or until an optical density (OD₆₀₀) of 0.9-1.0 was reached. The culture was cooled on ice for 20 min, and then induced by the addition of isopropyl β-D-1-thiogalactopyranoside (0.5 mL of 25 mM 1000× stork, final concentration=25 M) and L-arabinose (500 mg, final concentration=1 mg/mL). The culture was shaken at 250 rpm at 15 °C for 24 h and then cooled to 0 °C. The cells were harvested by centrifugation (4 °C, 10 min, 3234×g) and resuspended in tris buffer (50 mM, pH 8.5) to an OD₆₀₀ of around 60. Tris(2-carboxyethyl)phosphine hydrochloride (neutralized with 3.8 eq. of NaOH to make the pH around 8.5) was added to a final concentration of 10 mM. The suspension was disrupted by sonication cooled by ice. The crude lysate was centrifuged (4 °C, 10 min, 3234×g) again to make clarified lysate, which was kept at 0 °C and used within 1 h.

1 L non-beveled Erlenmeyer flask was cooled to 0 °C and charged with the above 2 clarified lysate (30 mL). FeSO₄·7H₂O (8.3 mg, 30 mol, 1 mM), ascorbic acid (26.4 mg, 150 mol, 5 mM) and disodium α-ketoglutarate dihydrate (102 mg, 450 mol, 15 mM) were added 4 with gentle shaking. Finally, substrate 20 (30.0 mg, 90 mol, dissolved in 1.5 mL DMSO) was added dropwise with gentle shaking. The flask was shaken at 160 rpm at 15 °C for 10 h. The reaction mixture was treated with Na₂SO₄ (solid, ~7 g) and extracted with EtOAc (30 mL×4). The combined organic phase was concentrated and the residue was purified by flash column chromatography (silica gel, hexanes:EtOAc=8:1 to 2:3) to give product 21 (20.9 mg, 67%) as colorless oil, together with byproduct 22 (7.3 mg, 27%).

Characterization data of product 21: R_{f}=0.15 (silica gel, hexanes:EtOAc=2:1); [α]_{D}^{25.9}=−18.7 (c=1.0 in EtOAc); ¹H NMR (400 MHz, CDCl₃): δ=5.81 (d, J=2.3 Hz, 1H), 4.74 (d, J=4.7 Hz, 1H), 3.82 (d, J=4.9 Hz, 1H), 3.41 (s, 3H), 3.41-3.36 (m, 2H), 3.27 (d, J=9.3 Hz, 1H), 2.74 (hept, J=6.8 Hz, 1H), 2.67 (qd, J=7.4, 1.8 Hz, 1H), 2.23-2.19 (m, 2H), 2.05 (ddt, J=12.3, 8.5, 7.4 Hz, 1H), 2.00-1.93 (m, 1H), 1.84 (ddd, J=12.5, 7.6, 6.4 Hz, 1H), 1.78 (dt, J=12.4, 7.2 Hz, 1H), 1.55 (dt, J=13.4, 7.3 Hz, 1H), 1.37 (dtd, J=12.0, 6.8, 6.3, 5.2 Hz, 1H), 1.28 (s, 3H), 1.08 (d, J=7.5 Hz, 3H), 0.97 (d, J=6.8 Hz, 3H), 0.81 (d, J=6.9 Hz, 3H) ppm; ¹³C NMR (100 MHz, CDCl₃): δ=211.7, 150.7, 140.6, 137.2, 133.6, 82.0, 78.0, 69.9, 59.5, 52.9, 18 50.9, 40.5, 39.7, 36.3, 31.0, 27.9, 27.2, 26.2, 21.1, 19.7, 14.5 ppm; HRMS (ESI, m/z): [M+H]⁺ calcd for C₂₁H₃₃O₄, 349.2373; found 349.2375.

Characterization data of byproduct 22: R_{f}=0.33 (silica gel, hexanes:EtOAc=2:1); [α]_{D}^{26.1}=−163.2 (c=1.0 in EtOAc); ¹H NMR (400 MHz, CDCl₃): δ=6.85 (d, J=1.9 Hz, 1H), 4.59 (d, J=3.2 Hz, 1H), 3.86 (d, J=4.7 Hz, 1H), 3.64 (dq, J=9.2, 1.7 Hz, 1H), 2.84 (qd, J=7.5, 2.2 Hz, 1H), 2.75 (hept, J=6.8 Hz, 1H), 2.44-2.12 (m, 5H), 1.96 (ddd, J=12.7, 8.0, 3.0 Hz, 1H), 1.79-1.72 (m, 1H), 1.71 (dt, J=12.6, 9.0 Hz, 1H), 1.34 (s, 3H), 1.07 (d, J=7.4 Hz, 3H), 0.97 (d, J=6.8 Hz, 3H), 0.85 (d, J=6.8 Hz, 3H) ppm; ¹³C NMR (100 MHz, CDCl₃): δ=210.9, 208.2, 150.6, 147.4, 135.5, 134.2, 70.6, 53.2, 52.0, 39.0, 36.8, 36.0, 28.5, 27.7, 27.1, 25.2, 20.9, 19.7, 14.1 ppm; HRMS (ESI, m/z): [M+H]⁺ calcd for C₁₉H₂₇O₃, 303.1955; found 303.1956.

### Synthesis of Compound 3 (Cotylenol)

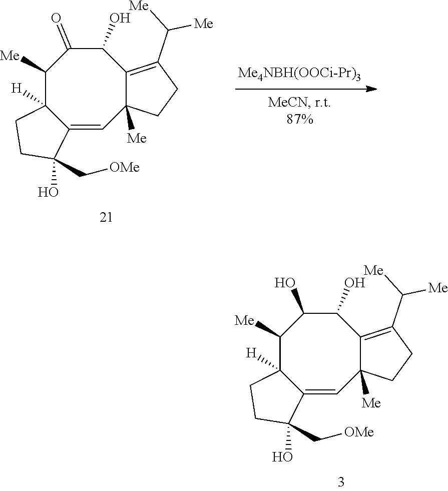 [SMILES: COC[C@@]1(O)CC[C@@H]2/C1=C\[C@@]1(C)CCC(C(C)C)=C1[C@@H](O)C(=O)[C@@H]2C ; COC[C@@]1(O)CC[C@@H]2/C1=C\[C@@]1(C)CCC(C(C)C)=C1[C@@H](O)[C@H](O)[C@@H]2C]

Me₄NBH(OOCi-Pr)₃ (1.0 M in MeCN) was prepared according to Nakada's procedure^{S6}. Ketone 21 (60.0 mg, 0.172 mmol) was dissolved in MeCN (0.1 mL) under Ar. Me₄NBH(OOCi-Pr)₃ (2 mL, 1.0 M in MeCN, 2 mmol) was added, and the reaction was stirred at room temperature for 24 h. MeOH (5 mL) and NH₄Cl (powder, 200 mg) were then added. After stirring at room temperature for 10 min, the suspension was concentrated. The residue was resuspended in MeOH (5 mL), stirred for 10 min and concentrated, and this procedure was repeated for 3 times. The residue was purified by flash column chromatography (hexanes EtOAc=6:1 to 2:1) to give product 3 (52.7 mg, 87%) as colorless oil.

Characterization data of 3: R_{f}=0.35 (silica gel, hexanes:EtOAc=1:1); [α]_{D}^{26.3}=−31.5 (c=1.0 in EtOAc) [comparison: natural^{S7}: [α]_{D}¹⁸=−26° (c=0.96 in MeOH); synthetic by Takeshita et al.: [α]_{D}³⁰=−30° (unknown concentration and solvent); synthetic by Nakada et al.: [α]_{D}²⁵=−29° (c=0.18 in MeOH)]; ¹H NMR (400 MHz, CDCl₃): δ=5.50 (d, J=2.6 Hz, 1H), 4.05 (d, J=10.0 Hz, 1H), 3.92 (dd, J=10.0, 4.3 Hz, 1H), 3.39 (s, 3H), 3.35 (d, J=9.5 Hz, 1H), 3.26 (hept, J=6.6 Hz, 1H), 3.07 (dd, J=9.5, 1.3 Hz, 1H), 2.92 (td, J=8.6, 2.5 Hz, 1H), 2.55 (s, 1H), 2.14 (ddd, J=16.4, 9.9, 6.5 Hz, 1H), 2.08 (ddd, J=15.6, 8.4, 2.4 Hz, 1H), 2.02-1.89 (m, 3H), 1.84 (ddd, J=12.0, 6.6, 2.4 Hz, 1H), 1.67 (ddd, J=12.0, 10.0, 8.4 Hz, 1H), 1.43-1.36 (m, 1H), 1.30-1.23 (m, 1H), 1.20 (s, 3H), 1.02 (d, J=6.7 Hz, 3H), 0.94 (d, J=6.8 Hz, 3H), 0.79 (d, J 18=7.3 Hz, 3H) ppm; ¹³C NMR (100 MHz, CDCl₃): δ=150.4, 139.8, 137.0, 134.4, 82.1, 77.6, 77.4, 67.9, 59.4, 51.9, 42.6, 41.7, 40.3, 35.4, 31.7, 28.1, 27.2, 26.6, 21.6, 20.4, 8.5 ppm; HRMS (ESI, m/z): [M+H]⁺ calcd for C₂₁H₃₃O₄, 351.2530; found 351.2531.

### Synthesis of Compound 23

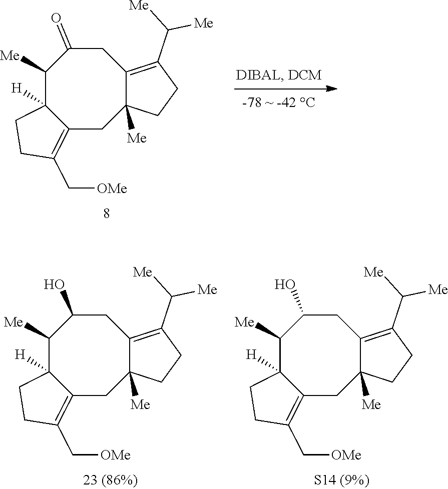 [SMILES: COCC1=C2C[C@@]3(C)CCC(C(C)C)=C3CC(=O)[C@H](C)[C@@H]2CC1 ; COCC1=C2C[C@@]3(C)CCC(C(C)C)=C3C[C@H](O)[C@H](C)[C@@H]2CC1 ; COCC1=C2C[C@@]3(C)CCC(C(C)C)=C3C[C@@H](O)[C@H](C)[C@@H]2CC1]

Ketone 8 (433 mg, 1.37 mmol) was dissolved in CH₂Cl₂ (12 mL). The solution was cooled to −78 °C under Ar and then treated with ⁱBu₂AlH (1.64 mL, 1 M in hexanes, 1.64 mmol, 1.2 eq.). The solution was stirred at −78 °C for 0.5 h, warmed up slowly to −42 °C and stirred for another 0.5 h. The reaction was quenched by the addition of MeOH (0.5 mL), and stirred at the same temperature for another 10 min, followed by the addition of AcOH (10% aq., 6 mL). After separation, the aqueous phase was extracted with ethyl acetate (2×6 mL). The combined organic phase was washed with NaHCO₃ (sat. aq., 12 mL) and concentrated in vacuo. The residue was purified by silica gel flash column chromatography (hexanes:ethyl acetate=20:1 to 10:1) to give product 23 (374 mg, 86%) as colorless oil, together with byproduct S14 (40 mg, 9%).

Characterization data of 23: R_{f}=0.35 (silica gel, hexanes:EtOAc=4:1); [α]_{D}^{22.6}=+52.3 (c=1.0 in EtOAc); ¹H NMR (400 MHz, CDCl₃): δ=4.00-3.90 (m, 2H), 3.82 (dt, J=11.8, 4.4 Hz, 1H), 3.28 (s, 3H), 2.75-2.63 (m, 2H), 2.38-1.90 (m, 10H), 1.66-1.27 (m, 4H), 0.99 (d, J=6.8 Hz, 3H), 1.02-0.93 (m, 3H), 0.92 (d, J=6.8 Hz, 3H), 0.90 (s, 3H) ppm; ¹³C NMR (100 MHz, CDCl₃): δ=142.1, 141.0, 136.3, 76.6, 69.2, 58.0, 50.1, 43.0, 37.5, 32.5, 31.7, 27.4, 27.0, 26.4, 21.2, 21.0, 6.5 ppm; HRMS (ESI, m/z): [M+H]⁺ calcd for C₂₁H₃₅O₂, 319.2632; found 319.2634.

Characterization data of S14: R_{f}=0.60 (silica gel, hexanes:EtOAc=4:1); [α]_{D}^{22.4}=+25.5 (c=1.0 in EtOAc); ¹H NMR (400 MHz, CDCl₃): δ=3.96 (s, 2H), 3.58 (br s, 1H), 3.28 (s, 3H), 2.86 (hept, J=6.8 Hz, 1H), 2.72 (br s, 1H), 2.45 (dd, J=14.4, 4.8 Hz, 1H), 2.39-2.25 (m, 3H), 2.21-2.16 (m, 3H), 2.07 (d, J=13.8 Hz, 1H), 1.90 (dtd, J=12.3, 7.6, 1.6 Hz, 1H), 1.72 (ddd, J=11.4, 6.0, 2.4 Hz, 1H), 1.66-1.60 (m, 1H), 1.50 (dt, J=11.9, 9.8 Hz, 1H), 1.43-1.32 (m, 1H), 1.05 (d, J=6.9 Hz, 3H), 1.02 (d, J=6.8 Hz, 3H), 0.87 (s, 3H), 0.87 (d, J=6.9 Hz, 3H) ppm; ¹³C NMR (100 MHz, CDCl₃): δ=144.8, 140.9, 135.6, 134.7, 73.6, 69.6, 58.1, 51.0, 39.6, 16 38.8, 37.8, 32.7, 29.2, 27.3, 27.0, 25.6, 21.4, 21.2, 16.8 ppm; HRMS (ESI, m/z): [M+H]⁺ calcd for C₂₁H₃₅O₂, 319.2632; found 319.2633.

### Synthesis of Compound 4 (Brassicicene I)

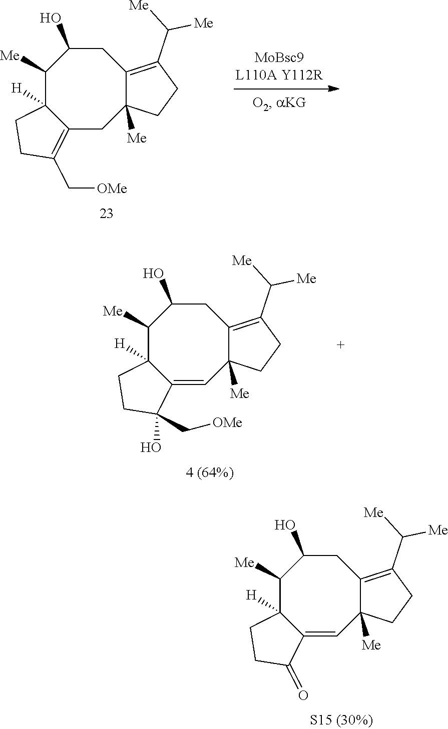 [SMILES: COCC1=C2C[C@@]3(C)CCC(C(C)C)=C3C[C@H](O)[C@H](C)[C@@H]2CC1 ; COC[C@@]1(O)CC[C@@H]2/C1=C\[C@@]1(C)CCC(C(C)C)=C1C[C@H](O)[C@@H]2C ; CC(C)C1=C2C[C@H](O)[C@H](C)[C@@H]3CCC(=O)/C3=C/[C@@]2(C)CC1]

For a similar procedure with detailed pictorial guides, see ref S5.

### Procedure for Enzyme Expression and Lysate Preparation of MoBsc9 L110A Y112R:

Recombinant E. coli BL21(DE3) harboring plasmids pET22b(+)-MoBsc9 L110A Y112R and pGro7 (groES/groEL) was grown in LB (4 mL, with 50 g/mL ampicillin and 25 g/mL chloramphenicol) for 12 h, and the culture was used to inoculate 500 mL×2 terrific broth media (in two 2 L non-beveled Erlenmeyer flasks, with 50 g/mL ampicillin and 25 μg/mL chloramphenicol). The flasks were shaken at 250 rpm at 37 °C for roughly 3 hours or until an optical density (OD₆₀₀) of 0.9-1.0 was reached. The culture was cooled on ice for 20 min, and then induced by the addition of isopropyl β-D-1-thiogalactopyranoside (25 mM 1000× stork, 0.5 mL for each flask, final concentration=25 M) and L-arabinose (500 mg for each flask, final concentration=1 mg/mL). The culture was shaken at 250 rpm at 15 °C for 24 h and then cooled to 0 °C. The cells were harvested by centrifugation (4 °C, 10 min, 3234×g) and resuspended in tris buffer (50 mM, pH 8.5) to an OD₆₀₀ of around 60. Tris(2-carboxyethyl)phosphine hydrochloride (neutralized with 3.8 eq. of NaOH to make the pH around 8.5) was added to a final concentration of 10 mM. The suspension was disrupted by sonication cooled by ice. The crude lysate was centrifuged (4 °C, 10 min, 3234×g) again to make clarified lysate, which was kept at 0 °C and used within 1 h.

1 L non-beveled Erlenmeyer flask was cooled to 0 °C and charged with the above clarified lysate (130 mL). FeSO₄·7H₂O (36.1 mg, 130 mol, 1 mM), ascorbic acid (115 mg, 650 μmol, 5 mM) and disodiuma-ketoglutarate dihydrate (441 mg, 1.95 mmol, 15 mM) was added with gentle shaking. Finally, substrate 23 (130.0 mg, 408 mol, dissolved in 6.5 mL DMSO) was added dropwise with gentle shaking. The flask was shaken at 160 rpm at 15 °C for 10 h. The reaction mixture was saturated with Na₂SO₄ (solid, ~25 g) and extracted with EtOAc (130 mL×4). The combined organic phase was concentrated and the residue was purified by flash column chromatography (silica gel, hexanes:EtOAc=4:1 to 1:1, then dichloromethane:EtOAc=15:1 to 4:1) to give product 4 (88.1 mg, 64%) as colorless oil, together with byproduct S15 (35.1 mg, 30%), and recovered substrate 23 (2.1 mg, 2%).

Characterization data of product 4: R_{f}=0.20 (silica gel, hexanes:EtOAc=2:1); [α]_{D}^{22.3}=+13.9 (c=1.0 in EtOAc) [comparison: natural^{S9}: [α]_{D}²⁷=+26.9° (c=0.11 in MeOH)]; ¹H NMR (400 MHz, CDCl₃): δ=5.59 (d, J=2.5 Hz, 1H), 3.89 (dt, J=11.5, 3.9 Hz, 1H), 3.42 (s, 3H), 3.39 (d, J=9.5 Hz, 1H), 3.14 (dd, J=9.5, 1.2 Hz, 1H), 2.89 (td, J=8.0, 2.5 Hz, 1H), 2.78 (hept, J=6.8 Hz, 1H), 2.31 (ddd, J=13.3, 3.6, 2.0 Hz, 1H), 2.13-1.96 (m, 5H), 1.83-1.77 (m, 2H), 1.64 (dt, J=12.1, 8.5 Hz, 1H), 1.47-1.39 (m, 1H), 1.30-1.23 (m, 2H), 1.13 (s, 3H), 0.99 (d, J=7.0 Hz, 3H), 0.97 (d, J=7.0 Hz, 3H), 0.80 (d, J=7.1 Hz, 3H) ppm; ¹³C NMR (100 MHz, CDCl₃): δ=146.0, 139.5, 135.6, 132.7, 82.1, 77.7, 76.4, 59.5, 52.9, 44.7, 42.1, 40.5, 35.7, 32.1, 28.6, 27.5, 27.2, 26.6, 21.0, 20.9, 8.1 ppm; HRMS (ESI, m/z): [M+H]⁺ calcd for C₂₁H₃₅O₃, 335.2581; found 335.2581.

Characterization data of byproduct S15: R_{f}=0.21 (silica gel, hexanes:EtOAc=2:1); [α]_{D}^{22.7}=+19.3 (c=0.8 in EtOAc); ¹H NMR (400 MHz, CDCl₃): δ=6.72 (d, J=2.1 Hz, 1H), 3.96 (ddd, J=11.5, 4.7, 3.0 Hz, 1H), 3.17 (d, J=9.0 Hz, 1H), 2.79 (hept, J=6.8 Hz, 1H), 2.45-2.33 (m, 1H), 2.32-2.14 (m, 5H), 2.12-2.03 (m, 2H), 1.81 (ddd, J=12.5, 7.8, 4.8 Hz, 1H), 1.75-1.64 (m, 2H), 1.51 (br s, 1H), 1.15 (s, 3H), 1.06 (d, J=6.9 Hz, 3H), 0.97 (d, J=6.8 Hz, 3H), 0.81 (d, J=7.1 Hz, 3H) ppm; ¹³C NMR (100 MHz, CDCl₃): δ=209.4, 146.6, 145.3, 134.3, 34 134.2, 76.2, 53.2, 46.3, 39.7, 37.9, 36.4, 29.7, 28.7, 27.6, 27.1, 26.1, 21.0, 7.8 ppm; HRMS (ESI, m/z): [M+H]⁺ calcd for C₁₉H₂₉O₂, 289.2162; found 289.2160.

### Synthesis of Compound 24

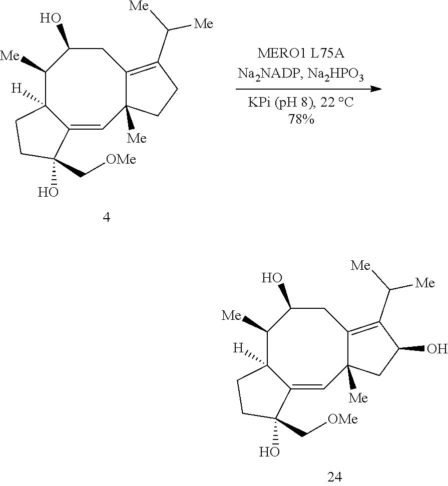 [SMILES: COC[C@@]1(O)CC[C@@H]2/C1=C\[C@@]1(C)CCC(C(C)C)=C1C[C@H](O)[C@@H]2C ; COC[C@@]1(O)CC[C@@H]2/C1=C\[C@@]1(C)C[C@H](O)C(C(C)C)=C1C[C@H](O)[C@@H]2C]

### Procedure for Enzyme Expression and Lysate Preparation of MERO1 L75A:

Recombinant E. coli BL21(DE3) harboring plasmids pET22b(+)—P450_{BM3} MERO1 L75A and pRSF-Opt13 was grown in LB (4 mL, with 50 μg/mL ampicillin and 25 μg/mL kanamycin) for 12 h, and 2 mL of the culture was used to inoculate 500 mL terrific broth media (in 2 L non-beveled Erlenmeyer flask, with 50 g/mL ampicillin, 25 g/mL kanamycin and trace metal mix (FeCl₃ 50 μM, CaCl₂) 20 μM, MnSO₄ 10 μM, ZnSO₄ 10 μM, CoSO₄ 2 μM, CuCl₂ 2 μM, NiCl₂ 2 μM, Na₂MoO₄ μ2 M, H₃BO₃ 2 μM)). The culture was shaken at 250 rpm at 37 °C for roughly 3 hours or until an optical density (OD₆₀₀) of 0.9-1.0 was reached. The culture was cooled on ice for 20 min, and then induced by the addition of isopropyl β-D-1-thiogalactopyranoside (0.5 M 1000× stork, 0.5 mL, final concentration=0.5 mM) and 5-aminolevulinic acid (1.0 M 1000× stork, 0.5 mL, final concentration=1.0 mM). The culture was shaken at 250 rpm at 23 °C for 24 h and then cooled to 0 °C. The cells were harvested by centrifugation (4 °C, 10 min, 3234×g) and resuspended in KH₂PO₄—K₂HPO₄ buffer (50 mM, pH 8.0) to an OD₆₀₀ of 20 (about 250 mL in total). The suspension was disrupted by sonication (1 s on/4 s off, total time 3 min, 50% amplification), cooled in an ice bath. The crude lysate was centrifuged (4 °C, 10 min, 3234×g) again to make clarified lysate, which was kept at 0 °C until its use.

Procedure for Enzymatic Reaction with MERO1 L75A:

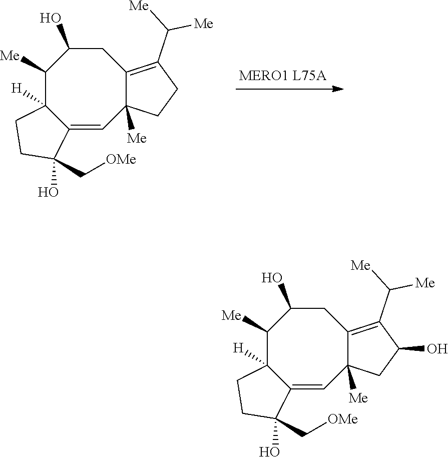 [SMILES: COC[C@@]1(O)CC[C@@H]2/C1=C\[C@@]1(C)CCC(C(C)C)=C1C[C@H](O)[C@@H]2C ; COC[C@@]1(O)CC[C@@H]2/C1=C\[C@@]1(C)C[C@H](O)C(C(C)C)=C1C[C@H](O)[C@@H]2C]

A 1 L non-beveled Erlenmeyer flask was charged with the above clarified lysate (245 mL). Na₂NADP (115 mg, 0.15 mmol, 0.2 eq.), Na₂HPO₃·5H₂O (3.16 g, 14.6 mmol, 20 eq.) and substrate 4 (245 mg, 0.73 mmol) in DMSO (12.25 mL) were sequentially added with gentle shaking. The flask was shaken at 160 rpm at 22 °C for 16 h. The reaction mixture was treated with Na₂SO₄ (solid, ~40 g), pyridine (5 mL), and stirred until Na₂SO₄ was fully dissolved. The aqueous phase was then extracted with EtOAc (250 mL×4). The combined organic phase was washed with Na₂SO₄ (sat. aq., 100 mL) and concentrated. The residue was purified by flash column chromatography (silica gel, hexanes:EtOAc=1:1 to 0:1) to give product 24 (200 mg, 78%) as white solid. The product is unstable towards weak acid and moderate base. It should be stored as a CH₂Cl₂ solution (stabilized by 1% pyridine) in the freezer.

Characterization data of product 24: R_{f}=0.13 (silica gel, hexanes:EtOAc=1:1); ¹H NMR (600 MHz, CD₃COCD₃): δ=5.58 (d, J=2.5 Hz, 1H), 4.68 (qd, J=6.7, 1.9 Hz, 1H), 3.87 (dq, J=11.4, 3.8 Hz, 1H), 3.68 (d, J=3.6 Hz, 1H), 3.39-3.37 (m, 3H), 3.34 (s, 3H), 3.07 (dd, J=9.8, 1.1 Hz, 1H), 2.90 (hept, J=7.0 Hz, 1H), 2.80 (td, J=8.2, 2.5 Hz, 1H), 2.36 (dddd, J=13.4, 3.5, 1.9, 1.0 Hz, 1H), 2.14 (dd, J=11.7, 10.8 Hz, 1H), 2.13 (dd, J=11.7, 4.2 Hz, 1H), 1.96-1.88 (m, 2H), 1.78 (qd, J=7.1, 3.8 Hz, 1H), 1.54 (dd, J=12.0, 6.8 Hz, 1H), 1.36-1.31 (m, 1H), 1.27-1.22 (m, 1H), 1.19 (s, 3H), 1.18 (d, J=7.0 Hz, 3H), 1.15 (d, J=7.0 Hz, 3H), 0.81 (d, J=7.1 Hz, 3H) ppm; ¹³C NMR (151 MHz, CD₃COCD₃): 6=147.7, 140.4, 137.4, 135.3, 82.6, 78.2, 76.1, 16 75.2, 59.3, 53.7, 50.6, 45.5, 41.5, 35.9, 32.5, 29.4, 28.6, 28.1, 22.8, 19.7, 8.3 ppm; HRMS (ESI, m/z): [M+H]⁺ calcd for C₂₁H₃₅O₄, 351.2530; found 351.2526.

### Synthesis of Compound 5 (Brassicicene A)

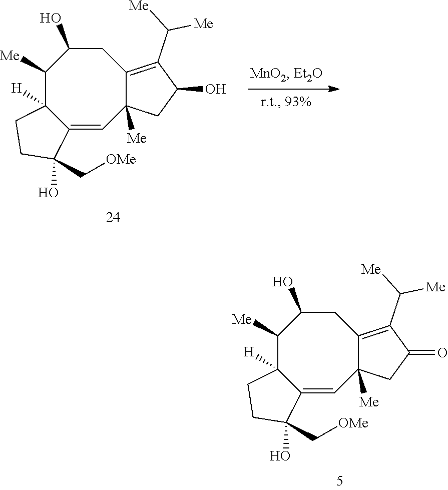 [SMILES: COC[C@@]1(O)CC[C@@H]2/C1=C\[C@@]1(C)C[C@H](O)C(C(C)C)=C1C[C@H](O)[C@@H]2C ; COC[C@@]1(O)CC[C@@H]2/C1=C\[C@@]1(C)CC(=O)C(C(C)C)=C1C[C@H](O)[C@@H]2C]

To a solution of substrate 24 (122.2 mg, 0.35 mmol) in Et₂O (3.5 mL) was added MnO₂ (4.8 g, m_{sub}×40, activated). The slurry was stirred vigorously at room temperature for 4 h, and the reaction was monitored by TLC (silica gel, CH₂Cl₂:MeOH=9:1, co-spot when developed by hexanes/EtOAc). Once completed, the slurry was diluted by acetone (10 mL) and stirred for 10 more mins. The suspension was filtered through a short bilayer celite (top)/silica gel (bottom) column and washed with EtOAc. The filtrate was concentrated to give product 5 (113.3 mg, 93%) as a white solid.

Characterization data of product 5: R_{f}=0.3 (silica gel, EtOAc); [α]_{D}²⁰=+49.2° (c=1.0 in EtOAc) [Lit.^{S10}: [α]_{D}²³=+44.4° (c=0.1350 in MeOH). Lit.^{S11}: [α]_{D}²⁰=+47.8° (c=0.09 in MeOH)]; ¹H NMR (400 MHz, CD₃OD): δ=5.75 (d, J=2.3 Hz, 1H), 3.94 (dt, J=10.4, 4.0 Hz, 1H), 3.44 (d, J=9.9 Hz, 1H), 3.39 (s, 3H), 3.28 (d, J=10.0 Hz, 1H), 2.86 (hept, J=6.8 Hz, 1H), 2.72 (ddd, J=8.5, 5.8, 2.2 Hz, 1H), 2.62-2.52 (m, 2H), 2.48 (d, J=18.5 Hz, 1H), 2.28 (d, J=18.5 Hz, 1H), 2.06-1.94 (m, 2H), 1.90 (qd, J=7.2, 4.5 Hz, 1H), 1.51-1.37 (m, 2H), 1.33 (s, 3H), 1.26 (d, J=7.0 Hz, 3H), 1.18 (d, J=6.9 Hz, 3H), 0.91 (d, J=7.0 Hz, 3H) ppm; ¹³C NMR (100 MHz, CD₃OD): δ=209.8, 175.9, 146.9, 142.1, 134.7, 83.3, 78.3, 76.5, 59.6, 53.4, 48.5, 46.1, 42.2, 36.5, 32.8, 31.4, 28.0, 27.3, 20.0, 19.9, 8.4 ppm; HRMS (ESI, m/z): [M+H]⁺ calcd for C₂₁H₃₃O₄, 349.2373; found 349.2375.

An Alternative Synthesis of Compound 5 (Brassicicene A) with Chemical Method.

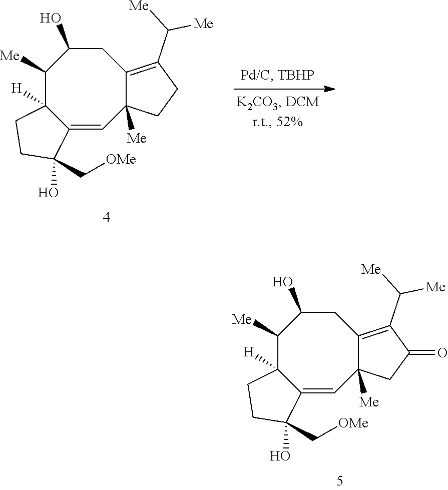 [SMILES: COC[C@@]1(O)CC[C@@H]2/C1=C\[C@@]1(C)CCC(C(C)C)=C1C[C@H](O)[C@@H]2C ; COC[C@@]1(O)CC[C@@H]2/C1=C\[C@@]1(C)CC(=O)C(C(C)C)=C1C[C@H](O)[C@@H]2C]

To a solution of substrate 4 (12.0 mg, 35.9 mol) in CH₂Cl₂ (0.5 mL) were added palladium on carbon (10% w/w, reduced, 1 mg), K₂CO₃ (14.9 mg, 0.11 mmol, 3.0 eq.) and tert-butylhydroproxide (13 L, 5.5 M in nonane, 71.7 mol, 2.0 eq.). The suspension was stirred at room temperature for 12 h, and then filtered through a short silica gel pad, eluting with EtOAc. The filtrate was concentrated and the residue was purified by flash column chromatography (hexanes:EtOAc=2:1 to 1:2) to give product 5 (6.5 mg, 52%) as colorless oil.

### Synthesis of Compound 25 (Brassicicene R)

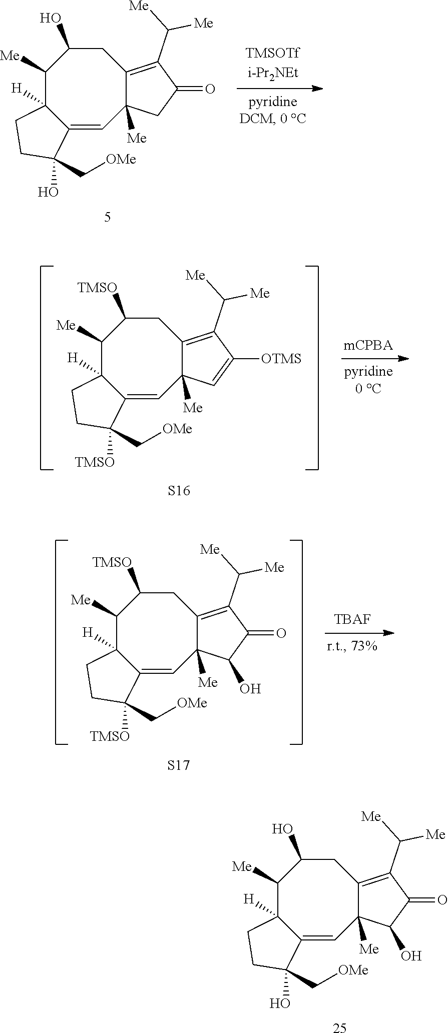

A solution of substrate 5 (12.0 mg, 34.4 mol) in CH₂Cl₂ (0.5 mL) was cooled to 0 °C and treated with pyridine (13.6 mg, 0.17 mmol, 5.0 eq.), N-ethyl diisopropylamine (22.2 mg, 0.17 mmol, 5.0 eq.) and trimethylsilyl trifluoromethanesulfonate (38.3 mg, 0.17 mmol, 5.0 eq.) in a dropwise manner. The mixture was stirred at 0 °C for 1 h. During this period, the solution turned yellowish brown. As S16 decomposes readily on TLC plates, only hydrolyzed diTMS-ketone could be observed (R_{f}=0.6, silica gel, hexanes:EtOAc=10:1). Then tert-butanol (12.7 mg, 0.17 mmol, 5.0 eq.) was added at the same temperature, followed by pyridine (0.2 mL). After another 10 min, a solution of purified meta-chloroperoxybenzoic acid in CH₂Cl₂ (0.4 mL (=0.28 mmol, 8 eq.) of 0.7 mol/L pure mCPBA in CH₂Cl₂, made by dissolving 172.5 mg of commercially available mCPBA (70-75% purity) in 1 mL CH₂Cl₂ and washing with 1 mL K₂HPO₄—KH₂PO₄ buffer (0.5 mol/L, pH=7) once) was added in one portion with vigorous stirring, and the color of the solution faded rapidly to nearly colorless. After stirring at 0 °C for another 15 min, triphenylphosphine (90.3 mg, 0.34 mmol, 10 eq.) was added and the solution was warmed to room temperature. After stirring for another 15 min, tetra-n-butylammonium fluoride (0.34 mL, 1 mmol/L in tetrahydrofuran, 10 eq.) was added, and the solution was stirred at room temperature for 12 h before it was diluted with EtOAc (4 mL) and washed with NaHCO₃ (sat. aq., 4 mL). The aqueous phase was extracted with EtOAc (4 mL) and the combined organic phase was filtered through a silica gel pad and eluted with EtOAc. The eluent was concentrated and purified by flash column chromatography (hexanes:EtOAc=2:1 to 1:2) to give product (9.1 mg, 73%) as colorless oil, together with recovered 5 (0.7 mg, 6%).

Characterization data of 25: R_{f}=0.22 (silica gel, EtOAc); [α]_{D}²⁰=+43.2° (c=0.75 in EtOAc) [lit.: [α]_{D}²⁰=+41.1° (c=0.2 in MeOH).^{S12}]; ¹H NMR (600 MHz, CD₃OD): δ=5.84 (d, J=2.0 Hz, 1H), 3.90 (s, 1H), 3.87 (ddd, J=10.0, 4.6, 3.2 Hz, 1H), 3.51 (d, J=9.8 Hz, 1H), 3.41 (d, J=9.8 Hz, 1H), 3.41 (s, 3H), 2.88 (hept, J=6.9 Hz, 1H), 2.66-2.63 (m, 1H), 2.55-2.48 (m, 2H), 2.11-2.04 (m, 1H), 1.99 (ddd, J=13.2, 9.5, 6.9 Hz, 1H), 1.94-1.90 (m, 1H), 1.57 (ddd, J=13.1, 7.5, 5.3 Hz, 1H), 1.51-1.46 (m, 1H), 1.24 (d, J=6.9 Hz, 3H), 1.19 (d, J=7.0 Hz, 3H), 1.14 (s, 3H), 0.93 (d, J=7.0 Hz, 3H) ppm; ¹³C NMR (151 MHz, CD₃OD): δ=208.1, 173.7, 145.3, 143.4, 133.6, 83.8, 83.3, 78.7, 77.3, 59.7, 53.1, 45.4, 42.5, 37.3, 33.4, 31.9, 27.0, 24.3, 20.4, 19.5, 8.9 ppm; HRMS (ESI, m/z): [M+H]⁺ calcd for C₂₁H₃₃O₅, 365.2323; found 365.2323.

### Synthesis of Compound 26

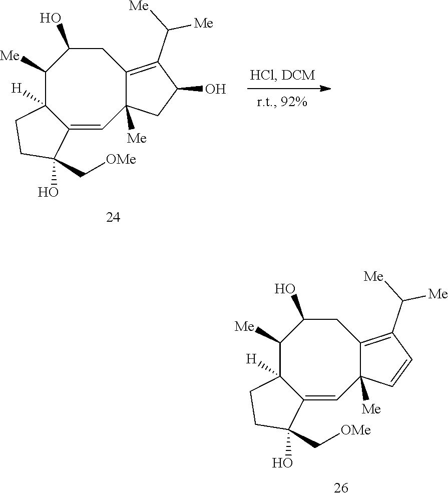

A solution of substrate 24 (30.0 mg, 85.6 mol) in CH₂Cl₂ (40 mL) was treated with hydrogen chloride (4 μL, 4 mol/L in dioxane, 16 mol, working concentration 0.4 mmol/L, 0.19 eq.). The solution was stirred in the dark at room temperature for 12 h and then quenched by the addition of triethylamine (0.01 mL). The solution was loaded onto silica gel column and eluted by hexanes:EtOAc (4:1 to 1:1) to give product 26 (26.2 mg, 92%) as colorless oil. The product is unstable and should be stored as a CH₂Cl₂ solution in the freezer.

Characterization data of 26: R_{f}=0.45 (silica gel, hexanes:EtOAc=1:1); [α]_{D}²⁰=+170° (c=0.5 in EtOAc); ¹H NMR (600 MHz, CDCl₃): δ=6.17 (d, J=5.5 Hz, 1H), 6.16 (d, J=5.5 Hz, 1H), 5.74 (d, J=2.3 Hz, 1H), 3.92 (dt, J=11.3, 3.7 Hz, 1H), 3.42 (s, 3H), 3.41 (d, J=9.5 Hz, 1H), 3.21 (d, J=9.5 Hz, 1H), 2.85-2.78 (m, 2H), 2.49 (dd, J=13.5, 3.4 Hz, 1H), 2.39 (dd, J=13.5, 11.4 Hz, 1H), 1.96-1.90 (m, 2H), 1.85 (qd, J=6.9, 3.5 Hz, 1H), 1.65 (br s, 1H), 1.44 (tdd, J=9.9, 7.7, 4.9 Hz, 1H), 1.29-1.23 (m, 1H), 1.21 (s, 3H), 1.12 (d, J=6.8 Hz, 3H), 1.02 (d, J=6.8 Hz, 3H), 0.90 (d, J=7.0 Hz, 3H) ppm; ¹³C NMR (151 MHz, CDCl₃): δ=147.0, 143.9, 140.0, 139.2, 129.0, 126.5, 82.0, 77.6, 77.5, 60.2, 59.5, 46.1, 41.1, 36.1, 31.9, 28.8, 26.5, 22.9, 22.2, 21.9, 8.3 ppm; HRMS (ESI, m/z): [M+H]⁺ calcd for C₂₁H₃₃O₃, 333.2424; found 333.2423.

### Synthesis of Compound 27

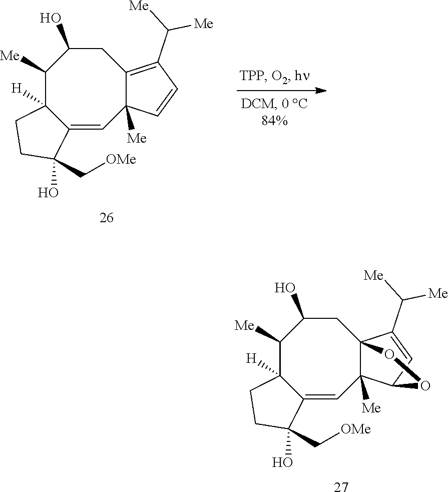

A solution of substrate 26 (27.2 mg, 81.8 mol) in CH₂Cl₂ (8.2 mL) was cooled to 0 °C and treated with meso-tetraphenylporphyrin (1 mg, 1.6 mol, 0.02 eq.). The solution was bubbled with oxygen and stirred in ice bath for 30 min, while being irradiated by a 150 W incandescent lamp at 0.5 m away. The solution was loaded onto silica gel column and eluted with hexanes:EtOAc (2:1 to 1:1) to give product 27 (25.1 mg, 84%) as white solid.

Characterization data of 27: R_{f}=0.25 (silica gel, hexanes:EtOAc=1:1; [α]_{D}²⁰=+86.4° (c=1.0 in EtOAc); ¹H NMR (600 MHz, CDCl₃): δ=6.06 (dd, J=3.0, 1.4 Hz, 1H), 5.57 (d, J=2.9 Hz, 1H), 4.57 (dd, J=2.8, 1.4 Hz, 1H), 4.51 (dt, J=12.0, 4.0 Hz, 1H), 3.39 (s, 3H), 3.29 (d, J=9.5 Hz, 1H), 2.93 (dd, J=9.6, 1.4 Hz, 1H), 2.91 (hept d, J=6.7, 1.5 Hz, 1H), 2.60 (br s, 1H), 2.47 (ddd, J=11.1, 8.6, 2.9 Hz, 1H), 2.24 (ddt, J=14.5, 4.4, 1.3 Hz, 1H), 2.08-2.04 (m, 1H), 1.98-1.93 (m, 3H), 1.31-1.25 (m, 2H), 1.21 (s, 3H), 1.14 (d, J=6.8 Hz, 3H), 1.12 (d, J=6.6 Hz, 3H), 0.82 (d, J=7.3 Hz, 3H) ppm; ¹³C NMR (151 MHz, CDCl₃): δ=155.1, 140.9, 126.9, 126.8, 93.4, 90.9, 81.9, 72.0, 66.1, 59.4, 44.0, 41.3, 34.6, 31.1, 29.0, 28.4, 24.0, 20.9, 17.7, 7.3 ppm; HRMS (ESI, m/z): [M+H]⁺ calcd for C₂₁H₃₃O₅, 365.2323; found 365.2325.

### Synthesis of Compound 6 (Brassicicene L)

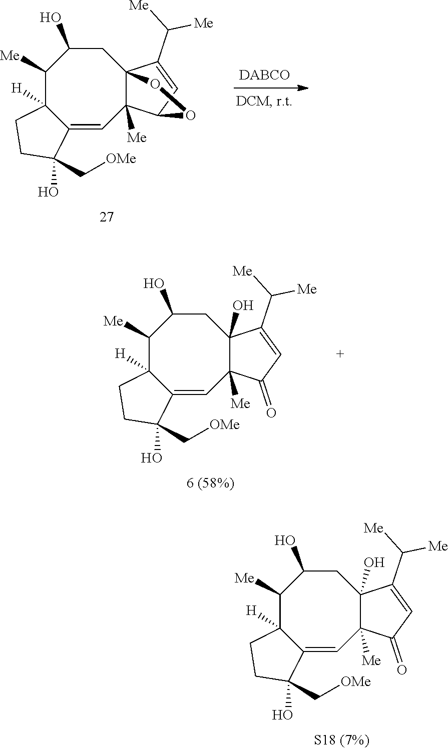

A solution of substrate 27 (9.7 mg, 26.6 mol) in CH₂Cl₂ (0.5 mL) was treated with 1,4-diazabicyclo[2.2.2]octane (29.8 mg, 0.26 mmol, 10.0 eq.). The flask was cooled to −78 °C, evacuated and charged with N₂, repeat degassing for three times. The solution was stirred at room temperature in the dark for 48 h and then quenched by the addition of NH₄Cl (sat. aq., 1.5 mL) and EtOAc (1 mL). The biphasic mixture was separated and the aqueous phase was extracted with EtOAc (1.5 mL×3). The combined organic phase was concentrated and the residue was purified by silica gel column, hexanes:EtOAc (1:1 to 0:1) to give product 6 (5.6 mg, 58%) as a white solid, together with diastereomer S18 (0.7 mg, 7%).

Characterization data of 6: R_{f}=0.3 (silica gel, EtOAc); [α]_{D}²⁰=−136.5 (c=0.2 in EtOAc) [lit.: [α]_{D}²⁰=−143.4° (c=0.07 in MeOH)^{S11}]; ¹H NMR (600 MHz, CD₃OD): δ=6.16 (s, 1H), 5.44 (d, J=2.2 Hz, 1H), 3.49 (d, J=9.8 Hz, 1H), 3.44 (d, J=9.8 Hz, 1H), 3.39 (s, 3H), 3.20-3.18 (m, 1H), 2.82 (hept, J=6.8 Hz, 1H), 2.53-2.50 (m, 1H), 2.29 (dd, J=14.5, 6.0 Hz, 1H), 2.05-1.98 (m, 2H), 1.69 (qt, J=6.6, 3.0 Hz, 1H), 1.64-1.59 (m, 1H), 1.55-1.48 (m, 2H), 1.29 (s, 3H), 1.28 (d, J=6.9 Hz, 3H), 1.24 (d, J=6.8 Hz, 3H), 0.88 (d, J=6.9 Hz, 3H) ppm; ¹³C NMR (151 MHz, CD₃OD): δ=210.4, 189.4, 152.4, 129.0, 126.6, 86.8, 83.8, 78.4, 72.8, 60.8, 59.6, 46.0, 44.4, 40.1, 37.6, 29.8, 29.3, 28.5, 23.8, 22.2, 9.0 ppm; HRMS (ESI, m/z): [M+H]⁺ calcd for C₂₁H₃₃O₅, 365.2323; found 365.2324.

Characterization data of S18: R_{f}=0.45 (silica gel, EtOAc); [α]_{D}²⁰=−137° (c=0.1 in EtOAc); ¹H NMR (600 MHz, CD₃OD): δ=5.99 (s, 1H), 5.52 (d, J=2.9 Hz, 1H), 4.28 (br s, 1H), 3.43 (d, J=9.8 Hz, 1H), 3.32 (s, 3H), 3.21 (d, J=9.7 Hz, 1H), 3.05 (br s, 1H), 2.85 (hept, J=6.8 Hz, 1H), 2.49 (s, 1H), 2.16 (s, 1H), 2.11-1.94 (m, 4H), 1.76 (dq, J=13.9, 6.8 Hz, 1H), 1.49 (dt, J=12.8, 8.0 Hz, 1H), 1.35 (s, 3H), 1.27 (d, J=6.8 Hz, 3H), 1.18 (d, J=6.8 Hz, 3H), 1.00 (d, J=7.0 Hz, 3H) ppm; ¹³C NMR (151 MHz, CD₃OD): δ=214.5, 192.1, 151.2, 129.1, 125.2, 85.0, 83.5, 79.2, 73.9, 59.5, 58.0, 45.3, 40.3, 36.7, 30.8, 28.6, 24.0, 24.0, 18.9 ppm; HRMS (ESI, m/z): [M+H]⁺ calcd for C₂₁H₃₃O₅, 365.2323; found 365.2324.

### Synthesis of Compound 28

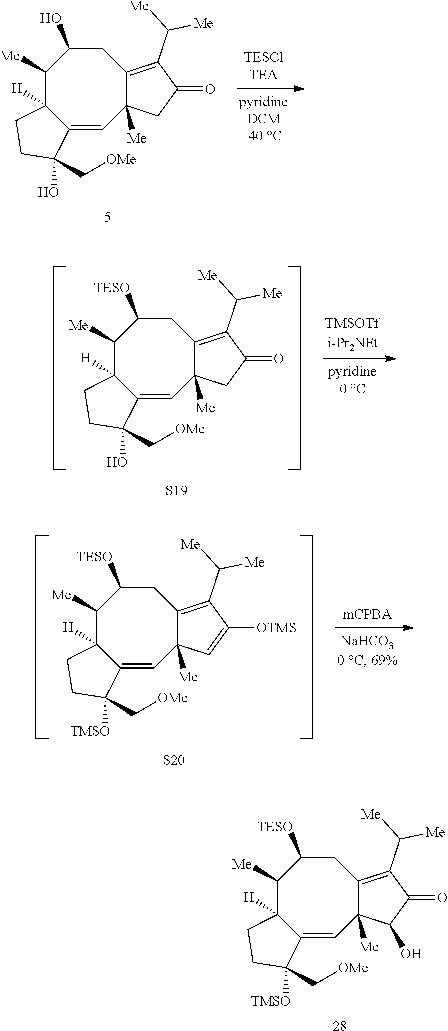

A solution of substrate 5 (30.2 mg, 86.7 mol) in CH₂Cl₂ (1 mL) was treated with pyridine (3.4 mg, 43.3 mol, 0.5 eq.), triethylamine (10.5 mg, 0.104 mmol, 1.2 eq.) and triethylchlorosilane (15.7 mg, 0.104 mmol, 1.2 eq.) in a dropwise manner. The mixture was stirred at 40 °C for 12 h, and TLC analysis showed the substrate was converted completely to intermediate S19. The reaction was cooled to 0 °C and treated with pyridine (34.3 mg, 0.43 mmol, 5.0 eq.), N-ethyl diisopropylamine (55.9 mg, 0.43 mmol, 5.0 eq.) and trimethylsilyl trifluoromethanesulfonate (96.3 mg, 0.43 mmol, 5.0 eq.) in a dropwise manner. The mixture was stirred at 0 °C for 1 h. During this period, the solution turned slowly to light brown. As S20 decomposes readily on TLC plates, only hydrolyzed TES-TMS-ketone could be observed (R_{f}=0.7, silica gel, hexanes:EtOAc=4:1). Tert-butanol (37 mg, 0.5 mmol, 5.7 eq.) was added at the same temperature. After another 10 min, NaHCO₃ (sat. aq., 2 mL) and a solution of purified meta-chloroperoxybenzoic acid in CH₂Cl₂ (1.0 mL (=0.7 mmol, 8 eq.) of 0.7 mol/L pure mCPBA in CH₂Cl₂, made by dissolving 172.5 mg of commercially available mCPBA (70-75% purity) in 1 mL CH₂Cl₂ and washing with 1 mL K₂HPO₄—KH₂PO₄ buffer (0.5 mol/L, pH=7) once) was added in one portion with vigorous stirring, and the color of the solution faded rapidly to nearly colorless. After stirring at 0 °C for another 15 min, Na₂SO₃ (s, about 100 mg) was added and the solution was stirred vigorously for another 15 min at room temperature. The biphasic mixture was separated and the aqueous phase was extracted with hexanes:EtOAc (1 1, 2 mL) and the combined organic phase was filtered through a short silica gel pad and eluted with hexanes:EtOAc (1:1). The eluent was concentrated and purified by flash column chromatography (hexanes:EtOAc=15:1 to 8:1) to give product 28 (32.9 mg, 69%) as colorless oil.

Characterization data of intermediate S19: R_{f}=0.5 (silica gel, hexanes:EtOAc=1:1); [α]_{D}²⁰=+18.3 (c=0.75 in EtOAc); ¹H NMR (600 MHz, CD₃COCD₃): 6=5.78 (d, J=2.3 Hz, 1H), 4.04 (ddd, J=11.2, 4.3, 3.1 Hz, 1H), 3.45 (d, J=9.8 Hz, 1H), 3.36 (s, 3H), 3.22 (d, J=9.9 Hz, 1H), 2.83 (hept, J=7.0 Hz, 1H), 2.75 (ddd, J=8.7, 6.0, 2.2 Hz, 1H), 2.62 (dd, J=12.9, 11.2 Hz, 1H), 2.49 (dd, J=12.9, 3.0 Hz, 1H), 2.30 (d, J=18.1 Hz, 1H), 2.20 (dd, J=18.1, 1.0 Hz, 1H), 2.00-1.91 (m, 2H), 1.85 (qd, J=6.9, 4.0 Hz, 1H), 1.42-1.31 (m, 2H), 1.33 (s, 3H), 1.29 (d, J=7.0 Hz, 3H), 1.17 (d, J=6.9 Hz, 3H), 1.00 (t, J=7.9 Hz, 9H), 0.93 (d, J=7.0 Hz, 3H), 0.69-0.65 (m, 6H) ppm; ¹³C NMR (151 MHz, CD₃COCD₃): 6=206.3, 172.6, 146.1, 141.9, 134.0, 82.6, 78.1, 76.8, 59.4, 53.1, 47.7, 46.9, 41.4, 36.4, 32.7, 32.1, 27.9, 27.2, 19.94, 19.86, 8.4, 7.2, 5.5 ppm; HRMS (ESI, m/z): [M+H]⁺ calcd for C₂₇H₄₇O₄Si, 463.3238; found 463.3234. Characterization data of product 28: R_{f}=0.55 (silica gel, hexanes:EtOAc=4:1); [α]_{D}²⁰=+3.7 (c=0.19 in EtOAc); ¹H NMR (600 MHz, CD₃COCD₃): 6=5.83 (d, J=2.3 Hz, 1H), 4.44 (d, J=5.2 Hz, 1H), 4.00 (ddd, J=10.8, 4.3, 2.8 Hz, 1H), 3.78 (d, J=5.2 Hz, 1H), 3.47 (d, J=10.0 Hz, 1H), 3.36 (s, 3H), 3.16 (d, J=9.9 Hz, 1H), 2.84 (hept, J=7.0 Hz, 1H), 2.78 (s, 1H), 2.69 (t, J=7.8 Hz, 1H), 2.60 (dd, J=12.8, 10.8 Hz, 1H), 2.49 (dd, J=12.9, 2.7 Hz, 1H), 2.01-1.95 (m, 1H), 1.88-1.84 (m, 1H), 1.47 (ddd, J=12.0, 9.4, 7.3 Hz, 1H), 1.43-1.35 (m, 1H), 1.26 (d, J=6.9 Hz, 3H), 1.17 (d, J=6.9 Hz, 3H), 1.17 (s, 3H), 1.00 (t, J=8.0 Hz, 9H), 0.96 (d, J=7.0 Hz, 3H), 0.67 (q, J=7.8 Hz, 6H), 0.07 (s, 9H) ppm; ¹³C NMR (151 MHz, CD₃COCD₃): 6=26 206.6, 171.6, 143.2, 143.0, 133.1, 86.1, 82.5, 77.9, 77.5, 59.1, 52.2, 45.3, 40.9, 35.8, 32.8, 32.0, 26.9, 23.3, 20.2, 19.7, 8.7, 7.2, 5.5, 2.5 ppm; HRMS (ESI, m/z): [M+H]⁺ calcd for C₃₀H₅₅O₅Si₂, 551.3583; found 551.3584.

### Syntheses of Compound 29 (Brassicicene K) and 31

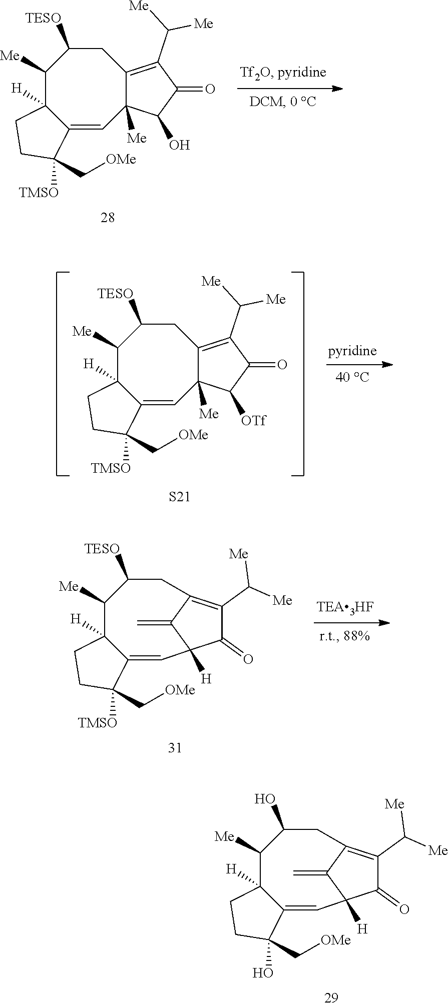

A solution of substrate 28 (24.0 mg, 43.6 mol) in CH₂Cl₂ (0.9 mL) was cooled to 0 °C, and treated with pyridine (15.8 mg, 0.2 mmol, 4.6 eq.) and trifluoromethanesulfonic anhydride (0.1 mL, 1 mol/L in CH₂Cl₂, 0.1 mmol, 2.3 eq.) in a dropwise manner. The mixture was stirred at 0 °C for 1 h, and TLC analysis showed complete conversion of substrate 28 to intermediate S21 (R_{f}=0.77, silica gel, hexanes:EtOAc=4:1). Then tert-butanol (4.8 mg, 65 mol, 1.5 eq.) was added and the resulting mixture was stirred for another 15 min. Pyridine (4 mL) was added and the solution was stirred at 40 °C for 16 h.

To obtain 29 in one pot, the pyridine solution was cooled down to room temperature and treated with triethylamine trihydrofluoride (0.25 mL, 1.53 mmol, 35 eq.). The solution was stirred for 24 h, concentrated under vacuum to about 0.5 mL, and purified by flash column chromatography (hexanes:EtOAc=1:1 to 0:1) to give product 29 (13.3 mg, 88%) as colorless oil.

To obtain compound 31, the pyridine solution after the rearrangement was concentrated in vacuo, and the residue was purified by silica gel flash column chromatography (hexanes:EtOAc=15:1 to 6:1) to give product 31 (from 31.1 mg 28 to 24.6 mg 31, the amount of reagents were recalculated accordingly, yield 82%) as colorless oil.

The yield of 29 is higher than that of 31, as during the rearrangement in pyridine, the material underwent partial TMS deprotection, which could be observed on TLC (R_{f}=0.1, silica gel, hexanes:EtOAc=4:1) and confirmed by ¹H NMR. This ‘byproduct’ was also converted to after global deprotection.

Characterization data of product 29: R_{f}=0.48 (silica gel, EtOAc); [α]_{D}²⁰=−65.6° (c=0.16 in EtOAc) [lit.: [α]_{D}²⁰=−69.7° (c=0.0070 in MeOH).^{S13} lit.: [α]=−79° (c=0.01 in MeOH).^{S14}]; ¹H NMR (600 MHz, CD₃OD): δ=6.27 (dd, J=10.5, 1.8 Hz, 1H), 5.25 (s, 1H), 5.10 (s, 1H), 3.99 (ddd, J=9.8, 6.2, 3.0 Hz, 1H), 3.65 (d, J=10.5 Hz, 1H), 3.36 (s, 3H), 3.33 (d, J=10.0 Hz, 1H), 3.30 (d, J=10.0 Hz, 1H), 3.09 (dd, J=12.2, 6.1 Hz, 1H), 2.95 (hept, J=6.9 Hz, 1H), 2.72 (d, J=7.0 Hz, 1H), 2.65 (dd, J=12.6, 10.2 Hz, 1H), 1.96 (tt, J=11.8, 7.0 Hz, 1H), 1.84 (qt, J=7.8, 2.4 Hz, 1H), 1.72 (ddd, J=13.1, 11.3, 6.1 Hz, 1H), 1.51 (ddd, J=13.0, 6.5, 3.9 Hz, 1H), 1.41 (ddt, J=12.6, 6.4, 3.4 Hz, 1H), 1.33 (d, J=6.9 Hz, 3H), 1.20 (d, J=6.9 Hz, 3H), 0.91 (d, J=7.5 Hz, 3H) ppm; ¹³C NMR (151 MHz, CD₃OD): δ=204.2, 165.5, 150.6, 150.5, 149.7, 125.6, 106.6, 83.5, 79.4, 75.6, 59.6, 54.9, 49.7, 46.8, 37.3, 36.0, 31.1, 28.0, 21.7, 19.9, 11.2 ppm; HRMS (ESI, m/z): [M+H]⁺ calcd for C₂₁H₃₁O₄, 347.2217; found 347.2217.

Characterization data of product 31: R_{f}=0.72 (silica gel, hexanes:EtOAc=4:1); [α]_{D}²⁰=−126.0° (c=0.1 in EtOAc); ¹H NMR (600 MHz, CD₃COCD₃): 6=6.21 (dd, J=10.6, 2.2 Hz, 1H), 5.25 (s, 1H), 5.08 (s, 1H), 4.06 (ddd, J=9.7, 6.2, 3.0 Hz, 1H), 3.63 (d, J=10.6 Hz, 1H), 3.32 (s, 3H), 3.31 (d, J=10.2 Hz, 1H), 3.06 (d, J=10.2 Hz, 1H), 3.01 (dd, J=12.3, 6.2 Hz, 1H), 2.94 (hept, J=6.9 Hz, 1H), 2.69 (dd, J=12.3, 10.4 Hz, 1H), 2.68-2.65 (m, 1H), 1.88 (dt, J=12.9, 6.6 Hz, 1H), 1.83 (qt, J=7.8, 2.2 Hz, 1H), 1.40 (dt, J=12.3, 7.1 Hz, 1H), 1.33 (d, J=6.9 Hz, 3H), 1.33-1.28 (m, 2H), 1.20 (d, J=6.9 Hz, 3H), 1.01 (t, J=7.9 Hz, 9H), 0.94 (d, J=7.6 Hz, 3H), 0.68 (q, J=8.1 Hz, 6H), 0.06 (s, 9H) ppm; ¹³C NMR (151 MHz, CD₃COCD₃): 6=201.0, 162.8, 150.2, 148.7, 147.9, 124.4, 105.8, 86.7, 78.4, 76.4, 59.1, 54.7, 50.5, 45.2, 36.7, 34.8, 31.8, 27.7, 21.6, 20.1, 10.3, 7.2, 5.4, 2.5 ppm; HRMS (ESI, m/z): [M+H]⁺ calcd for C₃₀H₅₃O₄Si₂, 533.3477; found 533.3477.

### Synthesis of Compound 7 (Brassicicene C)

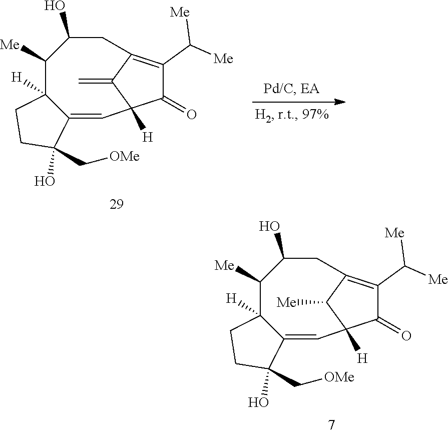 [SMILES: C=C1C2=C(C(C)C)C(=O)[C@@H]1/C=C1\[C@@H](CC[C@]1(O)COC)[C@@H](C)[C@@H](O)C2 ; "29" ; COC[C@@]1(O)CC[C@@H]2/C1=C\[C@H]1C(=O)C(C(C)C)=C(C[C@H](O)[C@@H]2C)[C@H]1C ; "7"]

Substrate 29 (3.3 mg, 9.5 mol) and palladium on carbon (10% w/w, reduced, 1.0 mg) was added to a reaction flask and charged with hydrogen through a balloon. EtOAc (0.5 mL) was added via syringe. The suspension was stirred vigorously at room temperature for 2 h, filtered through a short silica gel pad and eluted with EtOAc to give product 7 (3.2 mg, 97%) as colorless oil.

Characterization data of product 7: R_{f}=0.45 (silica gel, EtOAc); [α]_{D}²⁰=−63.3° (c=0.21 in EtOAc) [lit.: [α]_{D}²³=−66.6° (c=0.0150 in MeOH).^{S10} lit.: [α]_{D}²⁰=−126° (c=0.01 in MeOH)^{S13}; ¹H NMR (600 MHz, CD₃OD): δ=6.10 (dd, J=10.1, 1.7 Hz, 1H), 4.18 (ddd, J=11.4, 5.6, 3.6 Hz, 1H), 3.49 (d, J=9.8 Hz, 1H), 3.39 (d, J=9.8 Hz, 1H), 3.38 (s, 3H), 3.37 (dd, J=10.8, 4.8 Hz, 1H), 3.14 (tt, J=8.0, 6.2 Hz, 1H), 2.95-2.90 (m, 2H), 2.80 (d, J=7.8 Hz, 1H), 2.46 (dd, J=13.8, 11.4 Hz, 1H), 2.05 (qd, J=7.6, 3.5 Hz, 1H), 1.99 (tdd, J=12.0, 7.8, 6.9 Hz, 1H), 1.88 (td, J=12.6, 6.3 Hz, 1H), 1.55 (ddd, J=13.0, 6.7, 3.3 Hz, 1H), 1.45 (ddt, J=12.0, 5.9, 2.8 Hz, 1H), 1.33 (d, J=6.8 Hz, 3H), 1.26 (d, J=7.3 Hz, 3H), 1.16 (d, J=6.9 Hz, 3H), 1.01 (d, J=7.6 Hz, 3H) ppm; ¹³C NMR (151 MHz, CD₃OD): δ=207.4, 176.8, 151.2, 147.7, 127.8, 84.1, 79.5, 75.5, 59.6, 58.7, 50.9, 47.8, 44.8, 38.0, 36.7, 30.6, 28.9, 22.0, 19.0, 13.0, 12.4 ppm; HRMS (ESI, m/z): [M+H]⁺ calcd for C₂₁H₃₃O₄, 349.2373; found 349.2374.

### Synthesis of Compound 30 (Brassicicene H)

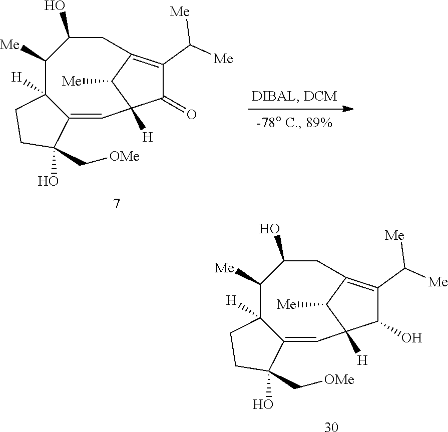 [SMILES: COC[C@@]1(O)CC[C@@H]2/C1=C\[C@H]1C(=O)C(C(C)C)=C(C[C@H](O)[C@@H]2C)[C@H]1C ; "7" ; COC[C@@]1(O)CC[C@@H]2/C1=C\[C@@H]1[C@H](C)C(=C(C(C)C)[C@@H]1O)C[C@H](O)[C@@H]2C ; "30"]

A solution of substrate 7 (1.9 mg, 5.5 mol) in CH₂Cl₂ (0.5 mL) was cooled to −78 °C and treated with diisobutylaluminum hydride (0.05 mL, 1 mol/L in hexanes, 0.05 mmol, 9.2 eq.). The solution was stirred at the same temperature for 1 h before it was quenched by the addition of MeOH (0.01 mL), pyridine (0.05 mL) and silica gel (about 100 mg). After stirring at room temperature for 15 min, the suspension was loaded directly onto silica gel and eluted with hexanes:EtOAc (1:1 to 0:1) to give product 30 (1.7 mg, 89%) as colorless oil.

Characterization data of product 30: R_{f}=0.35 (silica gel, EtOAc); [α]_{D}²⁰=−3° (c=0.1 in EtOAc) [lit.: [α]=_{D}²⁰=−2° (c=0.01 in MeOH).^{S14}]; ¹H NMR (600 MHz, CDCl₃): δ=5.83 (dd, J=9.5, 1.8 Hz, 1H), 4.80 (s, 1H), 4.29-4.25 (m, 1H), 3.51 (d, J=9.3 Hz, 1H), 3.43 (s, 3H), 3.39 (d, J=9.3 Hz, 1H), 3.34 (d, J=8.3 Hz, 1H), 3.27 (dt, J=10.0, 5.2 Hz, 1H), 2.89 (hept, J=6.9 Hz, 1H), 2.60-2.53 (m, 3H), 2.09-2.02 (m, 2H), 2.00 (qd, J=7.5, 3.5 Hz, 1H), 1.88 (td, J=12.7, 6.3 Hz, 1H), 1.67 (dddd, J=13.2, 6.8, 3.0, 0.8 Hz, 1H), 1.47 (ddt, J=12.0, 5.7, 2.7 Hz, 1H), 1.37 (d, J=7.0 Hz, 3H), 1.12 (d, J=7.4 Hz, 3H), 1.11 (d, J=7.2 Hz, 3H), 0.97 (d, J=7.5 Hz, 3H) ppm; ¹³C NMR (151 MHz, CDCl₃): δ=153.4, 144.2, 136.5, 126.0, 82.7, 80.1, 79.0, 75.3, 59.6, 52.3, 26 49.9, 48.4, 42.6, 37.0, 36.7, 29.8, 28.3, 21.9, 21.7, 13.8, 12.0 ppm; HRMS (ESI, m/z): [M+H]⁺ calcd for C₂₁H₃₅O₄, 351.2530; found 351.2532.

### Synthesis of Compound 32 (Brassicicene J)

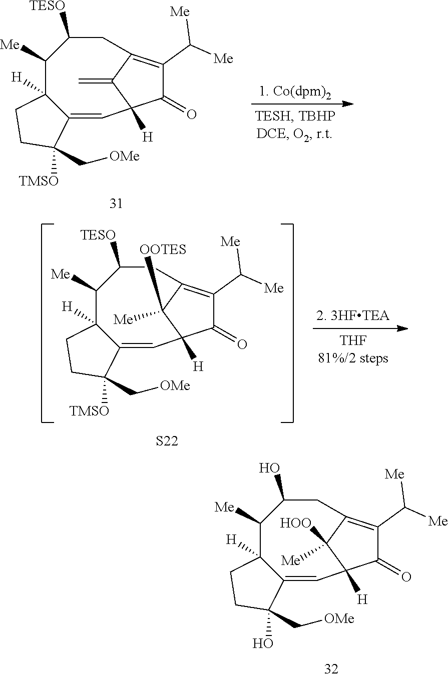

A solution of substrate 31 (5.4 mg, 10 mol) in (CH₂C1)₂ (1.9 mL) was treated with triethylsilane (0.1 mL, final conc. 0.31 mmol/L) and tert-butylhydroperoxide (0.1 μL, 5.5 mol/L in nonane, 0.55 mol, 0.055 eq.). The solution was bubbled with O₂ for 5 min with stirring at room temperature. Bis(2,2,6,6-tetramethyl-3,5-heptanedionato)cobalt(II) (0.4 mg, 1 mol, 0.1 eq.) was added and the solution was stirred vigorously under 02 for 3 h at which point TLC analysis showed complete conversion to S22 (R_{f}=0.65, silica gel, hexanes:EtOAc=8:1). The reaction mixture was loaded directly onto a short silica gel pad and, within one minute, eluated with hexanes:EtOAc=4:1. S22 is not stable upon prolonged exposure to silica gel and thus was not further purified. The eluant was concentrated and the residue was dissolved in THF (1 mL). Triethylamine trihydrofluoride (0.1 mL) was added, and the solution was stirred at room temperature for 6 h. Triethylamine (0.1 mL) was added, and the solution was loaded directly onto a short silica gel column and eluted with EtOAc. The eluant was concentrated and the residue was purified by silica gel column chromatography (hexanes:EtOAc, 1:1 to 0:1) to give product 32 (3.1 mg, 81%) as white solid.

Characterization data of product 32: R_{f}=0.4 (silica gel, EtOAc); [α]_{D}²⁰=−99.9° (c=0.3 in EtOAc) [lit.: [α]_{D}²=−112.1° (c=0.056 in MeOH)^{S13}, [α]_{D}²⁰=−110° (c=0.01 in MeOH)^{S14}]; ¹H NMR (600 MHz, CD₃OD): δ=6.12 (dd, J=10.5, 1.7 Hz, 1H), 4.15 (ddd, J=11.4, 5.4, 3.8 Hz, 1H), 3.48 (d, J=9.8 Hz, 1H), 3.40 (d, J=9.7 Hz, 1H), 3.38 (s, 3H), 3.22 (d, J=10.5 Hz, 1H), 2.97 (hept, J=7.0 Hz, 1H), 2.94 (dd, J=14.4, 5.5 Hz, 1H), 2.85 (d, J=7.8 Hz, 1H), 2.42 (dd, J=14.2, 11.6 Hz, 1H), 2.06 (ddt, J=11.4, 7.8, 3.5 Hz, 1H), 1.98 (tt, J=12.1, 7.2 Hz, 1H), 1.86 (td, J=12.3, 6.5 Hz, 1H), 1.60 (s, 3H), 1.54 (ddt, J=13.1, 6.8, 3.7 Hz, 1H), 1.45 (ddd, J=11.5, 5.8, 2.8 Hz, 1H), 1.36 (d, J=6.9 Hz, 3H), 1.23 (d, J=6.9 Hz, 3H), 1.02 (d, J=7.6 Hz, 3H) ppm; ¹³C NMR (151 MHz, CD₃OD): δ=205.3, 166.9, 152.6, 152.3, 126.4, 92.6, 83.9, 79.3, 75.6, 59.8, 59.6, 50.5, 47.6, 38.0, 36.5, 29.8, 29.4, 22.0, 19.0, 16.5, 12.1 ppm; HRMS (ESI, m/z): [M+H]⁺ calcd for C₂₁H₃₃O₆, 381.2272; found 381.2268.

### Synthesis of Compound S23

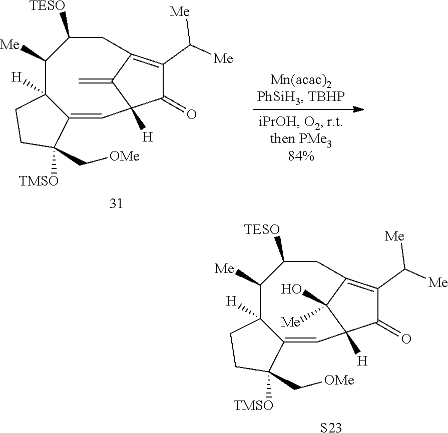

A solution of substrate 31 (5.2 mg, 9.8 mol) in iPrOH (1.9 mL) was treated with phenylsilane (0.1 mL, final conc. 0.4 mmol/L) and tert-butylhydroperoxide (0.09 μL, 5.5 mol/L in nonane, 0.5 mol, 0.05 eq.). The solution was bubbled with O₂ for 5 min with stirring at room temperature. Manganese(II) acetylacetonate (0.25 mg, 1 mol, 0.1 eq.) was added and the solution was stirred vigorously under 02 for 3 h at which point TLC analysis showed complete consumption of 31 (several new spots might be observed). The atmosphere was changed to air and PMe₃ (98 μL, 1 mol/L in THF, 98 mol, 10 eq.) was added. After stirring for another 10 min, the solution was concentrated in vacuo and the residue was purified by silica gel flash column chromatography (hexanes:EtOAc, 6:1 to 2:1) to give product S23 (4.5 mg, 84%) as colorless solid.

Characterization data of product S23: R_{f}=0.2 (silica gel, hexanes:EtOAc=4:1); [α]²⁰=−86.6° (c=0.2 in EtOAc); ¹H NMR (600 MHz, CD₃OD): δ=6.09 (dd, J=10.5, 2.0 Hz, 1H), 4.20 (ddd, J=11.3, 5.6, 3.8 Hz, 1H), 3.43 (d, J=10.1 Hz, 1H), 3.36 (s, 3H), 3.26 (d, J=10.1 Hz, 1H), 3.09 (d, J=10.5 Hz, 1H), 2.89 (hept, J=7.0 Hz, 1H), 2.86 (dd, J=14.6, 5.3 Hz, 1H), 2.78-2.75 (m, 1H), 2.50 (dd, J=14.3, 11.3 Hz, 1H), 2.01-1.96 (m, 2H), 1.90 (ddd, J=12.9, 8.9, 6.5 Hz, 1H), 1.57 (s, 3H), 1.51 (dt, J=13.1, 6.6 Hz, 1H), 1.39-1.35 (m, 1H), 1.35 (d, J=6.9 Hz, 3H), 1.23 (d, J=6.8 Hz, 3H), 1.02 (t, J=8.0 Hz, 9H), 1.01 (d, J=7.7 Hz, 3H), 0.67 (q, J=7.8 Hz, 6H), 0.06 (s, 9H) ppm; ¹³C NMR (151 MHz, CD₃OD): δ=206.4, 171.9, 149.8, 149.2, 126.6, 87.6, 81.0, 79.3, 76.7, 65.6, 59.4, 51.7, 46.4, 37.6, 35.7, 30.5, 29.6, 21.61, 21.56, 19.1, 11.3, 7.3, 5.9, 2.5 ppm; HRMS (ESI, m/z): [M+H]⁺ calcd for C₃₀H₅₅O₅Si₂, 551.3583; found 551.3584.

### Synthesis of Compound 33 (Brassicicene F)

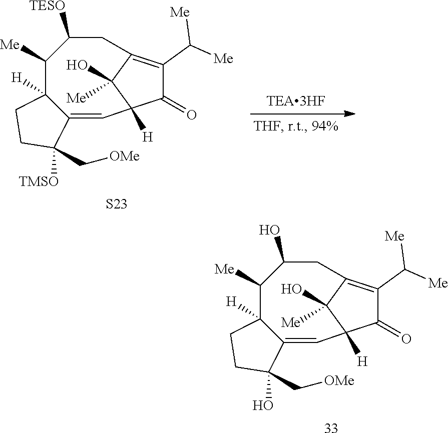

A solution of substrate S23 (2.7 mg, 4.9 mol) in THF (1.0 mL) was treated with trihydrofluoride triethylamine (0.1 mL) and the solution was stirred at room temperature for 6 h. Triethylamine (0.1 mL) was added, and the solution was loaded directly onto a short silica gel column and eluted with EtOAc. The eluant was concentrated and the residue was purified by silica gel column chromatography (EtOAc) to give product 33 (1.7 mg, 94%) as white solid.

Characterization data of product 33: R_{f}=0.08 (silica gel, EtOAc); [α]_{D}²⁰=−97.7° (c=0.1 in EtOAc) [lit.: [α]_{D}²³=−81.7° (c=0.06 in MeOH)^{S10}, [α]_{D}²⁰=−105° (c=0.01 in MeOH)^{S14}]; ¹H NMR (600 MHz, CD₃OD): δ=6.14 (dd, J=10.4, 1.8 Hz, 1H), 4.16 (ddd, J=11.6, 5.5, 3.6 Hz, 1H), 3.47 (d, J=9.8 Hz, 1H), 3.39 (d, J=9.8 Hz, 1H), 3.37 (s, 3H), 3.10 (d, J=10.4 Hz, 1H), 2.96 (dd, J=14.1, 5.5 Hz, 1H), 2.92 (hept, J=6.9 Hz, 1H), 2.85 (d, J=7.8 Hz, 1H), 2.45 (dd, J=14.1, 11.5 Hz, 1H), 2.07-2.02 (m, 1H), 1.97 (tt, J=12.4, 7.3 Hz, 1H), 1.86 (td, J=12.6, 6.2 Hz, 1H), 1.58 (s, 3H), 1.53 (ddd, J=13.0, 6.7, 3.3 Hz, 1H), 1.45 (ddt, J=12.0, 5.9, 2.9 Hz, 1H), 1.34 (d, J=6.9 Hz, 3H), 1.21 (d, J=7.0 Hz, 3H), 1.00 (d, J=7.5 Hz, 3H) ppm; ¹³C NMR (151 MHz, CD₃OD): δ=206.8, 172.4, 151.3, 149.8, 127.3, 84.0, 81.2, 79.4, 75.6, 65.4, 59.6, 50.5, 47.3, 38.0, 36.5, 29.3, 29.2, 21.7, 21.5, 18.9, 12.0 ppm; HRMS (ESI, m/z): [M+H]⁺ calcd for C₂₁H₃₃O₅, 365.2323; found 365.2323.

### Synthesis of Compound 34

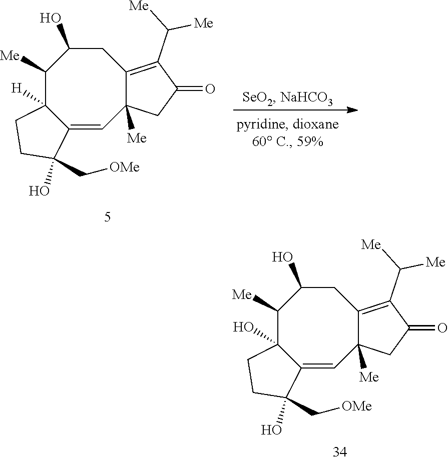 [SMILES: COC[C@@]1(O)CC[C@@H]2/C1=C\[C@@]1(C)CC(=O)C(C(C)C)=C1C[C@H](O)[C@@H]2C ; COC[C@@]1(O)CC[C@]2(O)/C1=C\[C@@]1(C)CC(=O)C(C(C)C)=C1C[C@H](O)[C@@H]2C]

A solution of substrate 5 (8.8 mg, 25.3 mol) in dioxane (1 mL) was treated with NaHCO₃ (192 mg, 2.29 mmol, 90 eq.), pyridine (1 mg, 13 mol, 0.5 eq.) and SeO₂ (85 mg, 0.76 mmol, 30 eq.). The suspension was stirred at 60 °C for 4 h and then cooled to room temperature. H₂O (2 mL) was added with vigorous stirring for 15 min. The solution was extracted with EtOAc (3 mL×3), and the combined organic phase was washed with brine (2 mL). After concentration under vacuum, the residue was purified by silica gel column, hexanes:EtOAc (1:1 to 0:1) to give product 34 (5.4 mg, 59%) as white solid.

Characterization data of product 34: R_{f}=0.27 (silica gel, EtOAc); [α]_{D}²⁰=+54.4° (c=0.25 in EtOAc); ¹H NMR (600 MHz, CD₃COCD₃): δ=5.84 (s, 1H), 4.52-4.47 (m, 1H), 3.90 (d, J=4.1 Hz, 1H), 3.56 (s, 1H), 3.41 (d, J=10.0 Hz, 1H), 3.35 (s, 3H), 3.19 (s, 1H), 3.09 (d, J=10.0 Hz, 1H), 2.84-2.78 (m, 1H), 2.58-2.53 (m, 2H), 2.29 (d, J=17.9 Hz, 1H), 2.17 (d, J=18.1 Hz, 1H), 2.15-2.12 (m, 1H), 1.99-1.96 (m, 1H), 1.90-1.84 (m, 1H), 1.72-1.64 (m, 2H), 1.31 (s, 3H), 1.20 (d, J=6.9 Hz, 3H), 1.13 (d, J=6.9 Hz, 3H), 0.97 (d, J=7.0 Hz, 3H) ppm; ¹³C NMR (151 MHz, CD₃COCD₃): δ=205.7, 173.5, 145.7, 143.8, 136.7, 83.5, 81.9, 77.8, 69.6, 59.4, 52.6, 48.6, 47.2, 41.3, 33.3, 31.5, 28.8, 26.7, 20.2, 20.1, 9.9 ppm; HRMS (ESI, m/z): [M+H]⁺ calcd for 4 C₂₁H₃₃O₅, 365.2323; found 365.2320.

### Synthesis of Compound 35

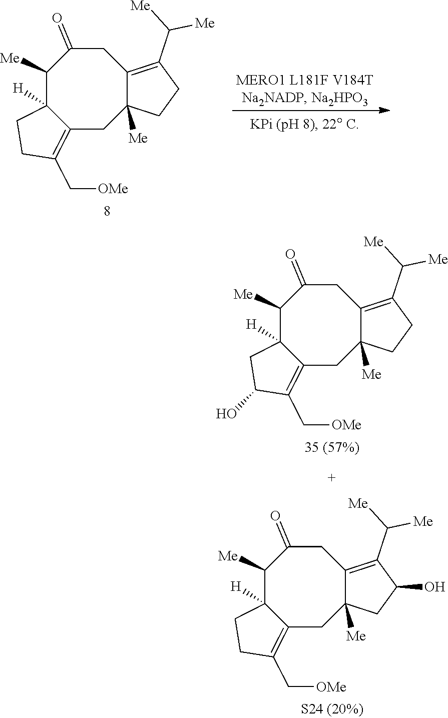 [SMILES: COCC1=C2C[C@@]3(C)CCC(C(C)C)=C3CC(=O)[C@H](C)[C@@H]2CC1 ; COCC1=C2C[C@@]3(C)CCC(C(C)C)=C3CC(=O)[C@H](C)[C@@H]2C[C@H]1O ; COCC1=C2C[C@@]3(C)C[C@H](O)C(C(C)C)=C3CC(=O)[C@H](C)[C@@H]2CC1]

### Procedure for Enzyme Expression and Lysate Preparation of MERO1 L181F V184T:

Recombinant E. coli BL21(DE3) harboring plasmids pET22b(+)—P450B_{M}3 MERO1 L181F V184T and pRSF-Opt13 was grown in LB (4 mL, with 50 g/mL ampicillin and 25 μg/mL kanamycin) for 12 h, and 0.2 mL of the culture was used to inoculate 50 mL terrific broth media (in 250 mL non-beveled Erlenmeyer flask, with 50 g/mL ampicillin, 25 g/mL kanamycin and trace metal mix (FeCl₃ 50 μM, CaCl₂ 20 μM, MnSO₄ 10 μM, ZnSO₄ 10 μM, CoSO₄ 2 μM, CuCl₂ 2 μM, NiCl₂ 2 μM, Na₂MoO₄ 2 μM, H₃BO₃ 2 μM)). The culture was shaken at 250 rpm at 37 °C for roughly 3 hours or until an optical density (OD₆₀₀) of 0.9-1.0 was reached. The culture was cooled on ice for 20 min, and then induced by the addition of isopropyl β-D-1-thiogalactopyranoside (final concentration=0.5 mM) and 5-aminolevulinic acid (final concentration=1.0 mM). The culture was shaken at 250 rpm at 23 °C for 24 h and then cooled to 0 °C. The cells were harvested by centrifugation (4 °C, 10 min, 3234×g) and resuspended in KH₂PO₄—K₂HPO₄ buffer (50 mM, pH 8.0) to an OD₆₀₀ of 60 (about 20 mL in total). The suspension was disrupted by sonication in an ice bath. The crude lysate was centrifuged (4 °C, 10 min, 3234×g) again to make clarified lysate, which was kept at 0 °C until its use.

Procedure for Enzymatic Reaction with MERO1 L181F V184T:

A 125 mL non-beveled Erlenmeyer flask was charged with the above clarified lysate (20 mL). Na₂NADP (11.1 mg, 13 mol, 0.2 eq.), Na₂HPO₃·5H₂O (273 mg, 1.3 mmol, 20 eq.), and substrate 8 (20 mg, 63 mol) in DMSO (1.0 mL) were sequentially added with gentle shaking. The flask was shaken at 160 rpm at 22 °C for 16 h. The reaction mixture was treated with Na₂SO₄ (solid, ~4 g), pyridine (1 mL) and stirred until Na₂SO₄ was fully dissolved. The aqueous phase was then extracted with EtOAc (20 mL×3). The combined organic phase was washed with Na₂SO₄ (sat. aq., 20 mL) and concentrated. The residue was purified by flash column chromatography (silica gel, hexanes:EtOAc=4:1 to 2:1) to give product 35 (11.9 mg, 57%) and S24 (4.2 mg, 20%).

Characterization data of product 35: R_{f}=0.32 (silica gel, hexanes:EtOAc=1:1); [α]_{D}²⁰=−126.6° (c=−0.5 in EtOAc); ¹H NMR (600 MHz, CD₃COCD₃): δ=4.65 (t, J=5.8 Hz, 1H), 4.14 (dd, J=10.8, 2.0 Hz, 1H), 4.03 (d, J=10.8 Hz, 1H), 3.39-3.34 (m, 2H), 3.32 (s, 3H), 3.14 (s, 1H), 3.04 (d, J=16.9 Hz, 1H), 2.56 (d, J=16.9 Hz, 1H), 2.52 (d, J=13.2 Hz, 1H), 2.46 (hept, J=7.1 Hz, 1H), 2.41-2.35 (m, 1H), 2.33 (d, J=13.3 Hz, 1H), 2.27 (ddd, J=15.9, 9.2, 4.6 Hz, 1H), 1.82 (ddd, J=13.3, 8.9, 4.6 Hz, 1H), 1.77-1.72 (m, 2H), 1.40 (ddd, J=13.8, 8.6, 7.0 Hz, 1H), 1.06 (s, 3H), 0.93 (d, J=6.8 Hz, 6H), 0.91 (d, J=6.8 Hz, 3H) ppm; ¹³C NMR (151 MHz, CD₃COCD₃): 6=212.0, 145.5, 142.9, 140.1, 135.2, 75.8, 68.7, 58.7, 52.3, 51.2, 42.9, 41.7, 39.4, 39.3, 34.8, 27.9, 27.8, 25.9, 21.0, 20.5, 14.1 ppm; HRMS (ESI, m/z): [M+H]⁺ calcd for C₂₁H₃₃O₃, 333.2424; found 333.2424.

Characterization data of product S24: R_{f}=0.35 (silica gel, hexanes:EtOAc=1:1); [α]_{D}²⁰=−145° (c=0.1 in EtOAc); ¹H NMR (600 MHz, CD₃COCD₃): 6=4.92 (q, J=6.3 Hz, 1H), 4.01 (d, J=11.5 Hz, 1H), 3.97 (d, J=11.5 Hz, 1H), 3.56 (d, J=6.8 Hz, 1H), 3.30 (s, 1H), 3.24 (s, 3H), 3.08 (d, J=17.0 Hz, 1H), 2.87 (s, 1H), 2.69 (d, J=16.9 Hz, 1H), 2.54 (d, J=13.3 Hz, 1H), 2.46 (hept, J=7.0 Hz, 1H), 2.34-2.24 (m, 2H), 2.20 (dd, J=13.1, 7.5 Hz, 1H), 2.16 (d, J=13.6 Hz, 1H), 1.86 (dtd, J=12.7, 7.9, 1.8 Hz, 1H), 1.64 (dd, J=13.1, 5.3 Hz, 1H), 1.12 (s, 3H), 1.09 (d, J=7.0 Hz, 3H), 1.08-1.06 (m, 1H), 1.07 (d, J=6.9 Hz, 3H), 0.93 (d, J=6.8 Hz, 3H) ppm; ¹³C NMR (151 MHz, CD₃COCD₃): 6=211.8, 147.3, 139.0, 138.7, 138.6, 75.5, 70.0, 58.1, 54.8, 51.0, 49.1, 43.6, 42.1, 40.3, 33.1, 27.9, 27.0, 24.5, 22.4, 19.7, 14.2 ppm; HRMS (ESI, m/z): [M+H]⁺ calcd for C₂₁H₃₃O₃, 333.2424; found 333.2422.

TABLE S6
¹H NMR Comparison of Cotylenol (in CDCl₃)

| | Synthetic | Synthetic | Synthetic | Synthetic |
|---|---|---|---|---|
| | (by Takeshita | (by Nakada | (by Shenvi | (the present |
| Natural^{S7} | et al.^{S8}) | et al.^{S6}) | et al.^{S15}) | disclosure) |
| 5.55 (m, 1H) | 5.52 (d, J = 2.5 | 5.52 (d, J = 2.6 | 5.52 (d, J = 2.5 | 5.50 (d, J = 2.6 |
| | Hz, 1H) | Hz, 1H) | Hz) | Hz, 1H) |
| 4.2-3.8 (m, 2H) | 4.07 (d, J = 10 | 4.07 (d, J =10.2 | 4.07 (d, J = 9.9 | 4.05 (d, J = 10.0 |
| | Hz, 1H) | Hz, 1H) | Hz) | Hz, 1H) |
| | 3.94 (dd, J = | 3.94 (dd, J = | 3.94 (dd, J = | 3.92 (dd, J = |
| | 10, 4.5 Hz, 1H) | 10.2, 4.4 Hz, 1H) | 9.9, 4.4 Hz) | 10.0, 4.3 Hz, 1H) |
| 3.42 (s, 3H) | 3.41 (s, 3H) | 3.40 (s, 3H) | 3.40 (s, 3H) | 3.39 (s, 3H) |
| 3.38 (AB type) | 3.36 (d, J = 9.5 | 3.36 (d, J = 9.7 | 3.36 (d, J = 9.3 | 3.35 (d, J = 9.5 |
| | Hz, 1H) | Hz, 1H) | Hz) | Hz, 1H) |
| 3.28 (m) | 3.27 (hept, J = 7 | 3.29-3.25 (m, 1H) | 3.27 (ddd, J = | 3.26 (hept, J = |
| | Hz, 1H) | | 13.6, 6.8, 6.7 Hz) | 6.6 Hz, 1H) |
| 3.08 (AB type) | 3.09 (dd, J = | 3.08 (dd, J = | 3.08 (dd, J = | 3.07 (dd, J = |
| | 9.5, 1 Hz, 1H) | 9.5, 1.3 Hz, 1H) | 9.4, 1.2 Hz) | 9.5, 1.3 Hz, 1H) |
| not reported | 2.96 (br s, 1H) | 2.95-2.92 (m, 2H) | 2.95-2.92 (m) | not observed |
| 2.94 (b.t, J = 7.5 | 2.94 (td, J = | | | 2.92 (td, J = |
| Hz, 1H) | 8.5, 2.5 Hz, 1H) | | | 8.6, 2.5 Hz, 1H) |
| 2.48 (br s, 3H) | 2.53 (br s, 1H) | 2.52 (br s, 1H) | 2.52 (br) | 2.55 (br s, 1H) |
| not reported | 2.22-1.90 (m, 5H) | 2.16-2.08 (m, 2H) | 2.18-2.08 (m, 2H) | 2.14 (ddd, J = |
| | | | | 16.4, 9.9, 6.5 |
| | | | | Hz, 1H) |
| | | | | 2.08 (ddd, J = |
| | | | | 15.6, 8.4, 2.4 |
| | | | | Hz, 1H) |
| | | 2.03-1.93 (m, 3H) | 2.03-1.92 (m, 3H) | 2.02-1.89 (m, |
| | | | | 3H) |
| | 1.85 (ddd, J = | 1.85 (ddd, J = | 1.85 (ddd, J = | 1.84 (ddd, J = |
| | 12, 6, 3 Hz, 1H) | 11.9, 6.8, 2.0 Hz, | 12.0, 6.8, 2.2 Hz) | 12.0, 6.6, 2.4 |
| | | 1H) | | Hz, 1H) |
| | 1.78 (br s, 1H) | 1.76 (s, 1H) | 1.75 (br) | not observed |
| | 1.69 (ddd, J = | 1.71-1.66 (m, | 1.69 (ddd, J = | 1.67 (ddd, J = |
| | 12, 10, 8.5 Hz, | 1H) | 11.9, 10.1, 8.3 Hz) | 12.0, 10.0, 8.4 |
| | 1H) | | | Hz, 1H) |
| | 1.46-1.25 (m, | 1.43-1.40 (m, | 1.42-1.38 (m) | 1.43-1.36 (m, |
| | 2H) | 1H) | | 1H) |
| | | 1.30-1.24 (m, | 1.30-1.27 (m) | 1.30-1.23 (m, |
| | | 1H) | | 1H) |
| 1.20 (s, 3H) | 1.22 (s, 3H) | 1.22 (s, 3H) | 1.22 (s, 3H) | 1.20 (s, 3H) |
| 1.01 (d, J = 7.0 | 1.04 (d, J = 7 | 1.04 (d, J = 6.9 | 1.04 (d, 6.8, 3H) | 1.02 (d, J = 6.7 |
| Hz, 3H) | Hz, 3H) | Hz, 3H) | | Hz, 3H) |
| 0.94 (d, J = 7.0 | 0.96 (d, J = 7 | 0.96 (d, J = 6.9 | 0.96 (d, 6.8, 3H) | 0.94 (d, J = 6.8 |
| Hz, 3H) | Hz, 3H) | Hz, 3H) | | Hz, 3H) |
| 0.79 (d, J = 6.5 | 0.81 (d, J = 7.5 | 0.81 (d, J = 7.2 | 0.81 (d, 7.3, 3H) | 0.79 (d, J = 7.3 |
| Hz, 3H) | Hz, 3H) | Hz, 3H) | | Hz, 3H) |

TABLE S7
¹³C NMR Comparison of Cotylenol (in CDCl₃)

| | Synthetic -1 | Synthetic -2 | Synthetic -3 | Synthetic -4 | δ |
|---|---|---|---|---|---|
| | Takeshita | Nakada | Shenvi | (the present | (syn4 − |
| Natural^{S7} | et al.^{S8} | et al.^{S6} | et al.^{S15} | disclosure) | syn3) |
| 149.1 | 150.4 | 150.4 | 150.5 | 150.4 | −0.1 |
| 139.2 | 139.7 | 139.7 | 139.8 | 139.8 | 0 |
| 136.5 | 136.9 | 136.9 | 137.0 | 137.0 | 0 |
| 133.9 | 134.3 | 134.2 | 134.4 | 134.4 | 0 |
| 81.7 | 82.0 | 81.9 | 82.0 | 82.1 | 0.1 |
| 77.3 | 77.5 | 77.5 | 77.6 | 77.6 | 0 |
| 77.3 | 77.4 | 77.3 | 77.4 | 77.4 | 0 |
| 67.4 | 67.9 | 67.8 | 68.0 | 67.9 | −0.1 |
| 59.1 | 59.3 | 59.3 | 59.4 | 59.4 | 0 |
| not | 51.8 | 51.8 | 51.9 | 51.9 | 0 |
| reported | 42.5 | 42.5 | 42.6 | 42.6 | 0 |
| | 41.7 | 41.6 | 41.7 | 41.7 | 0 |
| | 40.2 | 40.2 | 40.3 | 40.3 | 0 |
| | 35.3 | 35.3 | 35.4 | 35.4 | 0 |
| | 31.6 | 31.6 | 31.7 | 31.7 | 0 |
| | 28.1 | 28.0 | 28.2 | 28.1 | −0.1 |
| | 27.1 | 27.1 | 27.2 | 27.2 | 0 |
| | 26.5 | 26.5 | 26.6 | 26.6 | 0 |
| | 21.5 | 21.4 | 21.6 | 21.6 | 0 |
| | 20.3 | 20.3 | 20.4 | 20.4 | 0 |
| | 8.4 | 8.4 | 8.5 | 8.5 | 0 |

TABLE S8
¹H NMR Comparison of Brassicicene I (in CDCl₃)

| Natural^{S16} | Synthetic (the present disclosure) |
|---|---|
| 5.60 (d, J = 2.2 Hz, 1H) | 5.59 (d, J = 2.5 Hz, 1H) |
| 3.89 (ddd, J = 11.5, 3.8, 3.8 Hz, 1H) | 3.89 (dt, J = 11.5, 3.9 Hz, 1H) |
| 3.42 (s, 3H) | 3.42 (s, 3H) |
| 3.39 (d, J = 9.5 Hz, 1H) | 3.39 (d, J = 9.5 Hz, 1H) |
| 3.15 (d, J = 9.5 Hz, 1H) | 3.14 (dd, J = 9.5, 1.2 Hz, 1H) |
| 2.89 (dd, J = 7.7, 7.7 Hz, 1H) | 2.89 (td, J = 8.0, 2.5 Hz, 1H) |
| 2.79 (hept, J = 6.8 Hz, 1H) | 2.78 (hept, J = 6.8 Hz, 1H) |
| 2.32 (br d, J = 13.2 Hz, 1H) | 2.31 (ddd, J = 13.3, 3.6, 2.0 Hz, |
| | 1H) |
| 2.12 (m, 1H) | 2.13-1.96 (m, 5H) |
| 2.10 (m, 1H) | |
| 2.07 (m, 1H) | |
| 2.02 (m, 1H) | |
| 1.95 (m, 1H) | |
| 1.79 (m, 1H) | 1.83-1.77 (m, 2H) |
| 1.79 (m, 1H) | |
| 1.66 (m, 1H) | 1.64 (dt, J = 12.1, 8.5 Hz, 1H) |
| 1.44 (m, 1H) | 1.47-1.39 (m, 1H) |
| 1.25 (m, 1H) | 1.30-1.23 (m, 1H) |
| 1.13 (s, 3H) | 1.13 (s, 3H) |
| 0.99 (d, J = 7.0 Hz, 3H) | 0.99 (d, J = 7.0 Hz, 3H) |
| 0.98 (d, J = 7.0 Hz, 3H) | 0.97 (d, J = 7.0 Hz, 3H) |
| 0.81 (d, J = 7.0 Hz, 3H) | 0.80 (d, J = 7.1 Hz, 3H) |

TABLE S9
¹³C NMR Comparison of Brassicicene I

| | Synthetic | |
|---|---|---|
| | (the present | |
| Natural^{S16} | disclosure) | δ (syn. − nat.) |
| 145.9 | 146.0 | 0.1 |
| 139.3 | 139.5 | 0.2 |
| 135.5 | 135.6 | 0.1 |
| 132.5 | 132.7 | 0.2 |
| 82.0 | 82.1 | 0.1 |
| 77.8 | 77.7 | −0.1 |
| 76.3 | 76.4 | 0.1 |
| 59.4 | 59.5 | 0.1 |
| 52.8 | 52.9 | 0.1 |
| 44.5 | 44.7 | 0.2 |
| 42.0 | 42.1 | 0.1 |
| 40.4 | 40.5 | 0.1 |
| 35.5 | 35.7 | 0.2 |
| 32.0 | 32.1 | 0.1 |
| 28.5 | 28.6 | 0.1 |
| 27.4 | 27.5 | 0.1 |
| 27.1 | 27.2 | 0.1 |
| 26.5 | 26.6 | 0.1 |
| 20.9 | 21.0 | 0.1 |
| 20.8 | 20.9 | 0.1 |
| 7.9 | 8.1 | 0.2 |

TABLE S10
¹H NMR Comparison of Brassicicene A (in CD₃OD)

| Natural^{S10} | Synthetic (the present disclosure) |
|---|---|
| 5.74 (d, J = 2.5 Hz) | 5.75 (d, J = 2.3 Hz) |
| 3.93 (dt, J = 10.5, 3.5 Hz) | 3.94 (dt, J = 10.4, 4.0 Hz) |
| 3.43 (d, J = 10.0 Hz) | 3.44 (d, J = 9.9 Hz) |
| 3.39 (s, 3H) | 3.39 (s, 3H) |
| 3.27 (d, J = 10.0 Hz) | 3.28 (d, J = 10.0 Hz) |
| 2.85 (hept, J = 7.0 Hz) | 2.86 (hept, J = 6.8 Hz) |
| 2.71 (br d, J = 6.3 Hz) | 2.72 (ddd, J = 8.5, 5.8, 2.2 Hz) |
| 2.56 (m, 2H) | 2.62-2.52 (m, 2H) |
| Not reported | 2.48 (d, J = 18.5 Hz) |
| 2.27 (dd, J = 1.0, 18.5 Hz) | 2.28 (d, J = 18.5 Hz) |
| 1.97 (m, 2H) | 2.06-1.94 (m, 2H) |
| 1.89 (m) | 1.90 (qd, J = 7.2, 4.5 Hz) |
| 1.45 (m) | 1.51-1.37 (m, 2H) |
| 1.39 (m) | |
| 1.32 (s, 3H) | 1.33 (s, 3H) |
| 1.25 (d, J = 7.0 Hz, 3H) | 1.26 (d, J = 7.0 Hz, 3H) |
| 1.17 (d, J = 7.0 Hz, 3H) | 1.18 (d, J = 6.9 Hz, 3H) |
| 0.90 (d, J = 7.0 Hz, 3H) | 0.91 (d, J = 7.0 Hz, 3H) |

TABLE S11
¹³C NMR Comparison of Brassicicene A (in CD₃OD)

| Natural -1 | Natural -2 | Synthetic | |
|---|---|---|---|
| (by MacKinnon | (by Kenmoku | (the present | |
| et al.)^{S10} | et al.)^{S13} | disclosure) | δ (syn. − nat2) |
| 209.3 | 209.8 | 209.8 | 0 |
| 175.6 | 175.9 | 175.9 | 0 |
| 146.5 | 146.9 | 146.9 | 0 |
| 141.9 | 142.1 | 142.1 | 0 |
| 134.4 | 134.7 | 134.7 | 0 |
| 83.0 | 83.3 | 83.3 | 0 |
| 78.1 | 78.3 | 78.3 | 0 |
| 76.2 | 76.5 | 76.5 | 0 |
| 59.7 | 59.6 | 59.6 | 0 |
| 53.2 | 53.4 | 53.4 | 0 |
| 48.2 | 48.5 | 48.5 | 0 |
| 45.9 | 46.1 | 46.1 | 0 |
| 42.0 | 42.2 | 42.2 | 0 |
| 36.4 | 36.5 | 36.5 | 0 |
| 32.8 | 32.9 | 32.8 | −0.1 |
| 31.3 | 31.4 | 31.4 | 0 |
| 28.2 | 28.0 | 28.0 | 0 |
| 27.0 | 27.3 | 27.3 | 0 |
| 20.2 | 20.0 | 20.0 | 0 |
| 20.0 | 19.9 | 19.9 | 0 |
| 8.5 | 8.4 | 8.4 | 0 |

TABLE S12
¹H NMR Comparison of Brassicicene R (in CD₃OD)

| Natural^{S12} | Synthetic (the present disclosure) |
|---|---|
| 5.84 (d, J = 2.0 Hz) | 5.84 (d, J = 2.0 Hz) |
| 3.91 (s) | 3.90 (s) |
| 3.88 (m) | 3.87 (ddd, J = 10.0, 4.6, 3.2 Hz) |
| 3.51 (d, J = 9.8 Hz) | 3.51 (d, J = 9.8 Hz) |
| 3.41 (d, J = 9.8 Hz) | 3.41 (d, J = 9.8 Hz) |
| 3.41 (s, 3H) | 3.41 (s, 3H) |
| 2.88 (m) | 2.88 (hept, J = 6.9 Hz) |
| 2.65 (m) | 2.66-2.63 (m) |
| 2.51 (m, 2H) | 2.55-2.48 (m, 2H) |
| 2.06 (m) | 2.11-2.04 (m) |
| 1.98 (m) | 1.99 (ddd, J = 13.2, 9.5, 6.9 Hz) |
| 1.91 (m) | 1.94-1.90 (m) |
| 1.56 (m) | 1.57 (ddd, J = 13.1, 7.5, 5.3 Hz) |
| 1.48 (m) | 1.51-1.46 (m) |
| 1.24 (d, J = 6.9 Hz, 3H) | 1.24 (d, J = 6.9 Hz, 3H) |
| 1.19 (d, J = 6.9 Hz, 3H) | 1.19 (d, J = 7.0 Hz, 3H) |
| 1.14 (s, 3H) | 1.14 (s, 3H) |
| 0.93 (d, J = 7.0 Hz, 3H) | 0.93 (d, J = 7.0 Hz, 3H) |

TABLE S13
¹³C NMR Comparison of Brassicicene R (in CD₃OD)

| | Synthetic | |
|---|---|---|
| | (the present | |
| Natural^{S12} | disclosure) | δ (syn. − nat.) |
| 208.1 | 208.1 | 0 |
| 173.8 | 173.7 | −0.1 |
| 145.3 | 145.3 | 0 |
| 143.4 | 143.4 | 0 |
| 133.6 | 133.6 | 0 |
| 83.8 | 83.8 | 0 |
| 83.3 | 83.3 | 0 |
| 78.7 | 78.7 | 0 |
| 77.3 | 77.3 | 0 |
| 59.7 | 59.7 | 0 |
| 53.1 | 53.1 | 0 |
| 45.4 | 45.4 | 0 |
| 42.4 | 42.5 | 0.1 |
| 37.3 | 37.3 | 0 |
| 33.4 | 33.4 | 0 |
| 31.9 | 31.9 | 0 |
| 27.0 | 27.0 | 0 |
| 24.3 | 24.3 | 0 |
| 20.4 | 20.4 | 0 |
| 19.5 | 19.5 | 0 |
| 8.9 | 8.9 | 0 |

TABLE S14
¹H NMR Comparison of Brassicicene L (in CD₃OD)

| Natural^{S11} | Synthetic (the present disclosure) |
|---|---|
| 6.15 (s) | 6.16 (s) |
| 5.45 (d, J = 1.8 Hz) | 5.44 (d, J = 2.2 Hz) |
| 3.49 (d, J = 9.8 Hz) | 3.49 (d, J = 9.8 Hz) |
| 3.44 (d, J = 9.8 Hz) | 3.44 (d, J = 9.8 Hz) |
| 3.39 (s, 3H) | 3.39 (s, 3H) |
| 3.19 (br s) | 3.20-3.18 (m) |
| 2.82 (hept, J = 6.8 Hz) | 2.82 (hept, J = 6.8 Hz) |
| 2.51 (m) | 2.53-2.50 (m) |
| 2.29 (dd, J = 14.5, 6.0 Hz) | 2.29 (dd, J = 14.5, 6.0 Hz) |
| 2.01 (overlap) | 2.05-1.98 (m, 2H) |
| 2.01 (overlap) | |
| 1.70 (m) | 1.69 (qt, J = 6.6, 3.0 Hz) |
| 1.62 (m) | 1.64-1.59 (m) |
| 1.54 (d, J = 14.5 Hz) | 1.55-1.48 (m, 2H) |
| 1.49 (m) | |
| 1.29 (s, 3H) | 1.29 (s, 3H) |
| 1.28 (d, J = 6.8 Hz, 3H) | 1.28 (d, J = 6.9 Hz, 3H) |
| 1.24 (d, J = 6.8 Hz, 3H) | 1.24 (d, J = 6.8 Hz, 3H) |
| 0.88 (d, J = 6.9 Hz, 3H) | 0.88 (d, J = 6.9 Hz, 3H) |

TABLE S15
¹³C NMR Comparison of Brassicicene L (in CD₃OD)

| | Synthetic | |
|---|---|---|
| | (the present | |
| Natural^{S11} | disclosure) | δ (syn. − nat.) |
| 210.4 | 210.4 | 0 |
| 189.4 | 189.4 | 0 |
| 152.4 | 152.4 | 0 |
| 129.0 | 129.0 | 0 |
| 126.6 | 126.6 | 0 |
| 86.8 | 86.8 | 0 |
| 83.8 | 83.8 | 0 |
| 78.4 | 78.4 | 0 |
| 72.8 | 72.8 | 0 |
| 60.8 | 60.8 | 0 |
| 59.6 | 59.6 | 0 |
| 46.0 | 46.0 | 0 |
| 44.4 | 44.4 | 0 |
| 40.2 | 40.1 | −0.1 |
| 37.6 | 37.6 | 0 |
| 29.8 | 29.8 | 0 |
| 29.3 | 29.3 | 0 |
| 28.5 | 28.5 | 0 |
| 23.8 | 23.8 | 0 |
| 22.2 | 22.2 | 0 |
| 8.9 | 9.0 | 0.1 |

TABLE S16
¹H NMR Comparison of Brassicicene K (in CD₃OD)

| Natural -1 | Natural -2 | |
|---|---|---|
| (by Kenmoku et al.)^{S13} | (by Hu et al.)^{S14} | Synthetic (the present disclosure) |
| 6.33 (dd, J = 10.6, 1.8 Hz) | 6.27 (dd, J = 10.5, 1.9 Hz) | 6.27 (dd, J = 10.5, 1.8 Hz) |
| 5.15 (d, J = 0.7 Hz) | 5.25 (s) | 5.25 (s) |
| 5.05 (d, J = 0.7 Hz) | 5.10 (s) | 5.10 (s) |
| 4.06 (m) | 3.99 (m) | 3.99 (ddd, J = 9.8, 6.2, 3.0 Hz) |
| 3.65 (dd, J = 10.6, 1.0 Hz) | 3.65 (d, J = 10.6 Hz) | 3.65 (d, J = 10.5 Hz) |
| 3.40 (s, 3H) | 3.36 (s, 3H) | 3.36 (s, 3H) |
| 3.34 (d, J = 9.6 Hz) | 3.32 (m, 2H) | 3.33 (d, J = 10.0 Hz) |
| 3.30 (d, J = 9.5 Hz) | | 3.30 (d, J = 10.0 Hz) |
| 3.11 (dd, J = 12.2, 6.5 Hz) | 3.09 (m) | 3.09 (dd, J = 12.2, 6.1 Hz) |
| 2.90 (sept, J = 6.9 Hz) | 2.95 (m) | 2.95 (hept, J = 6.9 Hz) |
| 2.72 (m) | 2.72 (m) | 2.72 (d, J = 7.0 Hz) |
| 2.60 (m) | 2.64 (dd, J = 12.2, 10.6 Hz) | 2.65 (dd, J = 12.6, 10.2 Hz) |
| 1.98 (m) | 1.96 (m) | 1.96 (tt, J = 11.8, 7.0 Hz) |
| 1.81 (m) | 1.84 (m) | 1.84 (qt, J = 7.8, 2.4 Hz) |
| 1.69 (m) | 1.72 (m) | 1.72 (ddd, J = 13.1, 11.3, 6.1 Hz) |
| 1.56 (m) | 1.51 (m) | 1.51 (ddd, J = 13.0, 6.5, 3.9 Hz) |
| 1.41 (m) | 1.41 (m) | 1.41 (ddt, J = 12.6, 6.4, 3.4 Hz) |
| 1.33 (d, J = 6.9 Hz, 3H) | 1.33 (d, J = 7.0 Hz, 3H) | 1.33 (d, J = 6.9 Hz, 3H) |
| 1.22 (d, J = 6.9 Hz, 3H) | 1.20 (d, J = 7.0 Hz, 3H) | 1.20 (d, J = 6.9 Hz, 3H) |
| 0.90 (d, J = 6.9 Hz, 3H) | 0.91 (d, J = 7.6 Hz, 3H) | 0.91 (d, J = 7.5 Hz, 3H) |

TABLE S17
¹³C NMR Comparison of Brassicicene K (in CD₃OD)

| Natural -1 | | Synthetic | |
|---|---|---|---|
| (by Kenmoku | Natural-2 | (the present | |
| et al.)^{S13} | (by Hu et al.)^{S14} | disclosure) | δ (syn. − nat2) |
| 201.5 | 204.2 | 204.2 | 0 |
| 161.8 | 165.5 | 165.5 | 0 |
| 149.1 | 150.6 | 150.6 | 0 |
| 148.8 | 150.5 | 150.5 | 0 |
| 148.8 | 149.6 | 149.7 | 0.1 |
| 124.9 | 125.6 | 125.6 | 0 |
| 105.5 | 106.6 | 106.6 | 0 |
| 82.2 | 83.5 | 83.5 | 0 |
| 78.4 | 79.4 | 79.4 | 0 |
| 74.8 | 75.6 | 75.6 | 0 |
| 59.5 | 59.6 | 59.6 | 0 |
| 53.6 | 54.9 | 54.9 | 0 |
| 48.7 | 49.7 | 49.7 | 0 |
| 45.3 | 46.8 | 46.8 | 0 |
| 36.4 | 37.3 | 37.3 | 0 |
| 35.4 | 36.0 | 36.0 | 0 |
| 30.4 | 31.1 | 31.1 | 0 |
| 26.8 | 27.9 | 28.0 | 0.1 |
| 21.4 | 21.7 | 21.7 | 0 |
| 19.6 | 19.9 | 19.9 | 0 |
| 10.5 | 11.2 | 11.2 | 0 |

TABLE S18
¹H NMR Comparison of Brassicicene C (in CD₃OD)

| Natural (by MacKinnon et al.)^{S10} | Natural (by Hu et al.)^{S14} | Synthetic (the present disclosure) |
|---|---|---|
| 6.09 (dd, J = 10.0, 2.0 Hz) | 6.10 (dd, J = 10.2, 1.7 Hz) | 6.10 (dd, J = 10.1, 1.7 Hz) |
| 4.17 (ddd, J = 11.5, 5.5, 3.5 Hz) | 4.18 (m) | 4.18 (ddd, J = 11.4, 5.6, 3.6 Hz) |
| 3.48 (d, J = 10.0 Hz) | 3.49 (d, J = 9.8 Hz) | 3.49 (d, J = 9.8 Hz) |
| 3.38 (d, J = 10.0 Hz) | 3.39 (d, J = 9.8 Hz) | 3.39 (d, J = 9.8 Hz) |
| 3.37 (s, 3H) | 3.38 (s, 3H) | 3.38 (s, 3H) |
| 3.35 (dd, J = 10.0, 5.5 Hz) | 3.37 (m) | 3.37 (dd, J = 10.8, 4.8 Hz) |
| 3.14 (m) | 3.14 (m) | 3.14 (tt, J = 8.0, 6.2 Hz) |
| 2.92 (hept, J = 7.0 Hz) | 2.93 (m) | 2.95-2.90 (m, 2H) |
| 2.91 (m) | 2.91 (m) | |
| 2.80 (br d, J = 7.5 Hz) | 2.80 (m) | 2.80 (d, J = 7.8 Hz) |
| 2.45 (dd, J = 13.5, 11.5 Hz) | 2.45 (dd, J = 13.8, 11.5 Hz) | 2.46 (dd, J = 13.8, 11.4 Hz) |
| 2.03 (qd, J = 7.5, 3.5 Hz) | 2.05 (m) | 2.05 (qd, J = 7.6, 3.5 Hz) |
| 1.97 (m) | 1.98 (m) | 1.99 (tdd, J = 12.0, 7.8, 6.9 Hz) |
| 1.87 (m) | 1.88 (m) | 1.88 (td, J = 12.6, 6.3 Hz) |
| 1.53 (m) | 1.55 (m) | 1.55 (ddd, J = 13.0, 6.7, 3.3 Hz) |
| 1.44 (m) | 1.45 (m) | 1.45 (ddt, J = 12.0, 5.9, 2.8 Hz) |
| 1.32 (d, J = 7.0 Hz, 3H) | 1.33 (d, J = 6.9 Hz, 3H) | 1.33 (d, J = 6.8 Hz, 3H) |
| 1.25 (d, J = 7.5 Hz, 3H) | 1.27 (d, J = 7.3 Hz, 3H) | 1.26 (d, J = 7.3 Hz, 3H) |
| 1.15 (d, J = 7.0 Hz, 3H) | 1.16 (d, J = 6.9 Hz, 3H) | 1.16 (d, J = 6.9 Hz, 3H) |
| 1.00 (d, J = 7.5 Hz, 3H) | 1.01 (d, J = 7.6 Hz, 3H) | 1.01 (d, J = 7.6 Hz, 3H) |

TABLE S19
¹³C NMR Comparison of Brassicicene C (in CD₃OD)

| Natural -1 | | Synthetic | |
|---|---|---|---|
| (by MacKinnon | Natural -2 | (the present | |
| et al.)^{S10} | (by Hu et al.)^{S14} | disclosure) | δ (syn. − nat2) |
| 207.4 | 207.4 | 207.4 | 0 |
| 176.7 | 176.8 | 176.8 | 0 |
| 151.2 | 151.2 | 151.2 | 0 |
| 147.7 | 147.6 | 147.7 | 0.1 |
| 127.7 | 127.8 | 127.8 | 0 |
| 84.1 | 84.0 | 84.1 | 0.1 |
| 79.5 | 79.5 | 79.5 | 0 |
| 75.5 | 75.5 | 75.5 | 0 |
| 59.6 | 59.6 | 59.6 | 0 |
| 58.8 | 58.7 | 58.7 | 0 |
| 50.9 | 50.9 | 50.9 | 0 |
| 47.9 | 47.8 | 47.8 | 0 |
| 44.8 | 44.8 | 44.8 | 0 |
| 38.0 | 38.0 | 38.0 | 0 |
| 36.6 | 36.6 | 36.7 | 0.1 |
| 30.6 | 30.6 | 30.6 | 0 |
| 28.9 | 28.9 | 28.9 | 0 |
| 22.0 | 22.0 | 22.0 | 0 |
| 19.0 | 19.0 | 19.0 | 0 |
| 13.0 | 13.0 | 13.0 | 0 |
| 12.3 | 12.4 | 12.4 | 0 |

TABLE S20
¹H NMR Comparison of Brassicicene H (in CDCl₃)

| Natural^{S16} | Synthetic (the present disclosure) |
|---|---|
| 5.85 (dd, J = 9.5, 1.2 Hz) | 5.83 (dd, J = 9.5, 1.8 Hz) |
| 4.82 (br s) | 4.80 (s) |
| 4.29 (m) | 4.29-4.25 (m) |
| 3.53 (d, J = 9.3 Hz) | 3.51 (d, J = 9.3 Hz) |
| 3.45 (s, 3H) | 3.43 (s, 3H) |
| 3.42 (d, J = 9.3 Hz) | 3.39 (d, J = 9.3 Hz) |
| 3.35 (m) | 3.34 (d, J = 8.3 Hz) |
| 3.28 (m) | 3.27 (dt, J = 10.0, 5.2 Hz) |
| 2.90 (hept, J = 6.8 Hz) | 2.89 (hept, J = 6.9 Hz) |
| 2.56 (m, 3H) | 2.60-2.53 (m, 3H) |
| 2.09 (m) | 2.09-2.02 (m, 2H) |
| 2.07 (m) | |
| 2.02 (m) | 2.00 (qd, J = 7.5, 3.5 Hz) |
| 1.89 (ddd, J = 12.5, 12.5, 6.5 Hz) | 1.88 (td, J = 12.7, 6.3 Hz) |
| 1.68 (m) | 1.67 (dddd, J = 13.2, 6.8, 3.0, |
| | 0.8 Hz) |
| 1.49 (m) | 1.47 (ddt, J = 12.0, 5.7, 2.7 Hz) |
| 1.38 (d, J = 7.5 Hz, 3H) | 1.37 (d, J = 7.0 Hz, 3H) |
| 1.14 (d, J = 7.0 Hz, 3H) | 1.12 (d, J = 7.4 Hz, 3H) |
| 1.13 (d, J = 7.5 Hz, 3H) | 1.11 (d, J = 7.2 Hz, 3H) |
| 0.99 (d, J = 7.5 Hz, 3H) | 0.97 (d, J = 7.5 Hz, 3H) |

TABLE S21
¹³C NMR Comparison of Brassicicene H (in CDCl₃)

| | Synthetic | |
|---|---|---|
| | (the present | |
| Natural^{S16} | disclosure) | δ (syn. − nat.) |
| 153.1 | 153.4 | 0.3 |
| 144.2 | 144.2 | 0 |
| 136.5 | 136.5 | 0 |
| 126.4 | 126.0 | −0.4 |
| 83.0 | 82.7 | −0.3 |
| 80.3 | 80.1 | −0.2 |
| 79.1 | 79.0 | −0.1 |
| 75.4 | 75.3 | −0.1 |
| 59.7 | 59.6 | −0.1 |
| 52.5 | 52.3 | −0.2 |
| 50.0 | 49.9 | −0.1 |
| 48.5 | 48.4 | −0.1 |
| 42.6 | 42.6 | 0 |
| 37.0 | 37.0 | 0 |
| 36.9 | 36.7 | −0.2 |
| 29.9 | 29.8 | −0.1 |
| 28.3 | 28.3 | 0 |
| 22.0 | 21.9 | −0.1 |
| 21.7 | 21.7 | 0 |
| 13.8 | 13.8 | 0 |
| 12.2 | 12.0 | −0.2 |

TABLE S22
¹H NMR Comparison of Brassicicene J (in CD₃OD)

| Natural -1 (by Kenmoku et al.)^{S13} | Natural -2 (by Hu et al.)^{S14} | Synthetic (the present disclosure) |
|---|---|---|
| 6.11 (dd, J = 10.6, 1.8 Hz) | 6.11 (dd, J = 10.5, 1.7 Hz) | 6.12 (dd, J = 10.5, 1.7 Hz) |
| 4.15 (m) | 4.15 (m) | 4.15 (ddd, J = 11.4, 5.4, 3.8 Hz) |
| 3.47 (d, J = 9.9 Hz) | 3.48 (d, J = 9.7 Hz) | 3.48 (d, J = 9.8 Hz) |
| 3.40 (d, J = 9.9 Hz) | 3.40 (d, J = 9.7 Hz) | 3.40 (d, J = 9.7 Hz) |
| 3.37 (s, 3H) | 3.38 (s, 3H) | 3.38 (s, 3H) |
| 3.21 (d, J = 10.6 Hz) | 3.22 (d, J = 10.5 Hz) | 3.22 (d, J = 10.5 Hz) |
| 2.96 (hept, J = 7.0 Hz) | 2.97 (m) | 2.97 (hept, J = 7.0 Hz) |
| 2.93 (m) | 2.94 (m) | 2.94 (dd, J = 14.4, 5.5 Hz) |
| 2.84 (d, J = 7.6 Hz) | 2.85 (m) | 2.85 (d, J = 7.8 Hz) |
| 2.42 (dd, J = 14.1, 11.7 Hz) | 2.42 (dd, J = 14.2, 11.6 Hz) | 2.42 (dd, J = 14.2, 11.6 Hz) |
| 2.06 (m) | 2.07 (m) | 2.06 (ddt, J = 11.4, 7.8, 3.5 Hz) |
| 1.97 (m) | 1.98 (m) | 1.98 (tt, J = 12.1, 7.2 Hz) |
| 1.86 (ddd, J = 18.9, 12.6, 6.2 Hz) | 1.86 (m) | 1.86 (td, J = 12.3, 6.5 Hz) |
| 1.56 (s, 3H) | 1.60 (s, 3H) | 1.60 (s, 3H) |
| 1.53 (ddd, J = 12.4, 6.4, 3.4 Hz) | 1.54 (m) | 1.54 (ddt, J = 13.1, 6.8, 3.7 Hz) |
| 1.45 (m) | 1.45 (m) | 1.45 (ddd, J = 11.5, 5.8, 2.8 Hz) |
| 1.35 (d, J = 7.0 Hz, 3H) | 1.36 (d, J = 6.9 Hz, 3H) | 1.36 (d, J = 6.9 Hz, 3H) |
| 1.23 (d, J = 7.0 Hz, 3H) | 1.23 (d, J = 6.9 Hz, 3H) | 1.23 (d, J = 6.9 Hz, 3H) |
| 1.01 (d, J = 7.6 Hz, 3H) | 1.02 (d, J = 7.6 Hz, 3H) | 1.02 (d, J = 7.6 Hz, 3H) |

TABLE S23
¹³C NMR Comparison of Brassicicene J (in CD₃OD)

| | | Synthetic | |
|---|---|---|---|
| Natural -1 | Natural-2 | (the present | |
| (by Kenmoku et al.)^{S13} | (by Hu et al.)^{S14} | disclosure) | δ (syn. − nat2) |
| 205.3 | 205.2 | 205.3 | 0.1 |
| 166.9 | 166.9 | 166.9 | 0 |
| 152.6 | 152.6 | 152.6 | 0 |
| 152.3 | 152.3 | 152.3 | 0 |
| 126.4 | 126.4 | 126.4 | 0 |
| 92.6 | 92.5 | 92.6 | 0.1 |
| 83.9 | 83.9 | 83.9 | 0 |
| 79.3 | 79.3 | 79.3 | 0 |
| 75.6 | 75.6 | 75.6 | 0 |
| 59.8 | 59.8 | 59.8 | 0 |
| 59.6 | 59.6 | 59.6 | 0 |
| 50.5 | 50.5 | 50.5 | 0 |
| 47.6 | 47.6 | 47.6 | 0 |
| 38.0 | 38.0 | 38.0 | 0 |
| 36.5 | 36.5 | 36.5 | 0 |
| 29.8 | 29.8 | 29.8 | 0 |
| 29.4 | 29.4 | 29.4 | 0 |
| 22.0 | 22.0 | 22.0 | 0 |
| 19.0 | 19.0 | 19.0 | 0 |
| 16.5 | 16.5 | 16.5 | 0 |
| 12.1 | 12.1 | 12.1 | 0 |

TABLE S24
¹H NMR Comparison of Brassicicene F (in CD₃OD)

| Natural -1 (by MacKinnon et al.)^{S10} | Natural -2 (by Hu et al.)^{S14} | Synthetic (the present disclosure) |
|---|---|---|
| 6.13 (dd, J = 10.8, 1.8 Hz) | 6.14 (dd, J = 10.4, 1.7 Hz) | 6.14 (dd, J = 10.4, 1.8 Hz) |
| 4.16 (ddd, J = 11.7, 5.7, 3.9 Hz) | 4.17 (m) | 4.16 (ddd, J = 11.6, 5.5, 3.6 Hz) |
| 3.46 (d, J = 10.0 Hz) | 3.47 (d, J = 9.8 Hz) | 3.47 (d, J = 9.8 Hz) |
| 3.38 (d, J = 10.0 Hz) | 3.39 (d, J = 9.8 Hz) | 3.39 (d, J = 9.8 Hz) |
| 3.36 (s, 3H) | 3.38 (s, 3H) | 3.37 (s, 3H) |
| 3.09 (d, J = 10.8 Hz) | 3.10 (d, J = 10.4 Hz) | 3.10 (d, J = 10.4 Hz) |
| 2.93 (m) | 2.97 (m) | 2.96 (dd, J = 14.1, 5.5 Hz) |
| 2.90 (hept, J = 6.5 Hz) | 2.93 (m) | 2.92 (hept, J = 6.9 Hz) |
| 2.84 (br d, J = 7.5 Hz) | 2.85 (m) | 2.85 (d, J = 7.8 Hz) |
| 2.44 (dd, J = 14.3, 11.7 Hz) | 2.45 (dd, J = 14.1, 11.6 Hz) | 2.45 (dd, J = 14.1, 11.5 Hz) |
| 2.04 (m) | 2.05 (m) | 2.07-2.02 (m) |
| 1.95 (m) | 1.97 (m) | 1.97 (tt, J = 12.4, 7.3 Hz) |
| 1.85 (m) | 1.86 (m) | 1.86 (td, J = 12.6, 6.2 Hz) |
| 1.57 (s, 3H) | 1.58 (s, 3H) | 1.58 (s, 3H) |
| 1.52 (ddd, J = 13.0, 6.5, 3.4 Hz) | 1.53 (m) | 1.53 (ddd, J = 13.0, 6.7, 3.3 Hz) |
| 1.44 (m) | 1.45 (m) | 1.45 (ddt, J = 12.0, 5.9, 2.9 Hz) |
| 1.33 (d, J = 6.5 Hz, 3H) | 1.34 (d, J = 6.9 Hz, 3H) | 1.34 (d, J = 6.9 Hz, 3H) |
| 1.20 (d, J = 6.5 Hz, 3H) | 1.21 (d, J = 6.9 Hz, 3H) | 1.21 (d, J = 7.0 Hz, 3H) |
| 0.99 (d, J = 7.0 Hz, 3H) | 1.00 (d, J = 7.6 Hz, 3H) | 1.00 (d, J = 7.5 Hz, 3H) |

TABLE S25
¹³C NMR Comparison of Brassicicene F (in CD₃OD)

| Natural -1 | Natural -2 | | Synthetic | |
|---|---|---|---|---|
| MacKinnon | Kenmoku | Natural-3 | (the present | |
| et al.^{S10} | et al.^{S13} | Hu et al.^{S14} | disclosure) | δ (syn. − nat3) |
| 200.0 | 206.8 | 206.9 | 206.8 | −0.1 |
| 172.4 | 172.3 | 172.4 | 172.4 | 0 |
| 151.3 | 151.3 | 151.3 | 151.3 | 0 |
| 149.8 | 149.8 | 149.8 | 149.8 | 0 |
| 127.3 | 127.4 | 127.3 | 127.3 | 0.1 |
| 84.0 | 84.0 | 84.0 | 84.0 | 0 |
| 81.2 | 81.2 | 81.2 | 81.2 | 0 |
| 79.3 | 79.3 | 79.3 | 79.4 | 0.1 |
| 75.6 | 75.6 | 75.6 | 75.6 | 0 |
| 65.4 | 65.4 | 65.4 | 65.4 | 0 |
| 59.6 | 59.6 | 59.6 | 59.6 | 0 |
| 50.5 | 50.5 | 50.5 | 50.5 | 0 |
| 47.3 | 47.3 | 47.3 | 47.3 | 0 |
| 38.0 | 38.0 | 38.0 | 38.0 | 0 |
| 36.5 | 36.5 | 36.5 | 36.5 | 0 |
| 29.0 | 29.3 | 29.3 | 29.3 | 0 |
| 29.0 | 29.2 | 29.2 | 29.2 | 0 |
| 21.7 | 21.7 | 21.7 | 21.7 | 0.1 |
| 21.5 | 21.5 | 21.5 | 21.5 | 0 |
| 18.9 | 18.9 | 18.9 | 18.9 | 0 |
| 12.9 | 12.0 | 12.0 | 12.0 | 0 |

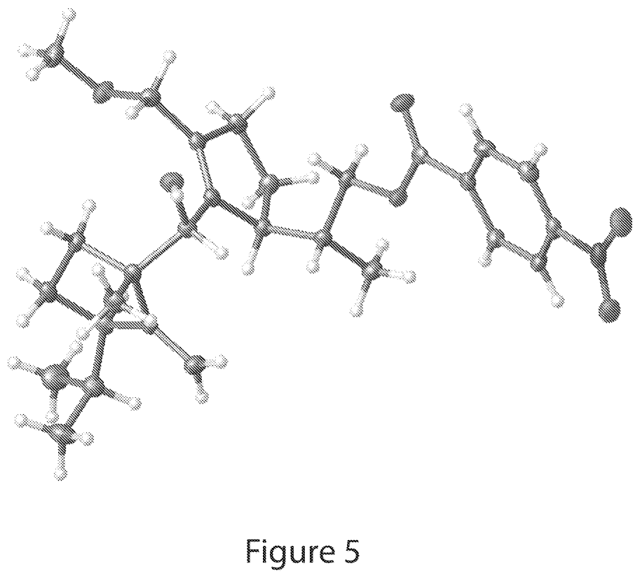

### X-Ray Structure of Compound S9 (FIG. 5).

| Report date | 2022 Mar. 11 | |
|---|---|---|
| Identification code | renata21 | |
| Empirical formula | C28 H39 N O6 | |
| Molecular formula | C28 H39 N O6 | |
| Formula weight | 485.60 | |
| Temperature | 100.00 K | |
| Wavelength | 1.54178 Å | |
| Crystal system | Monoclinic | |
| Space group | P 1 21 1 | |
| Unit cell dimensions | a = 6.5530(2) Å | α = 90°. |
| | b = 11.1324(3) Å | β = 95.7170(10)°. |
| | c = 18.2185(4) Å | γ = 90°. |

| | |
|---|---|
| Volume | 1322.44(6) Å³ |
| Z | 2 |
| Density (calculated) | 1.219 Mg/m³ |
| Absorption coefficient | 0.687 mm⁻¹ |
| F(000) | 524 |
| Crystal size | 0.18 × 0.05 × 0.05 mm³ |
| Crystal color, habit | colorless plank |
| Theta range for data collection | 2.437 to 68.764°. |
| Index ranges | −7 <= h <= 7,−13 <= k <= 13, −21 <= 1 <= 20 |
| Reflections collected | 18858 |
| Independent reflections | 4793 [R(int) = 0.0310] |
| Completeness to theta = 67.679° | 99.9% |
| Absorption correction | Semi-empirical from equivalents |
| Max. and min. transmission | 0.7531 and 0.6673 |
| Refinement method | Full-matrix least-squares on F² |
| Data / restraints / parameters | 4793 / 1 / 323 |
| Goodness-of-fit on F² | 1.020 |
| Final R indices [I > 2sigma(I)] | R1 = 0.0243, wR2 = 0.0605 |
| R indices (all data) | R1 = 0.0256, wR2 = 0.0614 |
| Absolute structure parameter | 0.04(5) |
| Extinction coefficient | 0.0021(4) |
| Largest diff. peak and hole | 0.166 and −0.130 e. Å⁻³ |

### Sequences of the Present Disclosure

TABLE S3
Primers for site mutation in the present disclosure. (bold denotes
targeted mutation, italic denotes other mutations from wild-type)

| | | | SEQ ID |
|---|---|---|---|
| Entry | Description | Sequence (5′-3′) | NO. |
| 1a | Bsc9 C304V For | GCCTAATGTGGTGCGTAGACTACTT | 1 |
| | | ATCCGGAGATCTGC | |
| 1b | Bsc9 C304V Rev | GTCTACGCACCACATTAGGCTGAAC | 2 |
| | | CTGTAGAGATTTTTC | |
| 2a | Bsc9 C304T For | GCCTAATACCGTGCGTAGACTACTT | 3 |
| | | ATCCGGAGATCTGC | |
| 2b | Bsc9 C304T Rev | GTCTACGCACGGTATTAGGCTGAA | 4 |
| | | CCTGTAGAGATTTTTC | |
| 3a | MoBsc9 Y112X | GCGAAGCTGGGGNDTTCCCTGATA | 5 |
| | For NDT | CCC | |
| 3b | MoBsc9 Y112X | GCGAAGCTGGGGVHGTCCCTGATA | 6 |
| | For VHG | CCC | |
| 3c | MoBsc9 Y112X | GCGAAGCTGGGGTGGTCCCTGATA | 7 |
| | For TGG | CCC | |
| 3d | MoBsc9 Y112X | CCCCAGCTTCGCCAGTATGCCCTTG | 8 |
| | Rev | | |
| 4a | MoBsc9 Y112M | GCATACTGGCGAAGNDTGGGATGTC | 9 |
| | L110X For NDT | CCTG | |
| 4b | MoBsc9 Y112M | GCATACTGGCGAAGVHGGGGATGT | 10 |
| | L110X For VHG | CCCTG | |
| 4c | MoBsc9 Y112M | GCATACTGGCGAAGTGGGGGATGT | 11 |
| | L110X For TGG | CCCTG | |
| 4d | MoBsc9 Y112M | CTTCGCCAGTATGCCCTTGCCTTCT | 12 |
| | L110X Rev | GGG | |
| 5a | MoBsc9 Y112R | CATACTGGCGAAGNDTGGGCGTTCC | 13 |
| | L110X For NDT | CTG | |
| 5b | MoBsc9 Y112R | CATACTGGCGAAGVHGGGGCGTTC | 14 |
| | L110X For VHG | CCTG | |
| 5c | MoBsc9 Y112R | CATACTGGCGAAATGGGGGCGTTCC | 15 |
| | L110X For TGG | CTG | |
| 5d | MoBsc9 Y112R | CTTCGCCAGTATGCCCTTGCCTTCT | 12 |
| | L110X Rev | GGG | |
| 6a | Bsc9 Loop DEL | GATTGATCGTGCGTCATATTCATCT | 16 |
| | For | CATGGGGAAAG | |
| 6b | Bsc9 Loop DEL | GAATATGACGCACGATCAATCCTTC | 17 |
| | Rev | GCCCG | |

Protein sequence of Bsc9 (Uniprot ID: D7UTD1)
(SEQ ID NO: 18)
MASTSSTSTDGHLNIRPMVHGSDKKLNFGAYITGLDLNNASDAEVDQLREAILRHKIVVI
KGQQAEKPDKNWEMIKKLDPMHHMITQEEFGQLFHPTGEGLIAMLKLATVPTTEHGHI
HLMGKGYQGDDHYGLKKLNLGEAFAGNYYSKPLAEEDFRAGVTRFQSWHMDGPLYK
VHPPYISSLRFIQLPDGEQTVEWADGSGLSLKTKPGRTAFFSTSQLYDMLTDEERAMVD
NSAVEYMYYPYEWIRGCRGNPNGLNVADEGREKPLDAMEEIARDERWTKTYPMVWFN
ELTKEKSLQVQPNCVRRLLIRRSADQKEPEIIEGPERVREFMNKLQQRIVRPEYVYVGPEE
EGDHVFWYNWGMMHSKIDYPIAYGPRIVHQGWIPSHRVPRGPTAVAH*
DNA sequence of codon optimized Bsc9
(SEQ ID NO: 19)
ATGGCTTCAACTAGTTCGACCTCGACCGATGGACATCTTAATATACGCCCTATGGTC
CACGGCTCCGATAAAAAACTGAATTTTGGTGCGTACATCACTGGACTTGACTTAAAT
AACGCATCGGATGCAGAAGTCGATCAGCTCCGCGAGGCGATTCTGCGCCATAAAAT
TGTTGTAATTAAAGGCCAGCAAGCGGAAAAACCAGACAAAAATTGGGAAATGATAA
AGAAATTAGATCCTATGCATCACATGATAACGCAAGAGGAATTTGGGCAGCTTTTTC
ACCCAACGGGCGAAGGATTGATCGCAATGCTGAAACTGGCCACTGTACCGACAACC
GAGCATGGGCATATTCATCTCATGGGGAAAGGCTATCAGGGCGACGATCATTACGG
GCTGAAGAAACTGAATCTAGGCGAGGCATTCGCCGGGAATTATTACAGTAAACCCTT
AGCGGAGGAGGACTTCCGCGCCGGAGTAACGCGTTTTCAATCTTGGCACATGGATG
GTCCATTATATAAAGTTCACCCGCCGTACATAAGCAGCCTACGGTTCATCCAACTTC
CGGATGGCGAGCAAACAGTCGAGTGGGCAGATGGCTCTGGCCTGAGTCTTAAAACG
AAGCCCGGACGCACCGCCTTTTTTAGCACCTCTCAGCTTTATGATATGTTAACAGAT
GAAGAACGAGCAATGGTGGATAACTCTGCAGTAGAGTATATGTATTATCCTTATGAG
TGGATTCGCGGTTGCCGCGGGAACCCGAACGGGTTAAACGTCGCCGACGAGGGTAG
AGAAAAACCCCTGGATGCAATGGAAGAAATCGCACGAGACGAACGCTGGACTAAA
ACCTATCCTATGGTATGGTTCAATGAACTGACTAAAGAAAAATCTCTACAGGTTCAG
CCTAATTGTGTGCGTAGACTACTTATCCGGAGATCTGCAGATCAGAAAGAGCCAGAA
ATTATTGAGGGCCCAGAAAGAGTACGTGAATTCATGAACAAGTTACAACAGAGAAT
TGTGCGGCCCGAATACGTCTATGTCGGTCCGGAGGAAGAAGGCGATCATGTTTTCTG
GTACAACTGGGGAATGATGCATAGTAAGATTGATTATCCCATTGCCTATGGTCCGCG
CATTGTTCATCAAGGTTGGATTCCAAGTCATCGTGTGCCGCGCGGACCTACGGCCGT
AGCACATTAA
Protein sequence of BscD (Uniprot ID: M2ZB93)
(SEQ ID NO: 20)
FGAYITGLDLNSASDAEIDQLREAILRHKVVIVKGQQAETPDRNWEVIKKLDPMHHMIT
QEEFGQLFHPTGEGLIVRHIHLMGKGYQGDDHYGLKKLDLGEAFAGNYYSKPLAEEDF
QAGITRFQSWHMDGPLYKVHPPYISSLRFIKLPQGEQTIEWADGSGMSLKAKPGRTGFFS
TSQLYEMLTDEEKVMVDNSSVEYMHYPYEWIRSCRGNPNGLGVADEGREKSPETMEEI
AREERWTKTLTGEKSLQVQPNCVRRLLIRRSVDQKEPEVIEGAEHARAFMHQLQQRILR
PEYVYVGPEEEGDHVFWYNWGMMHSKIDYPVSFGPRIVHQGWIPSHRVPQPPAVAAH*
DNA sequence of codon optimized BscD
(SEQ ID NO: 21)
ATGTTCGGGGCTTATATTACGGGGCTGGATTTAAATTCAGCGTCGGATGCTGAAATC
GACCAACTGCGGGAAGCAATTCTACGTCATAAGGTAGTGATTGTAAAAGGCCAACA
GGCAGAGACTCCGGATAGAAACTGGGAGGTGATTAAGAAACTAGATCCAATGCACC
ACATGATCACGCAGGAAGAATTTGGGCAGCTTTTCCATCCAACAGGTGAAGGATTG
ATTGTTCGTCACATACACTTGATGGGGAAAGGTTACCAGGGGGATGATCACTATGGT
CTTAAAAAACTGGATCTCGGGGAAGCCTTTGCCGGGAATTATTATTCAAAACCGCTT
GCTGAAGAGGATTTTCAGGCGGGTATTACTCGCTTCCAGTCATGGCACATGGATGGG
CCCCTGTATAAAGTTCATCCACCTTACATCAGTAGCCTGCGGTTTATTAAGCTGCCCC
AAGGCGAGCAGACTATCGAATGGGCCGATGGTAGTGGTATGTCCCTGAAGGCGAAA
CCGGGTCGAACGGGTTTTTTTAGCACTTCCCAACTGTATGAAATGCTGACCGATGAG
GAAAAGGTTATGGTTGATAATAGTTCTGTTGAATACATGCATTATCCATACGAATGG
ATTCGGTCATGCCGGGGCAACCCTAATGGCCTGGGTGTCGCGGATGAAGGCCGGGA
AAAAAGTCCTGAGACAATGGAAGAGATTGCCAGAGAAGAACGCTGGACGAAAACT
CTTACCGGAGAAAAGAGCCTACAGGTACAGCCCAACTGCGTTCGCCGGCTGCTTATC
CGCCGGAGCGTGGATCAGAAAGAGCCAGAAGTTATTGAGGGAGCGGAACATGCACG
CGCCTTTATGCATCAGCTGCAACAGAGAATTCTACGTCCCGAGTATGTGTATGTTGG
GCCTGAAGAAGAGGGTGATCATGTATTTTGGTACAATTGGGGTATGATGCACAGCAA
AATCGATTATCCTGTTAGTTTCGGTCCACGTATTGTCCATCAAGGTTGGATACCTAGT
CACCGCGTACCGCAACCGCCAGCCGTTGCAGCGCACTAA
Protein sequence of RsBsc9 (Uniprot ID: A0A1E1MJ48)
(SEQ ID NO: 22)
SPSRIDNVSSKFRISPLVHSPDKKCNFGATVEGIDLNNLTDEEILVLREAIWTHKFLVIKDQ
TALVPKKNWELVTRLDPEAPILNQMDFAKCFHPTGEGILKKITLTLLPGVEDIHLMGKGY
QGEDHYGVKNVTLNEAFSSAFHSTPLSEEDFENGNTRFQSWHMDGPGYRIDPPWFSSFR
TIKLPNGPDQTVNWDDGSGLSMKAKPGRTAFFSSAQLYDLLTDEEKTMADHSWVEHM
HHPYEWVRDCHGNPNGLSVACEGRETSMEEMDTFERDPSWTKKYPMVWVNPVTKEKS
FQVQPNIVRKIFIRNNATDTPKVIDDLTEVRAFLNDIQLRILKPEYITAQPQNEGDMLLWD
NYGMMHSRIDYPVKYGTRTAHQCWLGASRGPVGPVALPVDA*
DNA sequence of codon optimized RsBsc9
(SEQ ID NO: 23)
TCTCCTTCGCGTATCGATAACGTCAGCTCTAAGTTTCGGATAAGCCCGCTCGTACAC
AGCCCGGACAAGAAGTGCAATTTTGGCGCTACTGTTGAGGGTATAGATCTGAACAA
CTTAACAGACGAGGAGATTCTGGTGTTGCGTGAGGCAATTTGGACTCACAAGTTCTT
GGTTATCAAGGACCAAACGGCGCTGGTGCCTAAGAAGAATTGGGAGCTGGTTACGC
GCCTGGACCCTGAGGCTCCGATACTCAACCAAATGGACTTCGCCAAATGTTTTCATC
CTACAGGGGAAGGTATCCTTAAGAAAATTACCCTTACGCTGTTACCAGGCGTTGAGG
ACATCCATCTGATGGGCAAAGGATATCAAGGCGAGGACCACTATGGCGTGAAGAAT
GTCACGCTTAACGAGGCTTTCTCCTCTGCTTTTCATAGCACACCACTGTCGGAAGAG
GATTTCGAGAATGGGAACACTCGCTTCCAATCATGGCACATGGATGGCCCCGGCTAC
CGCATAGACCCACCTTGGTTTTCTTCCTTCCGCACGATCAAGCTTCCTAATGGCCCCG
ATCAAACCGTCAATTGGGATGACGGATCGGGACTTTCCATGAAGGCGAAACCAGGA
CGTACAGCATTCTTCTCATCTGCACAACTGTACGACCTTTTAACTGATGAGGAAAAG
ACTATGGCCGATCATTCATGGGTGGAACACATGCACCATCCGTATGAGTGGGTTCGT
GACTGTCACGGTAACCCGAACGGTTTATCTGTCGCCTGTGAGGGCCGGGAAACAAGT
ATGGAAGAGATGGACACATTTGAGCGGGACCCGTCTTGGACAAAGAAGTATCCAAT
GGTTTGGGTCAACCCAGTGACAAAAGAAAAGTCGTTTCAGGTTCAGCCTAACATAGT
TCGGAAGATCTTCATACGTAACAATGCCACAGACACCCCAAAGGTAATCGATGACCT
TACTGAGGTTCGCGCATTCTTAAATGACATCCAGTTGCGTATCTTAAAGCCCGAATA
TATCACGGCCCAACCTCAAAACGAAGGTGACATGCTTCTGTGGGACAACTATGGGAT
GATGCATAGTCGTATTGATTATCCTGTTAAGTATGGCACCCGTACAGCACACCAATG
TTGGCTGGGAGCCTCTCGCGGCCCTGTGGGTCCGGTGGCGCTCCCTGTCGACGCTTA
A
Protein sequence of BvBsc9 (Uniprot ID: W7E0J8)
(SEQ ID NO: 24)
MAFTSSNSTDGHLNIRPMVHGSDKKLNFGAYITGLDLNNASDDEVDQLREAILRHKVVV
VKGQQAETPDKNWEIITKLDPMHHMITQEEFGRLFHPTGEGLIAMLKLATVPTAQDGHI
HLMGKGYQGDDHYGLKKLTLGEAFAGNYYSKPLANEDFQAGIARFQSWHIDGPLYKV
HPPYISSLRFIQLPVGEQKVEWADGSGLSLKTKPGRTGFFSTSQLYEMLTDEERVMVDNS
TVEYMYYPYEWIRGCRGNPNGLSVADEGREKPLELMEEMARDEQWTKTYPMVWFNEL
TKEKSLQVQPNCARRLLIRRSADQKDPEIIEGPERVREFMDKLQQRIVRPEYIYVGPEEEG
DHVFWYNWGLMHSRIDYPVAYGPRIVHQGWVPTHRIPRGSPAVAH*
DNA sequence of codon optimized BvBsc9
(SEQ ID NO: 25)
ATGGCTTTCACTTCGAGCAATTCTACAGATGGGCATTTAAACATACGTCCAATGGTT
CACGGCAGTGACAAGAAGTTAAATTTCGGTGCGTATATCACCGGCCTGGACCTGAAC
AACGCTAGCGACGACGAGGTGGACCAGTTGAGAGAAGCCATACTCCGACACAAGGT
AGTAGTCGTTAAGGGGCAACAGGCCGAAACACCTGATAAGAACTGGGAGATTATCA
CCAAGCTTGATCCAATGCACCACATGATCACTCAAGAGGAATTCGGTCGTCTTTTCC
ATCCAACCGGAGAGGGGTTGATAGCAATGCTCAAGCTCGCTACTGTTCCTACAGCAC
AAGATGGCCACATTCACCTTATGGGTAAGGGGTACCAGGGTGACGATCATTACGGTT
TGAAGAAGTTAACACTGGGTGAGGCTTTCGCAGGAAATTATTATTCGAAGCCGCTGG
CAAACGAGGACTTCCAAGCTGGAATCGCCCGGTTTCAATCTTGGCACATTGACGGCC
CCCTCTACAAGGTACACCCACCTTATATCTCGTCCTTACGTTTCATCCAACTTCCCGT
CGGGGAACAAAAGGTGGAGTGGGCCGACGGTTCTGGCCTGTCCCTTAAAACCAAAC
CAGGGCGTACAGGCTTCTTCTCAACGAGTCAATTATACGAGATGCTGACGGACGAG
GAACGGGTCATGGTGGACAACTCAACAGTAGAGTACATGTATTACCCATACGAGTG
GATACGCGGGTGCCGAGGCAATCCGAATGGCTTATCGGTCGCTGACGAGGGGCGAG
AGAAGCCGCTGGAATTAATGGAAGAAATGGCTAGAGATGAACAGTGGACAAAGAC
CTATCCGATGGTGTGGTTTAACGAATTAACAAAGGAGAAATCGCTGCAAGTTCAACC
AAACTGCGCACGTCGCCTGCTTATACGCCGAAGCGCGGACCAAAAGGACCCTGAGA
TCATAGAGGGTCCTGAGCGTGTGAGAGAATTCATGGATAAGTTACAACAGCGTATTG
TACGGCCCGAATATATTTATGTCGGTCCGGAAGAAGAGGGAGACCATGTTTTCTGGT
ACAATTGGGGATTGATGCACAGTCGTATAGACTACCCGGTTGCGTATGGGCCTCGTA
TAGTACATCAAGGATGGGTCCCAACTCACCGTATTCCGCGAGGGTCGCCTGCAGTAG
CCCATTAA
Protein sequence of DhBsc9 (Uniprot ID: A0A2P5HP46)
(SEQ ID NO: 26)
MGSTEETFRVKPFGGDLTFGAEVYGLDLNEISDAGIDQLRDLLQQHLVLVIKGQQNELP
RKNWELLQKLDPGAPEFTDEQWAKFYNPEGKGILAKLGYNTIPDGGRLYLMGKGYQGE
DHYGLKNVNMSESFADLYYSKPLPKEDFQKGITRFQSWHMDGPQYAINPPLFTSFRCIKL
PEGEQTVDWADGSGLTKKIKPGRTGFFSTAQLYDMLSDEEKQMVDHSWCEYMYYPYE
WILGCRGNPNGINVACEGREVPEEVMEAMPRNPNDQHILPLVWMNSVTGKKHLQVQP
NVVRRLYIRSSPQDKPKVIEDVKEVRDFLTKLQIRILRPENIYVGPEEEGDHVLWYNWGV
MHTKIDYPVEFGPRSAHQGWLPATRKPSGPVPIPVSQ*
DNA sequence of DhBsc9
(SEQ ID NO: 27)
ATGGGATCTACTGAGGAAACTTTCCGTGTGAAACCTTTTGGTGGCGACCTTACCTTC
GGTGCAGAGGTGTACGGGTTAGATCTCAACGAGATCAGCGATGCCGGTATCGATCA
ATTGCGAGACCTTCTGCAACAGCACCTCGTTCTTGTAATTAAGGGGCAGCAAAACGA
ACTGCCGCGAAAGAACTGGGAGCTGCTGCAGAAATTAGACCCAGGGGCTCCAGAGT
TTACAGATGAACAATGGGCTAAATTCTACAACCCGGAAGGTAAAGGAATTCTGGCT
AAATTGGGCTACAACACAATCCCAGACGGTGGCCGCTTGTACCTCATGGGGAAAGG
CTACCAAGGAGAAGACCACTACGGATTGAAGAACGTTAACATGTCGGAGTCTTTCG
CGGACTTATACTACTCTAAACCACTGCCCAAAGAGGATTTCCAAAAGGGAATCACTC
GTTTCCAAAGCTGGCACATGGATGGTCCCCAATATGCTATCAACCCGCCGTTGTTCA
CGAGCTTCCGATGCATCAAGCTTCCCGAGGGTGAGCAAACCGTGGACTGGGCAGAT
GGTTCCGGCTTAACCAAGAAGATCAAGCCTGGGCGAACCGGGTTCTTTTCCACCGCG
CAACTGTATGATATGCTGAGCGACGAAGAGAAGCAAATGGTAGACCATTCATGGTG
TGAGTATATGTACTATCCATACGAGTGGATTCTGGGGTGCAGAGGTAACCCGAACGG
GATCAACGTAGCCTGCGAGGGCCGTGAAGTCCCCGAAGAAGTTATGGAAGCTATGC
CTCGAAACCCCAACGACCAACATATATTGCCCCTGGTATGGATGAATTCAGTCACCG
GTAAGAAGCACTTGCAAGTGCAACCGAACGTCGTACGTCGCTTGTACATACGGTCTT
CACCCCAAGACAAGCCTAAGGTTATTGAGGATGTTAAAGAGGTACGTGACTTTCTGA
CAAAGCTCCAGATACGCATCCTGCGCCCGGAGAATATCTATGTAGGGCCAGAGGAA
GAAGGGGACCACGTCTTATGGTACAACTGGGGCGTCATGCATACCAAGATAGACTA
CCCGGTAGAATTCGGGCCTCGTTCGGCCCACCAAGGTTGGTTACCCGCGACCCGTAA
ACCATCGGGTCCTGTGCCAATCCCAGTAAGCCAGTGA
Protein sequence of CcBsc9 (Uniprot ID: A0A1Q8S6A2)
(SEQ ID NO: 28)
MGSMPGFTVEPFDNGLKFGAEIHGLDINKITDSPEYQVLMTRPDSEVDKFRETIQKYLVV
VVKGQQDELPSKNWELLQKLDPGAPEFTDEEWARFYNPEGKGILAKLGYNAIPDAGRL
YLMGKGYQGKDHYGLKDVEINEPFADAYYSKPLPVEDFKKGVARFQSWHMDGPQYVI
NPPKFSSFRSIRVPKGEQTVEWADGSGLTKKVKPGRTAFISTAQMYDALSEEEKRMADH
SWCEYMYYPYEWILGCRGNPNGLNVACEGREVPEEVMEAMPRDPKHQQLLPLVWLND
FTGGKHLQVQPNIVRRIFVRSGPNSNPKIIEDVKEVRNFLTNLQGRILRPENVYVGPEDEG
DHLFWYNWGVMHSKIDYPVEFGVRTAHQGWLPGSKAPTGPVPIPAQR*
DNA sequence of CcBsc9
(SEQ ID NO: 29)
ATGGGATCAATGCCTGGCTTCACAGTCGAACCTTTTGATAACGGCTTAAAGTTCGGT
GCCGAGATACACGGACTGGACATCAATAAGATCACCGACAGTCCGGAGTACCAAGT
ACTGATGACCCGCCCGGATTCCGAGGTCGACAAGTTTCGCGAAACCATCCAGAAAT
ACTTAGTGGTGGTGGTCAAGGGGCAACAAGATGAGTTGCCTTCAAAGAACTGGGAG
CTGCTTCAGAAGCTGGACCCCGGCGCTCCGGAGTTTACTGACGAGGAGTGGGCACG
ATTCTACAACCCCGAAGGGAAGGGAATACTCGCGAAATTAGGATACAACGCAATCC
CGGATGCTGGGCGGTTGTACCTGATGGGCAAGGGGTACCAAGGTAAGGATCACTAT
GGACTGAAGGATGTGGAGATTAATGAGCCATTTGCAGACGCCTACTATTCGAAACCT
CTGCCAGTTGAAGACTTCAAGAAAGGCGTAGCACGTTTCCAGTCATGGCACATGGAT
GGGCCACAGTACGTAATTAACCCGCCGAAGTTTTCCTCTTTTCGCTCCATACGGGTTC
CAAAGGGTGAGCAAACAGTTGAGTGGGCTGACGGATCAGGCTTGACAAAGAAGGTG
AAGCCAGGGAGAACTGCTTTCATATCCACAGCACAGATGTACGATGCATTATCTGAA
GAGGAAAAGCGTATGGCGGACCACTCATGGTGTGAGTACATGTACTATCCTTACGA
GTGGATACTTGGCTGCCGGGGTAATCCTAACGGTTTAAACGTAGCTTGTGAGGGTAG
AGAGGTTCCTGAAGAGGTGATGGAAGCTATGCCCCGTGACCCAAAGCACCAACAAC
TGTTGCCGCTCGTATGGCTCAATGACTTCACCGGCGGGAAGCATCTGCAAGTTCAAC
CTAACATAGTCCGTCGCATATTCGTACGTAGCGGTCCTAACTCGAACCCGAAAATAA
TAGAAGACGTTAAAGAGGTGCGTAATTTCCTTACCAATCTGCAAGGAAGAATTCTGC
GCCCTGAGAACGTTTATGTGGGACCGGAAGACGAGGGCGACCATTTGTTTTGGTACA
ACTGGGGAGTAATGCACAGCAAAATTGATTACCCCGTAGAATTCGGCGTGCGGACC
GCGCACCAGGGATGGCTGCCGGGTTCTAAGGCCCCCACGGGACCAGTCCCTATCCCT
GCTCAACGTTAA
Protein sequence of MoBsc9 (Uniprot ID: A0A4P7NVE3)
(SEQ ID NO: 30)
MTTSATMTTSATMNTTTKANSFDVKPFGQGLNFGAEIRGLDLNNMTDDDVNKFREVIQ
QYLVVVVKGQGNELPSKNWELLRRLDPDSPELTDDEWAKMYNPEGKGILAKLGYSLIP
GAGRLYLMGKGYQGEDHFGLKDVTIEEAFADLYYSKPLPKEDFEKGITRFQSWHMDGP
QYVVHGPIYTSFRCIKLPEGEQTVDWADGSGLTKKVKPGRTAFFSTAQLYDMFTPEEQQ
MADHSWCEPMFYPYEWILGCRGNPNGLNVACQGREVPFDVMEAMARKPKNQLVLPLV
WMNSVTGKKHFQVQPNTVRRLFIRNNAEETPRVIEDVKEIRDFLTKLQIRVLRPENIYVG
PEEEGDHVFWYNWGVMHTKIDYPVKFGVRTAHQGWIPASRAPTGPVPIPGQE*
DNA sequence of MoBsc9: (bold denotes codon for L110 and Y112)
(SEQ ID NO: 31)
ATGACAACTTCAGCAACTATGACTACCAGCGCCACTATGAACACAACCACCAAGGC
TAACTCCTTCGACGTAAAGCCGTTCGGACAAGGATTAAATTTCGGTGCGGAGATACG
GGGACTGGACCTTAACAACATGACGGACGACGACGTTAACAAGTTTAGAGAGGTCA
TCCAACAATACCTCGTTGTCGTTGTGAAGGGCCAAGGTAACGAGCTGCCAAGCAAG
AATTGGGAGTTGCTTCGGCGCCTGGACCCGGATAGCCCCGAGTTAACCGACGACGA
ATGGGCGAAGATGTACAACCCAGAAGGCAAGGGCATACTGGCGAAGCTGGGGTAC
TCCCTGATACCCGGAGCCGGACGACTGTATCTTATGGGTAAGGGATATCAAGGGGA
AGACCACTTCGGATTAAAGGACGTGACCATTGAAGAGGCGTTTGCGGACCTGTATTA
TAGTAAGCCACTCCCAAAAGAGGACTTCGAGAAGGGAATAACTAGATTCCAGTCAT
GGCACATGGACGGACCACAATACGTGGTGCACGGCCCGATCTACACCAGCTTCCGCT
GCATCAAGTTACCAGAAGGGGAGCAAACTGTTGATTGGGCCGATGGCAGTGGTCTT
ACAAAGAAAGTTAAACCCGGGCGTACCGCTTTCTTCTCAACTGCCCAATTATACGAT
ATGTTCACGCCTGAGGAGCAACAAATGGCGGACCACAGTTGGTGCGAGCCGATGTT
CTACCCGTACGAGTGGATCTTAGGCTGCCGGGGCAACCCGAACGGTTTAAATGTAGC
TTGCCAGGGTCGTGAAGTTCCGTTCGACGTGATGGAAGCGATGGCGCGTAAACCAA
AGAATCAACTCGTTTTGCCGTTAGTGTGGATGAATTCCGTCACAGGAAAGAAACATT
TCCAGGTACAACCTAACACAGTGCGTAGACTGTTCATCCGCAATAATGCAGAGGAG
ACCCCACGGGTCATTGAGGACGTAAAGGAGATCAGAGACTTCTTAACCAAGCTGCA
AATCAGAGTATTACGACCGGAAAACATCTATGTCGGTCCTGAAGAGGAAGGTGACC
ACGTATTTTGGTATAACTGGGGAGTTATGCACACAAAGATTGATTATCCGGTAAAGT
TCGGGGTGCGTACAGCCCATCAGGGCTGGATACCGGCTTCTCGCGCCCCGACTGGCC
CAGTACCTATTCCTGGGCAGGAGTAA
Protein sequence of P450BM3 variant MERO1 L75A
(SEQ ID NO: 32)
MTIKEMPQPKTFGELKNLPLLNTDKPVQALMKIADELGEIFKFEAPGRVTRYLSSQRLIK
EACDESRFDKNLSQAAKFARDFGGDGLVTSWTHEKNWKKAHNILLPSFSQQAMKGYH
AMMVDIAVQLVQKWERLNADEHIEVSEDMTRLTLDTIGLCGFNYRFNSFYRDQPHPFIIS
MVRALDEVMNKLQRANPDDPAYDENKRQCQEDIKVMNDLVDKIIADRKARGEQSDDL
LTQMLNGKDPETGEPLDDGNISYQIITFLIAGHETTSGLLSFALYFLVKNPHVLQKVAEEA
ARVLVDPVPSYKQVKQLKYVGMVLNEALRLWPTAPAFSLYAKEDTVLGGEYPLEKGD
EVMVLIPQLHRDKTIWGDDVEEFRPERFENPSAIPQHAFKPFGNGQRACIGQQFALHEAT
LVLGMMLKHFDFEDHTNYELDIKETLTLKPEGFVVKAKSKKIPLGGIPSPSTEQSAKKVR
KKAENAHNTPLLVLYGSNMGTAEGTARDLADIAMSKGFAPQVATLDSHAGNLPREGA
VLIVTASYNGHPPDNAKQFVDWLDQASADEVKGVRYSVFGCGDKNWATTYQKVPAFI
DETLAAKGAENIADRGEADASDDFEGTYEEWREHMWSDVAAYFNLDIENSEDNKSTLS
LQFVDSAADMPLAKMHGAFSTNVVASKELQQPGSARSTRHLEIELPKEASYQEGDHLG
VIPRNYEGIVNRVTARFGLDASQQIRLEAEEEKLAHLPLAKTVSVEELLQYVELQDPVTR
TQLRAMAAKTVCPPHKVELEALLEKQAYKEQVLAKRLTMLELLEKYPACEMKFSEFIA
LLPSIRPRYYSISSSPRVDEKQASITVSVVSGEAWSGYGEYKGIASNYLAELQEGDTITCFI
STPQSEFTLPKDPETPLIMVGPGTGVAPFRGFVQARKQLKEQGQSLGEAHLYFGCRSPHE
DYLYQEELENAQSEGIITLHTAFSRMPNQPKTYVQHVMEQDGKKLIELLDQGAHFYICG
DGSQMAPAVEATLMKSYADVHQVSEADARLWLQQLEEKGRYAKDVWAG
Protein sequence of Bsc9 C304V
(SEQ ID NO: 33)
MASTSSTSTDGHLNIRPMVHGSDKKLNFGAYITGLDLNNASDAEVDQLREAILRHKIVVI
KGQQAEKPDKNWEMIKKLDPMHHMITQEEFGQLFHPTGEGLIAMLKLATVPTTEHGHIH
LMGKGYQGDDHYGLKKLNLGEAFAGNYYSKPLAEEDFRAGVTRFQSWHMDGPLYKV
HPPYISSLRFIQLPDGEQTVEWADGSGLSLKTKPGRTAFFSTSQLYDMLTDEERAMVDNS
AVEYMYYPYEWIRGCRGNPNGLNVADEGREKPLDAMEEIARDERWTKTYPMVWFNEL
TKEKSLQVQPNVVRRLLIRRSADQKEPEIIEGPERVREFMNKLQQRIVRPEYVYVGPEEE
GDHVFWYNWGMMHSKIDYPIAYGPRIVHQGWIPSHRVPRGPTAVAH*
DNA sequence of codon optimized Bsc9 C304V
(SEQ ID NO: 34)
ATGGCTTCAACTAGTTCGACCTCGACCGATGGACATCTTAATATACGCCCTATGGTCCA
CGGCTCCGATAAAAAACTGAATTTTGGTGCGTACATCACTGGACTTGACTTAAATAAC
GCATCGGATGCAGAAGTCGATCAGCTCCGCGAGGCGATTCTGCGCCATAAAATTGTTG
TAATTAAAGGCCAGCAAGCGGAAAAACCAGACAAAAATTGGGAAATGATAAAGAAAT
TAGATCCTATGCATCACATGATAACGCAAGAGGAATTTGGGCAGCTTTTTCACCCAAC
GGGCGAAGGATTGATCGCAATGCTGAAACTGGCCACTGTACCGACAACCGAGCATGG
GCATATTCATCTCATGGGGAAAGGCTATCAGGGCGACGATCATTACGGGCTGAAGAAA
CTGAATCTAGGCGAGGCATTCGCCGGGAATTATTACAGTAAACCCTTAGCGGAGGAGG
ACTTCCGCGCCGGAGTAACGCGTTTTCAATCTTGGCACATGGATGGTCCATTATATAAA
GTTCACCCGCCGTACATAAGCAGCCTACGGTTCATCCAACTTCCGGATGGCGAGCAAA
CAGTCGAGTGGGCAGATGGCTCTGGCCTGAGTCTTAAAACGAAGCCCGGACGCACCG
CCTTTTTTAGCACCTCTCAGCTTTATGATATGTTAACAGATGAAGAACGAGCAATGGTG
GATAACTCTGCAGTAGAGTATATGTATTATCCTTATGAGTGGATTCGCGGTTGCCGCGG
GAACCCGAACGGGTTAAACGTCGCCGACGAGGGTAGAGAAAAACCCCTGGATGCAA
TGGAAGAAATCGCACGAGACGAACGCTGGACTAAAACCTATCCTATGGTATGGTTCA
ATGAACTGACTAAAGAAAAATCTCTACAGGTTCAGCCTAATGTGGTGCGTAGACTACT
TATCCGGAGATCTGCAGATCAGAAAGAGCCAGAAATTATTGAGGGCCCAGAAAGAGT
ACGTGAATTCATGAACAAGTTACAACAGAGAATTGTGCGGCCCGAATACGTCTATGTC
GGTCCGGAGGAAGAAGGCGATCATGTTTTCTGGTACAACTGGGGAATGATGCATAGTA
AGATTGATTATCCCATTGCCTATGGTCCGCGCATTGTTCATCAAGGTTGGATTCCAAGT
CATCGTGTGCCGCGCGGACCTACGGCCGTAGCACATTAA
Protein sequence of Bsc9 C304T
(SEQ ID NO: 35)
MASTSSTSTDGHLNIRPMVHGSDKKLNFGAYITGLDLNNASDAEVDQLREAILRHKIVVI
KGQQAEKPDKNWEMIKKLDPMHHMITQEEFGQLFHPTGEGLIAMLKLATVPTTEHGHIH
LMGKGYQGDDHYGLKKLNLGEAFAGNYYSKPLAEEDFRAGVTRFQSWHMDGPLYKV
HPPYISSLRFIQLPDGEQTVEWADGSGLSLKTKPGRTAFFSTSQLYDMLTDEERAMVDNS
AVEYMYYPYEWIRGCRGNPNGLNVADEGREKPLDAMEEIARDERWTKTYPMVWFNEL
TKEKSLQVQPNTVRRLLIRRSADQKEPEIIEGPERVREFMNKLQQRIVRPEYVYVGPEEEG
DHVFWYNWGMMHSKIDYPIAYGPRIVHQGWIPSHRVPRGPTAVAH*
DNA sequence of codon optimized Bsc9 C304T
(SEQ ID NO: 36)
ATGGCTTCAACTAGTTCGACCTCGACCGATGGACATCTTAATATACGCCCTATGGTCCA
CGGCTCCGATAAAAAACTGAATTTTGGTGCGTACATCACTGGACTTGACTTAAATAAC
GCATCGGATGCAGAAGTCGATCAGCTCCGCGAGGCGATTCTGCGCCATAAAATTGTTG
TAATTAAAGGCCAGCAAGCGGAAAAACCAGACAAAAATTGGGAAATGATAAAGAAAT
TAGATCCTATGCATCACATGATAACGCAAGAGGAATTTGGGCAGCTTTTTCACCCAAC
GGGCGAAGGATTGATCGCAATGCTGAAACTGGCCACTGTACCGACAACCGAGCATGG
GCATATTCATCTCATGGGGAAAGGCTATCAGGGCGACGATCATTACGGGCTGAAGAAA
CTGAATCTAGGCGAGGCATTCGCCGGGAATTATTACAGTAAACCCTTAGCGGAGGAGG
ACTTCCGCGCCGGAGTAACGCGTTTTCAATCTTGGCACATGGATGGTCCATTATATAAA
GTTCACCCGCCGTACATAAGCAGCCTACGGTTCATCCAACTTCCGGATGGCGAGCAAA
CAGTCGAGTGGGCAGATGGCTCTGGCCTGAGTCTTAAAACGAAGCCCGGACGCACCG
CCTTTTTTAGCACCTCTCAGCTTTATGATATGTTAACAGATGAAGAACGAGCAATGGTG
GATAACTCTGCAGTAGAGTATATGTATTATCCTTATGAGTGGATTCGCGGTTGCCGCGG
GAACCCGAACGGGTTAAACGTCGCCGACGAGGGTAGAGAAAAACCCCTGGATGCAA
TGGAAGAAATCGCACGAGACGAACGCTGGACTAAAACCTATCCTATGGTATGGTTCA
ATGAACTGACTAAAGAAAAATCTCTACAGGTTCAGCCTAATACCGTGCGTAGACTACT
TATCCGGAGATCTGCAGATCAGAAAGAGCCAGAAATTATTGAGGGCCCAGAAAGAGT
ACGTGAATTCATGAACAAGTTACAACAGAGAATTGTGCGGCCCGAATACGTCTATGTC
GGTCCGGAGGAAGAAGGCGATCATGTTTTCTGGTACAACTGGGGAATGATGCATAGTA
AGATTGATTATCCCATTGCCTATGGTCCGCGCATTGTTCATCAAGGTTGGATTCCAAGT
CATCGTGTGCCGCGCGGACCTACGGCCGTAGCACATTAA
Protein sequence of Bsc9 ‘VR’ (with N-His6 tag)
(SEQ ID NO: 37)
MGSSHHHHHHSSGLVPRGSHMASTSSTSTDGHLNIRPMVHGSDKKLNFGAYITGLDLNN
ASDAEVDQLREAILRHKIVVIKGQQAEKPDKNWEMIKKLDPMHHMITQEEFGQLFHPTG
EGLIVRHIHLMGKGYQGDDHYGLKKLNLGEAFAGNYYSKPLAEEDFRAGVTRFQSWH
MDGPLYKVHPPYISSLRFIQLPDGEQTVEWADGSGLSLKTKPGRTAFFSTSQLYDMLTDE
ERAMVDNSAVEYMYYPYEWIRGCRGNPNGLNVADEGREKPLDAMEEIARDERWTKTY
PMVWFNELTKEKSLQVQPNCVRRLLIRRSADQKEPEIIEGPERVREFMNKLQQRIVRPEY
VYVGPEEEGDHVFWYNWGMMHSKIDYPIAYGPRIVHQGWIPSHRVPRGPTAVAH
DNA sequence of MERO1 L75A
ATGACAATTAAAGAAATGCCTCAGCCAAAAACGTTTGGAGAGCTTAAAAATTTACC
GTTATTAAACACAGATAAACCGGTTCAAGCTTTGATGAAAATTGCGGATGAATTAGG
AGAAATCTTTAAATTCGAGGCGCCTGGTCGTGTAACGCGCTACTTATCAAGTCAGCG
TCTAATTAAAGAAGCATGCGATGAATCACGCTTTGATAAAAACTTAAGTCAAGCGGC
CAAATTCGCGCGTGATTTTGGCGGAGACGGGTTAGTTACAAGCTGGACGCATGAAA
AAAATTGGAAAAAAGCGCATAATATCTTACTTCCAAGCTTTAGTCAGCAGGCAATGA
AAGGCTATCATGCGATGATGGTCGATATCGCCGTGCAGCTTGTTCAAAAGTGGGAGC
GTCTAAATGCAGATGAGCATATTGAAGTATCGGAAGACATGACACGTTTAACGCTTG
ATACAATTGGTCTTTGCGGCTTTAACTATCGCTTTAACAGCTTTTACCGAGATCAGCC
TCATCCATTTATTATAAGTATGGTCCGTGCACTGGATGAAGTAATGAACAAGCTGCA
GCGAGCAAATCCAGACGACCCAGCTTATGATGAAAACAAGCGCCAGTGTCAAGAAG
ATATCAAGGTGATGAACGACCTAGTAGATAAAATTATTGCAGATCGCAAAGCAAGG
GGTGAACAAAGCGATGATTTATTAACGCAGATGCTAAACGGAAAAGATCCAGAAAC
GGGTGAGCCGCTTGATGACGGGAACATTAGCTATCAAATTATTACATTCTTAATTGC
GGGACACGAAACAACAAGTGGTCTTTTATCATTTGCGCTGTATTTCTTAGTGAAAAA
TCCACATGTATTACAAAAAGTAGCAGAAGAAGCAGCACGAGTTCTAGTAGATCCTG
TTCCAAGCTACAAACAAGTCAAACAGCTTAAATATGTCGGCATGGTCTTAAACGAAG
CGCTGCGCTTATGGCCAACTGCGCCTGCGTTTTCCCTATATGCAAAAGAAGATACGG
TGCTTGGAGGAGAATATCCTTTAGAAAAAGGCGACGAAGTAATGGTTCTGATTCCTC
AGCTTCACCGTGATAAAACAATTTGGGGAGACGATGTGGAGGAGTTCCGTCCAGAG
CGTTTTGAAAATCCAAGTGCGATTCCGCAGCATGCGTTTAAACCGTTTGGAAACGGT
CAGCGTGCGTGTATCGGTCAGCAGTTCGCTCTTCATGAAGCAACGCTGGTACTTGGT
ATGATGCTAAAACACTTTGACTTTGAAGATCATACAAACTACGAGCTCGATATTAAA
GAAACTTTAACGTTAAAACCTGAAGGCTTTGTGGTAAAAGCAAAATCGAAAAAAAT
TCCGCTTGGCGGTATTCCTTCACCTAGCACTGAACAGTCTGCTAAAAAAGTACGCAA
AAAGGCAGAAAACGCTCATAATACGCCGCTGCTTGTGCTATACGGTTCAAATATGGG
AACAGCTGAAGGAACGGCGCGTGATTTAGCAGATATTGCAATGAGCAAAGGATTTG
CACCGCAGGTCGCAACGCTTGATTCACACGCCGGAAATCTTCCGCGCGAAGGAGCT
GTATTAATTGTAACGGCGTCTTATAACGGTCATCCGCCTGATAACGCAAAGCAATTT
GTCGACTGGTTAGACCAAGCGTCTGCTGATGAAGTAAAAGGCGTTCGCTACTCCGTA
TTTGGATGCGGCGATAAAAACTGGGCTACTACGTATCAAAAAGTGCCTGCTTTTATC
GATGAAACGCTTGCCGCTAAAGGGGCAGAAAACATCGCTGACCGCGGTGAAGCAGA
TGCAAGCGACGACTTTGAAGGCACATATGAAGAATGGCGTGAACATATGTGGAGTG
ACGTAGCAGCCTACTTTAACCTCGACATTGAAAACAGTGAAGATAATAAATCTACTC
TTTCACTTCAATTTGTCGACAGCGCCGCGGATATGCCGCTTGCGAAAATGCACGGTG
CGTTTTCAACGAACGTCGTAGCAAGCAAAGAACTTCAACAGCCAGGCAGTGCACGA
AGCACGCGACATCTTGAAATTGAACTTCCAAAAGAAGCTTCTTATCAAGAAGGAGA
TCATTTAGGTGTTATTCCTCGCAACTATGAAGGAATAGTAAACCGTGTAACAGCAAG
GTTCGGCCTAGATGCATCACAGCAAATCCGTCTGGAAGCAGAAGAAGAAAAATTAG
CTCATTTGCCACTCGCTAAAACAGTATCCGTAGAAGAGCTTCTGCAATACGTGGAGC
TTCAAGATCCTGTTACGCGCACGCAGCTTCGCGCAATGGCTGCTAAAACGGTCTGCC
CGCCGCATAAAGTAGAGCTTGAAGCCTTGCTTGAAAAGCAAGCCTACAAAGAACAA
GTGCTGGCAAAACGTTTAACAATGCTTGAACTGCTTGAAAAATACCCGGCGTGTGAA
ATGAAATTCAGCGAATTTATCGCCCTTCTGCCAAGCATACGCCCGCGCTATTACTCG
ATTTCTTCATCACCTCGTGTCGATGAAAAACAAGCAAGCATCACGGTCAGCGTTGTC
TCAGGAGAAGCGTGGAGCGGATATGGAGAATATAAAGGAATTGCGTCGAACTATCT
TGCCGAGCTGCAAGAAGGAGATACGATTACGTGCTTTATTTCCACACCGCAGTCAGA
ATTTACGCTGCCAAAAGACCCTGAAACGCCGCTTATCATGGTCGGACCGGGAACAG
GCGTCGCGCCGTTTAGAGGCTTTGTGCAGGCGCGCAAACAGCTAAAAGAACAAGGA
CAGTCACTTGGAGAAGCACATTTATACTTCGGCTGCCGTTCACCTCATGAAGACTAT
CTGTATCAAGAAGAGCTTGAAAACGCCCAAAGCGAAGGCATCATTACGCTTCATAC
CGCTTTTTCTCGCATGCCAAATCAGCCGAAAACATACGTTCAGCACGTAATGGAACA
AGACGGCAAGAAATTGATTGAACTTCTTGATCAAGGAGCGCACTTCTATATTTGCGG
AGACGGAAGCCAAATGGCACCTGCCGTTGAAGCAACGCTTATGAAAAGCTATGCTG
ACGTTCACCAAGTGAGTGAAGCAGACGCTCGCTTATGGCTGCAGCAGCTAGAAGAA
AAAGGCCGATACGCAAAAGACGTGTGGGCTGGGTAA
Protein sequence of P450BM3 variant MERO1 L181F V184T
MTIKEMPQPKTFGELKNLPLLNTDKPVQALMKIADELGEIFKFEAPGRVTRYLSSQRLIK
EACDESRFDKNLSQALKFARDFGGDGLVTSWTHEKNWKKAHNILLPSFSQQAMKGYH
AMMVDIAVQLVQKWERLNADEHIEVSEDMTRLTLDTIGLCGFNYRFNSFYRDQPHPFIIS
MVRAFDETMNKLQRANPDDPAYDENKRQCQEDIKVMNDLVDKIIADRKARGEQSDDL
LTQMLNGKDPETGEPLDDGNISYQIITFLIAGHETTSGLLSFALYFLVKNPHVLQKVAEEA
ARVLVDPVPSYKQVKQLKYVGMVLNEALRLWPTAPAFSLYAKEDTVLGGEYPLEKGD
EVMVLIPQLHRDKTIWGDDVEEFRPERFENPSAIPQHAFKPFGNGQRACIGQQFALHEAT
LVLGMMLKHFDFEDHTNYELDIKETLTLKPEGFVVKAKSKKIPLGGIPSPSTEQSAKKVR
KKAENAHNTPLLVLYGSNMGTAEGTARDLADIAMSKGFAPQVATLDSHAGNLPREGA
VLIVTASYNGHPPDNAKQFVDWLDQASADEVKGVRYSVFGCGDKNWATTYQKVPAFI
DETLAAKGAENIADRGEADASDDFEGTYEEWREHMWSDVAAYFNLDIENSEDNKSTLS
LQFVDSAADMPLAKMHGAFSTNVVASKELQQPGSARSTRHLEIELPKEASYQEGDHLG
VIPRNYEGIVNRVTARFGLDASQQIRLEAEEEKLAHLPLAKTVSVEELLQYVELQDPVTR
TQLRAMAAKTVCPPHKVELEALLEKQAYKEQVLAKRLTMLELLEKYPACEMKFSEFIA
LLPSIRPRYYSISSSPRVDEKQASITVSVVSGEAWSGYGEYKGIASNYLAELQEGDTITCFI
STPQSEFTLPKDPETPLIMVGPGTGVAPFRGFVQARKQLKEQGQSLGEAHLYFGCRSPHE
DYLYQEELENAQSEGIITLHTAFSRMPNQPKTYVQHVMEQDGKKLIELLDQGAHFYICG
DGSQMAPAVEATLMKSYADVHQVSEADARLWLQQLEEKGRYAKDVWAG
DNA sequence of MERO1 L181F V184T
ATGACAATTAAAGAAATGCCTCAGCCAAAAACGTTTGGAGAGCTTAAAAATTTACC
GTTATTAAACACAGATAAACCGGTTCAAGCTTTGATGAAAATTGCGGATGAATTAGG
AGAAATCTTTAAATTCGAGGCGCCTGGTCGTGTAACGCGCTACTTATCAAGTCAGCG
TCTAATTAAAGAAGCATGCGATGAATCACGCTTTGATAAAAACTTAAGTCAAGCGCT
TAAATTCGCGCGTGATTTTGGCGGAGACGGGTTAGTTACAAGCTGGACGCATGAAA
AAAATTGGAAAAAAGCGCATAATATCTTACTTCCAAGCTTTAGTCAGCAGGCAATGA
AAGGCTATCATGCGATGATGGTCGATATCGCCGTGCAGCTTGTTCAAAAGTGGGAGC
GTCTAAATGCAGATGAGCATATTGAAGTATCGGAAGACATGACACGTTTAACGCTTG
ATACAATTGGTCTTTGCGGCTTTAACTATCGCTTTAACAGCTTTTACCGAGATCAGCC
TCATCCATTTATTATAAGTATGGTCCGTGCATTCGATGAAACCATGAACAAGCTGCA
GCGAGCAAATCCAGACGACCCAGCTTATGATGAAAACAAGCGCCAGTGTCAAGAAG
ATATCAAGGTGATGAACGACCTAGTAGATAAAATTATTGCAGATCGCAAAGCAAGG
GGTGAACAAAGCGATGATTTATTAACGCAGATGCTAAACGGAAAAGATCCAGAAAC
GGGTGAGCCGCTTGATGACGGGAACATTAGCTATCAAATTATTACATTCTTAATTGC
GGGACACGAAACAACAAGTGGTCTTTTATCATTTGCGCTGTATTTCTTAGTGAAAAA
TCCACATGTATTACAAAAAGTAGCAGAAGAAGCAGCACGAGTTCTAGTAGATCCTG
TTCCAAGCTACAAACAAGTCAAACAGCTTAAATATGTCGGCATGGTCTTAAACGAAG
CGCTGCGCTTATGGCCAACTGCGCCTGCGTTTTCCCTATATGCAAAAGAAGATACGG
TGCTTGGAGGAGAATATCCTTTAGAAAAAGGCGACGAAGTAATGGTTCTGATTCCTC
AGCTTCACCGTGATAAAACAATTTGGGGAGACGATGTGGAGGAGTTCCGTCCAGAG
CGTTTTGAAAATCCAAGTGCGATTCCGCAGCATGCGTTTAAACCGTTTGGAAACGGT
CAGCGTGCGTGTATCGGTCAGCAGTTCGCTCTTCATGAAGCAACGCTGGTACTTGGT
ATGATGCTAAAACACTTTGACTTTGAAGATCATACAAACTACGAGCTCGATATTAAA
GAAACTTTAACGTTAAAACCTGAAGGCTTTGTGGTAAAAGCAAAATCGAAAAAAAT
TCCGCTTGGCGGTATTCCTTCACCTAGCACTGAACAGTCTGCTAAAAAAGTACGCAA
AAAGGCAGAAAACGCTCATAATACGCCGCTGCTTGTGCTATACGGTTCAAATATGGG
AACAGCTGAAGGAACGGCGCGTGATTTAGCAGATATTGCAATGAGCAAAGGATTTG
CACCGCAGGTCGCAACGCTTGATTCACACGCCGGAAATCTTCCGCGCGAAGGAGCT
GTATTAATTGTAACGGCGTCTTATAACGGTCATCCGCCTGATAACGCAAAGCAATTT
GTCGACTGGTTAGACCAAGCGTCTGCTGATGAAGTAAAAGGCGTTCGCTACTCCGTA
TTTGGATGCGGCGATAAAAACTGGGCTACTACGTATCAAAAAGTGCCTGCTTTTATC
GATGAAACGCTTGCCGCTAAAGGGGCAGAAAACATCGCTGACCGCGGTGAAGCAGA
TGCAAGCGACGACTTTGAAGGCACATATGAAGAATGGCGTGAACATATGTGGAGTG
ACGTAGCAGCCTACTTTAACCTCGACATTGAAAACAGTGAAGATAATAAATCTACTC
TTTCACTTCAATTTGTCGACAGCGCCGCGGATATGCCGCTTGCGAAAATGCACGGTG
CGTTTTCAACGAACGTCGTAGCAAGCAAAGAACTTCAACAGCCAGGCAGTGCACGA
AGCACGCGACATCTTGAAATTGAACTTCCAAAAGAAGCTTCTTATCAAGAAGGAGA
TCATTTAGGTGTTATTCCTCGCAACTATGAAGGAATAGTAAACCGTGTAACAGCAAG
GTTCGGCCTAGATGCATCACAGCAAATCCGTCTGGAAGCAGAAGAAGAAAAATTAG
CTCATTTGCCACTCGCTAAAACAGTATCCGTAGAAGAGCTTCTGCAATACGTGGAGC
TTCAAGATCCTGTTACGCGCACGCAGCTTCGCGCAATGGCTGCTAAAACGGTCTGCC
CGCCGCATAAAGTAGAGCTTGAAGCCTTGCTTGAAAAGCAAGCCTACAAAGAACAA
GTGCTGGCAAAACGTTTAACAATGCTTGAACTGCTTGAAAAATACCCGGCGTGTGAA
ATGAAATTCAGCGAATTTATCGCCCTTCTGCCAAGCATACGCCCGCGCTATTACTCG
ATTTCTTCATCACCTCGTGTCGATGAAAAACAAGCAAGCATCACGGTCAGCGTTGTC
TCAGGAGAAGCGTGGAGCGGATATGGAGAATATAAAGGAATTGCGTCGAACTATCT
TGCCGAGCTGCAAGAAGGAGATACGATTACGTGCTTTATTTCCACACCGCAGTCAGA
ATTTACGCTGCCAAAAGACCCTGAAACGCCGCTTATCATGGTCGGACCGGGAACAG
GCGTCGCGCCGTTTAGAGGCTTTGTGCAGGCGCGCAAACAGCTAAAAGAACAAGGA
CAGTCACTTGGAGAAGCACATTTATACTTCGGCTGCCGTTCACCTCATGAAGACTAT
CTGTATCAAGAAGAGCTTGAAAACGCCCAAAGCGAAGGCATCATTACGCTTCATAC
CGCTTTTTCTCGCATGCCAAATCAGCCGAAAACATACGTTCAGCACGTAATGGAACA
AGACGGCAAGAAATTGATTGAACTTCTTGATCAAGGAGCGCACTTCTATATTTGCGG
AGACGGAAGCCAAATGGCACCTGCCGTTGAAGCAACGCTTATGAAAAGCTATGCTG
ACGTTCACCAAGTGAGTGAAGCAGACGCTCGCTTATGGCTGCAGCAGCTAGAAGAA
AAAGGCCGATACGCAAAAGACGTGTGGGCTGGGTAA

### REFERENCES

S1. Li, J.; Li, F.; King-Smith, E.; Renata, H. Nat. Chem. 2020, 12, 173.
S2. Li, F.; Renata, H. J. Am. Chem. Soc. 2021, 143, 18280.
S3. Baek, M.; DiMaio, F.; Anishchenko, I.; Dauparas, J.; Ovchinnikov, S.; Lee, G. R.; Wang, J.; Cong, Q.; Kinch, L. N.; Schaeffer, R. D.; Millin, C.; Park, H.; Adams, C.; Glassman, C. R.; DeGiovanni, A.; Pereira, J. H.; Rodrigues, A. V.; van Dijk, A. A.; Ebrecht, A. C.; Opperman, D. J.; Sagmeister, T.; Buhlheller, C.; Pavkov-Keller, T.; Rathinaswamy, M. K.; Dalwadi, U.; Yip, C. K.; Burke, J. E.; Garcia, K. C.; Grishin, N. V; Adams, P. D.; Read, R. J.; Baker, D. Science, 2021, 373, 871.
S4. a) Lange, G. L.; Neidert, E. E.; Orrom, W. J.; Wallace, D. J. Can. J. Chem., 1978, 56, 1628. b) Mehta, G.; Krishnamurthy, N. Tetrahedron Lett., 1987, 28, 5945. c) Yu, X.; Xiao, L.; Wang, Z.; Luo, T. J. Am. Chem. Soc. 2019, 141, 3440.
S5. Zhang, X.; King-Smith, E.; Dong, L.-B.; Yang, L.-C.; Rudolf, J. D.; Shen, B.; Renata, H. Science, 2020, 369, 799.
S6. Uwamori, M.; Osada, R.; Sugiyama, R.; Nagatani, K.; Nakada, M. J. Am. Chem. Soc. 2020, 142, 5556.
S7. a) Sassa, T.; Negoro, T.; Ueki, H. Agr. Bio. Chem. 1972, 36, 2281; b) Sassa, T.; Takahama, A.; Shindo, T. Agr. Bio. Chem. 1975, 39, 1729.
S8. Okamoto, H.; Arita, H.; Kato, N.; Takeshita, H. Chem. Lett. 1994, 2335.
S9. Tazawa, A.; Ye, Y; Ozaki, T.; Liu, C.; Ogasawara, Y; Dairi, T.; Higuchi, Y; Kato, N.; Gomi, K.; Minami, A.; Oikawa, H. Org. Lett., 2018, 20, 6178.
S10. MacKinnon, S. L.; Keifer, P; Ayer, W. A. Phytochemistry, 1999, 51, 215.
S11. Tang, Y; Xue, Y; Du, G.; Wang, J.; Liu, J.; Sun, B.; Li, X.-N.; Yao, G.; Luo, Z.; Zhang, Y. Angew. Chem. Int. Ed. 2016, 55, 4069.
S12. Li, F.; Sun, W.; Guan, J.; Lu, Y; Lin, S.; Zhang, S.; Gao, W.; Liu, J.; Du, G.; Wang, J.; Zhu, H.; Qi, C.; Hu, Z.; Zhang, Y Org. Biomol. Chem. 2018, 16, 8751.
S13. Kenmoku, H.; Takeue, S.; Oogushi, M.; Yagi, Y; Sassa, T.; Toyota, M.; Asakawa, Y Nat. Prod. Comm. 2014, 9, 351.
S14. Hu, Z.; Sun, W.; Li, F.; Guan, J.; Lu, Y; Liu, J.; Tang, Y; Du, G.; Xue, Y; Luo, Z.; Wang, J.; Zhu, H.; Zhang, Y. Org. Lett. 2018, 20, 5198.
S15. Huffman, T. R.; Kuroo, A.; Sato, R.; Shenvi, R. A. ChemRxiv DOI: 10.26434/chemrxiv-2022-dcbd8.
S16. Pedras, M. S. C.; Chumala, P. B.; Jin, W.; Islam, M. S.; Hauck, D. W. Phytochemistry, 2009, 70, 394.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of preparing a compound of Formula I-1 or I-2: 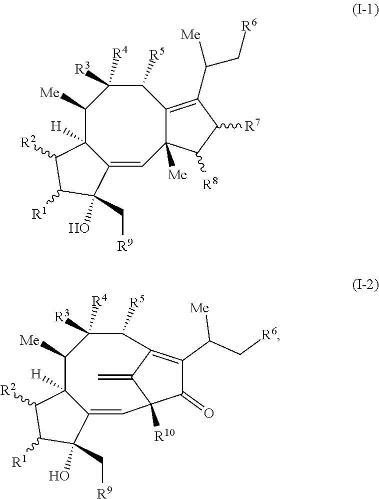
or a salt, solvate, hydrate, polymorph, co-crystal, tautomer, stereoisomer, isotopically labeled compound, or prodrug thereof, the method comprising incubating a first reaction mixture to produce the compound of Formula I-1 or I-2, respectively, or a salt, solvate, hydrate, polymorph, co-crystal, tautomer, stereoisomer, isotopically labeled compound, or prodrug thereof, wherein the first reaction mixture comprises:
(a) a compound of Formula A1 or A2, respectively: 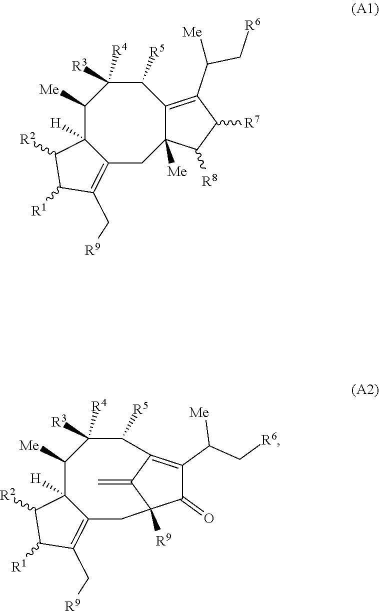
or a salt, solvate, hydrate, polymorph, co-crystal, tautomer, stereoisomer, isotopically labeled compound, or prodrug thereof; and
(b) a non-heme dioxygenase;
wherein:
each one of R¹, R², R⁶, R⁷, and R⁸ is independently hydrogen, —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, or —OS(═O)₂N(R^{a})₂;
R³ and R⁴ taken together form ═O;
or: R³ is —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, or —OS(═O)₂N(R^{a})₂, and R⁴ is hydrogen;
each one of R⁵, R⁹, and R¹⁰ is independently —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, or hydrogen; and
each R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl.

2. The method of claim 1, wherein the non-heme dioxygenase is a non-heme dioxygenase existing in a strain of the fungi Magnaporthe oryzae.

3. The method of claim 1 or 2, wherein the non-heme dioxygenase comprises an amino acid sequence that is at least 80% identical to the amino acid sequence of SEQ ID NO: 30, 26, 28, 24, 22, 35, 33, or 18.

4. The method of any one of claims 1-3, wherein the non-heme dioxygenase comprises an amino acid sequence that is at least 80% identical to the amino acid sequence of SEQ ID NO: 30.

5. The method of any one of claims 1-4, wherein the non-heme dioxygenase comprises a mutation.

6. The method of claim 5, wherein the mutation is a mutation of L110 or Y112, or a combination thereof.

7. The method of claim 5 or 6, wherein the mutation is L110A, Y112M, or Y112R, or a combination thereof.

8. The method of any one of claims 1-4, wherein the non-heme dioxygenase comprises an amino acid sequence of SEQ ID NO: 30.

9. The method of any one of claims 1-3, wherein the non-heme dioxygenase comprises an amino acid sequence of SEQ ID NO: 26, 28, 24, 22, 35, 33, or 18.

10. The method of any one of claims 1-9, wherein the method further comprises lysing a cell to provide a cell lysate, wherein the step of incubating a first reaction mixture is performed after the step of lysing a cell.

11. The method of claim 10, wherein the step of incubating a first reaction mixture is performed immediately after the step of lysing a cell.

12. The method of claim 10 or 11, wherein the cell lysate comprises the non-heme dioxygenase.

13. The method of any one of claims 1-12, wherein the non-heme dioxygenase is isolated from a cell lysate.

14. The method of any one of claims 10-13, wherein the cell is a cell of a fungus, yeast, plant, alga, or bacterium.

15. The method of any one of claims 10-14, wherein the cell is a cell of a strain of the bacteria Escherichia coli.

16. The method of any one of claims 10-14, wherein the cell is a cell of a strain of the fungi Magnaporthe oryzae.

17. The method of any one of claims 10-16, wherein the cell is modified to express the non-heme dioxygenase.

18. The method of any one of claims 1-17, wherein the non-heme dioxygenase is a recombinant non-heme dioxygenase.

19. The method of any one of claims 1-18, wherein R¹⁰ is —OR^{a}.

20. The method of any one of claims 1-18, wherein R¹⁰ is —OH.

21. The method of any one of claims 1-18, wherein R¹⁰ is hydrogen.

22. The method of any one of claims 1-18, wherein R⁷ is hydrogen.

23. The method of any one of claims 1-18, wherein R⁷ is —OR^{a}.

24. The method of any one of claims 1-18, wherein R⁷ is —OH.

25. The method of any one of claims 22 and 24 further comprising incubating a second reaction mixture to produce the compound of Formula III-1: 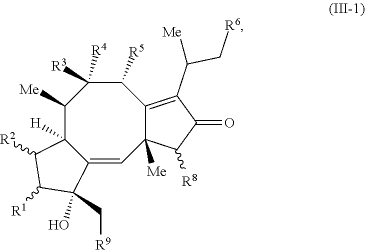
or a salt, solvate, hydrate, polymorph, co-crystal, tautomer, stereoisomer, isotopically labeled compound, or prodrug thereof, wherein the second reaction mixture comprises:
(a) a compound of Formula I-1, or a salt, solvate, hydrate, polymorph, co-crystal, tautomer, stereoisomer, isotopically labeled compound, or prodrug thereof; and
(b) an oxidizing agent.

26. The method of claim 25, wherein the oxidizing agent is a transition metal oxidizing agent.

27. The method of claim 26, wherein the transition metal is Mn(IV), Mn(VII), Cr(VI), Fe(III), Ru(VIII), Sb(V), Ce(IV), Os(VIII), Pt(VI), Tl(III), Pb(IV), or Bi(V).

28. The method of claim 26 or 27, wherein the transition metal oxidizing agent is MnO₂.

29. The method of claim 25, wherein the oxidizing agent is a peroxide.

30. The method of claim 29, wherein the peroxide is an organic peroxide, hydrogen peroxide, peroxy acid, main group peroxide, or metal peroxide.

31. The method of claim 29, wherein the peroxide is an organic hydroperoxide.

32. The method of any one of claims 29-31, wherein the peroxide is (unsubstituted C₁₋₆ alkyl)OOH.

33. The method of any one of claims 25-32, wherein Formula III-1 is:

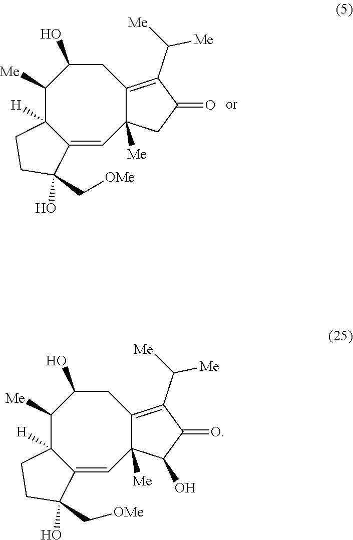 [SMILES: COC[C@@]1(O)CC[C@@H]2/C1=C\[C@@]1(C)CC(=O)C(C(C)C)=C1C[C@H](O)[C@@H]2C ; COC[C@@]1(O)CC[C@@H]2/C1=C\[C@]1(C)C(=C(C(C)C)C(=O)[C@H]1O)C[C@H](O)[C@@H]2C]

34. The method of any one of claims 1-21 further comprising incubating a third reaction mixture to produce the compound of Formula IV-2: 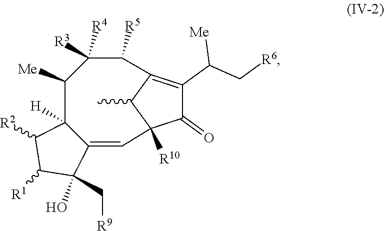
or a salt, solvate, hydrate, polymorph, co-crystal, tautomer, stereoisomer, isotopically labeled compound, or prodrug thereof, wherein the third reaction mixture comprises:
(a) a compound of Formula I-2, or a salt, solvate, hydrate, polymorph, co-crystal, tautomer, stereoisomer, isotopically labeled compound, or prodrug thereof; and
(b) a reducing agent.

35. The method of claim 34, wherein Formula IV-2 is:

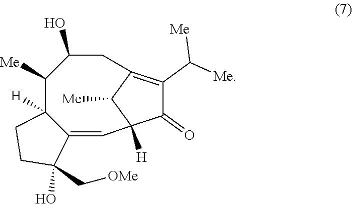 [SMILES: COC[C@@]1(O)CC[C@@H]2/C1=C\[C@H]1C(=O)C(C(C)C)=C(C[C@H](O)[C@@H]2C)[C@H]1C]

36. The method of any one of claims 34-35 further comprising incubating a fourth reaction mixture to produce the compound of Formula V-2: 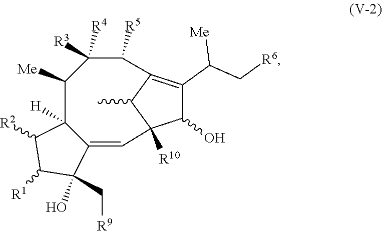
or a salt, solvate, hydrate, polymorph, co-crystal, tautomer, stereoisomer, isotopically labeled compound, or prodrug thereof, wherein the fourth reaction mixture comprises:
(a) a compound of Formula IV-2, or a salt, solvate, hydrate, polymorph, co-crystal, tautomer, stereoisomer, isotopically labeled compound, or prodrug thereof; and
(b) a reducing agent.

37. The method of claim 36, wherein Formula V-2 is:

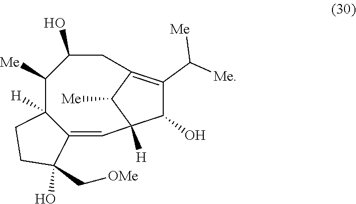 [SMILES: COC[C@@]1(O)CC[C@@H]2/C1=C\[C@@H]1[C@H](C)C(=C(C(C)C)[C@@H]1O)C[C@H](O)[C@@H]2C]

38. A method of preparing a compound of Formula II: 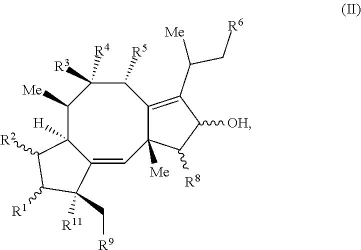
or a salt, solvate, hydrate, polymorph, co-crystal, tautomer, stereoisomer, isotopically labeled compound, or prodrug thereof, the method comprising incubating a first reaction mixture to produce the compound of Formula II, or a salt, solvate, hydrate, polymorph, co-crystal, tautomer, stereoisomer, isotopically labeled compound, or prodrug thereof, wherein the first reaction mixture comprises:
(a) a compound of Formula B: 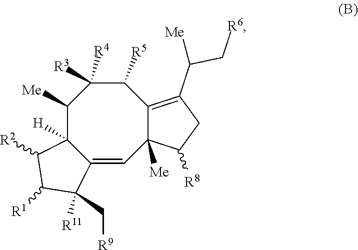
or a salt, solvate, hydrate, polymorph, co-crystal, tautomer, stereoisomer, isotopically labeled compound, or prodrug thereof; and
(b) a cytochrome P450 hydroxylase;
wherein:
each one of R¹, R², R⁵, R⁶, and R⁸ is independently hydrogen, —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, -optionally substituted (=O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, or —OS(═O)₂N(R^{a})₂;
R³ is —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, or —OS(═O)₂N(R^{a})₂, and R⁴ is hydrogen;
or: R³ and R⁴ taken together form ═O;
each one of R⁹ and R¹¹ is independently —OR^{a}, —N(R^{a})₂, —SR^{a}, —SCN, —NO₂, —N₃, —NR^{a}C(═O)R^{a}, —NR^{a}C(═O)OR^{a}, —NR^{a}C(═O)N(R^{a})₂, —NR^{a}C(═NR^{a})R^{a}, —NR^{a}C(═NR^{a})OR^{a}, —NR^{a}C(═NR^{a})N(R^{a})₂, —OC(═O)R^{a}, —OC(═O)OR^{a}, —OC(═O)N(R^{a})₂, —OC(═NR^{a})R^{a}, —OC(═NR^{a})OR^{a}, —OC(═NR^{a})N(R^{a})₂, —NR^{a}S(═O)R^{a}, —NR^{a}S(═O)OR^{a}, —NR^{a}S(═O)N(R^{a})₂, —NR^{a}S(═O)₂R^{a}, —NR^{a}S(═O)₂OR^{a}, —NR^{a}S(═O)₂N(R^{a})₂, —OS(═O)R^{a}, —OS(═O)OR^{a}, —OS(═O)N(R^{a})₂, —OS(═O)₂R^{a}, —OS(═O)₂OR^{a}, —OS(═O)₂N(R^{a})₂, or hydrogen; and
each R^{a} is independently hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, a nitrogen protecting group when attached to a nitrogen atom, an oxygen protecting group when attached to an oxygen atom, or a sulfur protecting group when attached to a sulfur atom, or two instances of R^{a} on a nitrogen atom are joined with the nitrogen atom to form substituted or unsubstituted heterocyclyl or substituted or unsubstituted heteroaryl.

39. The method of claim 38, wherein 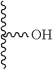
is

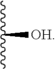

40. The method of claim 38 or 39, wherein the cytochrome P450 hydroxylase is a cytochrome P450 BM3 hydroxylase.

41. The method of any one of claims 38-40, wherein the cytochrome P450 hydroxylase comprises an amino acid sequence that is at least 80% identical to the amino acid sequence of SEQ ID NO: 32.

42. The method of any one of claims 38-41, wherein the cytochrome P450 hydroxylase comprises a mutation.

43. The method of claim 42, wherein the mutation is a mutation of L75.

44. The method of claim 42 or 43, wherein the mutation is L75A.

45. The method of any one of claims 38-44, wherein the cytochrome P450 hydroxylase comprises an amino acid sequence of SEQ ID NO: 32.

46. The method of any one of claims 38-45, wherein the method further comprises lysing a cell to provide a cell lysate, wherein the step of incubating a first reaction mixture is performed after the step of lysing a cell.

47. The method of claim 46, wherein the step of incubating a first reaction mixture is performed immediately after the step of lysing a cell.

48. The method of claim 46 or 47, wherein the cell lysate comprises the cytochrome P450 hydroxylase.

49. The method of any one of claims 46-48, wherein the cytochrome P450 hydroxylase is isolated from a cell lysate.

50. The method of any one of claims 46-49, wherein the cell is a cell of a fungus, yeast, plant, alga, or bacterium.

51. The method of any one of claims 46-50, wherein the cell is a cell of a strain of the bacteria Escherichia coli.

52. The method of any one of claims 46-51, wherein the cell is modified to express the cytochrome P450 hydroxylase.

53. The method of any one of claims 38-52, wherein the cytochrome P450 hydroxylase is a recombinant cytochrome P450 hydroxylase.

54. The method of any one of claims 1-53, wherein R¹ is hydrogen.

55. The method of any one of claims 1-53, wherein R¹ is —OR^{a}.

56. The method of any one of claims 1-53, wherein R¹ is —OH.

57. The method of any one of claims 1-56, wherein R² is hydrogen.

58. The method of any one of claims 1-56, wherein R² is —OR^{a}.

59. The method of any one of claims 1-56, wherein R² is —OH.

60. The method of any one of claims 1-59, wherein R³ and R⁴ taken together form ═O.

61. The method of any one of claims 1-59, wherein R³ is —OR^{a}; and R⁴ is hydrogen.

62. The method of any one of claims 1-59, wherein R³ is —OH; and R⁴ is hydrogen.

63. The method of any one of claims 1-62, wherein R⁵ is —OR^{a}.

64. The method of any one of claims 1-62, wherein R⁵ is —O(substituted or unsubstituted heterocyclyl).

65. The method of any one of claims 1-62, wherein R⁵ is —OH.

66. The method of any one of claims 1-62, wherein R⁵ is hydrogen.

67. The method of any one of claims 1-66, wherein R⁶ is hydrogen.

68. The method of any one of claims 1-66, wherein R⁶ is —OR^{a}.

69. The method of any one of claims 1-66, wherein R⁶ is —O(substituted or unsubstituted acyl).

70. The method of any one of claims 1-66, wherein R⁶ is —OH.

71. The method of any one of claims 1-70, wherein R⁸ is hydrogen.

72. The method of any one of claims 1-70, wherein R⁸ is —OR^{a}.

73. The method of any one of claims 1-70, wherein R⁸ is —OH.

74. The method of any one of claims 1-73, wherein R 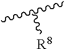
is

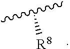

75. The method of any one of claims 1-74, wherein R⁹ is —OR^{a}.

76. The method of any one of claims 1-74, wherein R⁹ is —OCH₃.

77. The method of any one of claims 1-74, wherein R⁹ is -OBn.

78. The method of any one of claims 1-74, wherein R⁹ is —OH.

79. The method of any one of claims 1-74, wherein R⁹ is hydrogen.

80. The method of any one of claims 38-79, wherein R¹¹ is —OR^{a}.

81. The method of any one of claims 38-79, wherein R¹¹ is —OH.

82. The method of any one of claims 38-79, wherein R¹¹ is hydrogen.

83. The method of claim 1, wherein Formula I-1 is:

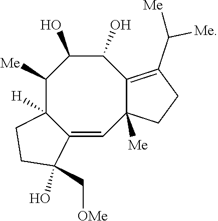 [SMILES: COC[C@@]1(O)CC[C@@H]2/C1=C\[C@@]1(C)CCC(C(C)C)=C1[C@@H](O)[C@H](O)[C@@H]2C]

84. The method of claim 1, wherein Formula I-1 is: 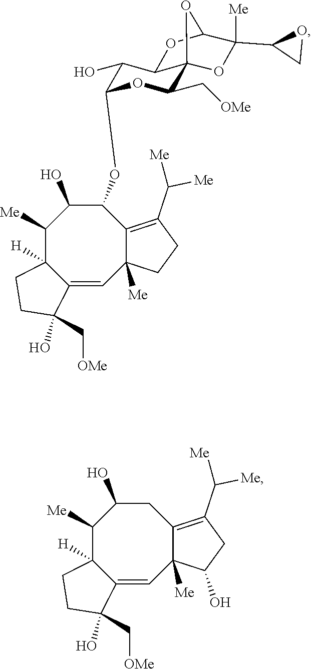
or 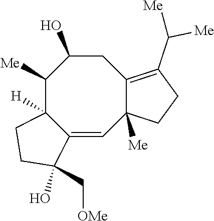 [SMILES: COC[C@@]1(O)CC[C@@H]2/C1=C\[C@@]1(C)CCC(C(C)C)=C1C[C@H](O)[C@@H]2C]
or Formula I-2 is:

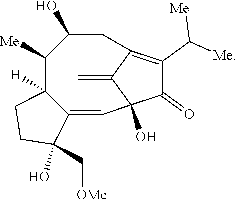 [SMILES: C=C1C2=C(C(C)C)C(=O)[C@]1(O)/C=C1\[C@@H](CC[C@]1(O)COC)[C@@H](C)[C@@H](O)C2]

85. The method of claim 1, wherein Formula I-1 is:

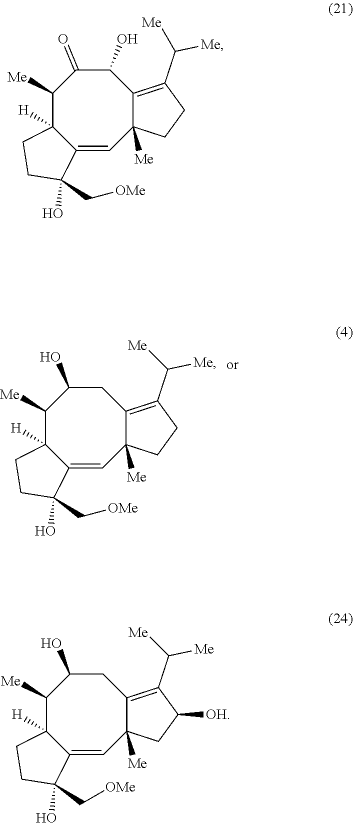 [SMILES: COC[C@@]1(O)CC[C@@H]2/C1=C\[C@@]1(C)CCC(C(C)C)=C1[C@@H](O)C(=O)[C@@H]2C ; COC[C@@]1(O)CC[C@@H]2/C1=C\[C@@]1(C)CCC(C(C)C)=C1C[C@H](O)[C@@H]2C ; COC[C@@]1(O)CC[C@@H]2/C1=C\[C@@]1(C)C[C@H](O)C(C(C)C)=C1C[C@H](O)[C@@H]2C]

86. The method of claim 1, wherein Formula I-2 is:

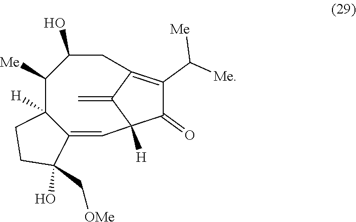 [SMILES: C=C1C2=C(C(C)C)C(=O)[C@@H]1/C=C1\[C@@H](CC[C@]1(O)COC)[C@@H](C)[C@@H](O)C2]

87. The method of claim 38, wherein Formula II is:

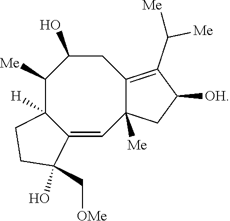 [SMILES: COC[C@@]1(O)CC[C@@H]2/C1=C\[C@@]1(C)C[C@H](O)C(C(C)C)=C1C[C@H](O)[C@@H]2C]

88. The method of any one of claims 1-37, 54-79, and 83-86, wherein the ratio of the non-heme dioxygenase to (a) of the first reaction mixture is between 10 mg:0.03 mmol and 10 mg:0.1 mmol, between 10 mg:0.1 mmol and 10 mg:0.3 mmol, between 10 mg:0.3 mmol and 10 mg:1 mmol, between 10 mg:1 mmol and 10 mg:3 mmol, between 10 mg:3 mmol and 10 mg:10 mmol, between 10 mg:10 mmol and 10 mg:30 mmol, between 10 mg:30 mmol and 10 mg:100 mmol, or between 10 mg:100 mmol and 10 mg:300 mmol, inclusive.

89. The method of any one of claims 1-88, wherein the first reaction mixture further comprises a reducing agent.

90. The method of claim 89, wherein the reducing agent is tris (2-carboxyethyl) phosphine, ascorbic acid, a borohydride, carbon, carbon monoxide, diborane, a dithionate, dithiothreitol, a ferrous salt, a ferrous compound, formic acid, hydrazine, a hydride, hydrogen, a hypophosphite, an iodide, oxalic acid, palladium(0) on carbon, a phosphite, phosphorous acid, red-Al, a reducing sugar, sodium amalgam, sodium-lead alloy, a stannous salt, a stannous compound, sulfur dioxide, a thiosulfate, or zinc amalgam, or a salt or solvate thereof.

91. The method of claim 89, wherein the reducing agent is tris (2-carboxyethyl) phosphine, or a salt or solvate thereof.

92. The method of claim 89, wherein the reducing agent is palladium(0) on carbon.

93. The method of claim 89, wherein the reducing agent is a hydride.

94. The method of any one of claims 89-93, wherein the molar ratio of the reducing agent to (a) of the first reaction mixture is between 1:1 and 10:1, between 10:1 and 100:1, between 100:1 and 1,000:1, or between 1,000:1 and 10,000:1, inclusive.

95. The method of any one of claims 1-94, wherein the first reaction mixture further comprises an aqueous buffer solution, wherein the pH of the aqueous buffer solution at about 25 °C is between 5 and 6, between 6 and 7, between 7 and 8, or between 8 and 9, inclusive.

96. The method of any one of claims 1-95, wherein the first reaction mixture is in vitro.

97. The method of any one of claims 1-96, wherein the temperature of the first reaction mixture is between 0 and 10, between 10 and 20, between 20 and 30, or between 30 and 40 °C, inclusive.

98. The method of any one of claims 1-97, wherein the pressure of the first reaction mixtures is about 0.5 and 1.1 atm, inclusive.

99. The method of any one of claims 1-98, wherein the first time duration is between 1 and 3 hours, between 3 and 6 hours, between 6 and 12 hours, between 12 and 24 hours, between 1 and 3 days, or between 3 and 7 days, inclusive.

100. The method of any one of claims 1-99, wherein the step of incubating a first reaction mixture further provides a compound of Formula U-1 or U-2: 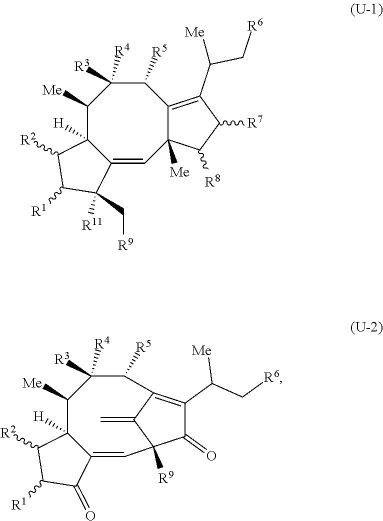
respectively, or a salt, solvate, hydrate, polymorph, co-crystal, tautomer, stereoisomer, isotopically labeled compound, or prodrug thereof.

101. The method of claim 100, wherein Formula U-1 is:

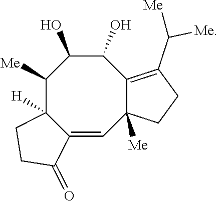 [SMILES: CC(C)C1=C2[C@@H](O)[C@H](O)[C@H](C)[C@@H]3CCC(=O)/C3=C/[C@@]2(C)CC1]

102. The method of claim 100 or 101, wherein the molar ratio of the compound of Formula I-1 or I-2, or a salt, solvate, hydrate, polymorph, co-crystal, tautomer, stereoisomer, isotopically labeled compound, or prodrug thereof, to the compound of Formula U-1 or U-2, respectively, or a salt, solvate, hydrate, polymorph, co-crystal, tautomer, stereoisomer, isotopically labeled compound, or prodrug thereof, is between 60:40 and 70:30, between 70:30 and 80:20, between 80:20 and 90:10, between 90:10 and 95:5, between 95:5 and 99:1, or between 99:1 and 99.9:0.1, inclusive.

103. The method of any one of claims 1-37, 54-79, 83-86, 88-102, wherein the rate of conversion of (a) of the first reaction mixture to the compound of Formula I-1 or I-2, or a salt, solvate, hydrate, polymorph, co-crystal, tautomer, stereoisomer, isotopically labeled compound, or prodrug thereof, is between 20% and 40%, between 40% and 50%, between 50% and 60%, between 60% and 80%, or between 80% and 99%, inclusive.

104. The method of any one of claims 1-37, 54-79, 83-86, 88-103, wherein the method further comprises purifying the compound of Formula I-1 or I-2, or a salt, solvate, hydrate, polymorph, co-crystal, tautomer, stereoisomer, isotopically labeled compound, or prodrug thereof.

105. The method of claim 104, wherein the step of purifying comprises liquid-liquid phase separation, drying, filtration, concentration, chromatography, decolorization, or recrystallization, or a combination thereof.

106. A kit comprising:
a non-heme dioxygenase comprising an amino acid sequence that is at least 80% identical to the amino acid sequence of SEQ ID NO: 30, 26, 28, 24, 22, 35, 33, or 18; and
instructions for using the non-heme dioxygenase in the method of any one of claims 1-105.

107. The kit of claim 106, wherein the non-heme dioxygenase comprises an amino acid sequence that is at least 80% identical to the amino acid sequence of SEQ ID NO: 30.

108. The kit of claim 106 or 107, wherein the non-heme dioxygenase comprises an amino acid sequence of SEQ ID NO: 30.

109. The kit of claim 106, wherein the non-heme dioxygenase comprises an amino acid sequence of SEQ ID NO: 26, 28, 24, 22, 35, 33, or 18.

110. A kit comprising:
a cytochrome P450 hydroxylase comprising an amino acid sequence that is at least 80% identical to the amino acid sequence of SEQ ID NO: 32; and
instructions for using the cytochrome P450 hydroxylase in the method of any one of claims 1-105.

111. The kit of claim 110, wherein the cytochrome P450 hydroxylase comprises an amino acid sequence of SEQ ID NO: 32.
